(19) United States
(12) Patent Application Publication
(10) Pub. No.: US 2025/0197818 A1
(43) Pub. Date: Jun. 19, 2025

(54) COMPOSITIONS AND METHODS FOR EPIGENETIC EDITING

(71) Applicant: nChroma Bio, Inc., Boston, MA (US)

(72) Inventors: Ari Friedland, Cambridge, MA (US); Mary Shirley Morrison, Allston, MA (US); Vic Myer, Arlington, MA (US)

(21) Appl. No.: 18/981,846

(22) Filed: Dec. 16, 2024

Related U.S. Application Data
(63) Continuation of application No. PCT/US2023/026140, filed on Jun. 23, 2023.
(60) Provisional application No. 63/354,931, filed on Jun. 23, 2022.

(51) Int. Cl. C12N 9/10; C12N 9/22; C12N 15/11

(52) U.S. Cl. CPC C12N 9/1007; C12N 9/22; C12N 15/11; C07K 2319/09; C07K 2319/80; C12N 2310/20; C12Y 201/01037

(57) ABSTRACT

Disclosed herein are compositions and methods comprising epigenetic editing systems for epigenetic editing or cells, nucleic acids, and vectors comprising the same. Also disclosed are epigenetically modified chromosomes.

### CROSS REFERENCE

This application is a continuation of International Application No. PCT/US2023/026140 filed Jun. 23, 2023, which claims the benefit of U.S. Provisional Application No. 63/354,931, filed Jun. 23, 2022, each of which is incorporated by reference in its entirety.

### SEQUENCE LISTING STATEMENT

This Application contains a Sequence Listing which has been submitted electronically in XML file format is hereby incorporated by reference in its entirety. Said XML copy, created on Oct. 11, 2023, is named 59073-718.602_SL.xml and is 340,194 bytes in size.

### BACKGROUND

Genome editing has been considered a promising therapeutic approach for treatment of genetic disease for over a decade. However, manipulation on the DNA level remains risky given the potential for undesired double stranded breaks, heterogenous repair including large and small insertions and deletions at the intended site, and toxicity.

### SUMMARY

Provided herein are compositions for epigenetic modification related to epigenetic editing systems and methods of using the same to generate epigenetic modification in target genomes, including those in host cells and organisms, without introducing changes to genomic sequences.

Described herein is an epigenetic editing system comprising: (a) a fusion protein comprising a DNA-binding domain, a DNA methyltransferase (DNMT) domain, a repressor domain and two nuclear localization sequences (NLSs), wherein each of the two NLSs is positioned at the amino (N) terminus or at the carboxy (C) terminus of the fusion protein; or (b) a nucleic acid molecule encoding the fusion protein of (a).

In some embodiments, the fusion protein comprises one or more NLSs at the C terminus and one or more NLSs at the N terminus of the fusion protein, optionally wherein the fusion protein comprises two NLSs at the N terminus and two NLSs at the C terminus of the fusion protein. In some embodiments, the DNMT domain is from a bacterial species. In some embodiments, the DNMT domain is a mammalian DNMT domain. In some embodiments, the DNMT domain is a mouse DNMT domain. In some embodiments, the DNMT domain is a human DNMT domain. In some embodiments, the DNMT domain is a DNMT3A domain. In some embodiments, the DNMT domain is a DNMT3L domain. In some embodiments, the fusion protein comprises both a DNMT3A domain and a DNMT3L domain.

In some embodiments, the DNMT3L domain is from a species selected from the group consisting of Ailuropoda melanoleuca, Carlito syrichta, Meriones unguiculatus, Ochotona princeps, Neosciurus carolinensis, Bison bison, Equus przewalskii, Mus caroli and Pan troglodytes; optionally comprising one of SEQ ID NOs: 72-80 and 101-109, or an amino acid sequence at least 90%, optionally at least 95%, homologous thereto.

In some embodiments, the repressor domain comprises a KRAB domain of a ZFP28, ZN627, KAP1, MeCP2, HP1b, CBX8, CDYL2, TOX, Tox3, Tox4, EED, RBBP4, RCOR1, or SCML2 protein, or a fusion of the N- and C-terminal regions of ZIM3 and KOX1 KRAB domains.

Described herein is an epigenetic editing system comprising: (a) a fusion protein comprising a DNA-binding domain, a DNA methyltransferase (DNMT) domain from a bacterial species, wherein the DNMT domain is fused to the N terminus of the DNA binding domain; or (b) a nucleic acid molecule encoding the fusion protein of (a).

In some embodiments, the fusion protein functions to methylate mammalian target DNA in a cell. In some embodiments, the DNMT domain of the fusion protein does not comprise any one of SEQ ID NOs: 81-93. In some embodiments, the bacterial species is not M. penetrans, S. monbiae, H. parainfluenzae, A. luteus, H. aegyptius, H. haemolyticus, Moraxella, E. coli, T. aquaticus, C. crescentus, or C. difficile.

In some embodiments, the DNMT domain from the bacterial species is derived from (a) M.Sss1, optionally comprising SEQ ID NO: 40, or an amino acid sequence at least 90%, optionally at least 95%, homologous thereto; NQZ29229, optionally comprising SEQ ID NO: 41, or an amino acid sequence at least 90%, optionally at least 95%, homologous thereto; WP_131599610, optionally comprising SEQ ID NO: 42, or an amino acid sequence at least 90%, optionally at least 95%, homologous thereto; or WP_208057179, optionally comprising SEQ ID NO: 43, or an amino acid sequence at least 90%, optionally at least 95%, homologous thereto.

Described herein is an epigenetic editing system comprising: (a) a fusion protein comprising a DNA-binding domain, and a DNMT3L domain, wherein the DNMT3L domain is from a species selected from the group consisting of Ailuropoda melanoleuca, Carlito syrichta, Meriones unguiculatus, Ochotona princeps, Neosciurus carolinensis, Bison bison, Equus przewalskii, Mus caroli and Pan troglodytes; optionally comprising one of SEQ ID NOs: 72-80 and 101-109, or an amino acid sequence at least 90%, optionally at least 95%, homologous thereto; or (b) a nucleic acid molecule encoding the fusion protein of (a).

In some embodiments, (i) the fusion protein further comprises a repressor domain, or (ii) the system further comprises an additional fusion protein comprising a DNA-binding domain and a repressor domain, or a nucleic acid molecule encoding the additional fusion protein. In some embodiments, the repressor domain comprises a KRAB domain, optionally derived from KOX1, ZIM3, ZFP28, or ZN627.

In some embodiments, the KRAB domain is derived from human KOX1, optionally comprising SEQ ID NO: 94, or an amino acid sequence at least 90%, optionally at least 95%, homologous thereto, or comprising SEQ ID NO: 100, or an amino acid sequence at least 90%, optionally at least 95%, homologous thereto. In some embodiments, the repressor domain is derived from KAP1, MECP2, HP1a/CBX5, HP1b, CBX8, CDYL2, TOX, TOX3, TOX4, EED, EZH2, RBBP4, RCOR1, or SCML2.

Described herein is an epigenetic editing system comprising: (a) a fusion protein comprising a DNA-binding domain, and a repressor domain, wherein the repressor domain comprises a KRAB domain of a ZFP28, ZN627, KAP1, MeCP2, HP1b, CBX8, CDYL2, TOX, Tox3, Tox4, EED, RBBP4, RCOR1, or SCML2 protein, or a fusion of the N- and C-terminal regions of ZIM3 and KOX1 KRAB domains; or (b) a nucleic acid molecule encoding the fusion protein of (a).

In some embodiments, the repressor domain comprises one of SEQ ID NOs: 44-57, or an amino acid sequence at least 90%, optionally at least 95%, homologous thereto. In some embodiments, the fusion protein further comprises a DNA methyltransferase (DNMT) domain, or the system further comprises an additional fusion protein comprising a DNA-binding domain and a DNMT domain, or a nucleic acid molecule encoding the additional fusion protein.

In some embodiments, the fusion protein or the system comprises a human DNMT3A domain and a human DNMT3L domain, or a human DNMT3A domain and a mouse DNMT3L domain, optionally (a) wherein the human DNMT3A domain comprises SEQ ID NO: 12, or an amino acid sequence at least 90%, optionally at least 95%, homologous thereto; (b) wherein the human DNMT3L domain comprises SEQ ID NO: 13, or an amino acid sequence at least 90%, optionally at least 95%, homologous thereto, and/or (c) wherein the mouse DNMT3L domain comprises SEQ ID NO: 15, or an amino acid sequence at least 90%, optionally at least 95%, homologous thereto.

In some embodiments, the epigenetic editing system further comprises a fusion protein comprising, from N terminus to C terminus, one or more NLSs, the DNMT3A domain, the DNMT3L domain, the DNA-binding domain, the repressor domain, and one or more NLSs, optionally wherein the fusion protein comprises a peptide linker between adjacent domains. In some embodiments, the fusion protein comprises, from N terminus to C terminus, two NLSs, the DNMT3A domain, ADD, the DNMT3L domain, a first peptide linker, the DNA-binding domain, a second peptide linker, the repressor domain, and two NLSs.

In some embodiments, the DNMT3A domain is a human DNMT3A domain and/or the DNMT3L domain is a human DNMT3L domain. In some embodiments, the repressor domain is a KRAB domain from mammalian, optionally human, ZFP28, ZF627, or KOX1.

In some embodiments, the first peptide linker is XTEN80 (SEQ ID NO: 3) and/or the second peptide linker is XTEN16 (SEQ ID NO: 2). In some embodiments, the system comprises an expression construct encoding the fusion protein, wherein the expression construct comprises a WPRE sequence in a 3′ noncoding region and upstream from a poly-adenylation site.

In some embodiments, the DNA-binding domain is a dCas9 domain. In some embodiments, the dCas9 domain comprises SEQ ID NO: 9, or an amino acid sequence at least 90%, optionally at least 95%, homologous thereto. In some embodiments, the epigenetic editing system further comprises one or more guide RNAs (gRNAs) or nucleic acid molecule(s) coding for the gRNAs. In some embodiments, the DNA-binding domain is a zinc finger protein (ZFP) domain.

Described herein is a method of modifying an epigenetic state of a target gene in a mammalian cell, comprising contacting the cell with the epigenetic editing system of the disclosure. Described herein is a method of modulating expression of a target gene in a mammalian cell, comprising contacting the cell with the epigenetic editing system of the disclosure. Described herein is a method of treating a disease in a subject in need thereof, comprising administering to the subject the epigenetic editing system of the disclosure.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (“FIG.” or “FIGs.” herein), of which:

FIG. 1A shows schematic illustrations of fusion protein constructs with variant NLS configurations. FIG. 1B shows schematic illustrations of additional fusion protein constructs with variant KRAB domains.

FIGS. 2A-2B show the percentage of PCSK9 protein levels measured after treatment with fusion protein constructs with various NLS placements in HeLa cells using 6.25 ng RNA (FIG. 2A) or 2.5 ng RNA (FIG. 2B). Human and murine DNMT3L sequences are indicated as h3L and m3L, respectively.

FIG. 3 shows constructs with 2×NLSs are 3× more efficient than CRISPRoff in silencing mPcsk9 in Hepa1-6 cells.

FIG. 4A shows constructs with 2×NLSs are more efficient than CRISPRoff in silencing mPcsk9 in Huh7 cells. FIGS. 4B-4C show constructs with 2×NLSs are also more efficient than CRISPR-Off in silencing mPcsk9 in Huh7 cells at different doses both at day 5 (FIG. 4B) and day 15 (FIG. 4C).

FIG. 5 shows that, in Huh7 cells, in a CRISPR-off-like format in which dCas9 is replaced with a zinc finger, 2XNLS offers improvements across multiple ZFs.

FIGS. 6A-6D show that methylation of CTLA4 promoter with a bacterial DNMT protein can induce epigenetic silencing of the locus.

FIG. 7 shows methylation profiles at the VIM3 locus of cells treated with different constructs carrying bacterial DNA methyltransferases fused to dCas9, day 30. Samples treated with M.SssI are methylated by 20%.

FIG. 8 shows methylation profiles by hybridization capture at the CLTA locus of cells comparing M. SssI to murine DNMT3A/3L in dCas9 fusions, day 29.

FIG. 9A-9D show alternative KRAB domains tested for epi-silencing activity against CRISPRoff when using 0.5 ng effector DNA using CLTA-GFP as a marker (FIG. 9A), 3 ng effector DNA using GFP as a marker (FIG. 9B), and 0.5 ng effector DNA using GFP as a marker (FIG. 9C). FIG. 9D shows results after 30 days using varying nanogram amounts of effector DNA.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F., and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J., and Kahn, A. (1996) DNA Isolation and Sequencing: Essential Techniques, John Wiley & Sons; Polak, J. M., and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M., and Dahlberg, J. E. (1992) Methods in Enzymology: DNA Structures Part A: Synthesis and Physical Analysis of DNA, Academic Press. Each of these general texts is herein incorporated by reference in its entirety.

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms, “clinic,” “clinical setting,” “laboratory” or “laboratory setting” refer to a hospital, a clinic, a pharmacy, a research institution, a pathology laboratory, or other commercial business setting where trained personnel are employed to process and/or analyze biological and/or environmental samples. These terms are contrasted with point of care, a remote location, a home, a school, and otherwise non-business, non-institutional setting.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing is relative or absolute. “Detecting the presence of” can include determining the amount of something present in addition to determining whether it is present or absent depending on the context.

The terms “subject,” “patient”, or “individual” are often used interchangeably herein. A “subject” may be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. A subject may or may not have been exposed to a pathogen of interest as described herein and may by symptomatic or symptomatic of a disease or condition associated with infection of or exposure to a pathogen as described herein. In some embodiments, a subject is suspected to have been exposed to a pathogen, e.g. a virus. In some embodiments, a subject has been exposed to an antigen or a protein representative or cross-reacts with antigens of a particular pathogen, e.g. a virus. In some embodiments, a subject has one or more symptoms that are indicative of a disease or condition associated with infection of or exposure to a pathogen as described herein. In some embodiments, the subject is currently infected by a pathogen, e.g. a virus described herein. In some embodiments, the subject is previously infected by a pathogen described herein. In some embodiments, a subject is a carrier of a virus described herein. In some embodiments, a subject is a carrier of fragments or remnants of a virus described herein. In some instances, a subject is carrier of adaptive immunity stemmed from previously or currently being infected by a virus described herein. In some embodiments, a subject is a carrier of adaptive immunity stemmed from previous or current exposure to a different virus or pathogen other than a virus or pathogen of interest.

The term “subject” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs).

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. Accordingly, the terms “polynucleotide” and “oligonucleotide” can refer to a polymer or oligomer of nucleotide or nucleoside monomers consisting of naturally occurring bases, sugars and intersugar (backbone) linkages. The terms “polynucleotide” and “oligonucleotide” can also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

The “nucleic acid” described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates).

The nucleic acid described herein may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. Backbone modifications can include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) allows prevents nuclease recognition and degradation. Backbone modifications can also include having peptide bonds instead of phosphorous in the backbone structure (e.g., N-(2-aminoethyl)-glycine units linked by peptide bonds in a peptide nucleic acid), or linking groups including carbamate, amides, and linear and cyclic hydrocarbon groups. Oligonucleotides with modified backbones are reviewed in Micklefield, Backbone modification of nucleic acids: synthesis, structure and therapeutic applications, Curr. Med. Chem., 8 (10): 1157-79, 2001 and Lyer et al., Modified oligonucleotides-synthesis, properties and applications, Curr. Opin. Mol. Ther., 1 (3): 344-358, 1999. Nucleic acid molecules described herein may contain a sugar moiety that comprises ribose or deoxyribose, as present in naturally occurring nucleotides, or a modified sugar moiety or sugar analog. The examples of modified sugar moieties include, but are not limited to, 2′-O-methyl, 2′-O-methoxyethyl, 2′-O-aminoethyl, 2′-Flouro, N3′→P5′ phosphoramidate, 2′dimethylaminooxyethoxy, 2′ 2′dimethylaminoethoxyethoxy, 2′-guanidinidium, 2′-O-guanidinium ethyl, carbamate modified sugars, and bicyclic modified sugars. 2′-O-methyl or 2′-O-methoxyethyl modifications promote the A-form or RNA-like conformation in oligonucleotides, increase binding affinity to RNA, and have enhanced nuclease resistance. Modified sugar moieties can also include having an extra bridge bond (e.g., a methylene bridge joining the 2′-O and 4′-C atoms of the ribose in a locked nucleic acid) or sugar analog such as a morpholine ring (e.g., as in a phosphorodiamidate morpholino).

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994).

The present disclosure encompasses isolated or substantially purified nucleic acid molecules and compositions containing those molecules. As used herein, an “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in some embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

As used herein, the terms “protein,” “polypeptide,” and “peptide” are used interchangeably and refer to a polymer of amino acid residues linked via peptide bonds and which may be composed of two or more polypeptide chains. The terms “polypeptide,” “protein,” and “peptide” refer to a polymer of at least two amino acid monomers joined together through amide bonds. An amino acid may be the L-optical isomer or the D-optical isomer. More specifically, the terms “polypeptide,” “protein,” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

A protein or a variant thereof can be naturally occurring or recombinant. Methods for detection and/or measurement of polypeptides in biological material are well known in the art and include, but are not limited to, Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

As used herein, the terms “fragment,” or equivalent terms can refer to a portion of a protein that has less than the full length of the protein and optionally maintains the function of the protein. Further, when the portion of the protein is blasted against the protein, the portion of the protein sequence can align, for example, at least with 80% identity to a part of the protein sequence.

Any systems, methods, and platforms described herein are modular and not limited to sequential steps. Accordingly, terms such as “first” and “second” do not necessarily imply priority, order of importance, or order of acts.

The term “modulate” refers to a change in the quantity, degree or extent of a function. For example, the compositions for epigenetic modification disclosed herein may modulate the activity of a promoter sequence by binding to a motif within the promoter, thereby inducing, enhancing or suppressing transcription of a gene operatively linked to the promoter sequence. Alternatively, modulation may include inhibition of transcription of a gene wherein the epigenetic editing system binds to the structural gene and blocks DNA dependent RNA polymerase from reading through the gene, thus inhibiting transcription of the gene. The structural gene may be a normal cellular gene or an oncogene, for example. Alternatively, modulation may include inhibition of translation of a transcript. Thus, “modulation” of gene expression includes both gene activation and gene repression.

The term “Administering” and its grammatical equivalents as used herein can refer to providing one or more replication competent recombinant adenovirus or pharmaceutical compositions described herein to a subject or a patient. By way of example and without limitation, “administering” can be performed by intravenous (i.v.) injection, sub-cutaneous (s.c.) injection, intradermal (i.d.) injection, intraperitoneal (i.p.) injection, intramuscular (i.m.) injection, intravascular injection, infusion (inf.), oral routes (p.o.), topical (top.) administration, or rectal (p.r.) administration. One or more such routes can be employed. Parenteral administration can be, for example, by bolus injection or by gradual perfusion over time.

The terms “treat,” “treating,” or “treatment,” and grammatical equivalents as used herein, can include alleviating, abating, or ameliorating at least one symptom of a disease or a condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. “Treating” may refer to administration of a vector, nucleic acid (e.g. mRNA), or LNP composition to a subject after the onset, or suspected onset, of a disease or condition. “Treating” includes the concepts of “alleviating,” which refers to lessening the frequency of occurrence or recurrence, or the severity, of any symptoms or other ill effects related to a disease or condition and/or the side effects associated with the disease or condition. The term “treating” also encompasses the concept of “managing” which refers to reducing the severity of a particular disease or disorder in a patient or delaying its recurrence, e.g., lengthening the period of remission in a patient who had suffered from the disease. The term “treating” further encompasses the concept of “prevent,” “preventing,” and “prevention.” It is appreciated that, although not precluded, treating a disorder or condition does not require that the disorder, condition, or symptoms associated therewith be completely eliminated. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition.

By “treating or preventing a condition” is meant ameliorating any of the conditions or signs or symptoms associated with the disorder before or after it has occurred. For example, as compared with an equivalent untreated control, alleviating a symptom of a disorder may involve reduction or degree of prevention at least 3%, 5%, 10%, 20%, 40%, 50%, 60%, 80%, 90%, 95%, 98%, 99%, 99.5%, 99.9%, or 100% as measured by any standard technique. In some embodiments, alleviating a symptom of a disorder may involve reduction or degree of prevention by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with an equivalent untreated control.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, nontoxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to a subject without causing undesirable biological effects or interacting in a deleterious manner with any of the components of the pharmaceutical composition in which it is contained.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

A “pharmaceutically acceptable salt” may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the term “therapeutically effective amount” means an amount of an agent to be delivered (e.g., nucleic acid, drug, payload, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

The term “repressor domain” or “transcriptional repressor domain” refers to a transcription repression protein or a portion thereof, such as a transcription factor, which can complex with one or more DNA binding domains to act as a negative regulatory domain. Repressor domains block the recruitment of RNA polymerase in order to suppress the transcription of certain genes. A repressor domain allows for the precise control of gene expression by inhibiting the activation of transcription through interaction with other cellular components such as but not limited to basal transcription factors, effector molecules, activator or coactivator proteins, repressors, and corepressors.

The term “KRAB” refers to Krüppel associated box, a transcription repression protein domain. KRAB refers to homologs, orthologs and mutants of a KRAB domain that have a conserved or enhanced basic function of inhibiting the transcription of genes. A KRAB domain is one of a group of transcriptional repression domains present in approximately 400 human zinc finger protein-based transcription factors. KRAB domains typically include about 45 to about 75 amino acid residues. A description of KRAB domains, including their function and use, may be found, for example, in Ecco, G., Imbeault, M., Trono, D., KRAB zinc finger proteins, Development 144, (15): 2719-2017.

The term “DNMT” refers to a DNA methyltransferase. As used herein, this term encompasses an enzyme that catalyzes the transfer of a methyl group to DNA such as canonical cytosine-5 DNMTs that catalyze the addition of methyl groups to genomic DNA (e.g., DNMT1, DNMT3A, DNMT3B, and DNMT3C). This term also encompasses non-canonical family members that do not catalyze methylation themselves but that recruit (including activate) catalytically active DNMTs, with non-limiting examples of such DNA methyltransferases including DNMT3L. See, e.g., Lyko, Nat Review. (2018) 19:81-92. Unless otherwise indicated, a DNMT domain may refer to a polypeptide domain derived from a catalytically active DNMT (e.g., DNMT1, DNMT3A, and DNMT3B) or from a catalytically inactive DNMT (e.g., DNMT3L).

The term “DNA binding domain” refers to DNA-binding domains from proteins selected from the family of CRISPR proteins, TAL proteins, zinc fingers and other transcriptional regulators, their homologs, orthologs, and mutants, which maintain or enhance the basic function of DNA-binding proteins.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

The term “ameliorate” as used herein can refer to decrease, suppress, attenuate, diminish, arrest, or stabilize the development or progression of a disease.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein, “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir.

The term “parenteral” as used herein includes subcutaneous, intracutaneous, intravenous, intramuscular, intraarticular, intraarterial, intrasynovial, intrastemal, intrathecal, intralesional, and intracranial injection or infusion techniques. In addition, it can be administered to the subject via injectable depot routes of administration such as using 1-, 3-, or 6-month depot injectable or biodegradable materials and methods.

It will be understood that in addition to the specific proteins and nucleotides mentioned herein, the present invention also contemplates the use of variants, derivatives, homologues and fragments thereof. As used herein, a variant of any given sequence is a sequence in which the specific sequence of residues (whether amino acid or nucleic acid residues) has been modified in such a manner that the polypeptide or polynucleotide in question substantially retains at least one of its endogenous functions. A variant sequence can be obtained by addition, deletion, substitution, modification, replacement and/or variation of at least one residue present in the naturally occurring protein. As used herein, a derivative of any given sequence as contemplated includes any substitution of, variation of, modification of, replacement of, deletion of and/or addition of one (or more) amino acid residues from or to the sequence providing that the resultant protein or polypeptide substantially retains at least one of its endogenous functions. Amino acid substitutions may be made, for example from 1, 2 or 3 to 10 or 20 substitutions provided that the modified sequence substantially retains the required activity or ability. Amino acid substitutions may include the use of non-naturally occurring analogues. Proteins used in the present disclosure may also have deletions, insertions or substitutions of amino acid residues which do not affection function of the protein and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

As used herein, a homologue of any herein contemplated protein or nucleic acid sequence includes sequences having a certain homology with the wild type amino acid and nucleic sequence. A homologous sequence may include a sequence, e.g. an amino acid sequence which may be at least 50%, 55%, 65%, 75%, 85% or 90% identical to the subject sequence. In particular embodiments, a homologous sequence may include an amino acid sequence at least 95% or 97% or 99% identical to the subject sequence.

Sequence identity may be measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

### Epigenetic Editing Systems

Described herein are epigenetic editing systems for epigenetic modification and expression regulation of target genes. The epigenetic editing systems used for gene repression can include fusion proteins or nucleic acids encoding the fusion proteins. In some embodiments, the fusion protein comprises a DNA-binding domain. In some embodiments, the fusion protein comprises a DNA methyltransferase (DNMT) domain. In some embodiments, the fusion protein comprises a nuclear localization sequence (NLS). In some embodiments, the fusion protein comprises a repressor domain.

As used herein, an epigenetic editing system can be any agent that binds a target polynucleotide and has epigenetic modulation activity. In some embodiments, the epigenetic editing system binds the polynucleotide at a specific sequence using a DNA binding domain. In some embodiments, the epigenetic editing system binds the polynucleotide at a specific sequence using a nucleic acid guided DNA binding protein. In some embodiments, the epigenetic editing system comprises an effector domain capable of modulating epigenetic state of a nucleic acid sequence at or adjacent to the target polynucleotide. In some embodiments, the epigenetic editing system is capable of depositing an epigenetic editing mark on a chromatin region, a nucleic acid sequence, or a histone amino acid residue, at or adjacent to the target polynucleotide. For example, the epigenetic editing system can be capable of methylating, demethylating, acetylating, deacetylating, ubiquitinating or deubiquitinating a chromatin region, a nucleic acid sequence, or a histone amino acid residue, at or adjacent to the target polynucleotide. In some embodiments, the epigenetic editing system is capable of recruiting one or more proteins or complexes involved in transcription regulation, for example, a transcription factor, a transcription activator, a transcription repressor, or an insulator to a chromatin region, a nucleic acid sequence, or a histone amino acid residue, at or adjacent to the target polynucleotide.

Epigenetic editing systems provided herein can comprise one or more effector domains as described. In some embodiments, an epigenetic editing system comprises multiple effector domains. In some embodiments, an epigenetic editing system comprises one effector domain. In some embodiments, the epigenetic editing system comprises at least 2, 3, 4, 5, 6, 7, 8, 9, 10 or more effector domains.

In some embodiments, the epigenetic editing system comprises a DNA methylation domain, a repression domain, and a nucleic acid binding domain. In some embodiments, the epigenetic editing system comprises a DNA methylation domain and a nucleic acid binding domain. In some embodiments, the nucleic acid binding domain is at the C-terminus of the fusion protein. In some embodiments, the nucleic acid binding domain is at the N-terminus of the fusion protein. In some embodiments, the nucleic acid binding domain is in the middle of the fusion protein. In some embodiments, the DNA methylation domain is at the C-terminus of the fusion protein. In some embodiments, the DNA methylation domain is at the N-terminus of the fusion protein. In some embodiments, the DNA methylation domain is in the middle of the fusion protein. In some embodiments, the repression domain is at the C-terminus of the fusion protein. In some embodiments, the repression domain is at the N-terminus of the fusion protein. In some embodiments, the repression domain is in the middle of the fusion protein. In some embodiments, an epigenetic editing system comprises a DNA demethylation domain and a histone acetylation domain. In some embodiments, an epigenetic editing system comprises a DNA demethylation domain and an activation domain that recruits additional DNA demethylation or histone acetylation proteins. In some embodiments, an epigenetic editing system comprises a DNA demethylation domain, a histone acetylation domain, and a scaffold protein that recruits additional DNA demethylation or histone acetylation proteins. In some embodiments, an epigenetic editing system comprises two or more DNA demethylation domains, two or more histone acetylation domains, and/or two or more scaffold proteins that recruits additional DNA demethylation or histone deacetylation proteins.

Multiple fusion proteins or constructs may be used to effect activation or repression of a target gene or multiple target genes. For example, an epigenetic editing system comprises a fusion protein comprising a DNA binding domain (e.g. dCas9 domain) and a methylation domain and another fusion protein comprising a DNA binding domain and a repressor domain may be co-delivered with two or more guide RNAs, each targeting a different target DNA sequence. The two or more target DNA sequences may be in the same target gene or may be in different target genes.

### Effector Domains

Epigenetic editing systems provided herein may include one or more effector protein domains that modulate expression of a target gene. An effector domain can be used to contact a target polynucleotide sequence in a target gene to effect an epigenetic modification, for example, a change in methylation state of DNA nucleotides in the target gene. Accordingly, an epigenetic editing system with one or more effector domains may provide the effect of modulating expression of a target gene without altering the DNA sequence of the target gene. For example, in some embodiments, an effector domain results in repression or silencing of expression of a target gene. In some embodiments, an effector domain results in activation or increased expression of a target gene.

An epigenetic effector may deposit a chemical modification at the chromatin at the position of a target gene. Non limiting examples of chemical modifications include methylation, demethylation, acetylation, deacetylation, phosphorylation, SUMOylation and/or ubiquitination of the DNA or histone residues of the chromatin. In some embodiments, an epigenetic effector may make histone tail modifications. In some embodiments epigenetic effectors may add or remove active marks on histone tails. In some embodiments the active marks may include H3K4 methylation, H3K9 acetylation, H3K27 acetylation, H3K36 methylation, H3K79 methylation, H4K5 acetylation, H4K8 acetylation, H4K12 acetylation, H4K16 acetylation, and/or H4K20 methylation. In some embodiments epigenetic effectors may add or remove repressive marks on histone tails. In some embodiments these repressive marks may include H3K9 methylation and/or H3K27 methylation.

In some embodiments, an effector domain in an epigenetic editing system alters a chemical modification state of a target gene harboring a target sequence. For example, an effector domain may alter a chemical modification state of a nucleotide in the target gene. In some embodiments, an effector domain of an epigenetic editing system deposits a chemical modification at a nucleotide in the target gene. In some embodiments, an effector domain of an epigenetic editing system deposits a chemical modification of a histone associated with the target gene. In some embodiments, an effector domain of an epigenetic editing system removes a chemical modification at a nucleotide in the target gene. In some embodiments, an effector domain of an epigenetic editing system removes a chemical modification of a histone associated with the target gene. In some embodiments, the chemical modification increases expression of the target gene. For example, the epigenetic editing system may comprise an effector domain having histone acetyltransferase activity. In some embodiments, the chemical modification decreases expression of the target gene. For example, the epigenetic editing system may comprise an effector domain having DNA methyltransferase activity.

The epigenetic modification mediated by an epigenetic editing system may be in the vicinity of the target gene, or may be distant to the target gene, or spread from an initial epigenetic modification initiated by the epigenetic editing system at one or more nucleotides in a target sequence of the target gene.

In some embodiments, the alteration of the chemical modification state is a DNA methylation state. For example, methylation can be introduced by an effector domain having DNA methyltransferase activity or can be removed by an effector domain having DNA-demethylase activity. In some embodiments, the alteration of chemical modification, e.g., methylation, is at a hypomethylated nucleic acid sequence. For example, the chemically modified sequence in the target gene or chromosome region may lack methyl groups on the 5-methyl cytosine nucleotide (e.g., in CpG) as compared to a standard control. Hypomethylation may occur, for example, in aging cells or in cancer (e.g., early stages of neoplasia) relative to the younger cell or non-cancer cell, respectively. In some embodiments, the target polynucleotide sequence is within a CpG island. In some embodiments, the target gene is known to be associated with a disease or condition. In some embodiments, the target gene comprises a specific copy of disease related sequence. In some embodiments, the target gene harbors the target sequence which is related to a disease. In some embodiments, the alteration of chemical modification, e.g., methylation, is at a hypermethylated nucleic acid sequence. In some embodiments, the chemical modification is within a CpG island.

In some embodiments, the protein fusion construct can have 1 effector domain, 2 effector domains, 3 effector domains, 4 effector domains, 5 effector domains, 6 effector domains, 7 effector domains, 8 effector domains, 9 effector domains, or 10 effector domains.

### Methyltransferase Domains

In some embodiments, the effector domain comprises a histone methyltransferase domain. For example, repression (or silencing) may result from repressive chromatin markers, methylation of DNA, methylation of histone residues (e.g., H3K9, H3K27), or deacetylation of histone residues on chromatin containing a target nucleic acid sequence. Without intending to be bound by any theory, the method can be used to change epigenetic state by, for example, closing chromatin via methylation or introducing repressive chromatin markers on chromatin containing the target nuclei acid sequence (e.g., gene).

Specific epigenetic imprints direct gene transcription or gene silencing. For example, DNA methylation, histone modification, repressor proteins binding to silencer regions, and other transcriptional activities alter gene expression without changing the underlying DNA sequence. Thus, the transcriptional regulation allows for expression of specific genes in a particular manner, while repressing other genes. In certain instances, cell fate or function can be controlled, either for initial differentiation (e.g., during the organism's development) or to reprogram a cell or cell type (e.g., during disease such as cancer, chronic inflammation, auto-immune disease, illnesses related to various microbiomes of an organism, etc.). Histone modifications play a structural and biochemical role in gene transcription, in one avenue by formation or disruption of the nucleosome structure that binds to the histone and prevents gene transcription. Histones are basic proteins that are commonly found in the nucleus of eukaryotic cells, ranging from multicellular organisms including humans to unicellular organisms represented by fungi (mold and yeast) and ionically bind to genomic DNA. Histones usually consist of five components (H1, H2A, H2B, H3 and H4) and are highly similar across biological species. In the case of histone H4, for example, budding yeast histone H4 (full-length 102 amino acid sequence) and human histone H4 (full-length 102 amino acid sequence) are identical in 92% of the amino acid sequences and differ only in 8 residues. Among the natural proteins assumed to be present in several tens of thousands of organisms, histones are known to be proteins most highly preserved among eukaryotic species. Genomic DNA is folded with histones by ordered binding, and a complex of both forms a basic structural unit called a nucleosome. In addition, aggregation of the nucleosomes forms a chromosomal chromatin structure. Histones are subject to modifications, such as acetylation, methylation, phosphorylation, ubiquitination, SUMOylation and the like, at their N-terminal ends called histone tails, and maintain or specifically convert the chromatin structure, thereby controlling responses such as gene expression, DNA replication, DNA repair and the like, which occur on chromosomal DNA. Post-translational modification of histones is an epigenetic regulatory mechanism and is considered essential for the genetic regulation of eukaryotic cells. Recent studies have revealed that chromatin remodeling factors such as SWI/SNF, RSC, NURF, NRD and the like, which encourage DNA access to transcription factors by modifying the nucleosome structure, histone acetyltransferases (HATs) that regulate the acetylation state of histones, and histone deacetylases (HDACs), act as important regulators. DNA methylation occurs primarily at CpG sites (shorthand for “C-phosphate-G-” or “cytosine-phosphate-guanine”). Highly methylated areas of DNA tend to be less transcriptionally active than lesser methylated sites. Many mammalian genes have promoter regions near or including CpG islands (regions with a high frequency of CpG sites).

In particular, the unstructured N-termini of histones may be modified by at least one of acetylation, methylation, ubiquitylation, phosphorylation, sumoylation, ribosylation, citrullination O-GlcNAcylation, or crotonylation. For example, acetylation of K14 and K9 lysines of histone H3 by histone acetyltransferase enzymes may be linked to transcriptional competence in humans. Lysine acetylation may directly or indirectly create binding sites for chromatin-modifying enzymes that regulate transcriptional activation. For example, histone acetyltransferases (HATs) utilize acetyl-CoA as a cofactor and catalyze the transfer of an acetyl group to the epsilon amino group of the lysine side chains. This neutralizes the lysine's positive charge and weakens the interactions between histones and DNA, thus opening the chromosomes for transcription factors to bind and initiate transcription. Likewise, histone methylation of lysine 9 of histone H3 may be associated with heterochromatin, or transcriptionally silent chromatin. Particular DNA methylation patterns may be established and modified by at least one or more, two or more, three or more, four or more, or five or more independent DNA methyltransferases, including DNMT1, DNMT3A, and DNMT3B.

In some embodiments, the effector domain comprises a histone methyltransferase domain. In some embodiments, the effector domain comprises a DOT1L domain, a SET domain, a SUV39H1 domain, a G9a/EHMT2 protein domain, a EZH1 domain, a EZH2 domain, a SETDB1 domain, or any combination thereof. In some embodiments, the effector domain comprises a histone-lysine-N-methyltransferase SETDB1 domain.

In some embodiments, the effector domain comprises a DNA methyltransferase domain or a histone methyltransferase domain. DNA methyltransferase domains may mediate methylation at DNA nucleotides, for example at any of an A, T, G or C nucleotide. In some embodiments, the methylated nucleotide is a N6-methyladenosine (m6A). In some embodiments, the methylated nucleotide is a 5-methylcytosine (5mC). In some embodiments, the methylation is at a CG (or CpG) dinucleotide sequence. In some embodiments, the methylation is at a CHG or CHH sequence, where H is any one of A, T, or C.

In some embodiments, the effector domain comprises a DNA methyltransferase DNMT domain that catalyzes transfer of a methyl group to cytosine, thereby repressing expression of the target gene through the recruitment of repressive regulatory proteins. In some embodiments, the effector domain comprises a DNA methyltransferase (DNMT) family protein domain. In some embodiments, the effector domain comprises a DNMT1 domain. In some embodiments, the effector domain comprises a TRDMT1 domain. In some embodiments, the effector domain comprises a DNMT3 domain. In some embodiments, the effector domain comprises a DNMT3A domain. In some embodiments, the effector domain comprises a DNMT3B domain. In some embodiments, the effector domain comprises a DNMT3C domain. In some embodiments, the effector domain comprises a DNMT3L domain. In some embodiments, the effector domain comprises a fusion of DNMT3A-DNMT3L domain.

Exemplary DNA methyltransferases (DNMTs) that may be part of an epigenetic effector domain are provided in Table 1 below. In some embodiments, the epigenetic editing system herein contains one or more epigenetic effector domains selected from Table 1, or functional homologs, orthologs, or variants thereof.

TABLE 1
Exemplary DNA methyltransferase sequences that
may be used in epigenetic effector domains

| Protein Name | Species | Protein Sequence | Catalytic domain |
|---|---|---|---|
| DNMT3A (h3A) | Human | SEQ ID NO: 12 | |
| DNMT3L (h3L) | Human | SEQ ID NO: 13 | |
| Truncated DNMT3L | Human | SEQ ID NO: 14 | |
| (h3Ls) | | | |
| DNMT3L (m3L) | Mouse | SEQ ID NO: 15 | |
| Truncated DNMT3L | Mouse | SEQ ID NO: 16 | |
| (m3Ls) | | | |
| DNMT3L | Ailuropoda melanoleuca | SEQ ID NO: 72 | SEQ ID NO: 101 |
| DNMT3L | Carlito syrichta | SEQ ID NO: 73 | SEQ ID NO: 102 |
| DNMT3L | Meriones unguiculatus | SEQ ID NO: 74 | SEQ ID NO: 103 |
| DNMT3L | Ochotona princeps | SEQ ID NO: 75 | SEQ ID NO: 104 |
| DNMT3L | Neosciurus carolinensis | SEQ ID NO: 76 | SEQ ID NO: 105 |
| DNMT3L | Bison bison | SEQ ID NO: 77 | SEQ ID NO: 106 |
| DNMT3L | Equus przewalskii | SEQ ID NO: 78 | SEQ ID NO: 107 |
| DNMT3L | Mus caroli | SEQ ID NO: 79 | SEQ ID NO: 108 |
| DNMT3L | Pan troglodytes | SEQ ID NO: 80 | SEQ ID NO: 109 |
| DNA cytosine | Mycoplasmatales | SEQ ID NO: 41 | |
| methyltransferase | bacterium | | |
| DNA cytosine | Mycoplasma marinum | SEQ ID NO: 42 | |
| methyltransferase | | | |
| DNA cytosine | Spiroplasma chinense | SEQ ID NO: 43 | |
| methyltransferase | | | |

In some embodiments, a methyltransferase can be a mammalian methyltransferase. In some embodiments, a methyltransferase can be a plant methyltransferase. In some embodiments, a methyltransferase can be a fungal methyltransferase. In some embodiments, a methyltransferase can be a bacterial methyltransferase.

A bacterial DNA methyltransferase can be obtained from a bacterial species. A bacterial species can be a coccus bacterium. A bacterial species can be a bacillus bacterium. A bacterial species can be a spiral bacterium. A bacterium can be an intracellular, a gram-positive, or a gram-negative bacterium. Examples of bacterial genus from which a suitable DNA methyltransferase can be obtained include, but are not limited to: Acetobacter, Acinetobacter, Actinomyces, Agrobacterium, Anaplasma, Azorhizobium, Azotobacter, Bacillus, Viridans, Bacteroides, Bartonella, Bordetella, Borrelia, Brucella, Brukholderia, Calymmatobacterium, Campylobacter, Chlamydia, Chlamydophila, Clostridium, Corynebacterium, Coxiella, Ehrlichia, Eikenella, Enterobacter, Enterococcus, Escherichia, Fusobacterium, Gardnerella, Haemophilus, Helicobacter, Klebsiella, Lactobacillus, Legionella, Leptospira, Listeria, Methanobacterium, Microbacterium, Micrococcus, Moraxella, Mycobacterium, Mycoplasma, Mycoplasmatales, Neisseria, Pasteurella, Peptostreptococcus, Porphyromonas, Prevotella, Pseudomonas, Rhizobium, Rickettsia, Rochalimaea, Rothia, Salmonella, Shigella, Spirillum, Spiroplasma, Staphylococcus, Stenotrophomonas, Streptococcus, Treponema, Ureaplasma, Vibrio, Wolbachia, and Yersinia. In certain embodiments, a DNMT or a DNMT domain is used as an epigenetic effector sequence in a fusion protein provided herein that is derived from a bacterial species, wherein the bacterial species is Mycoplasmatales bacterium, Mycoplasma marinum, or Spiroplasma chinense. In certain embodiments, the bacterial species from which the DNMT is derived is not M. penetrans, S. monbiae, H. parainfluenzae, A. luteus, H. aegyptius, H. haemolyticus, Moraxella, E. coli, T. aquaticus, C. crescentus, or C. difficile.

In some embodiments a DNMT can be an animal DNMT. In some embodiments, a DNMT can be a mammalian DNMT. In some embodiments, a DNMT can be a primate DNMT. In some embodiments, a DNMT can be a human DNMT. In some embodiments, a DNMT can be a Pan troglodytes DNMT. In some embodiments, a DNMT can be a Carlito syrichta DNMT. In some embodiments, a DNMT can be a rodent DNMT. In some embodiments, a DNMT can be a mouse DNMT. In some embodiments, a DNMT can be a Mus caroli DNMT. In some embodiments, a DNMT can be a Mus musculus DNMT. In some embodiments, a DNMT can be a Neosciurus carolinensis DNMT. In some embodiments, a DNMT can be a Meriones unguiculatus DNMT. In some embodiments, a DNMT can be a horse DNMT. In some embodiments, a DNMT can be a Equus przewalskii DNMT. In some embodiments, a DNMT can be a bovine DNMT. In some embodiments, a DNMT can be a Bison DNMT. In some embodiments, a DNMT can be an Ochotona princeps DNMT. In some embodiments, a DNMT can be a feline DNMT. In some embodiments, a DNMT can be a canine DNMT. In some embodiments, a DNMT can be an ursine DNMT. In some embodiments, a DNMT can be a Ailuropoda melanoleuca DNMT.

In some embodiments, a DNMT can comprise SEQ ID NO: 12. In some embodiments, a DNMT can be SEQ ID NO: 12. In some embodiments, a DNMT can be at least 99%, 98%, 97%, 96%, 95%, 90%, 85%, or 80% similar to SEQ ID NO: 12.

In some embodiments, a DNMT can comprise SEQ ID NO: 13. In some embodiments, a DNMT can be SEQ ID NO: 13. In some embodiments, a DNMT can be at least 99%, 98%, 97%, 96%, 95%, 90%, 85%, or 80% similar to SEQ ID NO: 13.

In some embodiments, a DNMT can comprise SEQ ID NO: 14. In some embodiments, a DNMT can be SEQ ID NO: 14. In some embodiments, a DNMT can be at least 99%, 98%, 97%, 96%, 95%, 90%, 85%, or 80% similar to SEQ ID NO: 14.

In some embodiments, a DNMT can comprise SEQ ID NO: 15. In some embodiments, a DNMT can be SEQ ID NO: 15. In some embodiments, a DNMT can be at least 99%, 98%, 97%, 96%, 95%, 90%, 85%, or 80% similar to SEQ ID NO: 15.

In some embodiments, a DNMT can comprise SEQ ID NO: 16. In some embodiments, a DNMT can be SEQ ID NO: 16. In some embodiments, a DNMT can be at least 99%, 98%, 97%, 96%, 95%, 90%, 85%, or 80% similar to SEQ ID NO: 16.

In some embodiments, a DNMT can comprise SEQ ID NO: 72. In some embodiments, a DNMT can be SEQ ID NO: 72. In some embodiments, a DNMT can be at least 99%, 98%, 97%, 96%, 95%, 90%, 85%, or 80% similar to SEQ ID NO: 72.

In some embodiments, a DNMT can comprise SEQ ID NO: 73. In some embodiments, a DNMT can be SEQ ID NO: 73. In some embodiments, a DNMT can be at least 99%, 98%, 97%, 96%, 95%, 90%, 85%, or 80% similar to SEQ ID NO: 73.

In some embodiments, a DNMT can comprise SEQ ID NO: 74. In some embodiments, a DNMT can be SEQ ID NO: 74. In some embodiments, a DNMT can be at least 99%, 98%, 97%, 96%, 95%, 90%, 85%, or 80% similar to SEQ ID NO: 74.

In some embodiments, a DNMT can comprise SEQ ID NO: 75. In some embodiments, a DNMT can be SEQ ID NO: 75. In some embodiments, a DNMT can be at least 99%, 98%, 97%, 96%, 95%, 90%, 85%, or 80% similar to SEQ ID NO: 75.

In some embodiments, a DNMT can comprise SEQ ID NO: 76. In some embodiments, a DNMT can be SEQ ID NO: 76. In some embodiments, a DNMT can be at least 99%, 98%, 97%, 96%, 95%, 90%, 85%, or 80% similar to SEQ ID NO: 76.

In some embodiments, a DNMT can comprise SEQ ID NO: 77. In some embodiments, a DNMT can be SEQ ID NO: 77. In some embodiments, a DNMT can be at least 99%, 98%, 97%, 96%, 95%, 90%, 85%, or 80% similar to SEQ ID NO: 77.

In some embodiments, a DNMT can comprise SEQ ID NO: 78. In some embodiments, a DNMT can be SEQ ID NO: 78. In some embodiments, a DNMT can be at least 99%, 98%, 97%, 96%, 95%, 90%, 85%, or 80% similar to SEQ ID NO: 78.

In some embodiments, a DNMT can comprise SEQ ID NO: 79. In some embodiments, a DNMT can be SEQ ID NO: 79. In some embodiments, a DNMT can be at least 99%, 98%, 97%, 96%, 95%, 90%, 85%, or 80% similar to SEQ ID NO: 79.

In some embodiments, a DNMT can comprise SEQ ID NO: 80. In some embodiments, a DNMT can be SEQ ID NO: 80. In some embodiments, a DNMT can be at least 99%, 98%, 97%, 96%, 95%, 90%, 85%, or 80% similar to SEQ ID NO: 80.

In some embodiments, a DNMT can comprise SEQ ID NO: 101. In some embodiments, a DNMT can be SEQ ID NO: 101. In some embodiments, a DNMT can be at least 99%, 98%, 97%, 96%, 95%, 90%, 85%, or 80% similar to SEQ ID NO: 101.

In some embodiments, a DNMT can comprise SEQ ID NO: 102. In some embodiments, a DNMT can be SEQ ID NO: 102. In some embodiments, a DNMT can be at least 99%, 98%, 97%, 96%, 95%, 90%, 85%, or 80% similar to SEQ ID NO: 102.

In some embodiments, a DNMT can comprise SEQ ID NO: 103. In some embodiments, a DNMT can be SEQ ID NO: 103. In some embodiments, a DNMT can be at least 99%, 98%, 97%, 96%, 95%, 90%, 85%, or 80% similar to SEQ ID NO: 103.

In some embodiments, a DNMT can comprise SEQ ID NO: 104. In some embodiments, a DNMT can be SEQ ID NO: 104. In some embodiments, a DNMT can be at least 99%, 98%, 97%, 96%, 95%, 90%, 85%, or 80% similar to SEQ ID NO: 104.

In some embodiments, a DNMT can comprise SEQ ID NO: 105. In some embodiments, a DNMT can be SEQ ID NO: 105. In some embodiments, a DNMT can be at least 99%, 98%, 97%, 96%, 95%, 90%, 85%, or 80% similar to SEQ ID NO: 105.

In some embodiments, a DNMT can comprise SEQ ID NO: 106. In some embodiments, a DNMT can be SEQ ID NO: 106. In some embodiments, a DNMT can be at least 99%, 98%, 97%, 96%, 95%, 90%, 85%, or 80% similar to SEQ ID NO: 106.

In some embodiments, a DNMT can comprise SEQ ID NO: 107. In some embodiments, a DNMT can be SEQ ID NO: 107. In some embodiments, a DNMT can be at least 99%, 98%, 97%, 96%, 95%, 90%, 85%, or 80% similar to SEQ ID NO: 107.

In some embodiments, a DNMT can comprise SEQ ID NO: 108. In some embodiments, a DNMT can be SEQ ID NO: 108. In some embodiments, a DNMT can be at least 99%, 98%, 97%, 96%, 95%, 90%, 85%, or 80% similar to SEQ ID NO: 108.

In some embodiments, a DNMT can comprise SEQ ID NO: 109. In some embodiments, a DNMT can be SEQ ID NO: 109. In some embodiments, a DNMT can be at least 99%, 98%, 97%, 96%, 95%, 90%, 85%, or 80% similar to SEQ ID NO: 109.

In some embodiments, a DNMT (e.g., a DNMT3) can comprise an ADD domain. An ADD domain can have the sequence of SEQ ID NO: 98. An ADD domain can inhibit DNMT activity in specific genomic regions (e.g., regions having a specific histone signature).

### Repressor Domains

In some embodiments, the effector domain recruits one or more protein domains that repress expression of the target gene. In some embodiments, the effector domain interacts with a scaffold protein domain that recruits one or more protein domains that repress expression of the target gene. For example, the effector domain may recruit or interact with a scaffold protein domain that recruits a PRMT protein, a HDAC protein, a SETDB1 protein, or a NuRD protein domain. In some embodiments, the effector domain comprises a Krüppel associated box (KRAB) repression domain; a Repressor Element Silencing Transcription Factor (REST) repression domain, KRAB-associated protein 1 (KAP1) domain, a MAD domain, a FKHR (forkhead in rhabdosarcoma gene) repressor domain, aEGR-1 (early growth response gene product-1) repressor domain, a ets2 repressor factor repressor domain (ERD), a MAD smSIN3 interaction domain (SID), a WRPW motif of the hairy-related basic helix-loop-helix (bHLH) repressor proteins; an HP1 alpha chromo-shadow repression domain, or any combination thereof (e.g., a KRAB domain derived from KOX1 (aka ZNF10), ZIM3 (aka ZNF657 or ZNF264), or ZN627). In some embodiments, the effector domain comprises a KRAB domain. In some embodiments, the effector domain comprises a Tripartite motif containing 28 (TRIM28, TIF1-beta, or KAP1) protein.

In some embodiments, an effector domain comprises a protein domain that represses expression of the target gene, also referred to herein as a “functional repression domain,” as a “repression domain,” or as a “repressor domain.” For example, the effector domain may comprise a functional repression domain derived from a zinc finger repressor protein.

In some embodiments, the effector domain comprises a KOX1/ZNF10 repression domain, a KOX8/ZNF708 repression domain, a ZNF43 repression domain, a ZNF184 repression domain, a ZNF91 repression domain, a HPF4 repression domain, a HTF10 repression domain, a HTF34 or any combination thereof. In some embodiments repression domain, a the effector domain comprises a ZIM3 repression domain, a ZNF436 repression domain, a ZNF257 repression domain, a ZNF675 repression domain, a ZNF490 repression domain, a ZNF320 repression domain, a ZNF331 repression domain, a ZNF816 repression domain, a ZNF680 repression domain, a ZNF41 repression domain, a ZNF189 repression domain, a ZNF528 repression domain, a ZNF543 repression domain, a ZNF554 repression domain, a ZNF140 repression domain, a ZNF610 repression domain, a ZNF264 repression domain, a ZNF350 repression domain, a ZNF8 repression domain, a ZNF582 repression domain, a ZNF30 repression domain, a ZNF324 repression domain, a ZNF98 repression domain, a ZNF669 repression domain, a ZNF677 repression domain, a ZNF596 repression domain, a ZNF214 repression domain, a ZNF37A repression domain, a ZNF34 repression domain, a ZNF250 repression domain, a ZNF547 repression domain, a ZNF273 repression domain, a ZNF354A repression domain, a ZFP82 repression domain, a ZNF224 repression domain, a ZNF33A repression domain, a ZNF45 repression domain, a ZNF175 repression domain, a ZNF595 repression domain, a ZNF184 repression domain, a ZNF419 repression domain, a ZFP28-1 repression domain, a ZFP28-2 repression domain, a ZNF18 repression domain, a ZNF213 repression domain, a ZNF394 repression domain, a ZFP1 repression domain, a ZFP14 repression domain, a ZNF416 repression domain, a ZNF557 repression domain, a ZNF566 repression domain, a ZNF729 repression domain, a ZIM2 repression domain, a ZNF254 repression domain, a ZNF764 repression domain, a ZNF785 repression domain or any combination thereof. In some embodiments, the effector domain comprises a ZIM3 repression domain, a ZNF554 repression domain, a ZNF264 repression domain, a ZNF324 repression domain, a ZNF354A repression domain, a ZNF189 repression domain, a ZNF543 repression domain, a ZFP82, ZNF669, ZNF582 repression domain or any combination thereof. In some embodiments, the effector domain comprises a ZIM3 repression domain, a ZNF554 repression domain, a ZNF264 repression domain, a ZNF324 repression domain, or a ZNF354A repression domain or any combination thereof. In some embodiments, the effector domain is a ZIM3 repression domain.

In some embodiments, the repression domain is a KRAB domain. In some embodiments, the effector domain comprises a functional repression domain that comprises, or is derived from, a KOX1/ZNF10 KRAB domain, a KOX8/ZNF708 KRAB domain, a ZNF43 KRAB domain, a ZNF184 KRAB domain, a ZNF91 KRAB domain, a HPF4 KRAB domain, a HTF10 KRAB domain or a HTF34 KRAB domain or any combination thereof. In some embodiments, the effector domain comprises a functional repression domain derived from a ZIM3 KRAB domain, a ZNF436 KRAB domain, a ZNF257 KRAB domain, a ZNF675 KRAB domain, a ZNF490 KRAB domain, a ZNF320 KRAB domain, a ZNF331 KRAB domain, a ZNF816 KRAB domain, a ZNF680 KRAB domain, a ZNF41 KRAB domain, a ZNF189 KRAB domain, a ZNF528 KRAB domain, a ZNF543 KRAB domain, a ZNF554 KRAB domain, a ZNF140 KRAB domain, a ZNF610 KRAB domain, a ZNF264 KRAB domain, a ZNF350 KRAB domain, a ZNF8 KRAB domain, a ZNF582 KRAB domain, a ZNF30 KRAB domain, a ZNF324 KRAB domain, a ZNF98 KRAB domain, a ZNF669 KRAB domain, a ZNF677 KRAB domain, a ZNF596 KRAB domain, a ZNF214 KRAB domain, a ZNF37A KRAB domain, a ZNF34 KRAB domain, a ZNF250 KRAB domain, a ZNF547 KRAB domain, a ZNF273 KRAB domain, a ZNF354A KRAB domain, a ZFP82 KRAB domain, a ZNF224 KRAB domain, a ZNF33A KRAB domain, a ZNF45 KRAB domain, a ZNF175 KRAB domain, a ZNF595 KRAB domain, a ZNF184 KRAB domain, a ZNF419 KRAB domain, a ZFP28-1 KRAB domain, a ZFP28-2 KRAB domain, a ZNF18 KRAB domain, a ZNF213 KRAB domain, a ZNF394 KRAB domain, a ZFP1 KRAB domain, a ZFP14 KRAB domain, a ZNF416 KRAB domain, a ZNF557 KRAB domain, a ZNF566 KRAB domain, a ZNF729 KRAB domain, a ZIM2 KRAB domain, a ZNF254 KRAB domain, a ZNF764 KRAB domain, a ZNF785 KRAB domain or any combination thereof. In some embodiments, the domain is a ZIM3 KRAB domain, a ZNF554 KRAB domain, a ZNF264 KRAB domain, a ZNF324 KRAB domain, a ZNF354A KRAB domain, a ZNF189 KRAB domain, a ZNF543 KRAB domain, a ZFP82 KRAB domain, a ZNF669 KRAB domain, or a ZNF582 KRAB domain or any combination thereof. In some embodiments, the domain is a ZIM3 KRAB domain, a ZNF554 KRAB domain, a ZNF264 KRAB domain, a ZNF324 KRAB domain, or a ZNF354A KRAB domain or any combination thereof. In some embodiments, the domain is a ZIM3 KRAB domain.

Sequences of exemplary functional repression domains that reduce or silence target gene expression are provided in Table 2 below. In some embodiments, the epigenetic editing system herein comprises one or more repression domains selected from Table 2, or functional homologs, orthologs, or variants thereof. Further examples of suitable repressors and repressor domains can be found in PCT/US2021/030643; and in Tycko et al., High-Throughput Discovery and Characterization of Human Transcriptional Effectors. Cell. 2020 Dec. 23; 183 (7): 2020-2035., each of which is incorporated herein by reference in its entirety.

TABLE 2
Exemplary protein sequences comprising repression
domains suitable for use as epigenetic effector
domains that may reduce or silence gene expression

| Protein | Protein Sequence |
|---|---|
| KRAB | SEQ ID NO: 9 |
| N-Terminal KRAB | SEQ ID NO: 10 |
| C-Terminal KRAB | SEQ ID NO: 11 |
| ZIM3 | SEQ ID NO: 100 |
| ZFP28 | SEQ ID NO: 44 |
| ZN627 | SEQ ID NO: 45 |
| KAP1 | SEQ ID NO: 46 |
| MeCP2 | SEQ ID NO: 47 |
| HP1b | SEQ ID NO: 48 |
| CBX8 | SEQ ID NO: 49 |
| CDYL2 | SEQ ID NO: 50 |
| TOX | SEQ ID NO: 51 |
| TOX3 | SEQ ID NO: 52 |
| TOX4 | SEQ ID NO: 53 |
| EED | SEQ ID NO: 54 |
| RBBP4 | SEQ ID NO: 55 |
| RCOR1 | SEQ ID NO: 56 |
| SCML2 | SEQ ID NO: 57 |
| KOX1 (ZNF10) | SEQ ID NO: 94 |

In some embodiments, an effector domain can comprise SEQ ID NO: 9. In some embodiments, a DNMT can be SEQ ID NO: 9. In some embodiments, a DNMT can be at least 99%, 98%, 97%, 96%, 95%, 90%, 85%, or 80% similar to SEQ ID NO: 9.

In some embodiments, an effector domain can comprise SEQ ID NO: 10. In some embodiments, a DNMT can be SEQ ID NO: 10. In some embodiments, a DNMT can be at least 99%, 98%, 97%, 96%, 95%, 90%, 85%, or 80% similar to SEQ ID NO: 10.

In some embodiments, an effector domain can comprise SEQ ID NO: 11. In some embodiments, a DNMT can be SEQ ID NO: 11. In some embodiments, a DNMT can be at least 99%, 98%, 97%, 96%, 95%, 90%, 85%, or 80% similar to SEQ ID NO: 11.

In some embodiments, an effector domain can comprise SEQ ID NO: 100. In some embodiments, a DNMT can be SEQ ID NO: 100. In some embodiments, a DNMT can be at least 99%, 98%, 97%, 96%, 95%, 90%, 85%, or 80% similar to SEQ ID NO: 100.

In some embodiments, an effector domain can comprise SEQ ID NO: 44. In some embodiments, a DNMT can be SEQ ID NO: 44. In some embodiments, a DNMT can be at least 99%, 98%, 97%, 96%, 95%, 90%, 85%, or 80% similar to SEQ ID NO: 44.

In some embodiments, an effector domain can comprise SEQ ID NO: 45. In some embodiments, a DNMT can be SEQ ID NO: 45. In some embodiments, a DNMT can be at least 99%, 98%, 97%, 96%, 95%, 90%, 85%, or 80% similar to SEQ ID NO: 45.

In some embodiments, an effector domain can comprise SEQ ID NO: 46. In some embodiments, a DNMT can be SEQ ID NO: 46. In some embodiments, a DNMT can be at least 99%, 98%, 97%, 96%, 95%, 90%, 85%, or 80% similar to SEQ ID NO: 46.

In some embodiments, an effector domain can comprise SEQ ID NO: 47. In some embodiments, a DNMT can be SEQ ID NO: 47. In some embodiments, a DNMT can be at least 99%, 98%, 97%, 96%, 95%, 90%, 85%, or 80% similar to SEQ ID NO: 47.

In some embodiments, an effector domain can comprise SEQ ID NO: 48. In some embodiments, a DNMT can be SEQ ID NO: 48. In some embodiments, a DNMT can be at least 99%, 98%, 97%, 96%, 95%, 90%, 85%, or 80% similar to SEQ ID NO: 48.

In some embodiments, an effector domain can comprise SEQ ID NO: 49. In some embodiments, a DNMT can be SEQ ID NO: 49. In some embodiments, a DNMT can be at least 99%, 98%, 97%, 96%, 95%, 90%, 85%, or 80% similar to SEQ ID NO: 49.

In some embodiments, an effector domain can comprise SEQ ID NO: 50. In some embodiments, a DNMT can be SEQ ID NO: 50. In some embodiments, a DNMT can be at least 99%, 98%, 97%, 96%, 95%, 90%, 85%, or 80% similar to SEQ ID NO: 50.

In some embodiments, an effector domain can comprise SEQ ID NO: 51. In some embodiments, a DNMT can be SEQ ID NO: 51. In some embodiments, a DNMT can be at least 99%, 98%, 97%, 96%, 95%, 90%, 85%, or 80% similar to SEQ ID NO: 51.

In some embodiments, an effector domain can comprise SEQ ID NO: 52. In some embodiments, a DNMT can be SEQ ID NO: 52. In some embodiments, a DNMT can be at least 99%, 98%, 97%, 96%, 95%, 90%, 85%, or 80% similar to SEQ ID NO: 52.

In some embodiments, an effector domain can comprise SEQ ID NO: 53. In some embodiments, a DNMT can be SEQ ID NO: 53. In some embodiments, a DNMT can be at least 99%, 98%, 97%, 96%, 95%, 90%, 85%, or 80% similar to SEQ ID NO: 53.

In some embodiments, an effector domain can comprise SEQ ID NO: 54. In some embodiments, a DNMT can be SEQ ID NO: 54. In some embodiments, a DNMT can be at least 99%, 98%, 97%, 96%, 95%, 90%, 85%, or 80% similar to SEQ ID NO: 54.

In some embodiments, an effector domain can comprise SEQ ID NO: 55. In some embodiments, a DNMT can be SEQ ID NO: 55. In some embodiments, a DNMT can be at least 99%, 98%, 97%, 96%, 95%, 90%, 85%, or 80% similar to SEQ ID NO: 55.

In some embodiments, an effector domain can comprise SEQ ID NO: 56. In some embodiments, a DNMT can be SEQ ID NO: 56. In some embodiments, a DNMT can be at least 99%, 98%, 97%, 96%, 95%, 90%, 85%, or 80% similar to SEQ ID NO: 56.

In some embodiments, an effector domain can comprise SEQ ID NO: 57. In some embodiments, a DNMT can be SEQ ID NO: 57. In some embodiments, a DNMT can be at least 99%, 98%, 97%, 96%, 95%, 90%, 85%, or 80% similar to SEQ ID NO: 57.

In some embodiments, an effector domain can comprise SEQ ID NO: 94. In some embodiments, a DNMT can be SEQ ID NO: 94. In some embodiments, a DNMT can be at least 99%, 98%, 97%, 96%, 95%, 90%, 85%, or 80% similar to SEQ ID NO: 94.

In some embodiments, an effector domain comprises a fusion of two effector domains (e.g., KOX1KRAB and ZIM3). In some embodiments, an effector domain comprises a fusion of 2, 3, 4, 5, 6, 7, 8, 9, or 10 effector domains. In some embodiments, an effector domain comprises a fusion of a truncated form of an effector domain and a second effector domain. In some embodiments, an effector domain comprises a fusion of the truncated forms of two effector domains. In some embodiments, a fusion effector domain comprises at least one truncated form of an effector domain.

In some embodiments, an effector domain comprises a functional domain that represses or silences gene expression, and the functional domain is a part of a larger protein, e.g., a zinc finger repressor protein. Functional domains that are capable of modulating gene expression, e.g., repress or increase gene expression can be identified from the larger protein with known methods and methods provided herein. For example, functional effector domains that can reduce or silence target gene expression may be identified based on sequences of repressor or activator proteins. Amino acid sequences of proteins having the function of modulating gene expression may be obtained from available genome browsers, such as UCSD genome browser or Ensembl genome browser.

### Linkers

Epigenetic editing systems provided herein may comprise one or more linkers that connect one or more components of the epigenetic editing systems. A linker may be a covalent bond or a polymeric linker with many atoms in length. A linker may be a peptide linker or a non-peptide linker.

In certain embodiments, linkers may be used to link any of the peptides or peptide domains of the epigenetic editing system. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is a non-peptide linker. For example, the linker may be a carbon bond, a disulfide bond, or carbon-heteroatom bond. In certain embodiments, the linker is a carbon-nitrogen bond of an amide linkage. In certain embodiments, the linker is a cyclic or acyclic, substituted or unsubstituted, branched or unbranched aliphatic or heteroaliphatic linker.

In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, alkyl halides, aryl halides, acyl halides, and isothiocyanates.

In some embodiments, one or more linkers of an epigenetic editing system provided herein is a peptide linker. For example, a zinc finger array and a repressor domain may be connected by a peptide linker, forming a zinc finger-repressor fusion protein. A peptide linker can be any length applicable to the epigenetic editing system fusion proteins described herein. In some embodiments, the linker can comprise a peptide between 1 and 200 amino acids. In some embodiments, a DNA binding domain, e.g., a zinc finger array and an effector domain are fused via a linker that comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. Longer or shorter linkers are also contemplated. In some embodiments, the peptide linker is 4, 16, 32, or 104 amino acids in length. In some embodiments, the peptide linker is a flexible linker. In some embodiments, the peptide linker is a rigid linker.

A linker can be 3, 4, 5, 6, 7, 8, 9, 10, 11, 12, 13, 14, 15, 16, 17, 18, 19, 20, 25, 30, 25, 40, 45, 50, 55, 60, 65, 70, 75, 80, 85, 90, 95, or 100 amino acids in length. In some embodiments, the linker is 5 amino acids in length. In some embodiments, the linker is 16 amino acids in length. In some embodiments, the linker is 20 amino acids in length. In some embodiments, the linker is 26 amino acids in length. In some embodiments, the linker is 80 amino acids in length.

In some embodiments, the peptide linker comprises the amino acid sequence of SEQ ID NOs: 1-5.

In some embodiments, the peptide linker is a XTEN linker. In some embodiments, the linker comprises an XTEN16 linker. In some embodiments, the linker comprises the amino acid sequence of SEQ ID NO: 2. In some embodiments, the linker comprises an amino acid sequence with at least 99%, 98%, 97%, 96%, 95%, 94%, 93%, 92%, 91% or 90% sequence similarity to SEQ ID NO: 2. In some embodiments, the peptide linker comprises an XTEN80 linker. In some embodiments, the peptide linker comprises the amino acid sequence of SEQ ID NO: 3. In some embodiments, the linker comprises an amino acid sequence with at least 99%, 98%, 97%, 96%, 95%, 94%, 93%, 92%, 91% or 90% sequence similarity to SEQ ID NO: 3.

Various linker lengths and flexibilities between a effector domain (e.g., a repressor domain) and a DNA binding protein (e.g., a Cas9 domain), between a effector domain and a second effector domain, or between any two components of an epigenetic editing system can be employed (e.g., ranging from very flexible linkers such as glycine/serine-rich linkers to more rigid linkers in order to achieve the optimal length for effector domain activity for the specific application. In some embodiments, the flexible linkers are glycine/serine-rich linkers (GS-rich linkers), where more than 45% (e.g., more than 48, 50, 55, 60, 70, 80, or 90%) of the residues are glycine or serine residues. Non-limiting examples of the GS-rich linkers are (GGGGS)n (SEQ ID NO: 4) and (G)n. In some embodiments, the more rigid linkers comprise (EAAAK)n, (SGGS)n, and (XP)n. In the aforementioned formulae of flexible and rigid linkers, n is any integer between 1 and 30. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, the linker comprises a (GGGGS)n motif, wherein n is 4 (SEQ ID NO: 4).

In some embodiments, a linker in an epigenetic editing system comprises a nuclear localization signal, for example, of peptide sequence SEQ ID NO: 7. In some embodiments, a linker in an epigenetic editing system comprises an expression tag, e.g., a detectable tag such as a green fluorescence protein.

In some embodiments, a linker comprises a nucleic acid. For example, one or more linkers of an epigenetic editing system may include a nucleic acid that is capable of binding to, interacting with, associating with, or forming a complex with a polypeptide. In some embodiments, the nucleic acid linker may be a RNA linker capable of binding to and/or interacting with a RNA binding protein domain, e.g. a phase derived RNA binding domain. In some embodiments, the nucleic acid linker may be fused to a guide polynucleotide capable of binding to a Cas protein of an epigenetic editing system. In some embodiments, the nucleic acid linker comprises a K homology (KH) domain binding sequence, a MS2 coat protein binding sequence, a PP7 coat protein binding sequence, a SfMu COM coat protein binding sequence, a telomerase Ku binding motif binding sequence, a sm7 protein binding sequence, or other RNA recognition motif binding sequence thereof.

In some embodiments, a linker comprises an affinity domain that specifically binds a component of an epigenetic effector. For example, an epigenetic effector may comprise a programmable DNA binding domain, a linker comprising an affinity domain having specific binding affinity to an epigenetic effector domain. The affinity domain may comprise an antibody, a single chain antibody, a nanobody, and antigen binding sequence, an antibody, a nanobody, a functional antibody fragment, a single chain variable fragment (scFv), a Fab, a single-domain antibody (sdAb), a VH domain, a VL domain, a VNAR domain, a VHH domain, a bispecific antibody, a diabody, or a functional fragment or a combination thereof. In some embodiments, an epigenetic effector domain comprises a programmable DNA binding domain and a KAP1 antibody which binds to a KAP1 protein. In some embodiments, an epigenetic effector domain comprises a programmable DNA binding domain and a KRAB antibody which binds to a KRAB protein. In some embodiments, an epigenetic effector domain comprises a programmable DNA binding domain and a DNMT1 antibody which binds to a DNMT1 protein. In some embodiments, an epigenetic effector domain comprises a programmable DNA binding domain and a DNMT3A antibody which binds to a DNMT3A protein. In some embodiments, an epigenetic effector domain comprises a programmable DNA binding domain and a DNMT3L antibody which binds to a DNMT3L protein. In some embodiments, an epigenetic effector domain comprises a programmable DNA binding domain and a ZIM3 antibody which binds to a ZIM3 protein. In some embodiments, an epigenetic effector domain comprises a programmable DNA binding domain and a TET1 antibody which binds to a TET1 protein. In some embodiments, an epigenetic effector domain comprises a programmable DNA binding domain and a VP16 or VP64 antibody which binds to a VP16 or VP64 protein.

In some embodiments, a linker comprises a repeat peptide array. In some embodiments, a linker comprises an epitope tag, for example, a SunTag. In some embodiments, an epigenetic editing system comprises one or more peptide arrays comprising multiple copies of an epitope tag that can link multiple effector domains attached to or fused to peptide recognizing the epitope tag. For example, an epitope tag array can link a DNA binding domain and multiple effector domains or multiple copies of effector domains fused to or attached to antibody sequences recognizing the epitope tag. In some embodiments, an epigenetic editing system comprises at least 1, 2, 3, 4, 5, 6, 7, 8, 9, 10 or more epitope tag repeats that link at least 1, 2, 3, 4, 5, 6, 7, 8, 9, 10 or more effector domains or copies of effector domains. In some embodiments, an epigenetic editing system comprises multiple epitope tag repeats that link multiple effector domains and detectable expression tag domains, e.g. GFPs. In some embodiments, the repeat peptide array comprises gene control non-depressible 4 (GCN4) peptide sequences. In some embodiments, the repeat peptide arrays are further linked by linking peptide sequences of 15 to 50 amino acids. Repeat peptide arrays as described in US patent application No. US20170219596 and U.S. Pat. No. 10,612,044 are incorporated herein by reference in its entirety.

### Nuclear Localization Sequences

In some embodiments, the epigenetic editing systems provided herein comprise one or more nuclear targeting sequences. In some embodiments, the epigenetic editing systems provided herein comprise at least 1, 2, 3, 4, 5, 6, 7, 8, 9, 10 or more nuclear targeting sequences. For example, a zinc finger-repressor fusion protein described herein may further comprise one or more nuclear targeting sequences, for example, a nuclear localization sequence (NLS). In some embodiments, the fusion protein comprises multiple NLSs. In some embodiments, the fusion protein comprises a NLS at the N-terminus or the C-terminus of the fusion protein. In some embodiments, the fusion protein comprises a NLS at both the N-terminus and the C-terminus. In some embodiments, the NLS is embedded in the middle of the fusion protein. In some embodiments, a NLS comprises an amino acid sequence that facilitates the importation of a protein, that comprises an NLS, into the cell nucleus. In some embodiments, the NLS is fused to the N-terminus of the fusion protein. In some embodiments, the NLS is fused to the C-terminus of the fusion protein. In some embodiments, the NLS is fused to the N-terminus of the nucleic acid binding protein, e.g., the Cas9 or zinc finger array. In some embodiments, the NLS is fused to the C-terminus of the nucleic acid binding protein. In some embodiments, the NLS is fused to the N-terminus of an effector domain, e.g., a repressor domain. In some embodiments, the NLS is fused to the C-terminus of an effector domain, e.g., a repressor domain. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein.

In some embodiments, the epigenetic editing systems provided herein comprise two NLS sequences. In some embodiments, the fusion protein of the epigenetic editing system comprises one NLS at the N-terminus and one NLS at the C-terminus. In some embodiments, the fusion protein comprises two NLS at the N-terminus. In some embodiments, the fusion protein comprises two NLS at the C-terminus. In some embodiments, one NLS is located at the N-terminus and one NLS is embedded in the middle of the fusion protein. In some embodiments, one NLS is located at the C-terminus and one NLS is embedded in the middle of the fusion protein. In some embodiments, both NLS are embedded in the middle of the fusion protein.

In some embodiments, the fusion protein of the epigenetic editing system comprises two NLS sequences which flank a DNMT domain. In some embodiments, the fusion protein comprises two NLS sequences which flank a fusion DNMT domain. In some embodiments, the fusion protein comprises two NLS sequences that flank a DNA binding domain. In some embodiments, the fusion protein domain comprises two NLS sequences that flank an effector domain.

In some embodiments, the epigenetic editing systems provided herein comprise four NLS sequences. In some embodiments, the fusion protein of the epigenetic editing system comprises at least two NLS at the N terminus. In some embodiments, the fusion protein of the epigenetic editing system comprises at least two NLS at the C terminus. In some embodiments, the fusion protein of the epigenetic editing system comprises two NLS at the N terminus and two NLS at the C terminus. In some embodiments, at least one NLS in embedded in the middle of the fusion protein.

In some embodiments, a NLS comprises the amino acid sequence SEQ ID NO: 7. In some embodiments, a NLS sequence is an endogenous NLS sequence. Additional nuclear localization sequences are known in the art and would be apparent to the skilled artisan. Examples of suitable NLS sequences for inclusion in fusion proteins as provided herein include, without limitation, those described in Lu et al., Types of nuclear localization signals and mechanisms of protein import into the nucleus, Cell Commun Signal. 2021; 19:60, 2021; the entire contents of which are incorporated herein by reference.

In some embodiments the fusion protein comprising two NLS at the N terminus and two NLS at the C terminus can increase the efficiency of the epigenetic editor system by at least 5%, at least 10%, at least 15%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 5,000%, at least 10,000%, at least 50,000%, at least 100,000%, or more as compared to an epigenetic editor system that does not have two NLS at the N terminus and two NLS at the C terminus. In some embodiments the fusion protein comprising two NLS at the N terminus and two NLS at the C terminus can increase the efficiency of the epigenetic editor system by at most 100,000%, at most 50,000%, at most 10,000%, at most 5,000%, at most 1,000%, at most 900%, at most 800%, at most 700%, at most 600%, at most 500%, at most 400%, at most 300%, at most 200%, at most 100%, at most 90%, at most 80%, at most 70%, at most 60%, at most 50%, at most 40%, at most 30%, at most 20%, at most 15%, at most 10%, at most 5% or less as compared to an epigenetic editor system that does not have two NLS at the N terminus and two NLS at the C terminus.

### Tags

Epigenetic editing systems provided herein may comprise one or more additional sequences domains, tags, for tracking, detection, and localization of the editors. In some embodiments, an epigenetic editing system comprises one or more detectable tags. In some embodiments, the epigenetic editing system comprises 1, 2, 3, 4, 5, 6, 7, 8, 9, 10 or more detectable tags. Each of the detectable tags may be same or different.

For example, an epigenetic editing system fusion protein may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art.

In some embodiments, an epigenetic editing system comprises from 1 to 2 detectable tags. In aspects, the fusion protein comprises 1 detectable tag. In aspects, the fusion protein comprises 2 detectable tags. In aspects, the fusion protein comprises 3 detectable tags. In aspects, the fusion protein comprises 4 detectable tags. In aspects, the fusion protein comprises 5 detectable tags.

### Epigenetic Editing System Structures

Some aspects of this disclosure provide epigenetic editing systems. Exemplary, non-limiting structures of such editing systems are described in more detail herein. Additional suitable structures will be apparent to the skilled artisan based on the present disclosure, which is not limited in this respect.

The multiple components of epigenetic editing systems described herein may be in any order. In some embodiments, an epigenetic editing system comprises the structure: N′]-[D1]-[D2]-[C′, wherein any one of D1 and D2 is a DNA binding domain, an effector domain, or a nucleic acid binding domain. In these structural examples, N′ denotes the N-terminus, C′ denotes the C-terminus; and]-[denotes a linking element or a linker.

In some embodiments, an epigenetic editing system comprises the structure: N′]-[D1]-[D2]-[D3]-[C′, wherein any one of D1, D2, and D3 is a DNA binding domain, an effector domain, or a nucleic acid binding domain. In some embodiments, D1 is a DNA binding domain. In some embodiments, D2 is a DNA binding domain. In some embodiments, D3 is a DNA binding domain. In some embodiments, D1 is the only DNA binding domain. In some embodiments, D2 is the only DNA binding domain. In some embodiments, D3 is the only DNA binding domain.

In some embodiments, an epigenetic editing system comprises the structure: N′]-[D1]-[D2]-[D3]-[D4]-[C′, wherein any one of D1, D2, D3, and D4 is a DNA binding domain, an effector domain, or a nucleic acid binding domain. In some embodiments, D1 is a DNA binding domain. In some embodiments, D2 is a DNA binding domain. In some embodiments, D3 is a DNA binding domain. In some embodiments, D4 is a DNA binding domain. In some embodiments, D1 is the only DNA binding domain. In some embodiments, D2 is the only DNA binding domain. In some embodiments, D3 is the only DNA binding domain. In some embodiments, D4 is the only DNA binding domain.

In some embodiments, an epigenetic editing system comprises the structure: N′]-[D1]-[D2]-[D3]-[D4]-[D5]-[C′, wherein any one of D1, D2, D3, D4, and D5 is a DNA binding domain, an effector domain, or a nucleic acid binding domain. In some embodiments, D1 is a DNA binding domain. In some embodiments, D2 is a DNA binding domain. In some embodiments, D3 is a DNA binding domain. In some embodiments, D4 is a DNA binding domain. In some embodiments, D5 is a DNA binding domain. In some embodiments, D1 is the only DNA binding domain. In some embodiments, D2 is the only DNA binding domain. In some embodiments, D3 is the only DNA binding domain. In some embodiments, D4 is the only DNA binding domain. In some embodiments, D5 is the only DNA binding domain.

In some embodiments, the epigenetic editing system comprises at least one effector domain that is a DNMT domain. In some embodiments, the epigenetic editing system comprises at least one effector domain that is a KRAB domain. In some embodiments, the epigenetic editing system comprises at least one effector domain that is a ZIM KRAB domain. In some embodiments, the epigenetic effector comprises at least one effector domain that is a DNMT3A domain, or a truncated version thereof. In some embodiments, the epigenetic effector comprises at least one effector domain that is a DNMT3L domain, or a truncated version thereof.

Components of an epigenetic editing system may be structured in different configurations. For example, the DNA binding domain may be at the C terminus, the N terminus, or in between two or more epigenetic effector domains or additional domains. In some embodiments, the DNA binding domain is at the C terminus of the epigenetic editing system. In some embodiments, the DNA binding domain is at the N terminus of the epigenetic editing system. In some embodiments, the DNA binding domain is linked to one or more nuclear localization signals. In some embodiments, the DNA binding domain is linked to two or more nuclear localization signals. In some embodiments, the DNA binding domain is flanked by an epigenetic effector domain or an additional domain on both termini. In some embodiments, the epigenetic editing system comprises the configuration of N′]-[epigenetic effector domain 1]-[DNA binding domain]-[epigenetic effector domain 2]-[C′. In some embodiments, the epigenetic editing system comprises the configuration of N′]-[epigenetic effector domain 1]-[DNA binding domain]-[epigenetic effector domain 2]-[epigenetic effector domain 3]-[C′. In some embodiments, the epigenetic editing system comprises the configuration of N′]-[epigenetic effector domain 1]-[epigenetic effector domain 2]-[DNA binding domain]-[epigenetic effector domain 3]-[C′. In some embodiments, the epigenetic editing system comprises the configuration of N′]-[epigenetic effector domain 1]-[epigenetic effector domain 2]-[DNA binding domain]-[epigenetic effector domain 3]-[epigenetic effector domain 4]-[C′. In some embodiments, the epigenetic editing system comprises the configuration of N′]-[KRAB]-[DNA binding domain]-[Dnmt3A]-[C′. In some embodiments, the epigenetic editing system comprises the configuration of N′]-[KRAB]-[DNA binding domain]-[Dnmt3A]-[Dnmt3L]-[C′. In some embodiments, the epigenetic editing system comprises the configuration of N′]-[SETDB1]-[DNA binding domain]-[Dnmt3A]-[Dnmt3L]-[C′. In some embodiments, the epigenetic editing system comprises the configuration of N′]-[SETDB1]-[DNA binding domain]-[Dnmt3A]-[C′. In some embodiments, the epigenetic editing system comprises the configuration of N′]-[KRAB]-[DNA binding domain]-[Dnmt3A-Dnmt3L]-[C′, wherein Dnmt3A and Dnmt3L are directly fused via a peptide bond.

In some embodiments, a connecting structure “]-[” in any one of the epigenetic editing system structures is a linker, e.g., a peptide linker. In some embodiments, a connecting structure “]-[” in any one of the epigenetic editing system structures is a detectable tag. In some embodiments, a connecting structure “]-[” in any one of the epigenetic editing system structures is a peptide bond. In some embodiments, a connecting structure “]-[” in any one of the epigenetic editing system structures is a nuclear localization signal. In some embodiments, a connecting structure “]-[” in any one of the epigenetic editing system structures is a promoter or a regulatory sequence. In an epigenetic editing system structure, the multiple connecting structures “]-[” may be same or may each be a different linker, tag, NLS, or peptide bond.

The DNA binding domain (DBD) of an epigenetic editing system may comprise any one of the DNA binding domains described herein or known to those skilled in the art. In some embodiments, the DBD comprises one or more zinc finger arrays. In some embodiments, the DBD comprises a TALE DNA binding domain. In some embodiments, the DBD is a RNA guided programmable DNA binding domain, e.g. a CRISPR-Cas protein domain. Suitable Cas proteins has been provided herein, including nuclease inactive Cas proteins for the purpose of epigenetic editing without causing target DNA strand breaks. A Cas protein in an epigenetic editing system may be a nuclease inactive Cas9 (dCas9), a SaCas9d, a SpCas9d, a dCas9 with modified PAM specificity, a high-fidelity dCas9, a nuclease inactive Cpf1 (dCpf1), a dCpf1 with modified PAM specificity, a high-fidelity dCpf1, a dCas12e, a dCasY, or any other Cas protein as described herein.

In some embodiments, an epigenetic editing system comprises a DNA binding domain (DBD) and an effector domain that represses or silences expression of a target gene. In some embodiments, the epigenetic editing system comprises the configuration of N′]-[repression domain]-[DBD]-[-C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editing system comprises the configuration of N′]-[DBD]-[repression domain]-[-C′, wherein the connecting structure]-[is any one of the linkers as described herein, a detectable tag, an affinity domain, a peptide bond, a nuclear localization signal, a promoter, and/or a regulatory sequence.

In some embodiments, an epigenetic editing system comprises a DNA binding domain (DBD) and a DNA methyltransferase domain that deposits one or more methylation marks at a target gene, thereby repressing or silencing expression of the target gene. In some embodiments, the epigenetic editing system comprises the configuration of N′]-[DNA methyltransferase domain]-[DBD]-[-C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editing system comprises the configuration of N′]-[DBD]-[DNA methyltransferase domain]-[-C′, wherein the connecting structure]-[is any one of the linkers as described herein, a detectable tag, an affinity domain, a peptide bond, a nuclear localization signal, a promoter, and/or a regulatory sequence.

In some embodiments, an epigenetic editing system comprises a DNA binding domain (DBD), a DNA methyltransferase domain, and an effector domain that represses or silences expression of a target gene. In some embodiments, the epigenetic editing system comprises the configuration of N′]-[DNA methyltransferase domain]-[DBD]-[repression domain]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editing system comprises the configuration of N′]-[repression domain]-[DBD]-[DNA methyltransferase domain]-[C′, wherein the connecting structure]-[is any one of the linkers as described herein, a detectable tag, an affinity domain, a peptide bond, a nuclear localization signal, a promoter, and/or a regulatory sequence.

In some embodiments, the epigenetic editing system comprises the configuration of N′]-[DNA methyltransferase domain]-[repression domain]-[DBD]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editing system comprises the configuration of N′]-[repression domain]-[DNA methyltransferase domain]-[DBD]-[C′, wherein the connecting structure]-[is any one of the linkers as described herein, a detectable tag, an affinity domain, a peptide bond, a nuclear localization signal, a promoter, and/or a regulatory sequence.

The repression domain in an epigenetic editing system may comprise any one of the expression repression proteins known to those skilled in the art and as described herein, or any homologs or combination thereof. In some embodiments, the repression domain comprises a histone deacetylase domain. In some embodiments, the repression domain interacts with a scaffold protein domain that recruits one or more protein domains that repress expression of the target gene. For example, the repression domain may recruit or interact with a scaffold protein domain that recruits a PRMT protein, a HDAC protein, a SETDB1 protein, or a NuRD protein domain. In some embodiments, the repression domain interacts with epigenetically marked DNA nucleotides in a target gene thereby repressing or silencing expression of the target gene. In some embodiments, the repression domain comprises a MECP2 domain. In some embodiments, the repression domain comprises a KAP1 domain. In some embodiments, the repression domain comprises any one of the domains of Table 2 or Table 3, or any combination or homologs thereof.

The DNA methyltransferase domain in an epigenetic editing system may comprise any one of the DNA methyltransferase proteins known to those skilled in the art and as described herein, or any homologs or combination thereof. In some embodiments, the effector domain comprises a DNMT3 domain. In some embodiments, the DNA methyltransferase domain comprises a DNMT3A domain. In some embodiments, the DNA methyltransferase domain comprises a DNMT3B domain. In some embodiments, the DNA methyltransferase domain comprises a DNMT3C domain. In some embodiments, the DNA methyltransferase domain comprises a DNMT3L domain. In some embodiments, the DNA methyltransferase domain comprises a fusion of DNMT3A-DNMT3L domain. As described herein, the DNMT3A-DNMT3L fusion domain may be in either order, e.g., N-DNMT3A-DNMT3L-C, or N-DNMT3L-DNMT3A-C. In some embodiments, the DNA methyltransferase domain comprises any one of the domains of Table 1, or any combination or homologs thereof.

In some embodiments, an epigenetic editing system comprises a DNA binding domain (DBD) and an effector domain that increases expression of a target gene. In some embodiments, the epigenetic editing system comprises the configuration of N′]-[activation domain]-[DBD]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editing system comprises the configuration of N′]-[DBD]-[activation domain]-[C′, wherein the connecting structure]-[is any one of the linkers as described herein, a detectable tag, an affinity domain, a peptide bond, a nuclear localization signal, a promoter, and/or a regulatory sequence.

In some embodiments, an epigenetic editing system comprises a DNA binding domain (DBD) and a DNA demethylation domain that removes one or more methylation marks at a target gene, thereby increasing expression of the target gene. In some embodiments, the epigenetic editing system comprises the configuration of N′]-[DNA demethylase domain]-[DBD]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editing system comprises the configuration of N′]-[DBD]-[DNA demethylase domain]-[C′, wherein the connecting structure]-[is any one of the linkers as described herein, a detectable tag, an affinity domain, a peptide bond, a nuclear localization signal, a promoter, and/or a regulatory sequence.

In some embodiments, an epigenetic editing system comprises a DNA binding domain (DBD), a DNA demethylase domain, and an activation effector domain that increases expression of a target gene. In some embodiments, the epigenetic editing system comprises the configuration of N′]-[DNA demethylase domain]-[DBD]-[activation domain]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editing system comprises the configuration of N′]-[activation domain]-[DBD]-[DNA demethylase domain]-[C′, wherein the connecting structure]-[is any one of the linkers as described herein, a detectable tag, an affinity domain, a peptide bond, a nuclear localization signal, a promoter, and/or a regulatory sequence.

In some embodiments, the epigenetic editing system comprises the configuration of N′]-[DNA demethylase domain]-[activation domain]-[DBD]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editing system comprises the configuration of N′]-[activation domain]-[DNA demethylase domain]-[DBD]-[C′, wherein the connecting structure]-[is any one of the linkers as described herein, a detectable tag, an affinity domain, a peptide bond, a nuclear localization signal, a promoter, and/or a regulatory sequence.

In some embodiments, the epigenetic editing system that reduces or silences expression of a target gene comprises a DBD and an affinity domain that specifically binds to a repression domain. For example, the epigenetic editing system may comprise a DBD and a repression domain antibody. In some embodiments, the epigenetic editing system comprises a DBD and a KAP1 affinity domain. In some embodiments, the epigenetic editing system comprises a DBD and a KRAB affinity domain. In some embodiments, the epigenetic editing system comprises a DBD and a SETDB1 affinity domain. In some embodiments, the epigenetic editing system comprises a DBD and a MECP2 affinity domain. In some embodiments, the epigenetic editing system comprises a DNA methyltransferase and a repression domain binding affinity domain.

In some embodiments, the epigenetic editing system that reduces or silences expression of a target gene comprises a DBD and an affinity domain that specifically binds to a DNA methyltransferase domain. For example, the epigenetic editing system may comprise a DBD and a DNA methyltransferase antibody. In some embodiments, the epigenetic editing system comprises a DBD and a Dnmt3A affinity domain. In some embodiments, the epigenetic editing system comprises a DBD and a Dnmt3L affinity domain. In some embodiments, the epigenetic editing system comprises a repression domain and a DNA methyltransferase binding affinity domain. In some embodiments, the epigenetic editing system comprises a repression domain and a Dnmt3A binding affinity domain. In some embodiments, the epigenetic editing system comprises a repression domain and Dnmt3L affinity domain. In some embodiments, the epigenetic editing system comprises one or more of a KAP1, a KRAB and a MECP2 domain, and a Dnmt3 A binding affinity domain. In some embodiments, the epigenetic editing system comprises one or more of a KAP1 domain, and a Dnmt3A binding affinity domain. In some embodiments, the epigenetic editing system comprises one or more of a KAP1, a KRAB and a MECP2 domain, and a Dnmt3L binding affinity domain. In some embodiments, the epigenetic editing system comprises one or more of a KAP1 domain, and a Dnmt3L binding affinity domain. The affinity domain may be an antibody, a single chain antibody, a nanobody, and antigen binding sequence, an antibody, a nanobody, a functional antibody fragment, a single chain variable fragment (scFv), an Fab, a single-domain antibody (sdAb), a VH domain, a VL domain, a VNAR domain, a VHH domain, a bispecific antibody, a diabody, or a functional fragment or a combination thereof.

In some embodiments, the epigenetic editing system that reduces or silences expression of a target gene comprises a DBD and a first affinity domain that specifically binds to a DNA methyltransferase domain and a second affinity domain that specifically binds to a repression domain. For example, the epigenetic editing system may comprise a DBD and a DNA methyltransferase antibody and a repression domain antibody. In some embodiments, the epigenetic editing system comprises a DBD, a KAP1 affinity domain and a Dnmt3A affinity domain. In some embodiments, the epigenetic editing system comprises a DBD, a KAP1 affinity domain and a Dnmt3L affinity domain. In some embodiments, the epigenetic editing system comprises a DBD, a MECP2 affinity domain and a Dnmt3A affinity domain. In some embodiments, the epigenetic editing system comprises a DBD, a MECP2 affinity domain and a Dnmt3L affinity domain. In some embodiments, the epigenetic editing system comprises a DBD, a KRAB affinity domain and a Dnmt3 A affinity domain. In some embodiments, the epigenetic editing system comprises a DBD, a KRAB affinity domain and a Dnmt3L affinity domain. The affinity domain may be an antibody, a single chain antibody, a nanobody, and antigen binding sequence, an antibody, a nanobody, a functional antibody fragment, a single chain variable fragment (scFv), an Fab, a single-domain antibody (sdAb), a VH domain, a VL domain, a VNAR domain, a VHH domain, a bispecific antibody, a diabody, or a functional fragment or a combination thereof.

In some embodiments, the DNA methyltransferase may comprise any one of the DNMT domains provided herein, or any combinations or homologs thereof. In particular embodiments, the DNA methyltransferase domain comprises DNMT3A or a truncated version thereof, DNMT3L or a truncated version thereof, or both. In particular embodiments, the DBD is a catalytically inactive polynucleotide guided DNA-binding domain (e.g., a dCas9) or a ZFP domain. In certain embodiments, the repressor domain comprises any one of the repressor domains provided herein, or any combinations or homologs thereof. For example, in some embodiments, the repressor domain may be a KRAB domain. In certain embodiments, the repressor domain is a ZFP28, ZN627, KAP1, MeCP2, HP1b, CBX8, CDYL2, TOX, Tox3, Tox4, EED, RBBP4, RCOR1, or SCML2 KRAB domain, or a fusion of two of said domains (e.g., a fusion of the N- and C-terminal regions of ZIM3 and KOX1 KRAB domains)). In particular embodiments, the repressor domain is a KRAB domain from ZFP28, ZF627, ZIM3, or KOX1.

Particular constructs contemplated herein include:
DNMT3A-DNMT3L-XTEN80-NLS-dCas9-NLS-XTEN16-KOX1 KRAB (Configuration 1),
DNMT3A-DNMT3L-XTEN80-NLS-ZFP domain-NLS-XTEN16-KOX1 KRAB (Configuration 2),
NLS-DNMT3A-DNMT3L-XTEN80-dCas9-XTEN16-KOX1 KRAB-NLS (Configuration 3),
NLS-DNMT3A-DNMT3L-XTEN80-ZFP domain-XTEN16-KOX1 KRAB-NLS (Configuration 4),
NLS-NLS-DNMT3A-DNMT3L-XTEN80-dCas9-XTEN16-KOX1 KRAB-NLS-NLS (Configuration 5), and
NLS-NLS-DNMT3A-DNMT3L-XTEN80-ZFP domain-XTEN16-KOX1 KRAB-NLS-NLS (Configuration 6).

In particular embodiments, the fusion construct may have the configuration:
NLS-NLS-hDNMT3A-hDNMT3L-XTEN80-dCas9-XTEN16-KOX1 KRAB-NLS-NLS (Configuration 7),
NLS-NLS-DNMT3A-DNMT3L-XTEN80-ZFP domain-XTEN16-KOX1 KRAB-NLS-NLS (Configuration 8),
NLS-NLS-hDNMT3A-hDNMT3L-XTEN80-dCas9-XTEN16-ZFP28 KRAB-NLS-NLS (Configuration 9),
NLS-NLS-DNMT3A-DNMT3L-XTEN80-ZFP domain-XTEN16-ZFP28 KRAB-NLS-NLS (Configuration 10),
NLS-NLS-hDNMT3A-hDNMT3L-XTEN80-dCas9-XTEN16-ZN627 KRAB-NLS-NLS (Configuration 11), or
NLS-NLS-DNMT3A-DNMT3L-XTEN80-ZFP domain-XTEN16-ZN627 KRAB-NLS-NLS (Configuration 12).
NLS-NLS-DNMT3A-hDNMT3L-XTEN80-dCas9-NLS-XTEN16-ZIM3 KRAB-NLS-NLS (Configuration 13).
NLS-NLS-DNMT3A-hDNMT3L-XTEN80-dCas9-NLS-XTEN16-ZN627 KRAB-NLS-NLS (Configuration 14).
NLS-NLS-DNMT3A-hDNMT3L-XTEN80-dCas9-NLS-XTEN16-KOX1 KRAB-NLS-NLS (Configuration 15).
NLS-DNMT3A-hDNMT3L-XTEN80-dCas9-NLS-XTEN16-KOX1-KRAB (Configuration 16).

### DNA Binding Domains

Epigenetic editing systems and epigenetic editing complexes described herein may comprise one or more nucleic acid binding protein domains, e.g., DNA binding domains, that may direct the epigenetic editing system to a target gene associated with a certain condition.

As used herein, a target gene can comprise all nucleotide sequences of a gene of interest. For example, sequences or nucleotides of a target gene can include coding sequences and non-coding sequences. Sequence of a target gene can include exons or introns. Sequences of a target gene can include regulatory regions, including promoters, enhancers, terminators, 5′ or 3′ untranslated regions. In some embodiments, a sequence of a target gene comprises a remote enhancer sequence.

An epigenetic editing system as described herein can comprise any polynucleotide binding domain. In some embodiments, the nucleic acid binding domain comprises one or more DNA binding proteins, for example, zinc finger proteins (ZFPs) or transcription activator like effectors (TALEs). In some embodiments, the nucleic acid binding domain comprises a polynucleotide guided DNA binding protein, for example, a nuclease inactive CRISPR-Cas protein guided by a guide RNA.

The nucleic acid binding domain of epigenetic editing systems described herein may be capable of recognizing and binding any gene of interest, for example, target genes associated with a disease or disorder. In some embodiments, the target gene associated with a disease or disorder contains a mutation as compared to a wild type gene. In some embodiments, the target gene associated with a disease or disorder contains a copy that harbors a mutation associated with the disease or disorder. In some embodiments, the target gene associated with a disease or disorder has one or both copies of wild type DNA sequences.

A DNA binding domain maybe modular and/or programmable. In some embodiments, the DNA binding domain comprises a zinc finger domain, a transcription activator like effector (TALE) domain, a meganuclease DNA binding domain or a polynucleotide guided nucleic acid binding domain. Examples of DNA binding domains can be found in U.S. Pat. No. 11,162,114, which is incorporated by reference in its entirety.

Transcription activator-like effectors (TALEs) can be engineered to bind practically any desired DNA sequence. Methods for programming TALEs are familiar to one skilled in the art. For example, such methods are described in Carroll et al, Genetics Society of America, 188 (4): 773-782, 2011; Miller et al., Nature Biotechnology 25 (7): 778-785, 2007; Christian et al, Genetics 186 (2): 757-61, 2008; Li et al, Nucleic Acids Res. 39 (1): 359-372, 2010; and Moscou et al, Science 326 (5959): 1501, 2009, each of which are incorporated herein by reference.

A DNA binding domain may be directed by a nucleic acid sequence, for example, a RNA sequence, to identify the target gene. In some embodiments, the DNA binding domain comprises a programmable nuclease. In some embodiments, the DNA binding domain comprises a programmable nuclease with reduced or abrogated nuclease activity. For example, a programmable nuclease may harbor one or two mutations in its catalytic domain that renders the nuclease inactive but maintain DNA binding activity of the nuclease. In some embodiments, the DNA binding domain comprises a CRISPR-Cas protein domain. In some embodiments, the CRISPR-Cas protein domain lacks or has reduced nuclease activity.

In some embodiments, an epigenetic editing system provided herein comprises a Cas protein, e.g., a Cas9 protein domain. The Cas9 domain may be any of the Cas9 domains or Cas9 proteins (e.g., nuclease inactive Cas9 or Cas9 nickase, or a Cas9 variant from any species) provided herein. In some embodiments, any of the Cas domains or Cas proteins provided herein may be fused with one or more any effector protein domain as described herein. In some embodiments, any of the Cas protein domains provided herein may be fused with two or more effector protein domains as described herein. Cas9 can refer to a polypeptide with at least about 50%, 60%, 70%, 80%, 90%, 100% sequence identity and/or sequence similarity to a wild type exemplary Cas9 polypeptide (e.g., from S. pyogenes). Cas9 can refer to the wild type or a modified form of the Cas9 protein that can comprise an amino acid change such as a deletion, insertion, substitution, variant, mutation, fusion, chimera, or any combination thereof.

Cas9 sequences and structures of variant Cas9 orthologs have been described in various species. Exemplary species that the Cas9 protein or other components can be from include, but are not limited to, Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polar omonas naphthalenivorans, Polar omonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionium, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillator sp., Petrotoga mobilis, Thermosipho africanus, Streptococcus pasteurianus, Neisseria cinerea, Campylobacter lari, Parvibaculum lavamentivorans, Coryne bacterium diphtheria, or Acaryochloris marina. In some embodiments, the Cas9 protein is from Streptococcus pyogenes. In some embodiments, the Cas9 protein may be from Streptococcus thermophilus. In some embodiments, the Cas9 protein is from Staphylococcus aureus.

Additional suitable Cas9 proteins, orthologs, variants, including nuclease inactive variants and sequences will be apparent to those of skill in the art based on this disclosure, and such Cas9 nucleases and sequences include Cas9 sequences from the organisms and loci disclosed in Chylinski et al., (2013) RNA Biology 10:5, 726-737; which are incorporated herein by reference.

An epigenetic editing system may comprise a nuclease inactive Cas9 domain (dead Cas9 or dCas9). The dCas9 protein domain may comprise one, two, or more mutations as compared to a wild type Cas9 that abrogate its nuclease activity but retains the DNA binding activity. For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9. In some embodiments, the dCas9 comprises at least one mutation in the HNH subdomain and the RuvC subdomain that reduces or abrogates nuclease activity. In some embodiments, the dCas9 only comprises a RuvC subdomain. In some embodiments, the dCas9 only comprises a HNR subdomain. It is to be understood that any mutation that inactivates the RuvC or the HNH domain may be included in a dCas9, e.g., insertion, deletion, or single or multiple amino acid substitution in the RuvC domain and/or the HNH domain.

Additional suitable mutations that inactivate Cas9 will be apparent to those of skill in the art based on this disclosure and knowledge in the field and are within the scope of this disclosure. Such additional exemplary suitable nuclease inactive Cas9 domains include, but are not limited to, D839A, N863A, and/or K603R. Cas9, dCas9, or Cas9 variant also encompasses Cas9, dCas9, or Cas9 variants from any organism. Also appreciated is that dCas9, Cas9 nickase, or other appropriate Cas9 variants from any organisms may be used in accordance with the present disclosure.

In some embodiments, an epigenetic editing system comprises a high fidelity Cas9 domain. For example, high fidelity Cas9 domains comprising one or more mutations that decrease electrostatic interactions between the Cas9 domain and the sugar-phosphate backbone of DNA may be incorporated in an epigenetic editing system to confer increased target binding specificity as compared to a corresponding wild-type Cas9 domain. Without wishing to be bound by any particular theory, high fidelity Cas9 domains that have decreased electrostatic interactions with the sugar-phosphate backbone of DNA may have less off-target effects. In some embodiments, the Cas9 domain comprises one or more mutations that decreases the association between the Cas9 domain and the sugar-phosphate backbone of DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, or more. In some embodiments, a high fidelity Cas9 domain comprises one or more of N497X, R661X, Q695X, and/or Q926X mutation as numbered in the wild type Cas9 amino acid sequence Uniprot Reference Sequence: Q99ZW2 or a corresponding amino acid in another Cas9, wherein X is any amino acid. In some embodiments, a high fidelity Cas9 domain comprises one or more of N497A, R661A, Q695A, and/or Q926A mutation of the amino acid sequence provided in the wild type Cas9 sequence, or a corresponding mutation as numbered in the wild type Cas9 amino acid sequence Uniprot Reference Sequence: Q99ZW2 or a corresponding amino acid in another Cas9. It should be appreciated that any of the epigenetic editing systems provided herein, for example, any of the epigenetic activators or repressors provided herein, may be converted into high fidelity epigenetic editing systems by modifying the Cas9 domain as described. In preferred embodiments, the high fidelity Cas9 domain is a nuclease inactive Cas9 domain.

In some embodiments, a DNA binding domain in an epigenetic editing system is a CRISPR protein that recognizes a protospacer adjacent motif (PAM) sequence in a target gene. A CRISPR protein may recognize a naturally occurring or canonical PAM sequence or may have altered PAM specificities. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et ah, “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the Cas9 domain is a Cas9 domain from S. pyogenes (SpCas9). In some embodiments, a SpCas9 recognizes a canonical NGG PAM sequence where the “N” in “NGG” is adenine (A), thymine (T), guanine (G), or cytosine (C), and the G is guanine. In some embodiments, an epigenetic editing system or fusion protein provided herein contains a SpCas9 domain that is capable of binding a nucleotide sequence that does not contain a canonical (e.g., NGG) PAM sequence. In some embodiments, the SpCas9 domain, the nuclease inactive SpCas9 domain, or the SpCas9 nickase domain can bind to a nucleic acid sequence having a NGG, a NGA, or a NGCG PAM sequence. In some embodiments, the Cas9 domain is a modified SpCas9 domain having specificity for a 5′-NGCG-3′ PAM sequence, where N is any one of nucleotides A, G, C, or T. In some embodiments, the Cas9 domain is a modified SpCas9 domain having specificity for a 5′-NGAN-3′ or a 5-NGNG-3′ PAM sequence, where N is any one of nucleotides A, G, C, or T. In some embodiments, the Cas9 domain is a modified SpCas9 domain having specificity for a 5′-NGN-3′ PAM sequence, where N is any one of nucleotides A, G, C, or T. In some embodiments, the Cas9 domain is a modified SpCas9 domain having specificity for a 5′-NRN-3′ or a 5′-NYN-3′ PAM sequence, where N is any one of nucleotides A, G, C, or T, where R is nucleotide A or G, and where Y is nucleotide C or T.

In some embodiments, the Cas9 domain is a Cas9 domain from Staphylococcus aureus (SaCas9). In some embodiments, the SaCas9 domain is a nuclease inactive SaCas9 (dSacas9). In some embodiments, the SaCas9 domain, the nuclease inactive SaCas9 domain, or the SaCas9 nickase domain can bind to a nucleic acid sequence having a non-canonical PAM. In some embodiments, the SaCas9 domain, the SaCas9d domain, or the SaCas9n domain can bind to a nucleic acid sequence having a NNGRRT PAM sequence, where N=A, T, C, or G, and R=A or G. In some embodiments, the Cas9 domain is a Cas9 domain from Neisseria meningitidis (NmeCas9). In some embodiments, the NmeCas9 domain is a nuclease inactive NmeCas9 (dNmeCas9). An NmeCas9 may have specificity for a 5′-NNNGATT-3′ PAM, where N is any one of nucleotides A, G, C, or T. In some embodiments, the Cas9 domain is a Cas9 domain from Campylobacter jejuni (CjCas9). In some embodiments, the CjCas9 domain is a nuclease inactive CjCas9 (dCjCas9). A Cj Cas9 may have specificity for a 5′-NNNVRYM-3′ PAM, where N is any one of nucleotides A, G, C, or T, V is nucleotide A, C, or G, R is nucleotide A or G, Y is nucleotide C or T, and M is nucleotide A or C. In some embodiments, the Cas9 domain is a Cas9 domain from Streptococcus thermophilus (StCas9). In some embodiments, the StCas9 is encoded by St CRISPR1 loci of the Streptococcus thermophilus (St1Cas9). In some embodiments, the St1Cas9 domain is a nuclease inactive St1Cas9 (dSt1Cas9). An St1Cas9 may have specificity for a 5′-NNAGAAW-3′ PAM, where N is any one of nucleotides A, G, C, or T, and W is nucleotide A or T. In some embodiments, the StCas9 is encoded by St CRISPR3 loci of the Streptococcus thermophilus (St3Cas9). In some embodiments, the St3Cas9 domain is a nuclease inactive St3Cas9 (dSt3Cas9). An St3Cas9 may have specificity for a 5′-NGGNG-3′ PAM, where N is any one of nucleotides A, G, C, or T.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 sequences provided herein.

In some embodiments, an epigenetic editing system provided herein comprises a Cpf1 (or Cas12a) protein domain. For example, an epigenetic editing system can comprise a nuclease inactive Cpf1 protein or a variant thereof. The Cpf1 protein has a RuvC-like endonuclease domain that is similar to the RuvC domain of Cas9 but does not have a HNH endonuclease domain, and the N-terminal of Cpf1 does not have the alpha-helical recognition lobe of Cas9.

In some embodiments, the Cpf1 protein comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to the FnCpf1 sequence provided herein. It should be appreciated that Cpf1 from other bacterial species may also be used in accordance with the present disclosure.

In some embodiments, the Cpf1 is a Cpf1 protein from Lachnospiraceae bacterium (LbCpf1). A LbCpf1 may have specificity for a 5′-TTTV-3′ PAM sequence, where V is any one of nucleotides A, G, or C. In some embodiments, the LbCpf1 protein has reduced nuclease activity. In some embodiments, the nuclease activity of the LbCpf1 protein is abolished (dLbCpf1). In some embodiments, the Cpf1 is a Cpf1 protein from Acidaminococcus sp. (AsCpf1). A AsCpf1 may have specificity for a 5′-TTTV-3′ PAM sequence, where V is any one of nucleotides A, G, or C. In some embodiments, the AsCpf1 protein has reduced nuclease activity. In some embodiments, the nuclease activity of the AsCpf1 protein is abolished (dAsCpf10. In some embodiments, the dAsCpf1 or AsCpf1 protein further comprises mutations that improve fidelity of target recognition of the protein. In some embodiments, the dAsCpf1 or AsCpf1 protein further comprises mutations that result in altered PAM specificity of the protein.

In some embodiments, an epigenetic editing system provided herein comprises a Cas protein domain other than Cas9. In some embodiments, the Cas9 protein comprises an inactivated nuclease domain. In some embodiments, an epigenetic editing system comprises a Cas12a, a Cas12b, a Cas12c, a Cas12d, a Cas12e, a Cas12h, or a Cas12i domain. In some embodiments, the Cas9 protein is a RNA nuclease or an inactivated RNA nuclease. In some embodiments, an epigenetic editing system comprises a Cas12g, a Cas13a, a Cas13b, a Cas13c, or a Cas13d domain. In some embodiments, an epigenetic editing system comprises an Argonaut protein domain.

A CRISPR/Cas system or a Cas protein in an epigenetic editing system provided herein may comprise Class 1 or Class 2 Cas proteins. The Class 1 or Class 2 proteins used in an epigenetic editing system may be inactivated in its nuclease activity. In some embodiments, an epigenetic editing system comprises a Cas protein derived from a Type II, Type IIA, Type IIB, Type IIC, Type V, or Type VI Cas nuclease. In some embodiments, an epigenetic editing system comprises a Cas protein derived from a Class 2 Cas nucleases derived from Cas1, Cas1B, Cas2, Cas3, Cas4, Cas5, Cas6, Cas7, Cas8, Cas10, Cas14a, Cas14b, Cas14c, CasX, CasY, CasPhi, C2c4, C2c8, C2c9, C2c10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, or homologues or modified versions thereof. In some embodiments, a Cas protein in an epigenetic editing system is a nuclease inactivated Cas protein.

In some embodiments, the epigenetic editing system comprises a CasX (Cas12e) protein. A CasX protein may have specificity for a 5′-TTCN-3′ PAM sequence, where N is any one of nucleotides A, G, T, or C. In some embodiments, the CasX protein has reduced or abolished nuclease activity (dCasX). In some embodiments, the epigenetic editing system comprises a CasY (Cas12d) protein. A CasY protein may have specificity for a 5′-TA-3′ PAM sequence. In some embodiments, the CasY protein has reduced or abolished nuclease activity (dCasY). In some embodiments, the epigenetic editing system comprises a Casφ (CasPhi) protein. A Casφ protein may have specificity for a 5′-TTN-3′ PAM sequence, wherein N is any one of nucleotides A, T, G, or C. In some embodiments, the Casφ protein has reduced or abolished nuclease activity (dCasφ).

In some embodiments, the Cas protein is a circular permutant Cas protein. For example, an epigenetic editing system may comprise a circular permutant Cas9 as described in Oakes et al., Cell 176, 254-267 (2019), incorporated herein in its entirety. As used herein, the term “circular permutant” refers to a variant polypeptide (e.g., of a subject Cas protein) in which one section of the primary amino acid sequence has been moved to a different position within the primary amino acid sequence of the polypeptide, but where the local order of amino acids has not been changed, and where the three-dimensional architecture of the protein is conserved. For example, a circular permutant of a wild type 1000 amino acid polypeptide may have an N-terminal residue of residue number 500 (relative to the wild type protein), where residues 1-499 of the wild type protein are added the C-terminus. Such a circular permutant, relative to the wild type protein sequence would have, from N-terminus to C-terminus, amino acid numbers 500-1000 followed by 1-499, resulting in a circular permutant protein with amino acid 499 being the C-terminal residue. Thus, such an example circular permutant would have the same total number of amino acids as the wild type reference protein, and the amino acids would be in the same order locally in specific regions of the circular permutant, but the overall primary amino acid sequence is changed.

In some embodiments, an epigenetic editing system comprises a circular permuted Cas protein, e.g., a circular permuted Cas9 protein. In some embodiments, the epigenetic editing system comprises a fusion of a circular permuted Cas protein and an epigenetic effector domain, where the epigenetic effector domain is fused to the circular permuted Cas protein to a N-terminus or C-terminus that is different from that of wild type Cas protein.

In some embodiments, the circular permuted Cas protein comprises a N-terminal end of an N-terminal fragment of a wild type Cas protein fused to a C-terminus of a C-terminal fragment of the wild type Cas protein, hereby generating new N- and C-termini. Without wishing to be bound by any theory, the N-terminus and C-terminus of a wild type Cas protein may be locked in a small region, which may cause steric hinderance when the Cas protein is fused to an effect domain and reduced access to the target DNA sequence. In some embodiments, the epigenetic editing system comprising a circular permutant Cas protein has reduced steric incompatibility as compared to an epigenetic editing system comprising a wild type Cas protein counterpart. In some embodiments, the epigenetic editing system comprising a circular permutant Cas protein has improved effectiveness as compared to an epigenetic editing system comprising a wild type Cas protein counterpart. In some embodiments, the epigenetic editing system comprising a circular permutant Cas protein has improved epigenetic editing accuracy as compared to an epigenetic editing system comprising a wild type Cas protein counterpart. In some embodiments, the epigenetic editing system comprising a circular permutant Cas protein has reduced off-target editing effect as compared to an epigenetic editing system comprising a wild type Cas protein counterpart.

### Guide Polynucleotides

In some embodiments, an epigenetic editing system comprises a guide polynucleotide (or guide nucleic acid). For example, an epigenetic editing system with a DNA binding domain that includes a CRISPR-Cas protein may also include a guide nucleic acid that is capable of forming a complex with the CRISPR-Cas protein.

Methods of using guide nucleotide sequence-programmable DNA-binding protein, such as Cas9, for site-specific DNA targeting (e.g., to modify a genome) are known in the art. The guide RNA (gRNA) may guide the programmable DNA binding protein, e.g., a Class 2 Cas protein such as a Cas9 to a target sequence on a target nucleic acid molecule, where the gRNA hybridizes with and the programmable DNA binding protein and generates modification at or near the target sequence. In some embodiments, the gRNA and an epigenetic editing system fusion protein may form a ribonucleoprotein (RNP), e.g., a CRISPR/Cas complex.

A guide nucleotide sequence, e.g., a guide RNA sequence, may comprises two parts: 1) a nucleotide sequence that shares homology to a target nucleic acid (e.g., and directs binding of a guide nucleotide sequence-programmable DNA-binding protein to the target); and 2) a nucleotide sequence that binds a nucleic acid guided programmable DNA-binding protein, for example, a CRISPR-Cas protein. The nucleotide sequence in 1) may comprise a spacer sequence that hybridizes with a target sequence. The nucleotide sequence in 2) may be referred to as a scaffold sequence of a guide nucleic acid, a tracrRNA, or an activating region of a guide nucleic acid, and may comprise a stem-loop structure. The scaffold sequences of guide nucleic acids as described in Jinek et al., Science 337:816-821 (2012), U.S. Patent Application Publication US20160208288, and U.S. Patent Application Publication US20160200779 are each incorporated herein by reference in its entirety.

A guide polynucleotide may be a single molecule or may comprise two separate molecules. For example, parts 1) and 2) as described above may be fused to form one single guide (e.g., a single guide RNA, or sgRNA), or may be two separate molecules. In some embodiments, a guide polynucleotide is a dual polynucleotides connected by a linker. In some embodiments, a guide polynucleotide is a dual polynucleotides connected by a non-nucleic acid linker, for example, a peptide linker or a chemical linker.

Methods for selecting, designing, and validating gRNAs and targeting sequences (or spacer sequences) are described herein and known to those skilled in the art. Software tools can be used to optimize the gRNAs corresponding to a target nucleic acid sequence, e.g., to minimize total off-target activity across the genome. For example, DNA sequence searching algorithm can be used to identify a target sequence in crRNAs of a gRNA for use with Cas9. Exemplary gRNA design tools, including as described in Bae, et al., Cas-OFFinder: A fast and versatile algorithm that searches for potential off-target sites of Cas9 RNA-guided endonucleases. Bioinformatics 30, 1473-1475 (2014)), is herein incorporated in its entirety.

A guide polynucleotide may be of variant lengths. In some embodiments, the length of the spacer or targeting sequence depends on the CRISPR/Cas component of the epigenetic editing system and components used. For example, different Cas proteins from different bacterial species have varying optimal targeting sequence lengths. Accordingly, the spacer sequence may comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 nucleotides in length. In some embodiments, the spacer comprised 18-24 nucleotides in length. In some embodiments, the spacer comprises 19-21 nucleotides in length. In some embodiments, the spacer sequence comprises 20 nucleotides in length. In some embodiments, a guide nucleic acid (e.g., guide RNA) is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides that is complementary to a target sequence. In some embodiments, the target sequence is a DNA sequence. In some embodiments, the degree of complementarity between the targeting sequence of the gRNA and the target sequence on the target nucleic acid molecule is at least about 60%, 65%, 70%, 75%, 80%, 85%, 90%, 95%, 97%, 98%, 99%, or 100%. In some embodiments, the targeting sequence of the gRNA and the target sequence on the target nucleic acid molecule may be 100% complementary. In other embodiments, the targeting sequence of the gRNA and the target sequence on the target nucleic acid molecule may contain at least one mismatch. For example, the targeting sequence of the gRNA and the target sequence on the target nucleic acid molecule may contain 1, 2, 3, 4, 5, 6, 7, 8, 9, or 10 mismatches.

In some embodiments, the target sequence is a sequence in the genome of a mammal. In some embodiments, the target sequence is a sequence in the genome of a human. In some embodiments, the 3′ end of the target sequence is immediately adjacent to a canonical PAM sequence (NGG). In some embodiments, the guide nucleic acid (e.g., guide RNA) is complementary to a sequence associated with a disease or disorder.

In some embodiments, a guide RNA is truncated. The truncation can comprise any number of nucleotide deletions. For example, the truncation can comprise 1, 2, 3, 4, 5, 10, 15, 20, 25, 30, 40, 50 or more nucleotides. In some embodiments, a guide polynucleotide comprises RNA. In some embodiments, a guide polynucleotide comprises DNA. In some embodiments, a guide polynucleotide comprises a mixture of DNA and RNA.

A guide polynucleotide may be modified. The modifications can comprise chemical alterations, synthetic modifications, nucleotide additions, and/or nucleotide subtractions. Modified nucleosides or nucleotides can be present in a gRNA. For example, a gRNA can comprise one or more non-naturally and/or naturally occurring components or configurations that are used instead of or in addition to the canonical A, G, C, and U residues. A modified RNA can include one or more of an alteration or a replacement, of one or both of the non-linking phosphate oxygens and/or of one or more of the linking phosphate oxygens in the phosphodiester backbone linkage, an alterations of the ribose sugar, e.g., of the 2′ hydroxyl on the ribose sugar (an exemplary sugar modification), an alteration of the phosphate moiety, a modification or replacement of a naturally occurring nucleobase, replacement or modification of the ribose-phosphate backbone, a modification of the 3′ end or 5′ end of the oligonucleotide, or replacement of a terminal phosphate group or conjugation of a moiety, cap, or linker, or any combination thereof.

In some embodiments, the ribose group (or sugar) may be modified. In some embodiments, modified ribose group may control oligonucleotide binding affinity for complementary strands, duplex formation, or interaction with nucleases. Examples of chemical modifications to the ribose group include, but are not limited to, 2′-O-methyl (2′-OMe), 2′-fluoro (2′-F), 2′-deoxy, 2′-O-(2-methoxyethyl) (2′-MOE), 2′—NH2, 2′-O-Allyl, 2′-O-Ethylamine, 2′-O-Cyanoethyl, 2′-O-Acetalester, or a bicyclic nucleotide such as locked nucleic acid (LNA), 2′-(5-constrained ethyl (S-cEt)), constrained MOE, or 2′-0,4′-C-aminomethylene bridged nucleic acid (2′,4′-BNANC). In some embodiments, 2′-O-methyl modification can increase binding affinity of oligonucleotides. In some embodiments, 2′-O-methyl modification can enhance nuclease stability of oligonucleotides. In some embodiments, 2′-fluoro modification can increase oligonucleotide binding affinity and nuclease stability.

In some embodiments, the phosphate group may be chemically modified. Examples of chemical modifications to the phosphate group includes, but are not limited to, a phosphorothioate (PS), phosphonoacetate (PACE), thiophosphonoacetate (thioPACE), amide, triazole, phosphonate, or phosphotriester modification. In some embodiments, PS linkage can refer to a bond where a sulfur is substituted for one nonbridging phosphate oxygen in a phosphodiester linkage, e.g., between nucleotides. An “s” may be used to depict a PS modification in gRNA sequences. In some embodiments, a gRNA or an sgRNA may comprise a phosphorothioate (PS) linkage at a 5′ end or at a 3′ end. In some embodiments, a gRNA or an sgRNA may comprise a phosphorothioate (PS) linkage at a 5′ end. In some embodiments, a gRNA or an sgRNA may comprise a phosphorothioate (PS) linkage at a 3′ end. In some embodiments, a gRNA or an sgRNA may comprise a phosphorothioate (PS) linkage at a 5′ end and at a 3′ end. In some embodiments, a gRNA or an sgRNA may comprise one, two, or three, or more than three phosphorothioate linkages at the 5′ end or at the 3′ end. In some embodiments, a gRNA or an sgRNA may comprise three phosphorothioate (PS) linkages at the 5′ end or at the 3′ end. In some embodiments, a gRNA or an sgRNA may comprise three phosphorothioate linkages at the 3′ end. In some embodiments, a gRNA or an sgRNA may comprise two and no more than two (i.e., only two) contiguous phosphorothioate (PS) linkages at the 5′ end or at the 3′ end. In some embodiments, a gRNA or an sgRNA may comprise three contiguous phosphorothioate (PS) linkages at the 5′ end or at the 3′ end. In some embodiments, a gRNA or an sgRNA may comprise the sequence 5′-UsUsU-3′ at the 3′end or at the 5′ end, wherein U indicates a uridine and wherein s indicates a phosphorothioate (PS) linkage.

In some embodiments, the nucleobase may be chemically modified. Examples of chemical modifications to the nucleobase include, but are not limited to, 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, or halogenated aromatic groups.

Chemical modifications can be made at a part of a guide polynucleotide or the entire guide polynucleotide. In some embodiments, a total of 1, 2, 3, 4, 5, 6, 7, 8, 9, 10, 11, 12, 13, 14, 15, 16, 17, 18, 19, 20, 21, 22, 23, 24, or 25 base pairs of a guide RNA are chemically modified. In some embodiments, a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs of a guide RNA are chemically modified. In some embodiments, a total of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 base pairs of a guide RNA are chemically modified. Chemical modifications can be made in the protospacer region, the tracr RNA, the crRNA, the stem loop, or any combination thereof.

### Zinc Finger Proteins

In some embodiments, an epigenetic editing system described herein comprises a nucleic acid binding domain comprising a zinc finger domain.

Zinc finger proteins are DNA-binding proteins that contain one or more zinc fingers. In some embodiments, a zinc finger (ZF) comprises a relatively small polypeptide domain comprising approximately 30 amino acids. A zinc finger may comprise an α-helix adjacent an antiparallel β-sheet (known as a ββα-fold) which may co-ordinate with a zinc ion between four Cys and/or His residues, as described further below. In some embodiments, a ZF domain recognizes and binds to a nucleic acid triplet, or an overlapping quadruplet, in a double-stranded DNA target sequence. In certain embodiments, ZFs may also bind RNA and proteins.

As used herein, the term “zinc finger” (ZF) or “zinc finger motif” (ZF motif) refers to an individual “finger”, which comprises a beta-beta-alpha (ββα)-protein fold stabilized by a zinc ion as described elsewhere herein. In some embodiments, each finger includes approximately 30 amino acids. In some embodiments, ZF proteins or ZF protein domains are protein motifs that contain multiple fingers or finger-like protrusions that make tandem contacts with their target molecule. For example, a ZF finger may bind a triplet or (overlapping) quadruplet nucleotide sequence. Accordingly, a tandem array of ZF fingers may be designed for ZF proteins that do not naturally exist to bind desired targets.

Zinc finger proteins are widespread in eukaryotic cells. An exemplary motif characterizing one class of these proteins (C2H2 class) is-Cys-(X) 2-4-Cys-(X) 12-His-(X) 3-5His, where X is any amino acid. A single finger domain may be about 30 amino acids in length. In some embodiments, a single finger comprises an alpha helix containing the two invariant histidine residues coordinated through zinc with the two cysteines of a single beta turn.

In some embodiments, amino acid sequence of a zinc finger protein, e.g. a Zif268 protein may be altered by making amino acid substitutions at the helix positions (e.g., positions-1, 2, 3 and 6 of Zif268) on a zinc finger recognition helix. For example, modified zinc fingers with non-naturally occurring DNA recognition specificity may be generated by phage display and combinatorial libraries with randomized sidechains in either the first or middle finger of a Zif268 and then isolated with an altered Zif268 binding site in which the appropriate DNA sub-site was replaced by an altered DNA triplet.

In some embodiments, a zinc finger comprises a C2H2 finger. In some embodiments, a zinc finger protein comprises a ZF array that comprises sequential C2H2-ZFs each contacting three or more sequential bases. In some embodiments, Zinc finger protein structures, for example, zinc finger protein Zif268 and its variants bound to DNA show a semi-conserved pattern of interactions, in which typically three amino acids from the alpha-helix of the zinc finger contact three adjacent base pairs in the DNA. Accordingly, in embodiments, zinc finger DNA-binding domains function in a modular manner with a one-to-one interaction between a zinc finger and a three-base-pair tri-nucleotide sequence in a DNA sequence.

In some embodiments, an epigenetic editing system comprises a zinc finger motif comprising of a sequence: N′--(Helix 1)--(Helix 2)--(Helix 3)--(Helix 4)--(Helix 5)--(Helix 6)--C′, wherein the (Helix) is a-six contiguous amino acid residue peptide that forms a short alpha helix. In some embodiments, an epigenetic editing system comprises a zinc finger motif comprising of a sequence: N′--(Helix 1)--(Helix 2)--(Helix 3)--(Helix 4)--(Helix 5) - - - C′, wherein the (Helix) is a-six contiguous amino acid residue peptide that forms a short alpha helix.

In some embodiments, two or more zinc fingers are linked together in a tandem array to achieve specific recognition and binding of a contiguous DNA sequence. Zinc finger or zinc finger arrays in an epigenetic editing system may be naturally occurring or may be artificially engineered for desired DNA binding specificity. For example, DNA binding characteristics of individual zinc fingers may be engineered by randomizing the amino acids at the alpha-helical positions of the zinc fingers involved in DNA binding and using selection methodologies such as phage display to identify desired variants capable of binding to DNA target sites of interest.

Engineered zinc finger binding domain can have a novel binding specificity as compared to a naturally occurring zinc finger protein. Zinc fingers with desired DNA binding specificity can be designed and selected via various approaches. For example, databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence may be used to design zinc finger arrays for specific DNA sequences. See, for example, U.S. Pat. Nos. 6,453,242, 6,534,261, and 8,772,453, incorporated by reference herein in their entirety. In some embodiments, a zinc finger array may be designed and selected from a library of zinc fingers, e.g., a randomized zinc finger library. In some embodiments, a zinc finger with novel DNA binding specific is generated by selection-based methods on combinatorial libraries. For example, a zinc finger can be selected with phage display which involves displaying zinc finger proteins on the surface of filamentous phage, followed by sequential rounds of affinity selection with biotinylated target DNA to enrich for phage expressing proteins able to bind the specific target sequence. Bacterial-two-hybrid (B2H) system may also be used for selection of zinc fingers that bind specific target sites from randomized libraries. For example, a zinc finger binding site may be placed upstream of a weak promoter driving expression of two selectable markers in host cells, e.g. E. coli cells. A library of zinc fingers, fused to a fragment of the reporter protein, e.g. a yeast Gal11P protein, can be expressed in the cells and binding of a zinc finger to the target site recruits an RNA polymerase-Gal4 fusion, thus activating transcription and allowing survival of the cells on selective medium. Rational design and selection of zinc fingers as described in Maeder et al., 2008, Mol. Cell, 31:294-301; Joung et al., 2010, Nat. Methods, 7:91-92; Isalan et al., 2001, Nat. Biotechnol., 19:656-660, Rebar, et al., Science 263, 671-673 (1994), and Joung, et al. Proc Natl Acad Sci USA 97, 7382-7387 (2000), each of which incorporated herein by reference in its entirety.

In some embodiments, zinc fingers may be evolved and selected with a continuous evolution system (PACE) comprising a host cell, e.g. a E. coli cell, a “helper phagemid” present in all host cells and encoding all phage proteins except one phage protein (e.g. a g3p protein), an “accessory plasmid”, present in all host cells, that expresses the g3p protein in response to an active library member; and a “selection phagemid” expressing the library of proteins or nucleic acids being evolved, which is replicated and packaged into secreted phage particles. Helper and accessory plasmids can be combined into a single plasmid. New host cells can only be infected by phage particles that contain g3p. Fit selection phagemids encode library members that induce g3p expression from the accessory plasmid can be packaged into phage particles that contain g3p. g3p containing phage particles can infect new cells, leading to further replication of the fit selection phagemids, while g3p-deficient phage particles are non-infectious, and therefore low-fitness selection phagemids cannot propagate. The selection system, in combination with a continuous flow of host cells through a lagoon that permits replication of the phagemid but not the host cells, may be used to rapidly select zinc fingers. PACE system as described in U.S. Pat. No. 9,023,594 is incorporated by reference in its entirety.

A zinc finger DNA binding domain of an epigenetic editing system may include one or multiple zinc fingers. For example, a zinc finger DNA binding domain may include 2, 3, 4, 5, 6, 7, 8, 9, 10, 11, 12 or more zinc fingers. In some embodiments, a zinc finger DNA binding domain has at least three zinc fingers. In some embodiments, a zinc finger DNA binding domain has at least 4, 5, or 6 zinc fingers. In some embodiments, a zinc finger DNA binding domain has three zinc fingers. In some embodiments, a zinc finger DNA binding domain has at least two zinc fingers. In some embodiments, a zinc finger DNA binding domain has an array of two-finger units.

A zinc finger DNA binding domain of an epigenetic editing system may be designed for optimized specificity. In some embodiments, a sequential selection strategy is used to design a multi-finger ZF domain. For example, in a multi-finger ZF domain, a first finger may be randomized and selected with phage display, a small pool of selected fingers may be carried into the next stage, in which the second finger is randomized and selected. The process may be repeated multiple times depending on the number of fingers in the ZF domain. In some embodiments, a parallel optimization is used to design a multi-finger ZF domain. For example, a master randomized library may be interrogated using a B2H system under low selection stringency to identify a variety of individual fingers capable of binding each 3 base pair sub-site of the target site. The three selected populations may then be randomly shuffled to generate a library of multi-finger proteins, which may subsequently be interrogated under high-stringency selection conditions to identify three-finger proteins targeted to a specific nine base pair site. In additional embodiments, a large number of low-stringency selections may be used to generate a master library of single fingers, from which multi-finger proteins, e.g., three finger ZF proteins may be selected. For example, a master library or an archive may include pre-selected zinc finger pools each containing a mixture of fingers targeted to a different three base pair subsite of DNA sequences at a defined position within a three finger ZF protein. In certain embodiments, a zinc finger archive comprises at least 192 finger pools (64 potential three bp target subsites for each position in a three-finger protein). In some embodiments, a zinc finger archive comprises at least a zinc finger pool comprises at least at least 10, 20, 30, 40, 50, 60, 70, 80, 90, 95, 100 or more different fingers. In some embodiments, a smaller library is created form the archive for interrogation with a reporting system, e.g., a bacterial two-hybrid selection system.

In some embodiments, a multiple-finger ZF domain, e.g., a three-finger ZF domain may be designed and selected using two complementary libraries. For example, a three-finger ZF domain may be designed with two pre-made zinc finger phage-display libraries, where the first library contains randomized DNA-binding amino acid positions in fingers 1 and 2, and a second library contains randomized DNA-binding amino acid positions in fingers 2 and 3. The two libraries are complementary because the first library contains randomizations in all the base-contacting positions of finger 1 and certain base-contacting positions of finger 2, whereas the second library contains randomizations in the remaining base-contacting positions of finger 2 and all the base-contacting positions of finger 3. Selections of “one-and-a-half”′ fingers from each master library may be carried out in parallel using DNA sequences in which five nucleotides have been fixed to a sequence of interest. Subsequently, zinc finger encoding sequences may be amplified from the recovered phage using PCR and sets of “one-and-a-half” fingers can be paired to yield recombinant three-finger DNA-binding domains.

In some embodiments, a multi-finger ZF domain may be designed depending on the context effects of adjacent fingers. In some embodiments, a multi-finger ZF domain is designed and without selection. For example, a three-finger ZF domain may be assembled using N-terminal and C-terminal fingers identified in other arrays containing a common middle finger, using libraries containing an archive of three-finger ZF arrays comprising pre-selected and/or tested three-finger arrays.

Software for designing and selecting ZF arrays, for example, ZiFit (http://bindr.gdcb.iastate.edu/ZiFiT/; http://www.zincfingers.org/software-tools.htm) are available and known to those skilled in the art.

Accordingly, a zinc finger DNA binding domain of an epigenetic editing system may include one or multiple zinc fingers. For example, a zinc finger DNA binding domain may include 2, 3, 4, 5, 6, 7, 8, 9, 10, 11, 12 or more zinc fingers. In some embodiments, a zinc finger DNA binding domain has at least three zinc fingers. In some embodiments, a zinc finger DNA binding domain has at least 4, 5, or 6 zinc fingers. In some embodiments, a zinc finger DNA binding domain has three zinc fingers. In some embodiments, a zinc finger DNA binding domain comprising at least three zinc fingers recognizes a target DNA sequence of 9 or 10 nucleotides. In some embodiments, a zinc finger DNA binding domain comprising at least four zinc fingers recognizes a target DNA sequence of 12 to 14 nucleotides. In some embodiments, a zinc finger DNA binding domain comprising at least six zinc fingers recognizes a target DNA sequence of 18 to 21 nucleotides.

In some embodiments, an epigenetic editing system as disclosed herein comprises non-natural and suitably contain 3 or more zinc fingers. In some embodiments, an epigenetic editing system comprises 4, 5, 6, 8, 9, 10, 11, 12, 13, 14, 15, 16, 17, 18 or more (e.g. up to approximately 30 or 32) zinc fingers motifs arranged adjacent one another in tandem, forming arrays of ZF motifs. In some embodiments, an epigenetic editing system includes at least 3 ZF motifs, at least 4 ZF motifs, at least 5 ZF motifs, or at least 6 ZF motifs, at least 7 ZF motifs, at least 8 ZF motifs, at least 9 ZF motifs, at least 10 ZF motifs, at least 11 or at least 12 ZF motifs in the nucleic acid binding domain. In some embodiments, an epigenetic editing system includes up to 6, 7, 8, 10, 11, 12, 16, 17, 18, 22, 23, 24, 28, 29, 30, 34, 35, 36, 40, 41, 42, 46, 47, 48, 54, 55, 56, 58, 59, or 60 ZF motifs in the nucleic acid binding domain.

In some embodiments, a zinc finger or zinc finger array targeting a specific DNA sequence is designed with a modular assembly approach. For example, two or more pre-selected zinc fingers may be fused in a tandem fashion.

In some embodiments, a zinc finger array comprises multiple zinc fingers fused via peptide bonds. In some embodiments, a zinc finger array comprises multiple zinc fingers, one or more of which connected by peptide linkers. For example, zinc fingers in a multiple finger array can be linked by peptide linkers of 3, 4, 5, 6, 7, 8, 9, 10, 11, 12, 13, 14, 15 or more amino acids in length. In some embodiments, zinc fingers in a multiple finger array are linked by peptide linkers of 5 amino acids in length. In some embodiments, zinc fingers in a multiple finger array are linked by peptide linkers of 6 amino acids in length.

In some embodiments, ZF-containing proteins may contain ZF arrays of 2 or more ZF motifs, which may be directly adjacent one another (i.e., separated by a short (canonical) linker sequence), or may be separated by longer, flexible or structured polypeptide sequences. In some embodiments, directly adjacent fingers bind to contiguous nucleic acid sequences, i.e., to adjacent trinucleotides/triplets. In some embodiments, adjacent fingers cross-bind between each other's respective target triplets, which may help to strengthen or enhance the recognition of the target sequence and leads to the binding of overlapping quadruplet sequences. In some embodiments, distant ZF domains within the same protein may recognize (or bind to)non-contiguous nucleic acid sequences or even to different molecules (e.g., protein rather than nucleic acid).

In some embodiments, an epigenetic editing system comprises zinc fingers comprising more than 3-fingers. In some embodiments, an epigenetic editing system comprises at least 6 zinc fingers in the DNA binding domain. In some embodiments, an epigenetic editing system comprises 6 zinc fingers in the DNA binding domain that binds to a 18 bp target sequence. In some embodiments, the 18 bp target sequence is unique in the human genome. In some embodiments, an epigenetic editing system comprises zinc fingers comprising at least 7, 8, 9, 10, 11, 12, 13, 14, 15 or more zinc fingers. In some embodiments, the strong affinity of three-finger proteins would allow subsets of the longer array to bind DNA and therefore decrease specificity. Without wishing to be bound by any theory, zinc finger proteins comprising multiple two-finger units or three-finger units joined by extended linkers may confer higher DNA binding specificity as compared to fewer fingers, or an array with same number of fingers simply joined via peptide bonds. In some embodiments, an epigenetic editing system comprises at least three two-finger units connected by peptide linkers, where each of the two finger units binds a subsite in the target DNA sequence. In some embodiments, an epigenetic editing system comprises at least four two-finger units connected by peptide linkers, wherein each of the two finger units binds a subsite in the target DNA sequence. In some embodiments, an epigenetic editing system comprises at least five two-finger units connected by peptide linkers, wherein each of the two finger units binds a subsite in the target DNA sequence. In some embodiments, an epigenetic editing system comprises at least six, seven, eight, nine, ten, or more two-finger units connected by peptide linkers, wherein each of the two finger units binds a subsite in the target DNA sequence. In some embodiments, an epigenetic editing system comprises at least two three-finger units connected by peptide linkers, where each of the three finger units binds a subsite in the target DNA sequence. In some embodiments, an epigenetic editing system comprises at least three three-finger units connected by peptide linkers, where each of the three finger units binds a subsite in the target DNA sequence. In some embodiments, an epigenetic editing system comprises at least four three-finger units connected by peptide linkers, wherein each of the three finger units binds a subsite in the target DNA sequence. In some embodiments, an epigenetic editing system comprises at least five three-finger units connected by peptide linkers, wherein each of the three finger units binds a subsite in the target DNA sequence. In some embodiments, an epigenetic editing system comprises at least six, seven, eight, nine, ten, or more three-finger units connected by peptide linkers, wherein each of the three finger units binds a subsite in the target DNA sequence.

In some embodiments, multiple zinc fingers, each recognizing three specific DNA nucleotides, or trinucleotide “subsites”, are assembled to target specific DNA sequences in target genes. In some embodiments, such DNA subsites are contiguous sequences in a target gene. In some embodiments, one or more of the DNA subsites are separated by gaps in the target gene. for example, a multi-finger ZF may recognize DNA subsites that span a 1, 2, 3 or more base pairs of inter-subsite gaps between adjacent subsites. In some embodiments, zinc fingers in the multi-finger ZF are connect via peptide linkers. The peptide linkers may be of 2, 3, 4, 5, 6, 7, 8, 9, 10, 11, 12, 13, 14, 15, 16, 17, 18, 19, 20, or more amino acids in length. In some embodiments, a linker comprises 5 or more amino acids. In some embodiments, a linker comprises 7-17 amino acids. In some embodiments, the linker is a flexible linker. In some embodiments, the linker is a rigid linker, e.g., a linker comprising one or more Prolines.

Zinc finger arrays with sequence specific DNA binding activity may be fused to functional effector domains, e.g., epigenetic effector domains as described herein to confer epigenetic modifications to DNA sequences, or associated histones in a target gene. In some embodiments, an epigenetic editing system described herein comprises a zinc finger array having specificity for a target DNA sequence. In some embodiments, the two linkers of the zinc finger array are the same. In some embodiments, the two linkers of the zinc finger array are different.

In some embodiments, the programmable DNA binding protein comprises an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of −24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 July; 34 (7): 768-73. PubMed PMID: 27136078; Swarts et al., Nature. 507 (7491) (2014): 258-61; and Swarts et al., Nucleic Acids Res. 43 (10) (2015): 5120-9, each of which is incorporated herein by reference.

In some embodiments, the nucleic acid binding domain comprises a virus derived RNA-binding domain guided by an RNA sequence to bind the target gene. In some embodiments, the nucleic acid binding domain comprises a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or any other RNA recognition motifs.

In some embodiments, the nucleic acid binding domain comprises an inactivated nuclease, for example, an inactivated meganuclease. Additional non-limiting examples of DNA binding domains include tetracycline-controlled repressor (tetR) DNA binding domain, leucine zippers, helix-loophelix (HLH) domains, helix-turn-helix domains, zinc fingers, β-sheet motifs, steroid receptor motifs, bZIP domains homeodomains, and AT-hooks.

### Target Sequences

As used herein, a “target polynucleotide sequence” may be a nucleic acid sequence present in a gene of interest. The target sequence may be in a genome of, or expressed in, a cell. In an aspect, epigenetic editing systems provided herein are used to bind target polynucleotide sequences and effect epigenetic modifications and/or transcription modulation of the target gene. For example, a target sequence may be recognized by a zinc finger array of an epigenetic editing system or may hybridize with a guide RNA sequence complexed with a nuclease inactive CRISPR protein of an epigenetic editing system. In embodiments where the epigenetic editing system comprises a gRNA-dCas-effector domain complex, the gRNA is designed to have complementarity to the target sequence (or identity to the opposing strand of the target sequence, e.g., the protospacer sequence). In some embodiments, the gRNA comprises a spacer sequence is 100% identical to a protospacer sequence in the target sequence. In some embodiments, the gRNA sequence comprises a spacer sequence that is about 95%, 90%, 85%, or 80% identical to a protospacer sequence in the target sequence.

In some embodiments, the target sequence is an endogenous sequence of an endogenous gene of a host cell. In some embodiments, the target sequence is an exogenous sequence.

The target sequence may be any region of the polynucleotide (e.g., DNA sequence) suitable for epigenetic editing. For example, the target polynucleotide sequence may be any part of a target gene. In some embodiments, the target polynucleotide sequence is part of a transcriptional regulatory sequence. In some embodiment, the target polynucleotide sequence is part of a promoter, enhancer or silencer. In some embodiments, the target polynucleotide sequence is part of a promoter. In some embodiments, the target polynucleotide sequence is part of an enhancer. In some embodiments, the target polynucleotide sequence is part of a silencer. In some embodiments, the target polynucleotide sequence is within about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, or 100 base pairs (bp) flanking a transcription start site. In some embodiments, the target polynucleotide sequence is within about 1000, 900, 800, 700, 600, 500, 400, 300, 200, or 100 base pairs (bp) flanking a transcription start site. In some embodiments, the target polynucleotide sequence is within about 500, 400, 300, 200, or 100 base pairs (bp) flanking a transcription start site.

In some embodiments, the target polynucleotide sequence is within about 100 base pairs (bp) flanking a transcription start site.

In some embodiments, the target polynucleotide sequence is a hypomethylated nucleic acid sequence. In some embodiments, the target polynucleotide sequence is a hypermethylated nucleic acid sequence. In some embodiments, the target polynucleotide sequence is at, near, or within a promoter sequence. In some embodiments, the target polynucleotide sequence is at, near, or within a promoter sequence. In aspects, the target polynucleotide sequence is adjacent to a CpG island. In aspects, the target polynucleotide sequence is known to be associated with a disease or condition.

### Modulation of Expression of Target Gene

In some embodiments, the disclosure provides epigenetic editing system systems, compositions and methods for epigenetic modifications at a target polynucleotide in a target gene encoding a protein. In some embodiments, the epigenetic editing system results in epigenetic modification, e.g., DNA methylation, in a coding region of the target gene, thereby reducing or silencing expression of the target gene. In some embodiments, the epigenetic editing system results in epigenetic modification, e.g., DNA methylation, in a regulatory sequence such as a promoter or enhancer of the target gene, thereby reducing or silencing expression of the target gene. In some embodiments, the epigenetic editing system results in transcription repression or recruits a transcription repressor to a coding region of the target gene, thereby reducing or silencing expression of the target gene. In some embodiments, the epigenetic editing system recruits a transcription repressor to a regulatory sequence such as a promoter or enhancer of the target gene, thereby reducing or silencing expression of the target gene. In some embodiments, the epigenetic editing system results in epigenetic modification, e.g., DNA demethylation, in a coding region of the target gene, thereby increasing expression of the target gene. In some embodiments, the epigenetic editing system results in epigenetic modification, e.g., DNA demethylation, in a regulatory sequence such as a promoter or enhancer of the target gene, thereby increasing expression of the target gene. In some embodiments, the epigenetic editing system results in transcription activation or recruits a transcription activator to a coding region of the target gene, thereby increasing expression of the target gene. In some embodiments, the epigenetic editing system recruits a transcription activator to a regulatory sequence such as a promoter or enhancer of the target gene, thereby increasing expression of the target gene.

In some embodiments, the target gene and/or the protein encoded are associated with a disease, disorder, or pathogenic condition.

Epigenetic modifications effected by the epigenetic editing systems described herein are sequence specific. In some embodiments, the modification is at a specific site of the target polynucleotide. In some embodiments, the modification is at a specific allele of the target gene. Accordingly, the epigenetic modification may result in modulated expression, for example, reduced or increased expression, of one copy of a target gene harboring a specific allele, and not the other copy of the target gene. In some embodiments, the specific allele is associated with a disease, condition, or disorder.

Epigenetic modification may be made at any target genes of a genome of interest, for example, a prokaryote genome, a plant genome, mammalian or human genome. The target gene can be of or derived from any organism and genome thereof. For example, the target gene can be a prokaryotic gene, a eukaryotic gene, an animal gene, a plant gene, a mouse gene, a rat gene, a rabbit gene, a fish gene, an avian gene, a monkey gene, or a human gene. In some embodiments, the target gene is a reporter gene the expression of which can be readily tracked and monitored. Reporter genes and reporter systems include, for example, sequences encoding green fluorescence proteins, red fluorescence proteins, enhanced yellow or enhanced cyan proteins, or luciferase proteins. In some embodiments, the target gene encodes a selectable marker, for example, a beta-galactosidase, a Chloramphenicol acetyltransferase, or an antibiotic resistance marker. In some embodiments, the target gene is associated with, or harbors one or more mutations that are associated with a disease, condition, or disorder. Non-limiting exemplary target genes include HBB, HBA, hMSH2, HMLH1, growth factors GM-SCF, VEGF, EPO, Erb-B2, and hGH.

Target genes also include plant genes for which repression or activation leads to an improvement in plant characteristics, such as improved crop production, disease or herbicide resistance. For example, repression of expression of the FAD2-1 gene results in an advantageous increase in oleic acid and decrease in linoleic and linoleic acids.

In some embodiments, an epigenetic editing system provided herein effects an epigenetic modification in a gene that harbors a target sequence. In some embodiments, the epigenetic editing system modulates expression of a protein encoded by the gene. In some embodiments, the epigenetic editing system reduces the level of a protein encoded by the gene. In some embodiments, the epigenetic editing system increases the level of a protein encoded by the gene.

To generate epigenetic edits at a target gene, a target gene polynucleotide may be contacted with the epigenetic editing compositions disclosed herein comprising a target DNA binding domain, an epigenetic effector domain, e.g., an epigenetic repressor domain, wherein the DNA binding domain directs the epigenetic effector domain to a target polynucleotide sequence in the target gene, resulting in the epigenetic modification, e.g., a methylation state modification. In some embodiments, the epigenetic editing system effects an alteration in the methylation state of a target DNA sequence in the target gene. In some embodiments, the epigenetic editing system effects an alteration in the methylation state of a specific allele in the target gene. In some embodiments, the epigenetic editing system effects an alteration in the methylation state of a histone protein associated with the target gene.

In some embodiments, the epigenetic modification reduces transcription of the target gene harboring the target sequence. In some embodiments, the epigenetic modification abolishes transcription of the target gene harboring the target sequence. In some embodiments, the epigenetic modification reduces transcription of a copy of the target gene harboring a specific allele recognized by the epigenetic editing system. In some embodiments, the epigenetic modification abolishes transcription of a copy of the target gene harboring a specific allele recognized by the epigenetic editing system. In some embodiments, the epigenetic editing system reduces the level of a protein encoded by the target gene. In some embodiments, the epigenetic editing system eliminates expression of a protein encoded by the target gene. In some embodiments, the epigenetic editing system reduces the level of a protein encoded by a copy of the target gene harboring a specific allele recognized by the epigenetic editing system. In some embodiments, the epigenetic editing system eliminates expression of a protein encoded by a copy of the target gene harboring a specific allele recognized by the epigenetic editing system.

In some embodiments, the epigenetic modification increases transcription of the target gene harboring the target sequence. In some embodiments, the epigenetic modification increases transcription of a copy of the target gene harboring a specific allele recognized by the epigenetic editing system. In some embodiments, the epigenetic editing system increases the level of a protein encoded by the target gene. In some embodiments, the epigenetic editing system increases the level of a protein encoded by a copy of the target gene harboring a specific allele recognized by the epigenetic editing system.

The target gene may be epigenetically modified in vitro, ex vivo, or in vivo. Accordingly, epigenetic modification of the target gene may modulate expression of a target gene, or an allele thereof, in a cell ex vivo or in a subject in vivo. In some embodiments, the target polynucleotide sequence is the gene locus in the genomic DNA of a cell. In some embodiments, the cell is a cultured cell. In some embodiments, the cell is in vitro. In some embodiments, the cell is ex vivo. In some embodiments, the cell is in vivo. For example, an epigenetic editing system, e.g., a fusion protein comprising a zinc finger array and an effector domain, or a sgRNA complexed with a Cas protein-effector domain fusion, may be expressed in a cell where modulated expression of a target gene is desired to thereby allow contact of the target gene with the epigenetic editing system described herein. In some embodiments, the cell is from a mammal. In some embodiments, the mammal is a human. In some embodiments, the mammal is a rodent. In some embodiments, the rodent is a mouse. In some embodiments, the rodent is a rat.

In some embodiments, the epigenetic editing systems described herein reduces expression of a target gene by at least about 20%, at least about 30%, at least about 40%, at least about 50%, at least about 60%, at least about 70%, at least about 80%, at least about 90%, at least about 95%, at least about 99%, or more, as measured by transcription of the target gene in a cell, a tissue, or a subject as compared to a control cell, control tissue, or a control subject. In some embodiments, the epigenetic editing systems described herein reduces expression of a copy of target gene by at least about 20%, at least about 30%, at least about 40%, at least about 50%, at least about 60%, at least about 70%, at least about 80%, at least about 90%, at least about 95%, at least about 99%, or more, as measured by transcription of the copy of the target gene in a cell, a tissue, or a subject as compared to a control cell, control tissue, or a control subject. In some embodiments, the copy of the target gene harbors a specific sequence or allele recognized by the epigenetic editing system. In some embodiments, the epigenetically modified copy encodes a functional protein. Accordingly, in some embodiments, an epigenetic editing system composition disclosed herein reduces or abolishes expression and/or function of protein encoded by a target gene, by reducing or abolishing expression of a functional protein encoded by the target gene. For example, the methods and composition disclosed herein may reduce expression and/or function of a protein encoded by the target gene by at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold, at least 60-fold, at least 70-fold, at least 80-fold, at least 90-fold, or at least 100 fold in a cell, a tissue, or a subject as compared to a control cell, control tissue, or a control subject.

In some embodiments, the epigenetic editing systems described herein increases expression of a target gene by at least about 20%, at least about 30%, at least about 40%, at least about 50%, at least about 60%, at least about 70%, at least about 80%, at least about 90%, at least about 95%, at least about 99%, at least 100%, at least 110%, at least 120%, at least 130%, at least 140%, at least 150%, at least 160%, at least 170%, at least 180%, at least 190%, at least 200%, at least 250%, at least 300%, at least 350%, at least 400%, at least 450%, at least 500% or more, as measured by transcription of the target gene in a cell, a tissue, or a subject as compared to a control cell, control tissue, or a control subject. In some embodiments, the epigenetic editing systems described herein increases expression of a copy of target gene by at least about 20%, at least about 30%, at least about 40%, at least about 50%, at least about 60%, at least about 70%, at least about 80%, at least about 90%, at least about 95%, at least about 99%, at least 100%, at least 110%, at least 120%, at least 130%, at least 140%, at least 150%, at least 160%, at least 170%, at least 180%, at least 190%, at least 200%, at least 250%, at least 300%, at least 350%, at least 400%, at least 450%, at least 500% or more, as measured by transcription of the copy of the target gene in a cell, a tissue, or a subject as compared to a control cell, control tissue, or a control subject. In some embodiments, the copy of the target gene harbors a specific sequence or allele recognized by the epigenetic editing system. In some embodiments, the epigenetically modified copy encodes a functional protein. Accordingly, in some embodiments, an epigenetic editing system composition disclosed herein increases expression and/or function of protein encoded by a target gene, by increasing expression of a functional protein encoded by the target gene. For example, the methods and composition disclosed herein may increase expression and/or function of a protein encoded by the target gene by at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold, at least 60-fold, at least 70-fold, at least 80-fold, at least 90-fold, or at least 100 fold in a cell, a tissue, or a subject as compared to a control cell, control tissue, or a control subject.

Methods for determining the expression level of a gene, for example the target of an epigenetic editing system, are known in the art. For example, transcript level of a gene may be determined by reverse transcription PCR, quantitative RT-PCR, droplet digital PCR (ddPCR), Northern blot, RNA sequencing, DNA sequencing (e.g., sequencing of complementary deoxyribonucleic acid (cDNA) obtained from RNA); next generation (Next-Gen) sequencing, nanopore sequencing, pyrosequencing, or Nanostring sequencing. Protein level expressed from a gene may be determined by western blotting, enzyme linked immuno-absorbance assays, mass-spectrometry, immunohistochemistry, or flow cytometry analysis. Gene expression product levels may be normalized to an internal standard such as total messenger ribonucleic acid (mRNA) or the expression level of a particular gene, e.g., a house keeping gene.

In some embodiments, the effect of an epigenetic editing system in modulating target gene expression may be examined using a reporter system. For example, an epigenetic editing system may be designed to target a reporter gene encoding a reporter protein, e.g. a fluorescent protein. Expression of the reporter gene in such a model system may be monitored by, e.g., flow cytometry, fluorescence-activated cell sorting (FACS), or fluorescence microscopy. In some embodiments, a population of cells may be transfected with a vector which harbors a reporter gene. The vector may be constructed such that the reporter gene is expressed when the vector transfects a cell. Suitable reporter genes include genes encoding fluorescent proteins, for example green, yellow, cherry, cyan or orange fluorescent proteins. The population of cells carrying the reporter system may be transfected with DNA, mRNA, or vectors encoding the epigenetic editing system targeting the reporter gene. The level of expression of the reporter gene may be quantified using a suitable technique, such as FACS.

Epigenetic editing systems described herein may be expressed in a host cell transiently or may be integrated in a genome of the host cell. Both transiently expressed and integrated epigenetic editing systems can effect stable epigenetic modifications. For example, after introduction of an epigenetic editing system comprising a DNA binding domain specific for a target gene and an epigenetic repression domain to a host cell, the target gene in the host cell may be stably or permanently repressed. In some embodiments, expression of the target gene is reduced for at least 1 week, at least 2 weeks, at least 3 weeks, at least 4 weeks, at least 5 weeks, at least 6 weeks, at least 7 weeks, at least 2 months, at least 3 months, at least 5 months, at least 6 months, at least 1 year, at least 2 years, or for the entire lifetime of the cell or the subject carrying the cell, as compared to the level of expression in the absence of the epigenetic editing system. In some embodiments, expression of the target gene is silenced for at least 1 week, at least 2 weeks, at least 3 weeks, at least 4 weeks, at least 5 weeks, at least 6 weeks, at least 7 weeks, at least 2 months, at least 3 months, at least 5 months, at least 6 months, at least 1 year, at least 2 years, or for the entire lifetime of the cell or the subject carrying the cell as compared to the level of expression in the absence of the epigenetic editing system. In some embodiments, after introduction of an epigenetic editing system comprising a DNA binding domain specific for a target gene and an epigenetic activation domain to a host cell, the target gene in the host cell is stably or permanently activated. In some embodiments, expression of the target gene is increased for at least 1 week, at least 2 weeks, at least 3 weeks, at least 4 weeks, at least 5 weeks, at least 6 weeks, at least 7 weeks, at least 2 months, at least 3 months, at least 5 months, at least 6 months, at least 1 year, at least 2 years, or for the entire lifetime of the cell or the subject carrying the cell as compared to the level of expression in the absence of the epigenetic editing system.

The epigenetic modification described herein may be inherited by the progeny of host cells that are contacted or introduced with an epigenetic editing system. For example, in some embodiments, after introduction of an epigenetic editing system comprising a DNA binding domain specific for a target gene and an epigenetic repression domain to a stem cell, e.g., a hematopoietic stem cell, expression of the target gene is also repressed in cells differentiated from the stem cell compared to cells differentiated from a control stem cell in the absence of the epigenetic editing system. In some embodiments, expression of the target gene is silenced in cells differentiated from the stem cell. In some embodiments, after introduction of an epigenetic editing system comprising a DNA binding domain specific for a target gene and an epigenetic activation domain to a stem cell, e.g., a hematopoietic stem cell, expression of the target gene is also increased in cells differentiated from the stem cell compared to cells differentiated from a control stem cell in the absence of the epigenetic editing system.

Modulation of target gene expression can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP; changes in signal transduction; changes in phosphorylation and dephosphorylation; changes in receptor-ligand interactions; changes in concentrations of second messengers such as, for example, cGMP, CAMP, IP3, and Ca2+; changes in cell growth, changes in neovascularization, and/or changes in any functional effect of gene expression. Measurements can be made in vitro, in vivo, and/or ex vivo. Such functional effects can be measured by conventional methods, e.g., measurement of RNA or protein levels, measurement of RNA stability, and/or identification of downstream or reporter gene expression. Readout can be by way of, for example, chemiluminescence, fluorescence, colorimetric reactions, antibody binding, inducible markers, ligand binding assays; changes in intracellular second messengers such as cGMP and inositol triphosphate (IP3); changes in intracellular calcium levels; cytokine release, and the like.

To determine the level of gene expression modulation by a ZFP, cells contacted with ZFPs are compared to control cells, e.g., without the zinc finger protein or with a non-specific ZFP, to examine the extent of inhibition or activation. Control samples are assigned a relative gene expression activity value of 100%. Modulation/inhibition of gene expression is achieved when the gene expression activity value relative to the control is about 80%, preferably 50% (i.e., 0.5× the activity of the control), more preferably 25%, more preferably 5-0%. Modulation/activation of gene expression is achieved when the gene expression activity value relative to the control is 110%, more preferably 150% (i.e., 1.5× the activity of the control), more preferably 200-500%, more preferably 1000-2000% or more.

### Delivery

In an aspect, provided herein is a composition for gene expression modulation comprising the epigenetic editing system as provided herein that generates epigenetic modifications at target genes. The epigenetic editing system, or nucleic acid encoding the epigenetic editing system or components thereof (e.g., nucleic acids encoding an epigenetic editing system fusion protein comprising a zinc finger-repressor fusion, a Cas9-repressor fusion, and or nucleic acids encoding one or more guide RNAs) may be introduced to a cell via various ways known in the art. For example, in some embodiments, the epigenetic editing system is delivered to a host cell or integrated into the genome of the host cell, or for transient expression in the host cell.

In some embodiments, the nucleic acid encoding the epigenetic editing system or components thereof is operatively linked to a promoter and/or a regulatory sequence. The term “operably linked,” as used herein, means that the nucleotide sequence of interest is linked to regulatory sequence(s) in a manner that allows for expression of the nucleotide sequence. The term “regulatory sequence,” as used herein, includes, but is not limited to promoters, enhancers and other expression control elements. Such regulatory sequences are well known in the art and are described, for example, in Goeddel; Gene Expression Technology: Methods in Enzymology 185, Academic Press, San Diego, CA (1990).

In some embodiments, the composition further comprises a vector that comprises the nucleic acid sequence encoding an epigenetic editing system protein. In some embodiments, the vector may be an expression vector. In some embodiments, the vector is a plasmid or a viral vector. The term “vector,” as used herein, refers to a nucleic acid molecule capable of transporting another nucleic acid to which it has been linked. In some examples, a vector is an expression vector that is capable of directing the expression of nucleic acids to which they are operatively linked. Examples of expression vectors include, but are not limited to, plasmid vectors, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. In some embodiments, the vector is a virus-like particle (VLP).

Non-viral delivery systems include but are not limited to DNA delivery methods and RNA delivery methods such as transfection. Here, transfection includes a process using a non-viral vector to deliver a gene, a DNA fragment, a gene transcript, an RNA, an RNA fragment, a circularized DNA, or a circularized RNA to a target cell. Typical transfection methods can include but are not limited to electroporation, DNA biolistics, lipid-mediated transfection, compacted DNA-mediated transfection, liposomes, immunoliposomes, exosomes, lipofection, cationic agent-mediated transfection, or cationic facial amphiphiles (CFAs).

In some embodiments, the epigenetic editing system is delivered to a host cell for transient expression, e.g., via a transient expression vector. Transient expression of a epigenetic editing system may result in prolonged or permanent epigenetic modification of the target gene. For example, the epigenetic modification may be stable for at least 1, 2, 3, 4, 5, 6, 7, 8, 9, 11, 12 weeks, 3, 4, 5, 6, 7, 8, 9, 10, 11, 12 months or more after introduction of the epigenetic editing system into the host cell. The epigenetic modification may be maintained after one or more mitotic events of the host cell. The epigenetic modification may be maintained after one or more meiotic events of the host cell. In some embodiments, the epigenetic modification is maintained across generations in offspring generated or derived from the host cell.

In some embodiments, a nucleic acid sequence encoding an epigenetic editing system or components thereof is a DNA, an RNA or mRNA, or a modified nucleic acid sequence. For example, a mRNA sequence encoding an epigenetic editing system fusion protein may be chemically modified, or may comprise a 5′Cap, or one or more 3′ modifications.

Nucleic acids encoding epigenetic editing systems can be delivered directly to cells as naked DNA or RNA, for instance by means of transfection or electroporation, or can be conjugated to molecules (e.g., N-acetylgalactosamine) promoting uptake by the target cells. Nucleic acid vectors, such as the vectors can also be used. In particular embodiments, a polynucleotide, e.g., a mRNA encoding an epigenetic editing system, or a functional component thereof may be co-electroporated with a combination of multiple guide RNAs as described herein.

Nucleic acid vectors can comprise one or more sequences encoding a domain of a fusion protein or an epigenetic editing system as described herein. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40), and one or more effector domains such as repression domains.

In particular embodiments, a fusion protein, a protein domain, or a whole or a part of epigenetic editing system components is encoded by a polynucleotide present in a viral vector (e.g., adeno-associated virus (AAV), AAV3, AAV3b, AAV4, AAV5, AAV6, AAV7, AAV8, AAV9, AAVrh8, AAV10, and variants thereof), or a suitable capsid protein of any viral vector. Thus, in some aspects, the disclosure relates to the viral delivery of a fusion protein. Examples of viral vectors include retroviral vectors (e.g., Maloney murine leukemia virus, MML-V), adenoviral vectors (e.g., AD100), lentiviral vectors (HIV and FIV-based vectors), herpesvirus vectors (e.g., HSV-2).

In some embodiments, an epigenetic editing system protein is encoded by a polynucleotide present in an adeno-associated virus (AAV) vector. In some embodiments, the epigenetic editing system protein comprises a zinc finger array in the DNA binding domain. Without wishing to be bound by any theory, epigenetic editing systems using zinc finger array instead of larger DNA binding domains such as Cas protein domains can be conveniently packed in viral vectors, e.g. AAV vector, given the small size of zinc fingers. In some embodiments, the polynucleotide encoding the epigenetic editing system is of length of about 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or less. In some embodiments, the polynucleotide encoding the epigenetic editing system is of length of about 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb, 5 kb or less.

Any AAV serotype, e.g., human AAV serotype, can be used including, but not limited to, AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 11 (AAV11), a variant thereof, or a shuffled variant thereof (e.g., a chimeric variant thereof). In some embodiments, an AAV variant has at least 90%, e.g., 90%, 91%, 92%, 93%, 94%, 95%, 96%, 97%, 98%, 99% or more amino acid sequence identity to a wild-type AAV. An AAV1 variant can have at least 90%, e.g., 90%, 91%, 92%, 93%, 94%, 95%, 96%, 97%, 98%, 99% or more amino acid sequence identity to a wild-type AAV1. An AAV2 variant can have at least 90%, e.g., 90%, 91%, 92%, 93%, 94%, 95%, 96%, 97%, 98%, 99% or more amino acid sequence identity to a wild-type AAV2. An AAV3 variant can have at least 90%, e.g., 90%, 91%, 92%, 93%, 94%, 95%, 96%, 97%, 98%, 99% or more amino acid sequence identity to a wild-type AAV3. An AAV4 variant can have at least 90%, e.g., 90%, 91%, 92%, 93%, 94%, 95%, 96%, 97%, 98%, 99% or more amino acid sequence identity to a wild-type AAV4. An AAV5 variant can have at least 90%, e.g., 90%, 91%, 92%, 93%, 94%, 95%, 96%, 97%, 98%, 99% or more amino acid sequence identity to a wild-type AAV5. An AAV6 variant can have at least 90%, e.g., 90%, 91%, 92%, 93%, 94%, 95%, 96%, 97%, 98%, 99% or more amino acid sequence identity to a wild-type AAV6. An AAV7 variant can have at least 90%, e.g., 90%, 91%, 92%, 93%, 94%, 95%, 96%, 97%, 98%, 99% or more amino acid sequence identity to a wild-type AAV7. An AAV8 variant can have at least 90%, e.g., 90%, 91%, 92%, 93%, 94%, 95%, 96%, 97%, 98%, 99% or more amino acid sequence identity to a wild-type AAV8. An AAV9 variant can have at least 90%, e.g., 90%, 91%, 92%, 93%, 94%, 95%, 96%, 97%, 98%, 99% or more amino acid sequence identity to a wild-type AAV9. An AAV10 variant can have at least 90%, e.g., 90%, 91%, 92%, 93%, 94%, 95%, 96%, 97%, 98%, 99% or more amino acid sequence identity to a wild-type AAV10. An AAV11 variant can have at least 90%, e.g., 90%, 91%, 92%, 93%, 94%, 95%, 96%, 97%, 98%, 99% or more amino acid sequence identity to a wild-type AAV11. An AAV12 variant can have at least 90%, e.g., 90%, 91%, 92%, 93%, 94%, 95%, 96%, 97%, 98%, 99% or more amino acid sequence identity to a wild-type AAV12.

In some instances, one or more regions of at least two different AAV serotype viruses are shuffled and reassembled to generate an AAV chimera virus. For example, a chimeric AAV can comprise inverted terminal repeats (ITRs) that are of a heterologous serotype compared to the serotype of the capsid. The resulting chimeric AAV virus can have a different antigenic reactivity or recognition, compared to its parental serotypes. In some embodiments, a chimeric variant of an AAV includes amino acid sequences from 2, 3, 4, 5, or more different AAV serotypes.

Descriptions of AAV variants and methods for generating thereof are found, e.g., in Weitzman and Linden. Chapter 1-Adeno-Associated Virus Biology in Adeno-Associated Virus: Methods and Protocols Methods in Molecular Biology, vol. 807. Snyder and Moullier, eds., Springer, 2011; Potter et al., Molecular Therapy—Methods & Clinical Development, 2014, 1, 14034; Bartel et al., Gene Therapy, 2012, 19, 694-700; Ward and Walsh, Virology, 2009, 386 (2): 237-248; and Li et al., Mol Ther, 2008, 16 (7): 1252-1260, each incorporated herein by reference in its entirety. AAV virions (e.g., viral vectors or viral particle) described herein can be transduced into cells to introduce the epigenetic editing system or any component thereof into the cell. An epigenetic editing system can be packaged into an AAV viral vector according to any method known to those skilled in the art. Examples of useful methods are described in McClure et al., J Vis Exp, 2001, 57:3378.

A nucleic acid vector described herein can also include any suitable number of regulatory/control elements, e.g., promoters, enhancers, introns, polyadenylation signals, Kozak consensus sequences, or internal ribosome entry sites (IRES). These elements are well known in the art.

Nucleic acid vectors according to this disclosure include recombinant viral vectors. Exemplary viral vectors are set forth herein above. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g., lipid and/or polymer)nanoparticles can be suitable for use as delivery vehicles in certain embodiments of this disclosure.

In another aspect, provided herein is a lipid nanoparticle (LNP) comprising the composition as provided herein. As used herein, a “lipid nanoparticle (LNP) composition” or a “nanoparticle composition” is a composition comprising one or more described lipids. LNP compositions are typically sized on the order of micrometers or smaller and may include a lipid bilayer. Nanoparticle compositions encompass lipid nanoparticles (LNPs), liposomes (e.g., lipid vesicles), and lipoplexes. In some embodiments, a LNP refers to any particle that has a diameter of less than 1000 nm, 500 nm, 250 nm, 200 nm, 150 nm, 100 nm, 75 nm, 50 nm, or 25 nm. In some embodiments, a nanoparticle may range in size from 1-1000 nm, 1-500 nm, 1-250 nm, 25-200 nm, 25-100 nm, 35-75 nm, or 25-60 nm.

In some embodiments, an LNP may be made from cationic, anionic, or neutral lipids. In some embodiments, an LNP may comprise neutral lipids, such as the fusogenic phospholipid 1,2-Dioleoyl-sn-glycero-3-phosphoethanolamine (DOPE) or the membrane component cholesterol, as helper lipids to enhance transfection activity and nanoparticle stability. In some embodiments, an LNP may comprise hydrophobic lipids, hydrophilic lipids, or both hydrophobic and hydrophilic lipids. Any lipid or combination of lipids that are known in the art can be used to produce an LNP. Examples of lipids used to produce LNPs include, but are not limited to DOTMA (N[1-(2,3-dioleyloxy) propyl]-N,N,N-trimethylammonium chloride), DOSPA (N,N-dimethyl-N-([2-sperminecarboxamido]ethyl)-2,3-bis(dioleyloxy)-1-propaniminium pentahydrochloride), DOTAP (1,2-Dioleoyl-3-trimethylammonium propane), DMRIE (N-(2-hydroxyethyl)-N,N-dimethyl-2,3-bis(tetradecyloxy-1-propanaminiumbromide), DC-cholesterol (3β-[N-(N′,N′-dimethylaminoethane)-carbamoyl]cholesterol), DOTAP-cholesterol, GAP-DMORIE-DPyPE, and GL67A-DOPE-DMPE (,2-Bis(dimethylphosphino) ethane)-polyethylene glycol (PEG). Examples of cationic lipids include, but are not limited to, 98N12-5, C12-200, DLin-KC2-DMA (KC2), DLin-MC3-DMA (MC3), XTC, MD1, and 7C1. Examples of neutral lipids include, but are not limited to, DPSC, DPPC (Dipalmitoylphosphatidylcholine), POPC (1-palmitoyl-2-oleoyl-sn-glycero-3-phosphocholine), DOPE, and SM (sphingomyelin). Examples of PEG-modified lipids include, but are not limited to, PEG-DMG (Dimyristoyl glycerol), PEG-CerC14, and PEG-CerC20. In some embodiments, the lipids may be combined in any number of molar ratios to produce a LNP. In some embodiments, the polynucleotide may be combined with lipid(s) in a wide range of molar ratios to produce an LNP.

### Method of Treatment

Also provided herein are methods for treating or preventing a condition in a subject in need thereof, the method comprising administering to the subject the epigenetic editing system composition as described herein, wherein the epigenetic editing system complex or protein effects an epigenetic modification of a target polynucleotide in a target gene associated with a disease, condition or disorder in a subject and modulates expression of the target, thereby treating or preventing the disease, condition or disorder.

Epigenetic modifications effected by the epigenetic editing systems described herein are sequence specific. In some embodiments, the modification is at a specific site of the target polynucleotide. In some embodiments, the modification is at a specific allele of the target gene. Accordingly, the epigenetic modification may result in modulated expression, for example, reduced or increased expression, of one copy of a target gene harboring a specific allele, and not the other copy of the target gene. In some embodiments, the specific allele is associated with a disease, condition, or disorder.

In some embodiments, the epigenetic editing system reduces expression of a target gene associated with a disease, condition or disorder.

Epigenetic editing systems described herein may be administered to a subject in need thereof, in a therapeutically effective amount, to treat a disease, condition or disorder.

In another aspect, provided herein is a method for treating or preventing a condition in a subject in need thereof, the method comprising administering to the subject the epigenetic editing complex, vectors, nucleic acids, proteins, or compositions as provided herein, wherein the nucleic acid binding domain of the epigenetic editing system directs the effector domain to generate an epigenetic modification in a target polynucleotide sequence in a cell of the subject, thereby modulating expression of the target gene and treating or preventing the condition.

In some embodiments, the modification reduces expression of a functional protein encoded by the target gene in the subject.

A patient who is being treated for a condition, a disease or a disorder is one who a medical practitioner has diagnosed as having such a condition. Diagnosis may be by any suitable means. Diagnosis and monitoring may involve, for example, detecting the presence of diseased, dying or dead cells in a biological sample (e.g., tissue biopsy, blood test, or urine test), detecting the presence of plaques, detecting the level of a surrogate marker in a biological sample, or detecting symptoms associated with a condition. A patient in whom the development of a condition is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

A subject may have a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In some embodiments, the subject has hypercholesterolemia. In some embodiments, the subject has atherosclerotic vascular disease. In some embodiments, the subject has hypertriglyceridemia. In some embodiments, the subject has diabetes. In some embodiments, the subject is a mammal. In some embodiments, the subject is a non-human primate. In some embodiments, the subject is human. Alleviating a disease includes delaying the development or progression of the disease or reducing disease severity. Alleviating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir.

The therapeutic methods of the disclosure may be carried out on subjects displaying pathology resulting from a disease or a condition, subjects suspected of displaying pathology resulting from a disease or a condition, and subjects at risk of displaying pathology resulting from a disease or a condition. For example, subjects that have a genetic predisposition to a disease or a condition can be treated prophylactically. Subjects exhibiting symptoms associated with a condition, a disease or a disorder may be treated to decrease the symptoms or to slow down or prevent further progression of the symptoms. The physical changes associated with the increasing severity of a disease or a condition are shown herein to be progressive. Thus, in embodiments of the disclosure, subjects exhibiting mild signs of the pathology associated with a condition or a disease may be treated to improve the symptoms and/or prevent further progression of the symptoms.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Adjustment and manipulation of established dosages (e.g., frequency and duration) are well within the ability of those skilled in the art. The treatment, such as those disclosed herein, can be administered to the subject on a daily, twice daily, biweekly, monthly or any applicable basis that is therapeutically effective. In embodiments, the treatment is only on an as-needed basis, e.g., upon appearance of signs or symptoms of a condition or a disease.

Toxicity and therapeutic efficacy of the compositions of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects (the ratio LD50/ED50) is the therapeutic index. Agents that exhibit high therapeutic indices are preferred. The dosage of agents lies preferably within a range of circulating concentrations that include the ED50 with little or no toxicity. While agents that exhibit toxic side effects may be used, care should be taken to design a delivery system that targets such agents to the site of affected tissue in order to minimize potential damage to uninfected cells and, thereby, reduce side effects.

The skilled artisan will appreciate that certain factors may influence the dosage and frequency of administration required to effectively treat a subject, including but not limited to the severity of the disease or disorder, previous treatments, the general characteristics of the subject including health, sex, weight and/or age of the subject, and other diseases present. Moreover, treatment of a subject with a therapeutically effective amount of the compositions can include a single treatment or, preferably, can include a series of treatments. It will also be appreciated that the effective dosage of the composition of the disclosure used for treatment may increase or decrease over the course of a particular treatment. Changes in dosage may result and become apparent from the results of diagnostic assays as described herein. The therapeutically effective dosage will generally be dependent on the patient's status at the time of administration. The precise amount can be determined by routine experimentation but may ultimately lie with the judgment of the clinician, for example, by monitoring the patient for signs of disease and adjusting the treatment accordingly.

Frequency of administration may be determined and adjusted over the course of therapy, and is generally, but not necessarily, based on treatment and/or suppression and/or amelioration and/or delay of a disease. Alternatively, sustained continuous release formulations of a polypeptide or a polynucleotide may be appropriate. Various formulations and devices for achieving sustained release are known in the art. In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The dosing regimen (including a composition disclosed herein) can vary over time. In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the polypeptide or the polynucleotide (such as the half-life of the polypeptide or the polynucleotide, and other considerations well known in the art).

For the purpose of the present disclosure, the appropriate therapeutic dosage of a composition as described herein will depend on the specific agent (or compositions thereof) employed, the formulation and route of administration, the type and severity of the disease, whether the polypeptide or the polynucleotide is administered for preventive or therapeutic purposes, previous therapy, the subject's clinical history and response to the antagonist, and the discretion of the attending physician. Typically, the clinician will administer a polypeptide until a dosage is reached that achieves the desired result.

Administration of one or more compositions can be continuous or intermittent, depending, for example, upon the recipient's physiological condition, whether the purpose of the administration is therapeutic or prophylactic, and other factors known to skilled practitioners. The administration of a composition may be essentially continuous over a preselected period of time or may be in a series of spaced dose, e.g., either before, during, or after developing a disease.

The methods and compositions of the disclosure described herein including embodiments thereof can be administered with one or more additional therapeutic regimens or agents or treatments, which can be co-administered to the mammal. By “co-administering” is meant administering one or more additional therapeutic regimens or agents or treatments and the composition of the disclosure sufficiently close in time to enhance the effect of one or more additional therapeutic agents, or vice versa. In this regard, the composition of the disclosure described herein can be administered simultaneously with one or more additional therapeutic regimens or agents or treatments, at a different time, or on an entirely different therapeutic schedule (e.g., the first treatment can be daily, while the additional treatment is weekly). For example, in embodiments, the secondary therapeutic regimens or agents or treatments are administered simultaneously, prior to, or subsequent to the composition of the disclosure.

### Pharmaceutical Compositions, Dosage Forms and Administration

In some aspects, provided herein, is a pharmaceutical composition for epigenetic modification comprising an epigenetic editing system described herein, or one or more nucleic acid sequences encoding components of the epigenetic editing system, e.g., nucleic acids encoding an epigenetic editing system fusion protein and/or a guide RNA, and a pharmaceutically acceptable carrier. The composition for epigenetic modification described herein can be formulated into pharmaceutical compositions. Pharmaceutical compositions are formulated in a conventional manner using one or more pharmaceutically acceptable inactive ingredients that facilitate processing of the active compounds into preparations that can be used pharmaceutically. Suitable formulations for use in the present disclosure and methods of delivery are generally well known in the art. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition can be a mixture of an epigenetic editing system or nucleic acids encoding same as described herein and one or more other chemical components (i.e., pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the epigenetic editor, for example, a nucleic acid encoding a zinc finger-epigenetic effector fusion protein or a Cas9-epigenetic effector fusion protein and a gRNA or sgRNA described herein to an organism or a subject in need thereof.

The pharmaceutical compositions of the present disclosure can be administered to a subject using any suitable methods known in the art. The pharmaceutical compositions described herein can be administered to the subject in a variety of ways, including parenterally, intravenously, intradermally, intramuscularly, colonically, rectally, or intraperitoneally. In some embodiments, the pharmaceutical compositions can be administered by intraperitoneal injection, intramuscular injection, subcutaneous injection, or intravenous injection of the subject. In some embodiments, the pharmaceutical compositions can be administered parenterally, intravenously, intramuscularly, or orally.

For administration by inhalation, the adenovirus described herein can be formulated for use as an aerosol, a mist, or a powder. For buccal or sublingual administration, the pharmaceutical compositions may be formulated in the form of tablets, lozenges, or gels formulated in a conventional manner. In some embodiments, the adenovirus described herein can be prepared as transdermal dosage forms. In some embodiments, the adenovirus described herein can be formulated into a pharmaceutical composition suitable for intramuscular, subcutaneous, or intravenous injection. In some embodiments, the adenovirus described herein can be administered topically and can be formulated into a variety of topically administrable compositions, such as solutions, suspensions, lotions, gels, pastes, medicated sticks, balms, creams, or ointments. In some embodiments, the adenovirus described herein can be formulated in rectal compositions such as enemas, rectal gels, rectal foams, rectal aerosols, suppositories, jelly suppositories, or retention enemas. In some embodiments, the adenovirus described herein can be formulated for oral administration such as a tablet, a capsule, or liquid in the form of aqueous suspensions or solutions selected from the group including, but not limited to, aqueous oral dispersions, emulsions, solutions, elixirs, gels, and syrups.

In some embodiments, the pharmaceutical composition for epigenetic modification comprising an epigenetic editor described herein or nucleic acid sequences encoding the same further comprises a therapeutic agent. The additional therapeutic agent may modulate different aspects of the disease, disorder, or condition being treated and provide a greater overall benefit than administration of either the replication competent recombinant adenovirus or the therapeutic agent alone. Therapeutic agents include, but are not limited to, a chemotherapeutic agent, a radiotherapeutic agent, a hormonal therapeutic agent, and/or an immunotherapeutic agent. In some embodiments, the therapeutic agent may be a radiotherapeutic agent. In some embodiments, the therapeutic agent may be a hormonal therapeutic agent. In some embodiments, the therapeutic agent may be an immunotherapeutic agent. In some embodiments, the therapeutic agent is a chemotherapeutic agent. Preparation and dosing schedules for additional therapeutic agents can be used according to manufacturers' instructions or as determined empirically by a skilled practitioner. For example, preparation and dosing schedules for chemotherapy are also described in The Chemotherapy Source Book, 4th Edition, 2008, M. C. Perry, Editor, Lippincott, Williams & Wilkins, Philadelphia, PA.

The subjects that can be treated with epigenetic modification compositions can be any subject with a disease or a condition. For example, the subject may be a eukaryotic subject, such as an animal. In some embodiments, the subject is a mammal, e.g., human. In some embodiments, the subject is a human. In some embodiments, the subject is a non-human animal. In some embodiments, the subject is a fetus, an embryo, or a child. In some embodiments, the subject is a non-human primate such as chimpanzee, and other apes and monkey species; farm animals such as cattle, horses, sheep, goats, pigs; domestic animals such as rabbits, dogs, and cats; laboratory animals including rodents, such as rats, mice, and guinea pigs, and the like.

In some embodiments, the subject is prenatal (e.g., a fetus), a child (e.g., a neonate, an infant, a toddler, a preadolescent), an adolescent, a pubescent, or an adult (e.g., an early adult, a middle-aged adult, a senior citizen). The human subject can be between about 0 month and about 120 years old, or older. The human subject can be between about 0 and about 12 months old; for example, about 1, 2, 3, 4, 5, 6, 7, 8, 9, 10, 11, or 12 months old. The human subject can be between about 0 and 12 years old; for example, between about 0 and 30 days old; between about 1 month and 12 months old; between about 1 year and 3 years old; between about 4 years and 5 years old; between about 4 years and 12 years old; about 1, 2, 3, 4, 5, 6, 7, 8, 9, 10, 11, or 12 years old. The human subject can be between about 13 years and 19 years old; for example, about 13, 14, 15, 16, 17, 18, or 19 years old. The human subject can be between about 20 and about 39 years old; for example, about 20, 21, 22, 23, 24, 25, 26, 27, 28, 29, 30, 31, 32, 33, 34, 35, 36, 37, 38, or 39 years old. The human subject can be between about 40 to about 59 years old; for example, about 40, 41, 42, 43, 44, 45, 46, 47, 48, 49, 50, 51, 52, 53, 54, 55, 56, 57, 58, or 59 years old. The human subject can be greater than 59 years old; for example, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 years old. The human subjects can include male subjects and/or female subjects.

Also disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, and any packaging material suitable for a selected formulation and intended mode of administration and treatment.

For example, the container(s) include the composition of the disclosure, and optionally in addition with therapeutic regimens or agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### EXAMPLES

The following examples are included for illustrative purposes only and are not intended to limit the scope of the disclosure.

### Example 1: Fusion Proteins with Variant NLS Configurations

Several improved fusion protein constructs were developed using variant nuclear localization sequence (NLS) configurations to have significantly higher epi-silencing activity.

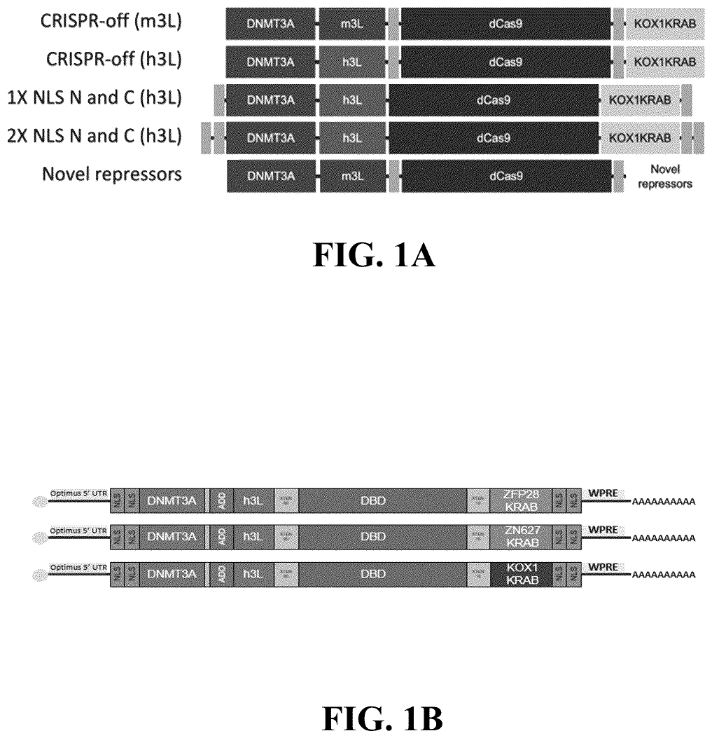

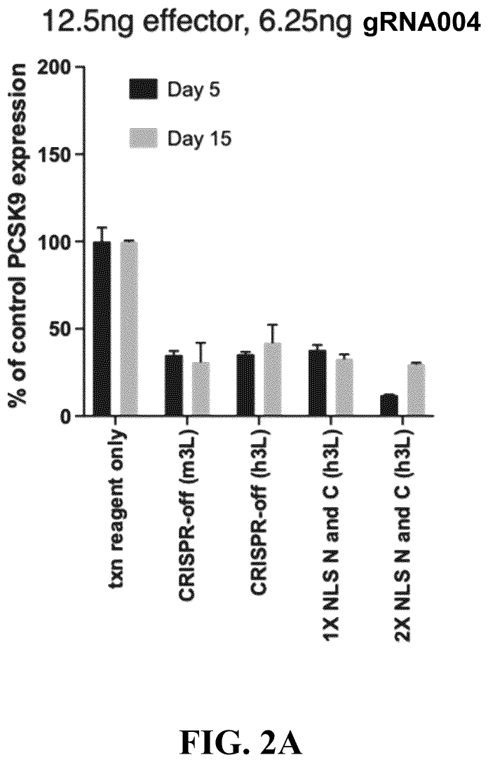

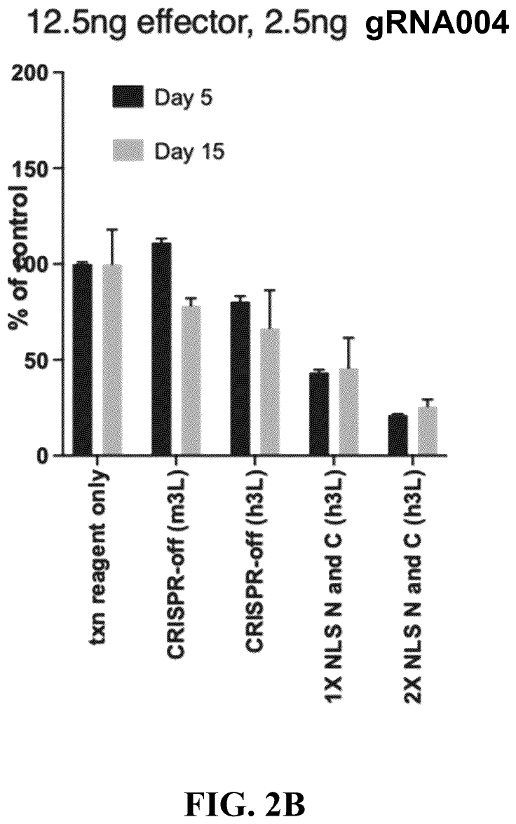

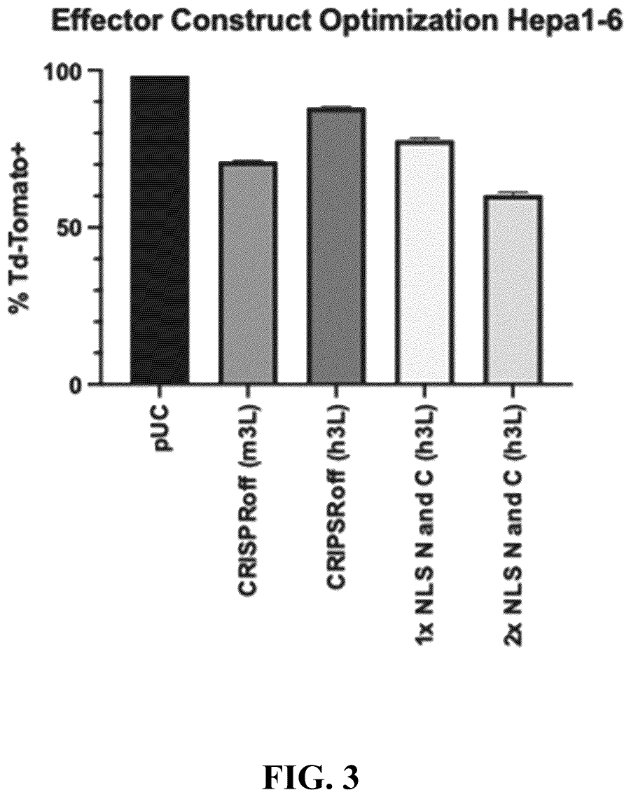

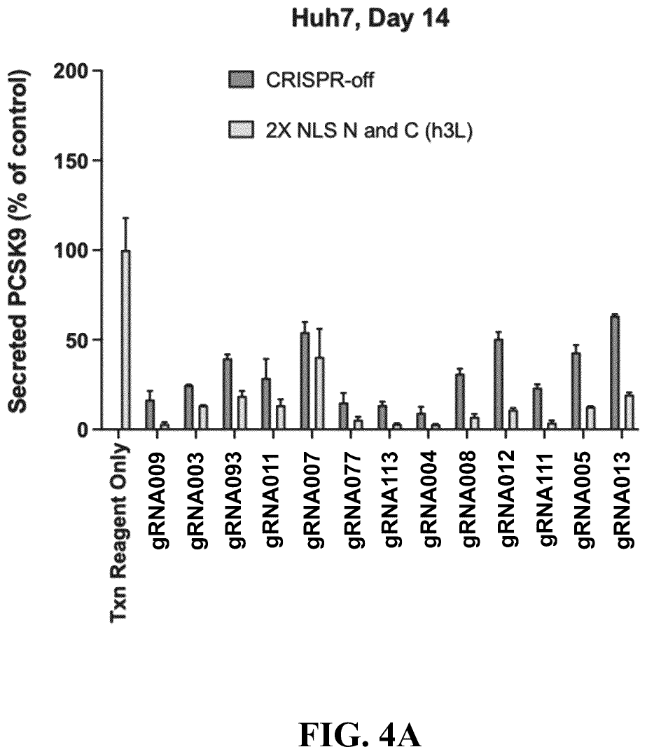

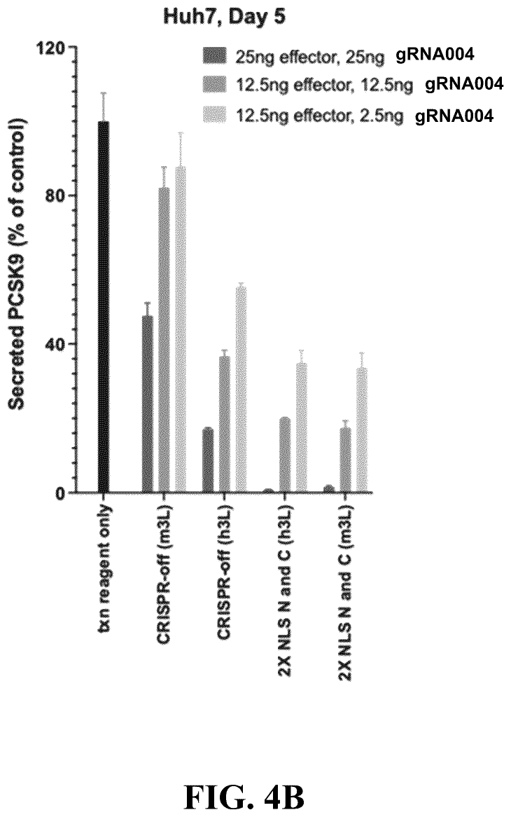

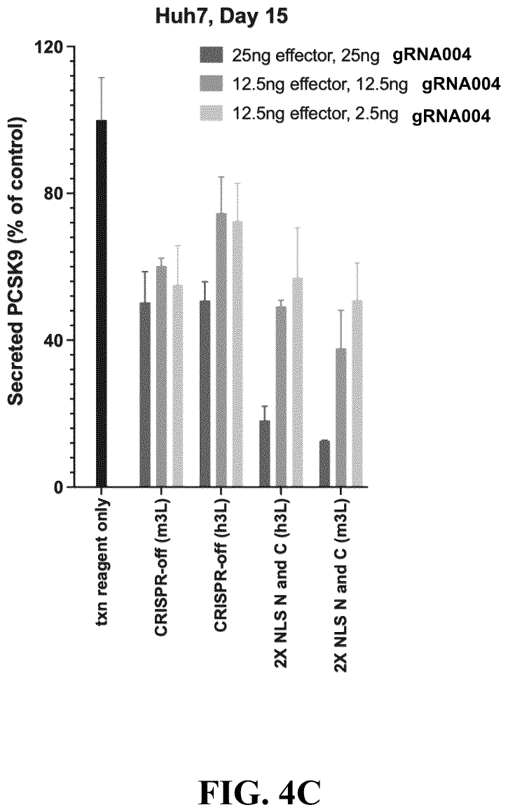

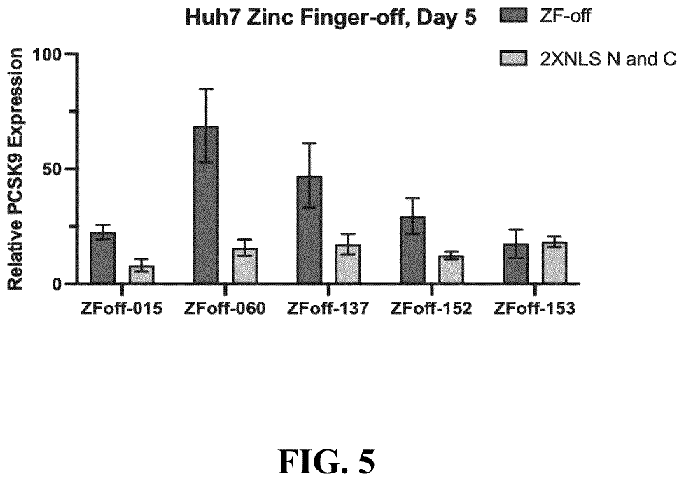

Several constructs with variant configurations of NLS domains (FIG. 1) were constructed and tested in Pcsk9 loci in HeLa cells (FIGS. 2A-2B). The constructs were additionally tested in Pcsk9 loci in Hepa1-6 (FIG. 3) and in HuH7 (FIGS. 4-5). Exemplary fusion protein construct DNA and amino acid sequences are seen in SEQ ID NOs: 17-24, 28-35, 110-117, and 160-167. Sequences from FIG. 4A can be found in SEQ ID NOs: 118-130. Sequences from FIG. 5 can be found in SEQ ID NOs: 131-150.

### Cell Culture and Transfection

HeLa (ATCC-CRM-CCL-2), Hepa1-6 (PCSK9-IRES-TdTomato) Huh7 (Sekisui XenoTech, LLC) and HEK293T Griptite (CLTA-GFP) cells were cultured in DMEM with 10% FBS. All experiments in HeLa and Huh7 cells were done using chemically synthesized guide RNA and in vitro transcribed effector construct. HeLa cells were reverse transfected using TransIT-X2 transfection reagent from Mirus (Cat #MIR6003). Huh7 cells were reverse transfected using MessengerMAX reagent from Invitrogen (Cat #LMRNA003). Secreted PCSK9 levels were measured at the indicated time points using LEGEND MAX™ Human PCSK9 ELISA Kit from Biolegend (Cat #443107). All ELISA data was normalized for cell numbers using CellTiter-Glo kit from Promega (Cat #G7571).

HEK293T Griptite cells with GFP knocked into the CLTA locus as an in-frame CLTA fusion were co-transfected with plasmids encoding effector construct and human CLTA guide RNA using TransIT-X2 transfection reagent from Mirus (Cat #MIR6003). GFP was measured by FACS for GFP expression as a surrogate for CLTA expression.

Hepa1-6 were co-transfected with plasmids encoding effector construct and mouse PCSK9 guide RNA using SF Cell Line 96-well Nucleofector Kit (Cat #V4SC-2096, program code: CM-138) in Amaxa 4D nucleofector device from Lonza. At the indicated timepoint, cells were FACS analyzed for TdTomato expression as a surrogate for PCSK9 levels.

In Vitro Transcription of Effector Constructs and Synthetic gRNA

1 ug of linearized effector template was used to setup in-vitro transcription reactions using T7 mScript™ Standard mRNA Production System from CellScript (Cat #C-MSC100625) according to manufacturer's instructions to obtain RNA that had a Cap 1 structure on the 5′ end and was 3′polyadnelylated. End-modified sgRNA that had three 2′O-Methyl modified nucleotides with phosphorothioate linkages on both 5′ and 3′ ends were obtained from Integrated DNA technologies.

### Methylation Profiling

Genomic DNA was extracted from each well of a 96-well culture plate using a DNAdvance DNA Extraction from Tissue Kit (Beckman Coulter). After quantification of genomic DNA via High-Sensitivity DNA 1× kit (Quant-IT), each genomic DNA sample was bisulfite converted using an EZ-96 DNA Methylation-Gold MagPrep kit (Zymo Research) according to manufacturer's instructions. For hybridization capture experiments, DNA libraries were prepared using the xGen™ Methyl-Seq DNA Library Prep Kit (IDT) and hybrid capture was conducted using the xGen™ Hybridization Capture of DNA libraries kit (IDT). For amplicon sequencing experiments, DNA libraries were prepared using the xGen™ Methyl-Seq DNA Library Prep Kit (IDT) and hybrid capture was conducted using the xGen™ Hybridization Capture of DNA libraries (IDT). Bisulfite-converted DNA from each sample was used to seed PCR corresponding to each of the two VIM amplicons using a Platinum Taq kit (Invitrogen). Pooled products were cleaned using the AMPure XP kit (Beckman Coulter) and fragment size assessment via D1000 screentape on a Tapestation 4200 (Agilent) prior to sequencing by commercial service (Azenta).

### Example 2: Bacterial DNA Methyltransferases

In this experiment, a panel of bacterial proteins were screened for DNA methyltransferase activity in mammalian cells. These bacterial DNA methyltransferases (Table 3) were tested for epigenetic silencing activity by fusing them N-terminally to a dCas9 domain using the experimental procedure of Example 1. These constructs were then transfected in a reporter cell line that expresses GFP under the control of the mammalian promoter of CTLA4.

TABLE 3
Bacterial DNA methyltransferases

| Description | SEQ ID NO |
|---|---|
| DNA-(cytosine C5)-methyltransferase, | 36 |
| Arthrobacter luteus (M. AluI) | |
| Cytosine-specific methyltransferase, | 37 |
| Moraxella Spp. (M. MspI) | |
| DNA cytosine methyltransferase, | 38 |
| Haemophilus influenzae (M. HaeIII) | |
| DNA cytosine methyltransferase, | 39 |
| Haemophilus haemolyticus(M. HhaI) | |
| DNA cytosine methyltransferase, | 40 |
| S. monobiae (M. SssI) | |

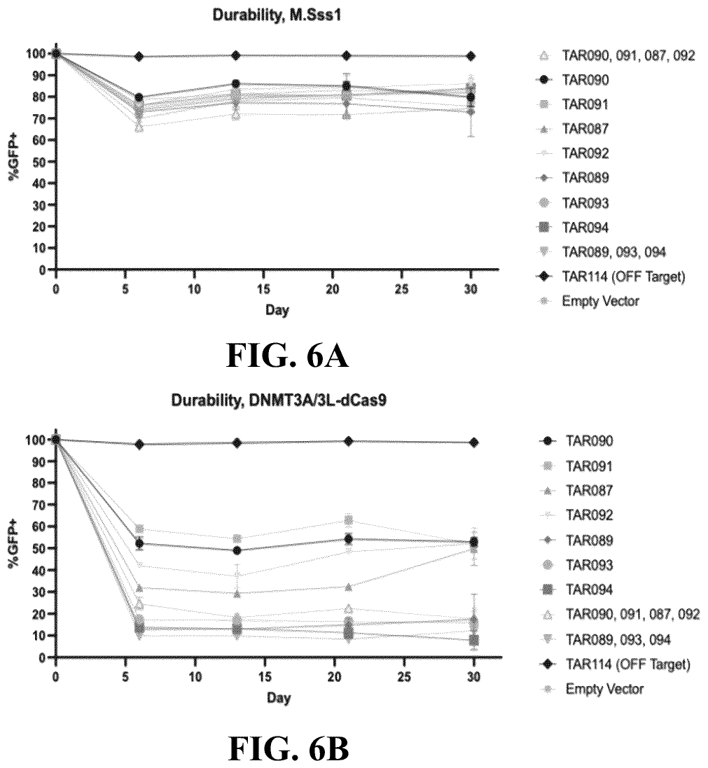

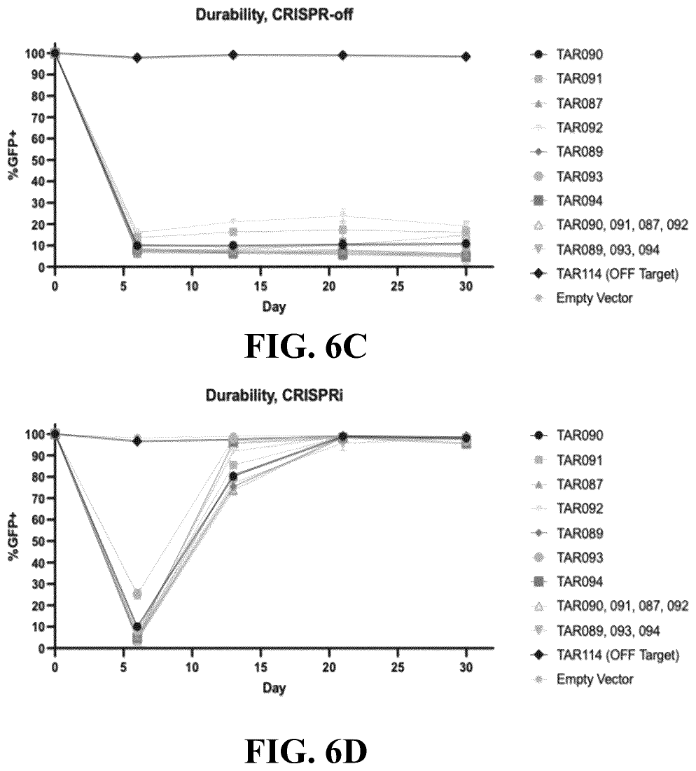

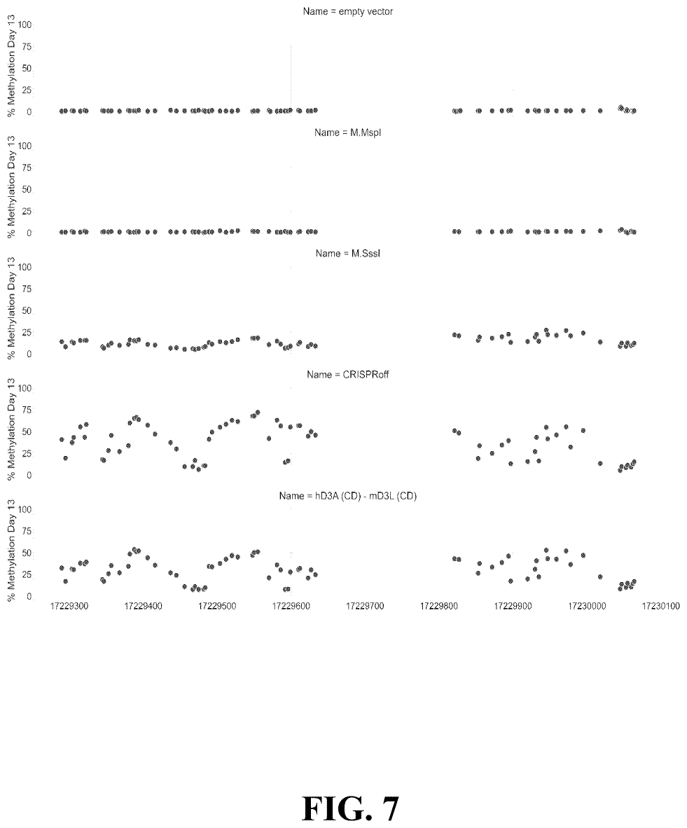

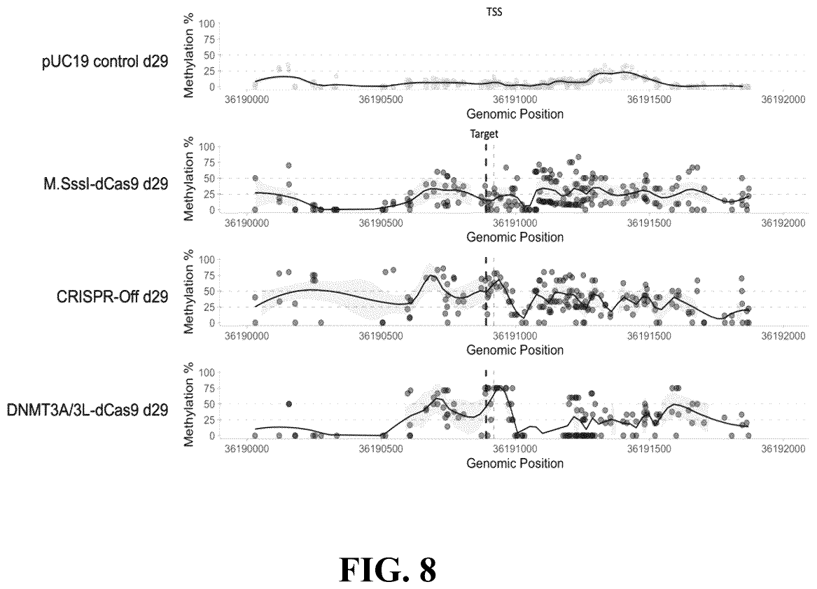

M.SssI DNA methyltransferase was able to efficiently methylate DNA in mammalian cells (FIGS. 6A-6D) with stable silencing up to 30 days. Sequences from FIGS. 6A-D can be found in SEQ ID NOs: 151-158. The methylation profile of these cells was also analyzed at day 29 confirming a 20% methylation of the target gene (FIGS. 7-8).

Another three orthologous DNA methyltransferases, predicted to be closely related to M. SssI, are identified and tested for epigenetic silencing activity using the experimental procedures of Example 1 (Table 4).

TABLE 4
Bacterial methyltransferases

| Description | SEQ ID NO |
|---|---|
| DNA cytosine methyltransferase | 41 |
| Mycoplasmatales bacterium | |
| DNA cytosine methyltransferase | 42 |
| Mycoplasma marinum | |
| DNA (cytosine-5-)-methyltransferase | 43 |
| Spiroplasma chinense | |

The DNA methyltransferases of Table 4 are predicted to have similar or improved function to M. SssI. Sequences are tested in the context of CRISPR-off, in place of murine DNMT3A/DNMT3L, and their function is compared with the function of M. SssI DNA methyltransferase in silencing the Pesk9 locus in a HeLa TdTomato system, to identify novel characteristics and improved function.

### Example 3: Alternative KRAB Domains

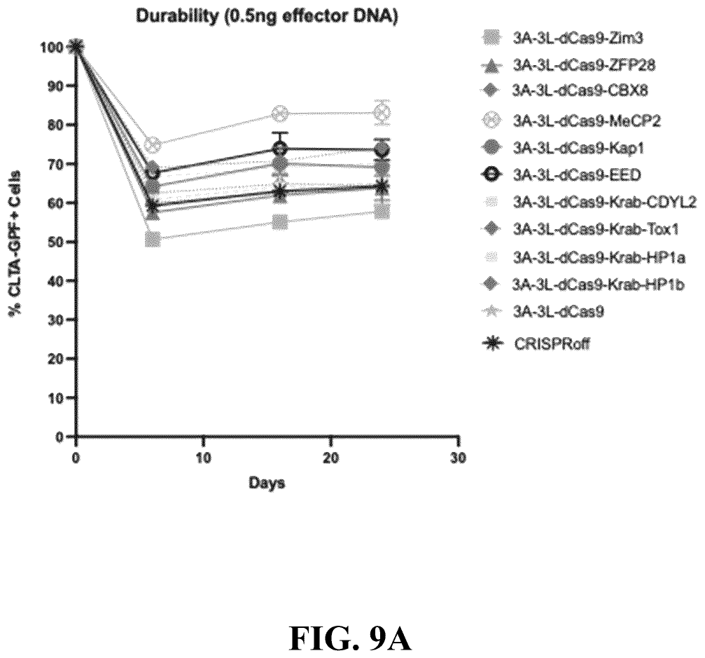

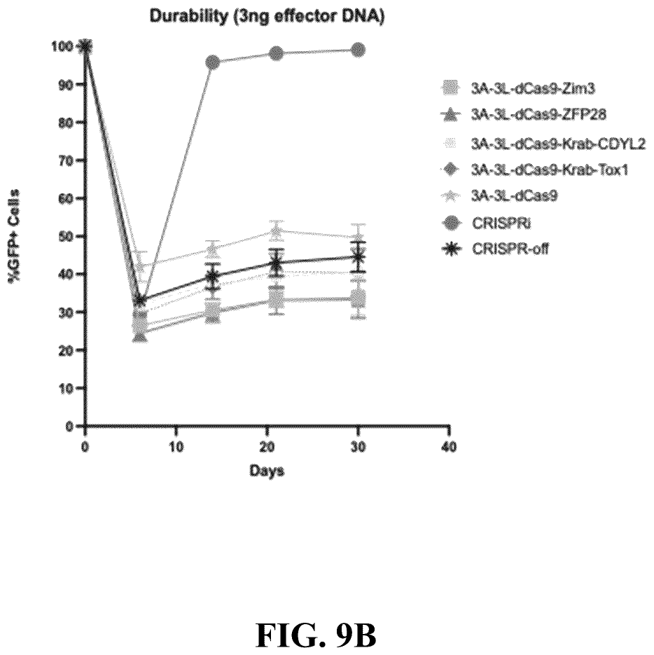

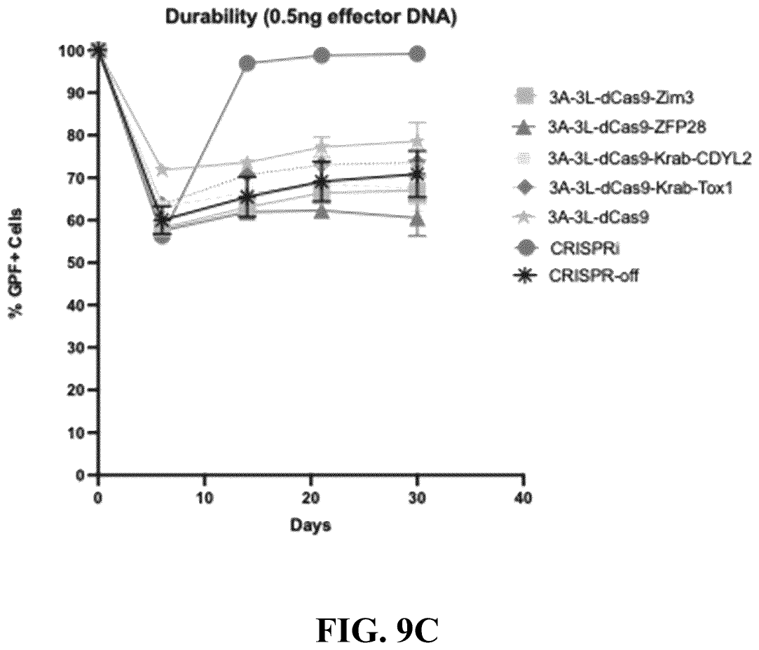

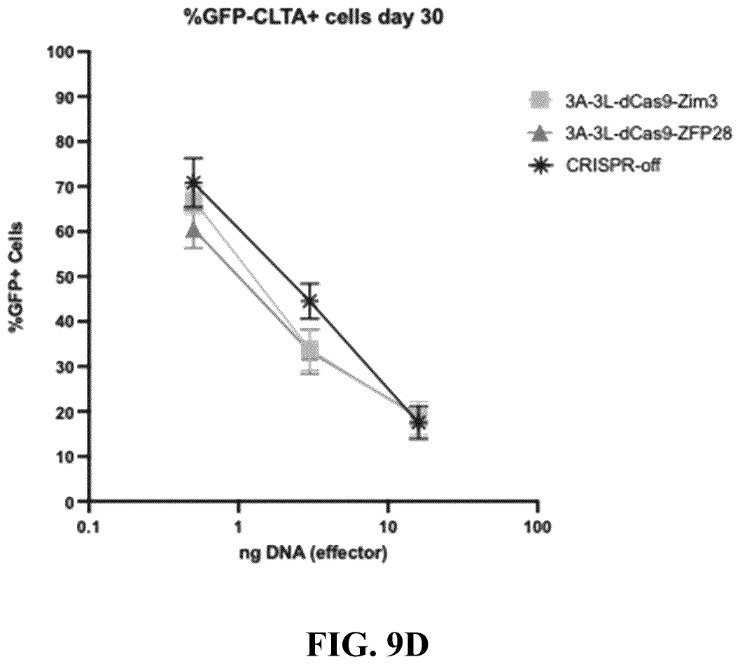

In this example, fusion proteins were constructed with alternative KRAB domains (Table 5) and showed improved activity as compared to CRISPRoff when tested using the experimental procedures of Example 1 (FIG. 9).

TABLE 5
Alternative KRAB Domains

| Description | SEQ ID NO |
|---|---|
| ZFP28 | 44 |
| ZN627 | 45 |
| KAP1 | 46 |
| MeCP2 | 47 |
| HP1b | 48 |
| CBX8 | 49 |
| CDYL2 | 50 |
| TOX | 51 |
| TOX3 | 52 |
| TOX4 | 53 |
| EED | 54 |
| RBBP4 | 55 |
| RCOR1 | 56 |
| SCML2 | 57 |

### Example 4: ZIM 3 Fusion Constructs

Novel fusions of ZIM3 and KOX1KRAB are generated. Both ZIM 3 and KOX1KRAB are KRAB family proteins with extensive homology. Thus, sequences are designed which represent halfway points between ZIM3 and KOX1KRAB. These KOX1KRAB and ZIM3 constructs encode a small region of KOX1KRAB and ZIM3 focused around the zinc finger domain of the protein. While the regions used of KOX1KRAB and ZIM3 are very similar within the first ˜75 bp of their sequence, ZIM3 also possesses a small alpha-helical region at the C-terminus, not present in KOX1KRAB. The KOX1KRAB-FL sequence includes the KOX1KRAB sequence equivalent of this extra piece, while the ZIM3 truncation has this extra piece removed from the ZIM3 sequence. The ZIM3/KOX1KRAB chimeras are fusions of the N- and C-terminal pieces of the two proteins. The ZIM3-like KOX1KRAB variants were both assembled by first, BLAST of ZIM3 or KOX1KRAB proteins from nonhuman species to assemble the closest 100 homologs (‘families’) of each gene; second, identifying the 3 members of the KOX1KRAB family that most closely resemble ZIM3 and the 3 members of the ZIM3 family that most closely resemble KOX1KRAB; and third, rationally modifying the KOX1KRAB-FL sequence to resemble each set of three (Table 6).

TABLE 6
ZIM-KOX1KRAB Chimera Proteins

| Description | SEQ ID NO |
|---|---|
| KOX1KRAB Residues 11-72 | 58 |
| KOX1KRAB-FL Residues 11-108 | 59 |
| ZIM3 Residues 1-100 | 60 |
| KOX1KRAB-ZIM3 chimera 1 | 61 |
| (Residues 11-72 of KOX1KRAB, | |
| Residues 68-100 of ZIM3) | |
| ZIM3-KOX1KRAB chimera 2 | 62 |
| (Residues 1-67 of ZIM3, | |
| Residues 73-108 of KOX1KRAB) | |
| Truncated ZIM3 (Residues 1-75 of ZIM3) | 63 |
| KOX1KRAB-FL Zim3-like | 64 |
| (Modified Residues 11-108) | |
| KOX1KRAB-like ZIM3 1 | 65 |
| KOX1KRAB-like ZIM3 2 | 66 |
| KOX1KRAB-like ZIM3 3 | 67 |
| KOX1KRAB-like ZIM3 4 | 68 |
| ZIM-like KOX1KRAB 1 | 69 |
| ZIM-like KOX1KRAB 2 | 70 |
| ZIM-like KOX1KRAB 3 | 71 |

While preferred embodiments of the present disclosure have been shown and described herein, it will be obvious to those skilled in the art that such embodiments are provided by way of example only. Numerous variations, changes, and substitutions will be apparent to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Sequence Table

| SEQ ID | | |
|---|---|---|
| NO | Description | Sequence |
| 1 | G Linker | GSGGG |
| 2 | XTEN16 Linker | SGSETPGTSESATPES |
| 3 | XTEN80 Linker | GGPSSGAPPPSGGSPAGSPTSTEEGTSESATPESGPGTSTEPSEGS |
| | | APGSPAGSPTSTEEGTSTEPSEGSAPGTSTEPSE |
| 4 | GX4 Linker | GGGGSGGGGSGGGGSGGGGS |
| 5 | W Linker | SSGNSNANSRGPSFSSGLVPLSLRGSH |
| 6 | HA Tag | YPYDVPDYA |
| 7 | SV40 NLS | PKKKRKV |
| 8 | dCas9 | MDKKYSIGLDIGTNSVGWAVITDEYKVPSKKFKVLGNTDRHSI |
| | | KKNLIGALLFDSGETAEATRLKRTARRRYTRRKNRICYLQEIFS |
| | | NEMAKVDDSFFHRLEESFLVEEDKKHERHPIFGNIVDEVAYHE |
| | | KYPTIYHLRKKLVDSTDKADLRLIYLALAHMIKFRGHFLIEGDL |
| | | NPDNSDVDKLFIQLVQTYNQLFEENPINASGVDAKAILSARLSK |
| | | SRRLENLIAQLPGEKKNGLFGNLIALSLGLTPNFKSNFDLAEDA |
| | | KLQLSKDTYDDDLDNLLAQIGDQYADLFLAAKNLSDAILLSDIL |
| | | RVNTEITKAPLSASMIKRYDEHHQDLTLLKALVRQQLPEKYKEI |
| | | FFDQSKNGYAGYIDGGASQEEFYKFIKPILEKMDGTEELLVKLN |
| | | REDLLRKQRTFDNGSIPHQIHLGELHAILRRQEDFYPFLKDNRE |
| | | KIEKILTFRIPYYVGPLARGNSRFAWMTRKSEETITPWNFEEVV |
| | | DKGASAQSFIERMTNFDKNLPNEKVLPKHSLLYEYFTVYNELT |
| | | KVKYVTEGMRKPAFLSGEQKKAIVDLLFKTNRKVTVKQLKED |
| | | YFKKIECFDSVEISGVEDRFNASLGTYHDLLKIIKDKDFLDNEEN |
| | | EDILEDIVLTLTLFEDREMIEERLKTYAHLFDDKVMKQLKRRRY |
| | | TGWGRLSRKLINGIRDKQSGKTILDFLKSDGFANRNFMQLIHDD |
| | | SLTFKEDIQKAQVSGQGDSLHEHIANLAGSPAIKKGILQTVKVV |
| | | DELVKVMGRHKPENIVIEMARENQTTQKGQKNSRERMKRIEEG |
| | | IKELGSQILKEHPVENTQLQNEKLYLYYLQNGRDMYVDQELDI |
| | | NRLSDYDVDHIVPQSFLKDDSIDNKVLTRSDKNRGKSDNVPSEE |
| | | VVKKMKNYWRQLLNAKLITQRKFDNLTKAERGGLSELDKAGF |
| | | IKRQLVETRQITKHVAQILDSRMNTKYDENDKLIREVKVITLKS |
| | | KLVSDFRKDFQFYKVREINNYHHAHDAYLNAVVGTALIKKYP |
| | | KLESEFVYGDYKVYDVRKMIAKSEQEIGKATAKYFFYSNIMNF |
| | | FKTEITLANGEIRKRPLIETNGETGEIVWDKGRDFATVRKVLSM |
| | | PQVNIVKKTEVQTGGFSKESILPKRNSDKLIARKKDWDPKKYG |
| | | GFDSPTVAYSVLVVAKVEKGKSKKLKSVKELLGITIMERSSFEK |
| | | NPIDFLEAKGYKEVKKDLIIKLPKYSLFELENGRKRMLASAGEL |
| | | QKGNELALPSKYVNFLYLASHYEKLKGSPEDNEQKQLFVEQHK |
| | | HYLDEIIEQISEFSKRVILADANLDKVLSAYNKHRDKPIREQAEN |
| | | IIHLFTLTNLGAPAAFKYFDTTIDRKRYTSTKEVLDATLIHQSITG |
| | | LYETRIDLSQLGGDS |
| 9 | CRISPRoff | TGRTLVTFKDVFVDFTREEWKLLDTAQQIVYRNVMLENYKNL |
| | KRAB | VSLGYQLTKPDVILRLEKGEEPLEINLWITKFVKD |
| 10 | N-Terminal | MYPYDVPDYASPKKKRKVGGGASMDAKSLTAWSRTLVTFKDV |
| | KRAB | FVDFTREEWKLLDTAQQIVYRNVMLENYKNLVSLGYQLTKPDVILR |
| | | LEKGEEPWLVEREIHQETHPDSETAFEIKSSV |
| 11 | C-Terminal | ALSPQHSAVTQGSIIKNKEGMDAKSLTAWSRTLVTFKDVFVDF |
| | KRAB | TREEWKLLDTAQQIVYRNVMLENYKNLVSLGYQLTKPDVILRL |
| | | EKGEEPWLVEREIHQETHPDSETAFEIKSSV |
| 12 | Human | TYGLLRRREDWPSRLQMFFANNHDQEFDPPKVYPPVPAEKRKP |
| | DNMT3A (h3A) | IRVLSLFDGIATGLLVLKDLGIQVDRYIASEVCEDSITVGMVRHQ |
| | | GKIMYVGDVRSVTQKHIQEWGPFDLVIGGSPCNDLSIVNPARK |
| | | GLYEGTGRLFFEFYRLLHDARPKEGDDRPFFWLFENVVAMGVS |
| | | DKRDISRFLESNPVMIDAKEVSAAHRARYFWGNLPGMNRPLAS |
| | | TVNDKLELQECLEHGRIAKFSKVRTITTRSNSIKQGKDQHFPVF |
| | | MNEKEDILWCTEMERVFGFPVHYTDVSNMSRLARQRLLGRSW |
| | | SVPVIRHLFAPLKEYFACV |
| 13 | Human DNMT3L | MAAIPALDPEAEPSMDVILVGSSELSSSVSPGTGRDLIAYEVKA |
| | (h3L) | NQRNIEDICICCGSLQVHTQHPLFEGGICAPCKDKFLDALFLYDD |
| | | DGYQSYCSICCSGETLLICGNPDCTRCYCFECVDSLVGPGTSGK |
| | | VHAMSNWVCYLCLPSSRSGLLQRRRKWRSQLKAFYDRESENP |
| | | LEMFETVPVWRRQPVRVLSLFEDIKKELTSLGFLESGSDPGQLK |
| | | HVVDVTDTVRKDVEEWGPFDLVYGATPPLGHTCDRPPSWYLF |
| | | QFHRLLQYARPKPGSPRPFFWMFVDNLVLNKEDLDVASRFLEM |
| | | EPVTIPDVHGGSLQNAVRVWSNIPAIRSRHWALVSEEELSLLAQ |
| | | NKQSSKLAAKWPTKLVKNCFLPLREYFKYFSTELTSSL |
| 14 | Human DNMT3L | ENPLEMFETVPVWRRQPVRVLSLFEDIKKELTSLGFLESGSDPG |
| | short (h3Ls) | QLKHVVDVTDTVRKDVEEWGPFDLVYGATPPLGHTCDRPPSW |
| | | YLFQFHRLLQYARPKPGSPRPFFWMFVDNLVLNKEDLDVASRF |
| | | LEMEPVTIPDVHGGSLQNAVRVWSNIPAIRSRHWALVSEEELSL |
| | | LAQNKQSSKLAAKWPTKLVKNCFLPLREYFKYFSTELTSSL |
| 15 | Mouse DNMT3L | MGSRETPSSCSKTLETLDLETSDSSSPDADSPLEEQWLKSSPALK |
| | (m3L) | EDSVDVVLEDCKEPLSPSSPPTGREMIRYEVKVNRRSIEDICLCC |
| | | GTLQVYTRHPLFEGGLCAPCKDKFLESLFLYDDDGHQSYCTICC |
| | | SGGTLFICESPDCTRCYCFECVDILVGPGTSERINAMACWVCFL |
| | | CLPFSRSGLLQRRKRWRHQLKAFHDQEGAGPMEIYKTVSAWK |
| | | RQPVRVLSLFRNIDKVLKSLGFLESGSGSGGGTLKYVEDVTNV |
| | | VRRDVEKWGPFDLVYGSTQPLGSSCDRCPGWYMFQFHRILQY |
| | | ALPRQESQRPFFWIFMDNLLLTEDDQETTTRFLQTEAVTLQDVR |
| | | GRDYQNAMRVWSNIPGLKSKHAPLTPKEEEYLQAQVRSRSKL |
| | | DAPKVDLLVKNCLLPLREYFKYFSQNSLPL |
| 16 | Mouse DNMT3L | MGPMEIYKTVSAWKRQPVRVLSLFRNIDKVLKSLGFLESGSGS |
| | short (m3Ls) | GGGTLKYVEDVTNVVRRDVEKWGPFDLVYGSTQPLGSSCDRC |
| | | PGWYMFQFHRILQYALPRQESQRPFFWIFMDNLLLTEDDQETT |
| | | TRFLQTEAVTLQDVRGRDYQNAMRVWSNIPGLKSKHAPLTPKE |
| | | EEYLQAQVRSRSKLDAPKVDLLVKNCLLPLREYFKYFSQNSLPL |
| 17 | Fusion Protein 1 | MPKKKRKVPKKKRKVNHDQEFDPPKVYPPVPAEKRKPIRVLSL |
| | Amino Acid | FDGIATGLLVLKDLGIQVDRYIASEVCEDSITVGMVRHQGKIMY |
| | Sequence | VGDVRSVTQKHIQEWGPFDLVIGGSPCNDLSIVNPARKGLYEGT |
| | NLS-3A-3L- | GRLFFEFYRLLHDARPKEGDDRPFFWLFENVVAMGVSDKRDIS |
| | dCas9-KRAB- | RFLESNPVMIDAKEVSAAHRARYFWGNLPGMNRPLASTVNDK |
| | NLS | LELQECLEHGRIAKFSKVRTITTRSNSIKQGKDQHFPVFMNEKE |
| | | DILWCTEMERVFGFPVHYTDVSNMSRLARQRLLGRSWSVPVIR |
| | | HLFAPLKEYFACVSSGNSNANSRGPSFSSGLVPLSLRGSHNPLE |
| | | MFETVPVWRRQPVRVLSLFEDIKKELTSLGFLESGSDPGQLKHV |
| | | VDVTDTVRKDVEEWGPFDLVYGATPPLGHTCDRPPSWYLFQF |
| | | HRLLQYARPKPGSPRPFFWMFVDNLVLNKEDLDVASRFLEMEP |
| | | VTIPDVHGGSLQNAVRVWSNIPAIRSRHWALVSEEELSLLAQNK |
| | | QSSKLAAKWPTKLVKNCFLPLREYFKYFSTELTSSLGGPSSGAP |
| | | PPSGGSPAGSPTSTEEGTSESATPESGPGTSTEPSEGSAPGSPAGS |
| | | PTSTEEGTSTEPSEGSAPGTSTEPSEMDKKYSIGLAIGTNSVGWA |
| | | VITDEYKVPSKKFKVLGNTDRHSIKKNLIGALLFDSGETAEATR |
| | | LKRTARRRYTRRKNRICYLQEIFSNEMAKVDDSFFHRLEESFLV |
| | | EEDKKHERHPIFGNIVDEVAYHEKYPTIYHLRKKLVDSTDKADL |
| | | RLIYLALAHMIKFRGHFLIEGDLNPDNSDVDKLFIQLVQTYNQL |
| | | FEENPINASGVDAKAILSARLSKSRRLENLIAQLPGEKKNGLFGN |
| | | LIALSLGLTPNFKSNFDLAEDAKLQLSKDTYDDDLDNLLAQIGD |
| | | QYADLFLAAKNLSDAILLSDILRVNTEITKAPLSASMIKRYDEH |
| | | HQDLTLLKALVRQQLPEKYKEIFFDQSKNGYAGYIDGGASQEE |
| | | FYKFIKPILEKMDGTEELLVKLNREDLLRKQRTFDNGSIPHQIHL |
| | | GELHAILRRQEDFYPFLKDNREKIEKILTFRIPYYVGPLARGNSR |
| | | FAWMTRKSEETITPWNFEEVVDKGASAQSFIERMTNFDKNLPN |
| | | EKVLPKHSLLYEYFTVYNELTKVKYVTEGMRKPAFLSGEQKKA |
| | | IVDLLFKTNRKVTVKQLKEDYFKKIECFDSVEISGVEDRFNASL |
| | | GTYHDLLKIIKDKDFLDNEENEDILEDIVLTLTLFEDREMIEERL |
| | | KTYAHLFDDKVMKQLKRRRYTGWGRLSRKLINGIRDKQSGKTI |
| | | LDFLKSDGFANRNFMQLIHDDSLTFKEDIQKAQVSGQGDSLHE |
| | | HIANLAGSPAIKKGILQTVKVVDELVKVMGRHKPENIVIEMARE |
| | | NQTTQKGQKNSRERMKRIEEGIKELGSQILKEHPVENTQLQNEK |
| | | LYLYYLQNGRDMYVDQELDINRLSDYDVDAIVPQSFLKDDSID |
| | | NKVLTRSDKNRGKSDNVPSEEVVKKMKNYWRQLLNAKLITQR |
| | | KFDNLTKAERGGLSELDKAGFIKRQLVETRQITKHVAQILDSRM |
| | | NTKYDENDKLIREVKVITLKSKLVSDFRKDFQFYKVREINNYHH |
| | | AHDAYLNAVVGTALIKKYPKLESEFVYGDYKVYDVRKMIAKS |
| | | EQEIGKATAKYFFYSNIMNFFKTEITLANGEIRKRPLIETNGETG |
| | | EIVWDKGRDFATVRKVLSMPQVNIVKKTEVQTGGFSKESILPK |
| | | RNSDKLIARKKDWDPKKYGGFDSPTVAYSVLVVAKVEKGKSK |
| | | KLKSVKELLGITIMERSSFEKNPIDFLEAKGYKEVKKDLIIKLPK |
| | | YSLFELENGRKRMLASAGELQKGNELALPSKYVNFLYLASHYE |
| | | KLKGSPEDNEQKQLFVEQHKHYLDEIIEQISEFSKRVILADANLD |
| | | KVLSAYNKHRDKPIREQAENIIHLFTLTNLGAPAAFKYFDTTIDR |
| | | KRYTSTKEVLDATLIHQSITGLYETRIDLSQLGGDSGSETPGTSE |
| | | SATPESTGRTLVTFKDVFVDFTREEWKLLDTAQQIVYRNVMLE |
| | | NYKNLVSLGYQLTKPDVILRLEKGEEPPKKKRKVPKKKRKV |
| 18 | Fusion Protein 1 | ATGCCAAAAAAGAAGAGAAAGGTACCGAAGAAAAAAAGAA |
| | DNA Sequence | AGGTCAACCATGATCAAGAATTCGACCCACCTAAAGTCTACC |
| | | CACCTGTGCCCGCCGAAAAAAGGAAACCCATAAGGGTGCTG |
| | | TCACTCTTTGATGGCATCGCCACTGGTCTCCTGGTTCTTAAGG |
| | | ATCTGGGAATTCAGGTCGATCGGTACATTGCTAGCGAGGTTT |
| | | GTGAGGATAGTATTACAGTGGGTATGGTGCGCCACCAGGGA |
| | | AAGATCATGTATGTTGGTGACGTTAGGAGCGTCACCCAGAA |
| | | ACATATCCAGGAGTGGGGACCCTTTGATTTGGTGATCGGAGG |
| | | TAGTCCCTGCAATGACCTTTCCATCGTGAATCCAGCCAGGAA |
| | | AGGGCTGTATGAAGGGACTGGTAGGCTCTTTTTCGAGTTTTA |
| | | TCGCCTGCTTCACGACGCTAGACCTAAGGAAGGTGACGATA |
| | | GGCCTTTCTTTTGGCTTTTTGAGAACGTCGTGGCAATGGGAG |
| | | TCTCCGACAAAAGGGACATTTCTCGCTTTCTGGAATCTAACC |
| | | CCGTTATGATCGATGCCAAGGAAGTTTCTGCCGCTCACAGGG |
| | | CAAGGTACTTCTGGGGCAATCTGCCCGGAATGAATCGCCCAC |
| | | TGGCCAGTACCGTGAATGACAAACTGGAGCTGCAGGAGTGC |
| | | CTGGAGCACGGAAGAATCGCAAAGTTTTCTAAAGTCAGGAC |
| | | CATTACCACTCGCAGTAACTCCATAAAACAGGGTAAGGACC |
| | | AGCATTTTCCCGTCTTCATGAATGAAAAGGAAGATATTCTGT |
| | | GGTGCACTGAAATGGAGAGAGTTTTCGGGTTTCCCGTGCACT |
| | | ATACCGATGTTTCCAACATGTCCCGCCTTGCAAGACAAAGGC |
| | | TTTTGGGCCGCTCTTGGTCTGTGCCAGTGATCCGGCACTTGTT |
| | | TGCTCCCCTCAAAGAGTACTTCGCTTGCGTCAGTTCCGGAAA |
| | | TTCAAACGCTAACTCTCGGGGTCCATCTTTCTCCAGTGGTCTC |
| | | GTGCCACTGTCTCTCCGGGGCTCTCACAATCCCCTGGAGATG |
| | | TTTGAGACAGTGCCAGTCTGGCGGAGGCAGCCCGTTCGCGTT |
| | | CTCTCTCTGTTCGAAGATATTAAAAAGGAACTCACCTCCCTT |
| | | GGGTTCCTGGAGAGCGGGAGCGACCCCGGACAGCTTAAGCA |
| | | CGTGGTCGACGTGACTGACACCGTCCGCAAAGACGTGGAGG |
| | | AATGGGGCCCCTTCGATCTGGTCTATGGGGCAACCCCTCCCC |
| | | TTGGGCATACATGTGATCGGCCTCCATCCTGGTACCTGTTCC |
| | | AGTTTCACAGACTCCTGCAGTATGCCAGGCCAAAGCCAGGG |
| | | AGCCCAAGGCCCTTTTTCTGGATGTTCGTCGACAACCTGGTC |
| | | CTGAACAAAGAAGATCTCGACGTTGCTAGTCGCTTTCTCGAA |
| | | ATGGAGCCCGTGACCATTCCCGACGTGCATGGCGGTTCCCTC |
| | | CAGAATGCAGTCAGGGTTTGGAGCAATATCCCTGCCATCAG |
| | | GTCAAGGCACTGGGCACTGGTTTCAGAGGAAGAGCTGTCCC |
| | | TCCTTGCCCAGAACAAGCAGTCATCCAAACTGGCAGCCAAG |
| | | TGGCCAACTAAGCTGGTCAAGAACTGCTTTCTTCCCCTCAGA |
| | | GAATATTTTAAGTATTTCAGTACTGAACTGACTAGCAGTCTG |
| | | GGAGGGCCGAGCTCTGGCGCACCCCCACCAAGTGGAGGGTC |
| | | TCCTGCCGGGTCCCCAACATCTACTGAAGAAGGCACCAGCG |
| | | AATCCGCAACGCCCGAGTCAGGCCCTGGTACCTCCACAGAA |
| | | CCATCTGAAGGTAGTGCGCCTGGTTCCCCAGCTGGAAGCCCT |
| | | ACTTCCACCGAAGAAGGCACGTCAACCGAACCAAGTGAAGG |
| | | ATCTGCCCCTGGGACCAGCACTGAACCATCTGAGATGGACA |
| | | AGAAGTACAGCATCGGCCTGGCCATCGGCACCAACTCTGTG |
| | | GGCTGGGCCGTGATCACCGACGAGTACAAGGTGCCCAGCAA |
| | | GAAATTCAAGGTGCTGGGCAACACCGACCGGCACAGCATCA |
| | | AGAAGAACCTGATCGGCGCCCTGCTGTTCGACAGCGGAGAA |
| | | ACAGCCGAGGCCACCCGGCTGAAGAGAACCGCCAGAAGAA |
| | | GATACACCAGACGGAAGAACCGGATCTGCTATCTGCAAGAG |
| | | ATCTTCAGCAACGAGATGGCCAAGGTGGACGACAGCTTCTTC |
| | | CACAGACTGGAAGAGTCCTTCCTGGTGGAAGAGGATAAGAA |
| | | GCACGAGCGGCACCCCATCTTCGGCAACATCGTGGACGAGG |
| | | TGGCCTACCACGAGAAGTACCCCACCATCTACCACCTGAGA |
| | | AAGAAACTGGTGGACAGCACCGACAAGGCCGACCTGCGGCT |
| | | GATCTATCTGGCCCTGGCCCACATGATCAAGTTCCGGGGCCA |
| | | CTTCCTGATCGAGGGCGACCTGAACCCCGACAACAGCGACG |
| | | TGGACAAGCTGTTCATCCAGCTGGTGCAGACCTACAACCAGC |
| | | TGTTCGAGGAAAACCCCATCAACGCCAGCGGCGTGGACGCC |
| | | AAGGCCATCCTGTCTGCCAGACTGAGCAAGAGCAGACGGCT |
| | | GGAAAATCTGATCGCCCAGCTGCCCGGCGAGAAGAAGAATG |
| | | GCCTGTTCGGCAACCTGATTGCCCTGAGCCTGGGCCTGACCC |
| | | CCAACTTCAAGAGCAACTTCGACCTGGCCGAGGATGCCAAA |
| | | CTGCAGCTGAGCAAGGACACCTACGACGACGACCTGGACAA |
| | | CCTGCTGGCCCAGATCGGCGACCAGTACGCCGACCTGTTTCT |
| | | GGCCGCCAAGAACCTGTCCGACGCCATCCTGCTGAGCGACA |
| | | TCCTGAGAGTGAACACCGAGATCACCAAGGCCCCCCTGAGC |
| | | GCCTCTATGATCAAGAGATACGACGAGCACCACCAGGACCT |
| | | GACCCTGCTGAAAGCTCTCGTGCGGCAGCAGCTGCCTGAGA |
| | | AGTACAAAGAGATTTTCTTCGACCAGAGCAAGAACGGCTAC |
| | | GCCGGCTACATCGATGGCGGAGCCAGCCAGGAAGAGTTCTA |
| | | CAAGTTCATCAAGCCCATCCTGGAAAAGATGGACGGCACCG |
| | | AGGAACTGCTCGTGAAGCTGAACAGAGAGGACCTGCTGCGG |
| | | AAGCAGCGGACCTTCGACAACGGCAGCATCCCCCACCAGAT |
| | | CCACCTGGGAGAGCTGCACGCCATTCTGCGGCGGCAGGAAG |
| | | ATTTTTACCCATTCCTGAAGGACAACCGGGAAAAGATCGAG |
| | | AAGATCCTGACCTTCCGCATCCCCTACTACGTGGGCCCTCTG |
| | | GCCAGGGGAAACAGCAGATTCGCCTGGATGACCAGAAAGAG |
| | | CGAGGAAACCATCACCCCCTGGAACTTCGAGGAAGTGGTGG |
| | | ACAAGGGCGCCAGCGCCCAGAGCTTCATCGAGCGGATGACC |
| | | AACTTCGATAAGAACCTGCCCAACGAGAAGGTGCTGCCCAA |
| | | GCACAGCCTGCTGTACGAGTACTTCACCGTGTACAACGAGCT |
| | | GACCAAAGTGAAATACGTGACCGAGGGAATGAGAAAGCCCG |
| | | CCTTCCTGAGCGGCGAGCAGAAAAAAGCCATCGTGGACCTG |
| | | CTGTTCAAGACCAACCGGAAAGTGACCGTGAAGCAGCTGAA |
| | | AGAGGACTACTTCAAGAAAATCGAGTGCTTCGACTCCGTGG |
| | | AAATCTCCGGCGTGGAAGATCGGTTCAACGCCTCCCTGGGCA |
| | | CATACCACGATCTGCTGAAAATTATCAAGGACAAGGACTTCC |
| | | TGGACAATGAGGAAAACGAGGACATTCTGGAAGATATCGTG |
| | | CTGACCCTGACACTGTTTGAGGACAGAGAGATGATCGAGGA |
| | | ACGGCTGAAAACCTATGCCCACCTGTTCGACGACAAAGTGA |
| | | TGAAGCAGCTGAAGCGGCGGAGATACACCGGCTGGGGCAGG |
| | | CTGAGCCGGAAGCTGATCAACGGCATCCGGGACAAGCAGTC |
| | | CGGCAAGACAATCCTGGATTTCCTGAAGTCCGACGGCTTCGC |
| | | CAACAGAAACTTCATGCAGCTGATCCACGACGACAGCCTGA |
| | | CCTTTAAAGAGGACATCCAGAAAGCCCAGGTGTCCGGCCAG |
| | | GGCGATAGCCTGCACGAGCACATTGCCAATCTGGCCGGCAG |
| | | CCCCGCCATTAAGAAGGGCATCCTGCAGACAGTGAAGGTGG |
| | | TGGACGAGCTCGTGAAAGTGATGGGCCGGCACAAGCCCGAG |
| | | AACATCGTGATCGAAATGGCCAGAGAGAACCAGACCACCCA |
| | | GAAGGGACAGAAGAACAGCCGCGAGAGAATGAAGCGGATC |
| | | GAAGAGGGCATCAAAGAGCTGGGCAGCCAGATCCTGAAAGA |
| | | ACACCCCGTGGAAAACACCCAGCTGCAGAACGAGAAGCTGT |
| | | ACCTGTACTACCTGCAGAATGGGCGGGATATGTACGTGGAC |
| | | CAGGAACTGGACATCAACCGGCTGTCCGACTACGATGTGGA |
| | | CGCTATCGTGCCTCAGAGCTTTCTGAAGGACGACTCCATCGA |
| | | TAACAAAGTGCTGACTCGGAGCGACAAGAACCGGGGCAAGA |
| | | GCGACAACGTGCCCTCCGAAGAGGTCGTGAAGAAGATGAAG |
| | | AACTACTGGCGCCAGCTGCTGAATGCCAAGCTGATTACCCAG |
| | | AGGAAGTTCGACAATCTGACCAAGGCCGAGAGAGGCGGCCT |
| | | GAGCGAACTGGATAAGGCCGGCTTCATCAAGAGACAGCTGG |
| | | TGGAAACCCGGCAGATCACAAAGCACGTGGCACAGATCCTG |
| | | GACTCCCGGATGAACACTAAGTACGACGAGAACGACAAACT |
| | | GATCCGGGAAGTGAAAGTGATCACCCTGAAGTCCAAGCTGG |
| | | TGTCCGATTTCCGGAAGGATTTCCAGTTTTACAAAGTGCGCG |
| | | AGATCAACAACTACCACCACGCCCACGACGCCTACCTGAAC |
| | | GCCGTCGTGGGAACCGCCCTGATCAAAAAGTACCCTAAGCT |
| | | GGAAAGCGAGTTCGTGTACGGCGACTACAAGGTGTACGACG |
| | | TGCGGAAGATGATCGCCAAGAGCGAGCAGGAAATCGGCAAG |
| | | GCTACCGCCAAGTACTTCTTCTACAGCAACATCATGAACTTT |
| | | TTCAAGACCGAGATTACCCTGGCCAACGGCGAGATCCGGAA |
| | | GCGGCCTCTGATCGAGACAAACGGCGAAACAGGCGAGATCG |
| | | TGTGGGATAAGGGCCGGGACTTTGCCACCGTGCGGAAAGTG |
| | | CTGTCTATGCCCCAAGTGAATATCGTGAAAAAGACCGAGGT |
| | | GCAGACAGGCGGCTTCAGCAAAGAGTCTATCCTGCCCAAGA |
| | | GGAACAGCGACAAGCTGATCGCCAGAAAGAAGGACTGGGA |
| | | CCCTAAGAAGTACGGCGGCTTCGACAGCCCCACCGTGGCCT |
| | | ATTCTGTGCTGGTGGTGGCCAAAGTGGAAAAGGGCAAGTCC |
| | | AAGAAACTGAAGAGTGTGAAAGAGCTGCTGGGGATCACCAT |
| | | CATGGAAAGAAGCAGCTTCGAGAAGAATCCCATCGACTTTC |
| | | TGGAAGCCAAGGGCTACAAAGAAGTGAAAAAGGACCTGATC |
| | | ATCAAGCTGCCTAAGTACTCCCTGTTCGAGCTGGAAAACGGC |
| | | CGGAAGAGAATGCTGGCCTCTGCCGGCGAACTGCAGAAGGG |
| | | AAACGAACTGGCCCTGCCCTCCAAATATGTGAACTTCCTGTA |
| | | CCTGGCCAGCCACTATGAGAAGCTGAAGGGCTCCCCCGAGG |
| | | ATAATGAGCAGAAACAGCTGTTTGTGGAACAGCACAAACAC |
| | | TACCTGGACGAGATCATCGAGCAGATCAGCGAGTTCTCCAA |
| | | GAGAGTGATCCTGGCCGACGCTAATCTGGACAAGGTGCTGA |
| | | GCGCCTACAACAAGCACAGAGACAAGCCTATCAGAGAGCAG |
| | | GCCGAGAATATCATCCACCTGTTTACCCTGACCAATCTGGGA |
| | | GCCCCTGCCGCCTTCAAGTACTTTGACACCACCATCGACCGG |
| | | AAGAGGTACACCAGCACCAAAGAGGTGCTGGACGCCACCCT |
| | | GATCCACCAGAGCATCACCGGCCTGTACGAGACACGGATCG |
| | | ACCTGTCTCAGCTGGGAGGCGACAGCGGAAGTGAGACCCCA |
| | | GGTACATCCGAATCAGCAACGCCTGAAAGCACCGGTCGGAC |
| | | ACTGGTGACCTTCAAGGATGTATTTGTGGACTTCACCAGGGA |
| | | GGAGTGGAAGCTGCTGGACACTGCTCAGCAGATCGTGTACA |
| | | GAAATGTGATGCTGGAGAACTATAAGAACCTGGTTTCCTTGG |
| | | GTTATCAGCTTACTAAGCCAGATGTGATCCTCCGGTTGGAGA |
| | | AGGGAGAAGAGCCCCCAAAAAAGAAGAGAAAGGTACCGAA |
| | | GAAAAAAAGAAAGGTC |
| 19 | Fusion Protein 2 | MNHDQEFDPPKVYPPVPAEKRKPIRVLSLFDGIATGLLVLKDLG |
| | Amino Acid | IQVDRYIASEVCEDSITVGMVRHQGKIMYVGDVRSVTQKHIQE |
| | Sequence | WGPFDLVIGGSPCNDLSIVNPARKGLYEGTGRLFFEFYRLLHDA |
| | 3A-3L-NLS- | RPKEGDDRPFFWLFENVVAMGVSDKRDISRFLESNPVMIDAKE |
| | dCas9-NLS- | VSAAHRARYFWGNLPGMNRPLASTVNDKLELQECLEHGRIAK |
| | KRAB | FSKVRTITTRSNSIKQGKDQHFPVFMNEKEDILWCTEMERVFGF |
| | | PVHYTDVSNMSRLARQRLLGRSWSVPVIRHLFAPLKEYFACVS |
| | | SGNSNANSRGPSFSSGLVPLSLRGSHNPLEMFETVPVWRRQPVR |
| | | VLSLFEDIKKELTSLGFLESGSDPGQLKHVVDVTDTVRKDVEE |
| | | WGPFDLVYGATPPLGHTCDRPPSWYLFQFHRLLQYARPKPGSP |
| | | RPFFWMFVDNLVLNKEDLDVASRFLEMEPVTIPDVHGGSLQNA |
| | | VRVWSNIPAIRSRHWALVSEEELSLLAQNKQSSKLAAKWPTKL |
| | | VKNCFLPLREYFKYFSTELTSSLGGPSSGAPPPSGGSPAGSPTST |
| | | EEGTSESATPESGPGTSTEPSEGSAPGSPAGSPTSTEEGTSTEPSE |
| | | GSAPGTSTEPSEPKKKRKVMDKKYSIGLAIGTNSVGWAVITDEY |
| | | KVPSKKFKVLGNTDRHSIKKNLIGALLFDSGETAEATRLKRTAR |
| | | RRYTRRKNRICYLQEIFSNEMAKVDDSFFHRLEESFLVEEDKKH |
| | | ERHPIFGNIVDEVAYHEKYPTIYHLRKKLVDSTDKADLRLIYLA |
| | | LAHMIKFRGHFLIEGDLNPDNSDVDKLFIQLVQTYNQLFEENPI |
| | | NASGVDAKAILSARLSKSRRLENLIAQLPGEKKNGLFGNLIALS |
| | | LGLTPNFKSNFDLAEDAKLQLSKDTYDDDLDNLLAQIGDQYAD |
| | | LFLAAKNLSDAILLSDILRVNTEITKAPLSASMIKRYDEHHQDLT |
| | | LLKALVRQQLPEKYKEIFFDQSKNGYAGYIDGGASQEEFYKFIK |
| | | PILEKMDGTEELLVKLNREDLLRKQRTFDNGSIPHQIHLGELHAI |
| | | LRRQEDFYPFLKDNREKIEKILTFRIPYYVGPLARGNSRFAWMT |
| | | RKSEETITPWNFEEVVDKGASAQSFIERMTNFDKNLPNEKVLPK |
| | | HSLLYEYFTVYNELTKVKYVTEGMRKPAFLSGEQKKAIVDLLF |
| | | KTNRKVTVKQLKEDYFKKIECFDSVEISGVEDRFNASLGTYHDL |
| | | LKIIKDKDFLDNEENEDILEDIVLTLTLFEDREMIEERLKTYAHLF |
| | | DDKVMKQLKRRRYTGWGRLSRKLINGIRDKQSGKTILDFLKSD |
| | | GFANRNFMQLIHDDSLTFKEDIQKAQVSGQGDSLHEHIANLAG |
| | | SPAIKKGILQTVKVVDELVKVMGRHKPENIVIEMARENQTTQK |
| | | GQKNSRERMKRIEEGIKELGSQILKEHPVENTQLQNEKLYLYYL |
| | | QNGRDMYVDQELDINRLSDYDVDAIVPQSFLKDDSIDNKVLTR |
| | | SDKNRGKSDNVPSEEVVKKMKNYWRQLLNAKLITQRKFDNLT |
| | | KAERGGLSELDKAGFIKRQLVETRQITKHVAQILDSRMNTKYD |
| | | ENDKLIREVKVITLKSKLVSDFRKDFQFYKVREINNYHHAHDA |
| | | YLNAVVGTALIKKYPKLESEFVYGDYKVYDVRKMIAKSEQEIG |
| | | KATAKYFFYSNIMNFFKTEITLANGEIRKRPLIETNGETGEIVWD |
| | | KGRDFATVRKVLSMPQVNIVKKTEVQTGGFSKESILPKRNSDK |
| | | LIARKKDWDPKKYGGFDSPTVAYSVLVVAKVEKGKSKKLKSV |
| | | KELLGITIMERSSFEKNPIDFLEAKGYKEVKKDLIIKLPKYSLFEL |
| | | ENGRKRMLASAGELQKGNELALPSKYVNFLYLASHYEKLKGSP |
| | | EDNEQKQLFVEQHKHYLDEIIEQISEFSKRVILADANLDKVLSA |
| | | YNKHRDKPIREQAENIIHLFTLTNLGAPAAFKYFDTTIDRKRYTS |
| | | TKEVLDATLIHQSITGLYETRIDLSQLGGDPKKKRKVSGSETPGT |
| | | SESATPESTGRTLVTFKDVFVDFTREEWKLLDTAQQIVYRNVM |
| | | LENYKNLVSLGYQLTKPDVILRLEKGEEP |
| 20 | Fusion Protein 2 | ATGAACCACGACCAGGAATTTGACCCTCCAAAGGTTTACCCA |
| | DNA Sequence | CCTGTCCCAGCTGAGAAGAGGAAGCCCATCCGGGTGCTGTCT |
| | | CTCTTTGATGGAATCGCTACAGGGCTCCTGGTGCTGAAGGAC |
| | | TTGGGCATTCAGGTGGACCGCTACATTGCCTCGGAGGTGTGT |
| | | GAGGACTCCATCACGGTGGGCATGGTGCGGCACCAGGGGAA |
| | | GATCATGTACGTCGGGGACGTCCGCAGCGTCACACAGAAGC |
| | | ATATCCAGGAGTGGGGCCCATTCGATCTGGTGATTGGGGGC |
| | | AGTCCCTGCAATGACCTCTCCATCGTCAACCCTGCTCGCAAG |
| | | GGCCTCTACGAGGGCACTGGCCGGCTCTTCTTTGAGTTCTAC |
| | | CGCCTCCTGCATGATGCGCGGCCCAAGGAGGGAGATGATCG |
| | | CCCCTTCTTCTGGCTCTTTGAGAATGTGGTGGCCATGGGCGT |
| | | TAGTGACAAGAGGGACATCTCGCGATTTCTCGAGTCCAACCC |
| | | TGTGATGATTGATGCCAAAGAAGTGTCAGCTGCACACAGGG |
| | | CCCGCTACTTCTGGGGTAACCTTCCCGGTATGAACAGGCCGT |
| | | TGGCATCCACTGTGAATGATAAGCTGGAGCTGCAGGAGTGT |
| | | CTGGAGCATGGCAGGATAGCCAAGTTCAGCAAAGTGAGGAC |
| | | CATTACTACGAGGTCAAACTCCATAAAGCAGGGCAAAGACC |
| | | AGCATTTTCCTGTCTTCATGAATGAGAAAGAGGACATCTTAT |
| | | GGTGCACTGAAATGGAAAGGGTATTTGGTTTCCCAGTCCACT |
| | | ATACTGACGTCTCCAACATGAGCCGCTTGGCGAGGCAGAGA |
| | | CTGCTGGGCCGGTCATGGAGCGTGCCAGTCATCCGCCACCTC |
| | | TTCGCTCCGCTGAAGGAGTATTTTGCGTGTGTGTCTAGCGGC |
| | | AATAGTAACGCTAACAGCCGCGGGCCGAGCTTCAGCAGCGG |
| | | CCTGGTGCCGTTAAGCTTGCGCGGCAGCCATAATCCCCTTGA |
| | | GATGTTCGAAACCGTGCCTGTGTGGAGGAGACAGCCAGTCC |
| | | GGGTGCTGTCCCTTTTTGAAGACATCAAGAAAGAGCTGACG |
| | | AGTTTGGGCTTTTTGGAAAGTGGTTCTGACCCGGGACAACTG |
| | | AAGCATGTGGTTGATGTCACAGACACAGTGAGGAAGGATGT |
| | | GGAGGAGTGGGGACCCTTCGATCTTGTGTACGGCGCCACAC |
| | | CTCCCCTGGGCCACACCTGTGACCGTCCTCCCAGCTGGTACC |
| | | TGTTCCAGTTCCACCGGCTCCTGCAGTACGCACGGCCCAAGC |
| | | CAGGCAGCCCCAGGCCCTTCTTCTGGATGTTCGTGGACAATC |
| | | TGGTGCTGAACAAGGAAGACCTGGACGTCGCATCTCGCTTCC |
| | | TGGAGATGGAGCCAGTCACCATCCCAGATGTCCACGGCGGA |
| | | TCCTTGCAGAATGCTGTCCGCGTGTGGAGCAACATCCCAGCC |
| | | ATAAGGAGCAGGCACTGGGCTCTGGTTTCGGAAGAAGAATT |
| | | GTCCCTGCTGGCCCAGAACAAGCAGAGCTCGAAGCTCGCGG |
| | | CCAAGTGGCCCACCAAGCTGGTGAAGAACTGCTTTCTCCCCC |
| | | TAAGAGAATATTTCAAGTATTTTTCAACAGAACTCACTTCCT |
| | | CTTTAGGAGGGCCGAGCTCTGGCGCACCCCCACCAAGTGGA |
| | | GGGTCTCCTGCCGGGTCCCCAACATCTACTGAAGAAGGCACC |
| | | AGCGAATCCGCAACGCCCGAGTCAGGCCCTGGTACCTCCAC |
| | | AGAACCATCTGAAGGTAGTGCGCCTGGTTCCCCAGCTGGAA |
| | | GCCCTACTTCCACCGAAGAAGGCACGTCAACCGAACCAAGT |
| | | GAAGGATCTGCCCCTGGGACCAGCACTGAACCATCTGAGCC |
| | | AAAAAAGAAGAGAAAGGTAATGGACAAGAAGTACAGCATC |
| | | GGCCTGGCCATCGGCACCAACTCTGTGGGCTGGGCCGTGATC |
| | | ACCGACGAGTACAAGGTGCCCAGCAAGAAATTCAAGGTGCT |
| | | GGGCAACACCGACCGGCACAGCATCAAGAAGAACCTGATCG |
| | | GCGCCCTGCTGTTCGACAGCGGAGAAACAGCCGAGGCCACC |
| | | CGGCTGAAGAGAACCGCCAGAAGAAGATACACCAGACGGA |
| | | AGAACCGGATCTGCTATCTGCAAGAGATCTTCAGCAACGAG |
| | | ATGGCCAAGGTGGACGACAGCTTCTTCCACAGACTGGAAGA |
| | | GTCCTTCCTGGTGGAAGAGGATAAGAAGCACGAGCGGCACC |
| | | CCATCTTCGGCAACATCGTGGACGAGGTGGCCTACCACGAG |
| | | AAGTACCCCACCATCTACCACCTGAGAAAGAAACTGGTGGA |
| | | CAGCACCGACAAGGCCGACCTGCGGCTGATCTATCTGGCCCT |
| | | GGCCCACATGATCAAGTTCCGGGGCCACTTCCTGATCGAGGG |
| | | CGACCTGAACCCCGACAACAGCGACGTGGACAAGCTGTTCA |
| | | TCCAGCTGGTGCAGACCTACAACCAGCTGTTCGAGGAAAAC |
| | | CCCATCAACGCCAGCGGCGTGGACGCCAAGGCCATCCTGTC |
| | | TGCCAGACTGAGCAAGAGCAGACGGCTGGAAAATCTGATCG |
| | | CCCAGCTGCCCGGCGAGAAGAAGAATGGCCTGTTCGGCAAC |
| | | CTGATTGCCCTGAGCCTGGGCCTGACCCCCAACTTCAAGAGC |
| | | AACTTCGACCTGGCCGAGGATGCCAAACTGCAGCTGAGCAA |
| | | GGACACCTACGACGACGACCTGGACAACCTGCTGGCCCAGA |
| | | TCGGCGACCAGTACGCCGACCTGTTTCTGGCCGCCAAGAACC |
| | | TGTCCGACGCCATCCTGCTGAGCGACATCCTGAGAGTGAACA |
| | | CCGAGATCACCAAGGCCCCCCTGAGCGCCTCTATGATCAAG |
| | | AGATACGACGAGCACCACCAGGACCTGACCCTGCTGAAAGC |
| | | TCTCGTGCGGCAGCAGCTGCCTGAGAAGTACAAAGAGATTTT |
| | | CTTCGACCAGAGCAAGAACGGCTACGCCGGCTACATCGATG |
| | | GCGGAGCCAGCCAGGAAGAGTTCTACAAGTTCATCAAGCCC |
| | | ATCCTGGAAAAGATGGACGGCACCGAGGAACTGCTCGTGAA |
| | | GCTGAACAGAGAGGACCTGCTGCGGAAGCAGCGGACCTTCG |
| | | ACAACGGCAGCATCCCCCACCAGATCCACCTGGGAGAGCTG |
| | | CACGCCATTCTGCGGCGGCAGGAAGATTTTTACCCATTCCTG |
| | | AAGGACAACCGGGAAAAGATCGAGAAGATCCTGACCTTCCG |
| | | CATCCCCTACTACGTGGGCCCTCTGGCCAGGGGAAACAGCA |
| | | GATTCGCCTGGATGACCAGAAAGAGCGAGGAAACCATCACC |
| | | CCCTGGAACTTCGAGGAAGTGGTGGACAAGGGCGCCAGCGC |
| | | CCAGAGCTTCATCGAGCGGATGACCAACTTCGATAAGAACC |
| | | TGCCCAACGAGAAGGTGCTGCCCAAGCACAGCCTGCTGTAC |
| | | GAGTACTTCACCGTGTACAACGAGCTGACCAAAGTGAAATA |
| | | CGTGACCGAGGGAATGAGAAAGCCCGCCTTCCTGAGCGGCG |
| | | AGCAGAAAAAAGCCATCGTGGACCTGCTGTTCAAGACCAAC |
| | | CGGAAAGTGACCGTGAAGCAGCTGAAAGAGGACTACTTCAA |
| | | GAAAATCGAGTGCTTCGACTCCGTGGAAATCTCCGGCGTGG |
| | | AAGATCGGTTCAACGCCTCCCTGGGCACATACCACGATCTGC |
| | | TGAAAATTATCAAGGACAAGGACTTCCTGGACAATGAGGAA |
| | | AACGAGGACATTCTGGAAGATATCGTGCTGACCCTGACACT |
| | | GTTTGAGGACAGAGAGATGATCGAGGAACGGCTGAAAACCT |
| | | ATGCCCACCTGTTCGACGACAAAGTGATGAAGCAGCTGAAG |
| | | CGGCGGAGATACACCGGCTGGGGCAGGCTGAGCCGGAAGCT |
| | | GATCAACGGCATCCGGGACAAGCAGTCCGGCAAGACAATCC |
| | | TGGATTTCCTGAAGTCCGACGGCTTCGCCAACAGAAACTTCA |
| | | TGCAGCTGATCCACGACGACAGCCTGACCTTTAAAGAGGAC |
| | | ATCCAGAAAGCCCAGGTGTCCGGCCAGGGCGATAGCCTGCA |
| | | CGAGCACATTGCCAATCTGGCCGGCAGCCCCGCCATTAAGA |
| | | AGGGCATCCTGCAGACAGTGAAGGTGGTGGACGAGCTCGTG |
| | | AAAGTGATGGGCCGGCACAAGCCCGAGAACATCGTGATCGA |
| | | AATGGCCAGAGAGAACCAGACCACCCAGAAGGGACAGAAG |
| | | AACAGCCGCGAGAGAATGAAGCGGATCGAAGAGGGCATCA |
| | | AAGAGCTGGGCAGCCAGATCCTGAAAGAACACCCCGTGGAA |
| | | AACACCCAGCTGCAGAACGAGAAGCTGTACCTGTACTACCT |
| | | GCAGAATGGGGGGATATGTACGTGGACCAGGAACTGGACA |
| | | TCAACCGGCTGTCCGACTACGATGTGGACGCTATCGTGCCTC |
| | | AGAGCTTTCTGAAGGACGACTCCATCGATAACAAAGTGCTG |
| | | ACTCGGAGCGACAAGAACCGGGGCAAGAGCGACAACGTGCC |
| | | CTCCGAAGAGGTCGTGAAGAAGATGAAGAACTACTGGCGCC |
| | | AGCTGCTGAATGCCAAGCTGATTACCCAGAGGAAGTTCGAC |
| | | AATCTGACCAAGGCCGAGAGAGGCGGCCTGAGCGAACTGGA |
| | | TAAGGCCGGCTTCATCAAGAGACAGCTGGTGGAAACCCGGC |
| | | AGATCACAAAGCACGTGGCACAGATCCTGGACTCCCGGATG |
| | | AACACTAAGTACGACGAGAACGACAAACTGATCCGGGAAGT |
| | | GAAAGTGATCACCCTGAAGTCCAAGCTGGTGTCCGATTTCCG |
| | | GAAGGATTTCCAGTTTTACAAAGTGCGCGAGATCAACAACT |
| | | ACCACCACGCCCACGACGCCTACCTGAACGCCGTCGTGGGA |
| | | ACCGCCCTGATCAAAAAGTACCCTAAGCTGGAAAGCGAGTT |
| | | CGTGTACGGCGACTACAAGGTGTACGACGTGCGGAAGATGA |
| | | TCGCCAAGAGCGAGCAGGAAATCGGCAAGGCTACCGCCAAG |
| | | TACTTCTTCTACAGCAACATCATGAACTTTTTCAAGACCGAG |
| | | ATTACCCTGGCCAACGGCGAGATCCGGAAGCGGCCTCTGAT |
| | | CGAGACAAACGGCGAAACAGGCGAGATCGTGTGGGATAAG |
| | | GGCCGGGACTTTGCCACCGTGCGGAAAGTGCTGTCTATGCCC |
| | | CAAGTGAATATCGTGAAAAAGACCGAGGTGCAGACAGGCGG |
| | | CTTCAGCAAAGAGTCTATCCTGCCCAAGAGGAACAGCGACA |
| | | AGCTGATCGCCAGAAAGAAGGACTGGGACCCTAAGAAGTAC |
| | | GGCGGCTTCGACAGCCCCACCGTGGCCTATTCTGTGCTGGTG |
| | | GTGGCCAAAGTGGAAAAGGGCAAGTCCAAGAAACTGAAGA |
| | | GTGTGAAAGAGCTGCTGGGGATCACCATCATGGAAAGAAGC |
| | | AGCTTCGAGAAGAATCCCATCGACTTTCTGGAAGCCAAGGG |
| | | CTACAAAGAAGTGAAAAAGGACCTGATCATCAAGCTGCCTA |
| | | AGTACTCCCTGTTCGAGCTGGAAAACGGCCGGAAGAGAATG |
| | | CTGGCCTCTGCCGGCGAACTGCAGAAGGGAAACGAACTGGC |
| | | CCTGCCCTCCAAATATGTGAACTTCCTGTACCTGGCCAGCCA |
| | | CTATGAGAAGCTGAAGGGCTCCCCCGAGGATAATGAGCAGA |
| | | AACAGCTGTTTGTGGAACAGCACAAACACTACCTGGACGAG |
| | | ATCATCGAGCAGATCAGCGAGTTCTCCAAGAGAGTGATCCT |
| | | GGCCGACGCTAATCTGGACAAGGTGCTGAGCGCCTACAACA |
| | | AGCACAGAGACAAGCCTATCAGAGAGCAGGCCGAGAATATC |
| | | ATCCACCTGTTTACCCTGACCAATCTGGGAGCCCCTGCCGCC |
| | | TTCAAGTACTTTGACACCACCATCGACCGGAAGAGGTACACC |
| | | AGCACCAAAGAGGTGCTGGACGCCACCCTGATCCACCAGAG |
| | | CATCACCGGCCTGTACGAGACACGGATCGACCTGTCTCAGCT |
| | | GGGAGGCGACCCAAAAAAGAAGAGAAAGGTAAGCGGAAGT |
| | | GAGACCCCAGGTACATCCGAATCAGCAACGCCTGAAAGCAC |
| | | CGGTCGGACACTGGTGACCTTCAAGGATGTATTTGTGGACTT |
| | | CACCAGGGAGGAGTGGAAGCTGCTGGACACTGCTCAGCAGA |
| | | TCGTGTACAGAAATGTGATGCTGGAGAACTATAAGAACCTG |
| | | GTTTCCTTGGGTTATCAGCTTACTAAGCCAGATGTGATCCTC |
| | | CGGTTGGAGAAGGGAGAAGAGCCC |
| 21 | Fusion Protein 3 | MNHDQEFDPPKVYPPVPAEKRKPIRVLSLFDGIATGLLVLKDLG |
| | Amino Acid | IQVDRYIASEVCEDSITVGMVRHQGKIMYVGDVRSVTQKHIQE |
| | Sequence | WGPFDLVIGGSPCNDLSIVNPARKGLYEGTGRLFFEFYRLLHDA |
| | 3A-ADD-h3L- | RPKEGDDRPFFWLFENVVAMGVSDKRDISRFLESNPVMIDAKE |
| | NLS-dCas9-NLS- | VSAAHRARYFWGNLPGMNRPLASTVNDKLELQECLEHGRIAK |
| | KRAB | FSKVRTITTRSNSIKQGKDQHFPVFMNEKEDILWCTEMERVFGF |
| | | PVHYTDVSNMSRLARQRLLGRSWSVPVIRHLFAPLKEYFACVS |
| | | SGNSNANSRGPSFSSGLVPLSLRGSHMEVKVNRRSIEDICLCCG |
| | | TLQVYTRHPLFEGGLCAPCKDKFLESLFLYDDDGHQSYCTICCS |
| | | GGTLFICESPDCTRCYCFECVDILVGPGTSERINAMACWVCFLC |
| | | LPFSRSGLLQRRKRWRHQLKAFHDQEGAGPMEIYKTVSAWKR |
| | | QPVRVLSLFRNIDKVLKSLGFLESGSGSGGGTLKYVEDVTNVV |
| | | RRDVEKWGPFDLVYGSTQPLGSSCDRCPGWYMFQFHRILQYA |
| | | LPRQESQRPFFWIFMDNLLLTEDDQETTTRFLQTEAVTLQDVRG |
| | | RDYQNAMRVWSNIPGLKSKHAPLTPKEEEYLQAQVRSRSKLD |
| | | APKVDLLVKNCLLPLREYFKYFSQNSLPLGGPSSGAPPPSGGSP |
| | | AGSPTSTEEGTSESATPESGPGTSTEPSEGSAPGSPAGSPTSTEEG |
| | | TSTEPSEGSAPGTSTEPSEPKKKRKVMDKKYSIGLAIGTNSVGW |
| | | AVITDEYKVPSKKFKVLGNTDRHSIKKNLIGALLFDSGETAEAT |
| | | RLKRTARRRYTRRKNRICYLQEIFSNEMAKVDDSFFHRLEESFL |
| | | VEEDKKHERHPIFGNIVDEVAYHEKYPTIYHLRKKLVDSTDKA |
| | | DLRLIYLALAHMIKFRGHFLIEGDLNPDNSDVDKLFIQLVQTYN |
| | | QLFEENPINASGVDAKAILSARLSKSRRLENLIAQLPGEKKNGLF |
| | | GNLIALSLGLTPNFKSNFDLAEDAKLQLSKDTYDDDLDNLLAQI |
| | | GDQYADLFLAAKNLSDAILLSDILRVNTEITKAPLSASMIKRYD |
| | | EHHQDLTLLKALVRQQLPEKYKEIFFDQSKNGYAGYIDGGASQ |
| | | EEFYKFIKPILEKMDGTEELLVKLNREDLLRKQRTFDNGSIPHQI |
| | | HLGELHAILRRQEDFYPFLKDNREKIEKILTFRIPYYVGPLARGN |
| | | SRFAWMTRKSEETITPWNFEEVVDKGASAQSFIERMTNFDKNL |
| | | PNEKVLPKHSLLYEYFTVYNELTKVKYVTEGMRKPAFLSGEQK |
| | | KAIVDLLFKTNRKVTVKQLKEDYFKKIECFDSVEISGVEDRFNA |
| | | SLGTYHDLLKIIKDKDFLDNEENEDILEDIVLTLTLFEDREMIEER |
| | | LKTYAHLFDDKVMKQLKRRRYTGWGRLSRKLINGIRDKQSGK |
| | | TILDFLKSDGFANRNFMQLIHDDSLTFKEDIQKAQVSGQGDSLH |
| | | EHIANLAGSPAIKKGILQTVKVVDELVKVMGRHKPENIVIEMAR |
| | | ENQTTQKGQKNSRERMKRIEEGIKELGSQILKEHPVENTQLQNE |
| | | KLYLYYLQNGRDMYVDQELDINRLSDYDVDAIVPQSFLKDDSI |
| | | DNKVLTRSDKNRGKSDNVPSEEVVKKMKNYWRQLLNAKLITQ |
| | | RKFDNLTKAERGGLSELDKAGFIKRQLVETRQITKHVAQILDSR |
| | | MNTKYDENDKLIREVKVITLKSKLVSDFRKDFQFYKVREINNY |
| | | HHAHDAYLNAVVGTALIKKYPKLESEFVYGDYKVYDVRKMIA |
| | | KSEQEIGKATAKYFFYSNIMNFFKTEITLANGEIRKRPLIETNGET |
| | | GEIVWDKGRDFATVRKVLSMPQVNIVKKTEVQTGGFSKESILP |
| | | KRNSDKLIARKKDWDPKKYGGFDSPTVAYSVLVVAKVEKGKS |
| | | KKLKSVKELLGITIMERSSFEKNPIDFLEAKGYKEVKKDLIIKLP |
| | | KYSLFELENGRKRMLASAGELQKGNELALPSKYVNFLYLASHY |
| | | EKLKGSPEDNEQKQLFVEQHKHYLDEIIEQISEFSKRVILADANL |
| | | DKVLSAYNKHRDKPIREQAENIIHLFTLTNLGAPAAFKYFDTTID |
| | | RKRYTSTKEVLDATLIHQSITGLYETRIDLSQLGGDPKKKRKVS |
| | | GSETPGTSESATPESTGRTLVTFKDVFVDFTREEWKLLDTAQQI |
| | | VYRNVMLENYKNLVSLGYQLTKPDVILRLEKGEEP |
| 22 | Fusion Protein 3 | ATGAACCATGACCAGGAATTTGACCCCCCAAAGGTTTACCCA |
| | DNA Sequence | CCTGTGCCAGCTGAGAAGAGGAAGCCCATCCGCGTGCTGTCT |
| | | CTCTTTGATGGGATTGCTACAGGGCTCCTGGTGCTGAAGGAC |
| | | CTGGGCATCCAAGTGGACCGCTACATTGCCTCCGAGGTGTGT |
| | | GAGGACTCCATCACGGTGGGCATGGTGCGGCACCAGGGAAA |
| | | GATCATGTACGTCGGGGACGTCCGCAGCGTCACACAGAAGC |
| | | ATATCCAGGAGTGGGGCCCATTCGACCTGGTGATTGGAGGC |
| | | AGTCCCTGCAATGACCTCTCCATTGTCAACCCTGCCCGCAAG |
| | | GGACTTTATGAGGGTACTGGCCGCCTCTTCTTTGAGTTCTAC |
| | | CGCCTCCTGCATGATGCGCGGCCCAAGGAGGGAGATGATCG |
| | | CCCCTTCTTCTGGCTCTTTGAGAATGTGGTGGCCATGGGCGT |
| | | TAGTGACAAGAGGGACATCTCGCGATTTCTTGAGTCTAACCC |
| | | CGTGATGATTGACGCCAAAGAAGTGTCTGCTGCACACAGGG |
| | | CCCGTTACTTCTGGGGTAACCTTCCTGGCATGAACAGGCCTT |
| | | TGGCATCCACTGTGAATGATAAGCTGGAGCTGCAAGAGTGT |
| | | CTGGAGCACGGCAGAATAGCCAAGTTCAGCAAAGTGAGGAC |
| | | CATTACCACCAGGTCAAACTCTATAAAGCAGGGCAAAGACC |
| | | AGCATTTCCCCGTCTTCATGAACGAGAAGGAGGACATCCTGT |
| | | GGTGCACTGAAATGGAAAGGGTGTTTGGCTTCCCCGTCCACT |
| | | ACACAGACGTCTCCAACATGAGCCGCTTGGCGAGGCAGAGA |
| | | CTGCTGGGCCGATCGTGGAGCGTGCCGGTCATCCGCCACCTC |
| | | TTCGCTCCGCTGAAGGAATATTTTGCTTGTGTGTCTAGCGGC |
| | | AATAGTAACGCTAACAGCCGCGGGCCGAGCTTCAGCAGCGG |
| | | CCTGGTGCCGTTAAGCTTGCGCGGCAGCCATATGGAAGTCAA |
| | | AGTGAACCGACGGAGCATTGAAGACATCTGCCTCTGCTGTG |
| | | GAACTCTCCAGGTGTACACTCGGCACCCCTTGTTTGAGGGAG |
| | | GGTTATGTGCCCCATGTAAGGATAAGTTCCTGGAGTCCCTCT |
| | | TCCTGTATGATGATGATGGACACCAGAGTTACTGCACCATCT |
| | | GCTGTTCCGGGGGTACCCTGTTCATCTGTGAGAGCCCCGACT |
| | | GTACCAGATGCTACTGTTTCGAGTGTGTGGACATCCTGGTGG |
| | | GCCCCGGGACCTCAGAGAGGATCAATGCCATGGCCTGCTGG |
| | | GTTTGCTTCCTGTGCCTGCCCTTCTCACGGAGTGGACTGCTGC |
| | | AGAGGCGCAAGAGGTGGCGGCACCAGCTGAAGGCCTTCCAT |
| | | GATCAAGAGGGAGCGGGCCCTATGGAGATATACAAGACAGT |
| | | GTCTGCATGGAAGAGACAGCCAGTGCGGGTACTGAGCCTCT |
| | | TCAGAAACATCGACAAGGTACTAAAGAGTTTGGGCTTCTTGG |
| | | AAAGCGGTTCTGGTTCTGGGGGAGGAACGCTGAAGTACGTG |
| | | GAAGATGTCACAAATGTCGTGAGGAGAGACGTGGAGAAATG |
| | | GGGCCCCTTTGACCTGGTGTACGGCTCGACGCAGCCCCTAGG |
| | | CAGCTCTTGTGATCGCTGTCCCGGCTGGTACATGTTCCAGTT |
| | | CCACCGGATCCTGCAGTATGCGCTGCCTCGCCAGGAGAGTCA |
| | | GCGGCCCTTCTTCTGGATATTCATGGACAATCTGCTGCTGAC |
| | | TGAGGATGACCAAGAGACAACTACCCGCTTCCTTCAGACAG |
| | | AGGCTGTGACCCTCCAGGATGTCCGTGGCAGAGACTACCAG |
| | | AATGCTATGCGGGTGTGGAGCAACATTCCAGGGCTGAAGAG |
| | | CAAGCATGCGCCCCTGACCCCAAAGGAAGAAGAGTATCTGC |
| | | AAGCCCAAGTCAGAAGCAGGAGCAAGCTGGACGCCCCGAAA |
| | | GTTGACCTCCTGGTGAAGAACTGCCTTCTCCCGCTGAGAGAG |
| | | TACTTCAAGTATTTTTCTCAAAACTCACTTCCTCTTGGAGGGC |
| | | CGAGCTCTGGCGCACCCCCACCAAGTGGAGGGTCTCCTGCC |
| | | GGGTCCCCAACATCTACTGAAGAAGGCACCAGCGAATCCGC |
| | | AACGCCCGAGTCAGGCCCTGGTACCTCCACAGAACCATCTG |
| | | AAGGTAGTGCGCCTGGTTCCCCAGCTGGAAGCCCTACTTCCA |
| | | CCGAAGAAGGCACGTCAACCGAACCAAGTGAAGGATCTGCC |
| | | CCTGGGACCAGCACTGAACCATCTGAGCCAAAAAAGAAGAG |
| | | AAAGGTAATGGACAAGAAGTACAGCATCGGCCTGGCCATCG |
| | | GCACCAACTCTGTGGGCTGGGCCGTGATCACCGACGAGTAC |
| | | AAGGTGCCCAGCAAGAAATTCAAGGTGCTGGGCAACACCGA |
| | | CCGGCACAGCATCAAGAAGAACCTGATCGGCGCCCTGCTGT |
| | | TCGACAGCGGAGAAACAGCCGAGGCCACCCGGCTGAAGAGA |
| | | ACCGCCAGAAGAAGATACACCAGACGGAAGAACCGGATCTG |
| | | CTATCTGCAAGAGATCTTCAGCAACGAGATGGCCAAGGTGG |
| | | ACGACAGCTTCTTCCACAGACTGGAAGAGTCCTTCCTGGTGG |
| | | AAGAGGATAAGAAGCACGAGCGGCACCCCATCTTCGGCAAC |
| | | ATCGTGGACGAGGTGGCCTACCACGAGAAGTACCCCACCAT |
| | | CTACCACCTGAGAAAGAAACTGGTGGACAGCACCGACAAGG |
| | | CCGACCTGCGGCTGATCTATCTGGCCCTGGCCCACATGATCA |
| | | AGTTCCGGGGCCACTTCCTGATCGAGGGCGACCTGAACCCCG |
| | | ACAACAGCGACGTGGACAAGCTGTTCATCCAGCTGGTGCAG |
| | | ACCTACAACCAGCTGTTCGAGGAAAACCCCATCAACGCCAG |
| | | CGGCGTGGACGCCAAGGCCATCCTGTCTGCCAGACTGAGCA |
| | | AGAGCAGACGGCTGGAAAATCTGATCGCCCAGCTGCCCGGC |
| | | GAGAAGAAGAATGGCCTGTTCGGCAACCTGATTGCCCTGAG |
| | | CCTGGGCCTGACCCCCAACTTCAAGAGCAACTTCGACCTGGC |
| | | CGAGGATGCCAAACTGCAGCTGAGCAAGGACACCTACGACG |
| | | ACGACCTGGACAACCTGCTGGCCCAGATCGGCGACCAGTAC |
| | | GCCGACCTGTTTCTGGCCGCCAAGAACCTGTCCGACGCCATC |
| | | CTGCTGAGCGACATCCTGAGAGTGAACACCGAGATCACCAA |
| | | GGCCCCCCTGAGCGCCTCTATGATCAAGAGATACGACGAGC |
| | | ACCACCAGGACCTGACCCTGCTGAAAGCTCTCGTGCGGCAG |
| | | CAGCTGCCTGAGAAGTACAAAGAGATTTTCTTCGACCAGAG |
| | | CAAGAACGGCTACGCCGGCTACATCGATGGCGGAGCCAGCC |
| | | AGGAAGAGTTCTACAAGTTCATCAAGCCCATCCTGGAAAAG |
| | | ATGGACGGCACCGAGGAACTGCTCGTGAAGCTGAACAGAGA |
| | | GGACCTGCTGCGGAAGCAGCGGACCTTCGACAACGGCAGCA |
| | | TCCCCCACCAGATCCACCTGGGAGAGCTGCACGCCATTCTGC |
| | | GGCGGCAGGAAGATTTTTACCCATTCCTGAAGGACAACCGG |
| | | GAAAAGATCGAGAAGATCCTGACCTTCCGCATCCCCTACTAC |
| | | GTGGGCCCTCTGGCCAGGGGAAACAGCAGATTCGCCTGGAT |
| | | GACCAGAAAGAGCGAGGAAACCATCACCCCCTGGAACTTCG |
| | | AGGAAGTGGTGGACAAGGGCGCCAGCGCCCAGAGCTTCATC |
| | | GAGCGGATGACCAACTTCGATAAGAACCTGCCCAACGAGAA |
| | | GGTGCTGCCCAAGCACAGCCTGCTGTACGAGTACTTCACCGT |
| | | GTACAACGAGCTGACCAAAGTGAAATACGTGACCGAGGGAA |
| | | TGAGAAAGCCCGCCTTCCTGAGCGGCGAGCAGAAAAAAGCC |
| | | ATCGTGGACCTGCTGTTCAAGACCAACCGGAAAGTGACCGT |
| | | GAAGCAGCTGAAAGAGGACTACTTCAAGAAAATCGAGTGCT |
| | | TCGACTCCGTGGAAATCTCCGGCGTGGAAGATCGGTTCAACG |
| | | CCTCCCTGGGCACATACCACGATCTGCTGAAAATTATCAAGG |
| | | ACAAGGACTTCCTGGACAATGAGGAAAACGAGGACATTCTG |
| | | GAAGATATCGTGCTGACCCTGACACTGTTTGAGGACAGAGA |
| | | GATGATCGAGGAACGGCTGAAAACCTATGCCCACCTGTTCG |
| | | ACGACAAAGTGATGAAGCAGCTGAAGCGGCGGAGATACACC |
| | | GGCTGGGGCAGGCTGAGCCGGAAGCTGATCAACGGCATCCG |
| | | GGACAAGCAGTCCGGCAAGACAATCCTGGATTTCCTGAAGT |
| | | CCGACGGCTTCGCCAACAGAAACTTCATGCAGCTGATCCACG |
| | | ACGACAGCCTGACCTTTAAAGAGGACATCCAGAAAGCCCAG |
| | | GTGTCCGGCCAGGGCGATAGCCTGCACGAGCACATTGCCAA |
| | | TCTGGCCGGCAGCCCCGCCATTAAGAAGGGCATCCTGCAGA |
| | | CAGTGAAGGTGGTGGACGAGCTCGTGAAAGTGATGGGCCGG |
| | | CACAAGCCCGAGAACATCGTGATCGAAATGGCCAGAGAGAA |
| | | CCAGACCACCCAGAAGGGACAGAAGAACAGCCGCGAGAGA |
| | | ATGAAGCGGATCGAAGAGGGCATCAAAGAGCTGGGCAGCCA |
| | | GATCCTGAAAGAACACCCCGTGGAAAACACCCAGCTGCAGA |
| | | ACGAGAAGCTGTACCTGTACTACCTGCAGAATGGGCGGGAT |
| | | ATGTACGTGGACCAGGAACTGGACATCAACCGGCTGTCCGA |
| | | CTACGATGTGGACGCTATCGTGCCTCAGAGCTTTCTGAAGGA |
| | | CGACTCCATCGATAACAAAGTGCTGACTCGGAGCGACAAGA |
| | | ACCGGGGCAAGAGCGACAACGTGCCCTCCGAAGAGGTCGTG |
| | | AAGAAGATGAAGAACTACTGGCGCCAGCTGCTGAATGCCAA |
| | | GCTGATTACCCAGAGGAAGTTCGACAATCTGACCAAGGCCG |
| | | AGAGAGGCGGCCTGAGCGAACTGGATAAGGCCGGCTTCATC |
| | | AAGAGACAGCTGGTGGAAACCCGGCAGATCACAAAGCACGT |
| | | GGCACAGATCCTGGACTCCCGGATGAACACTAAGTACGACG |
| | | AGAACGACAAACTGATCCGGGAAGTGAAAGTGATCACCCTG |
| | | AAGTCCAAGCTGGTGTCCGATTTCCGGAAGGATTTCCAGTTT |
| | | TACAAAGTGCGCGAGATCAACAACTACCACCACGCCCACGA |
| | | CGCCTACCTGAACGCCGTCGTGGGAACCGCCCTGATCAAAA |
| | | AGTACCCTAAGCTGGAAAGCGAGTTCGTGTACGGCGACTAC |
| | | AAGGTGTACGACGTGCGGAAGATGATCGCCAAGAGCGAGCA |
| | | GGAAATCGGCAAGGCTACCGCCAAGTACTTCTTCTACAGCA |
| | | ACATCATGAACTTTTTCAAGACCGAGATTACCCTGGCCAACG |
| | | GCGAGATCCGGAAGCGGCCTCTGATCGAGACAAACGGCGAA |
| | | ACAGGCGAGATCGTGTGGGATAAGGGCCGGGACTTTGCCAC |
| | | CGTGCGGAAAGTGCTGTCTATGCCCCAAGTGAATATCGTGAA |
| | | AAAGACCGAGGTGCAGACAGGCGGCTTCAGCAAAGAGTCTA |
| | | TCCTGCCCAAGAGGAACAGCGACAAGCTGATCGCCAGAAAG |
| | | AAGGACTGGGACCCTAAGAAGTACGGCGGCTTCGACAGCCC |
| | | CACCGTGGCCTATTCTGTGCTGGTGGTGGCCAAAGTGGAAAA |
| | | GGGCAAGTCCAAGAAACTGAAGAGTGTGAAAGAGCTGCTGG |
| | | GGATCACCATCATGGAAAGAAGCAGCTTCGAGAAGAATCCC |
| | | ATCGACTTTCTGGAAGCCAAGGGCTACAAAGAAGTGAAAAA |
| | | GGACCTGATCATCAAGCTGCCTAAGTACTCCCTGTTCGAGCT |
| | | GGAAAACGGCCGGAAGAGAATGCTGGCCTCTGCCGGCGAAC |
| | | TGCAGAAGGGAAACGAACTGGCCCTGCCCTCCAAATATGTG |
| | | AACTTCCTGTACCTGGCCAGCCACTATGAGAAGCTGAAGGG |
| | | CTCCCCCGAGGATAATGAGCAGAAACAGCTGTTTGTGGAAC |
| | | AGCACAAACACTACCTGGACGAGATCATCGAGCAGATCAGC |
| | | GAGTTCTCCAAGAGAGTGATCCTGGCCGACGCTAATCTGGAC |
| | | AAGGTGCTGAGCGCCTACAACAAGCACAGAGACAAGCCTAT |
| | | CAGAGAGCAGGCCGAGAATATCATCCACCTGTTTACCCTGAC |
| | | CAATCTGGGAGCCCCTGCCGCCTTCAAGTACTTTGACACCAC |
| | | CATCGACCGGAAGAGGTACACCAGCACCAAAGAGGTGCTGG |
| | | ACGCCACCCTGATCCACCAGAGCATCACCGGCCTGTACGAG |
| | | ACACGGATCGACCTGTCTCAGCTGGGAGGCGACCCAAAAAA |
| | | GAAGAGAAAGGTAAGCGGAAGTGAGACCCCAGGTACATCCG |
| | | AATCAGCAACGCCTGAAAGCACCGGTCGGACACTGGTGACC |
| | | TTCAAGGATGTATTTGTGGACTTCACCAGGGAGGAGTGGAA |
| | | GCTGCTGGACACTGCTCAGCAGATCGTGTACAGAAATGTGAT |
| | | GCTGGAGAACTATAAGAACCTGGTTTCCTTGGGTTATCAGCT |
| | | TACTAAGCCAGATGTGATCCTCCGGTTGGAGAAGGGAGAAG |
| | | AGCCC |
| 23 | Fusion Protein 4 | MNHDQEFDPPKVYPPVPAEKRKPIRVLSLFDGIATGLLVLKDLG |
| | Amino Acid | IQVDRYIASEVCEDSITVGMVRHQGKIMYVGDVRSVTQKHIQE |
| | Sequence | WGPFDLVIGGSPCNDLSIVNPARKGLYEGTGRLFFEFYRLLHDA |
| | 3A-ADD-h3L- | RPKEGDDRPFFWLFENVVAMGVSDKRDISRFLESNPVMIDAKE |
| | NLS-dCas9-NLS- | VSAAHRARYFWGNLPGMNRPLASTVNDKLELQECLEHGRIAK |
| | KRAB | FSKVRTITTRSNSIKQGKDQHFPVFMNEKEDILWCTEMERVFGF |
| | | PVHYTDVSNMSRLARQRLLGRSWSVPVIRHLFAPLKEYFACVS |
| | | SGNSNANSRGPSFSSGLVPLSLRGSHMEVKANQRNIEDICICCGS |
| | | LQVHTQHPLFEGGICAPCKDKFLDALFLYDDDGYQSYCSICCSG |
| | | ETLLICGNPDCTRCYCFECVDSLVGPGTSGKVHAMSNWVCYLC |
| | | LPSSRSGLLQRRRKWRSQLKAFYDRESENPLEMFETVPVWRRQ |
| | | PVRVLSLFEDIKKELTSLGFLESGSDPGQLKHVVDVTDTVRKDV |
| | | EEWGPFDLVYGATPPLGHTCDRPPSWYLFQFHRLLQYARPKPG |
| | | SPRPFFWMFVDNLVLNKEDLDVASRFLEMEPVTIPDVHGGSLQ |
| | | NAVRVWSNIPAIRSRHWALVSEEELSLLAQNKQSSKLAAKWPT |
| | | KLVKNCFLPLREYFKYFSTELTSSLGGPSSGAPPPSGGSPAGSPT |
| | | STEEGTSESATPESGPGTSTEPSEGSAPGSPAGSPTSTEEGTSTEP |
| | | SEGSAPGTSTEPSEPKKKRKVMDKKYSIGLAIGTNSVGWAVITD |
| | | EYKVPSKKFKVLGNTDRHSIKKNLIGALLFDSGETAEATRLKRT |
| | | ARRRYTRRKNRICYLQEIFSNEMAKVDDSFFHRLEESFLVEEDK |
| | | KHERHPIFGNIVDEVAYHEKYPTIYHLRKKLVDSTDKADLRLIY |
| | | LALAHMIKFRGHFLIEGDLNPDNSDVDKLFIQLVQTYNQLFEEN |
| | | PINASGVDAKAILSARLSKSRRLENLIAQLPGEKKNGLFGNLIAL |
| | | SLGLTPNFKSNFDLAEDAKLQLSKDTYDDDLDNLLAQIGDQYA |
| | | DLFLAAKNLSDAILLSDILRVNTEITKAPLSASMIKRYDEHHQDL |
| | | TLLKALVRQQLPEKYKEIFFDQSKNGYAGYIDGGASQEEFYKFI |
| | | KPILEKMDGTEELLVKLNREDLLRKQRTFDNGSIPHQIHLGELH |
| | | AILRRQEDFYPFLKDNREKIEKILTFRIPYYVGPLARGNSRFAW |
| | | MTRKSEETITPWNFEEVVDKGASAQSFIERMTNFDKNLPNEKV |
| | | LPKHSLLYEYFTVYNELTKVKYVTEGMRKPAFLSGEQKKAIVD |
| | | LLFKTNRKVTVKQLKEDYFKKIECFDSVEISGVEDRFNASLGTY |
| | | HDLLKIIKDKDFLDNEENEDILEDIVLTLTLFEDREMIEERLKTY |
| | | AHLFDDKVMKQLKRRRYTGWGRLSRKLINGIRDKQSGKTILDF |
| | | LKSDGFANRNFMQLIHDDSLTFKEDIQKAQVSGQGDSLHEHIA |
| | | NLAGSPAIKKGILQTVKVVDELVKVMGRHKPENIVIEMARENQ |
| | | TTQKGQKNSRERMKRIEEGIKELGSQILKEHPVENTQLQNEKLY |
| | | LYYLQNGRDMYVDQELDINRLSDYDVDAIVPQSFLKDDSIDNK |
| | | VLTRSDKNRGKSDNVPSEEVVKKMKNYWRQLLNAKLITQRKF |
| | | DNLTKAERGGLSELDKAGFIKRQLVETRQITKHVAQILDSRMNT |
| | | KYDENDKLIREVKVITLKSKLVSDFRKDFQFYKVREINNYHHA |
| | | HDAYLNAVVGTALIKKYPKLESEFVYGDYKVYDVRKMIAKSE |
| | | QEIGKATAKYFFYSNIMNFFKTEITLANGEIRKRPLIETNGETGEI |
| | | VWDKGRDFATVRKVLSMPQVNIVKKTEVQTGGFSKESILPKRN |
| | | SDKLIARKKDWDPKKYGGFDSPTVAYSVLVVAKVEKGKSKKL |
| | | KSVKELLGITIMERSSFEKNPIDFLEAKGYKEVKKDLIIKLPKYSL |
| | | FELENGRKRMLASAGELQKGNELALPSKYVNFLYLASHYEKLK |
| | | GSPEDNEQKQLFVEQHKHYLDEIIEQISEFSKRVILADANLDKVL |
| | | SAYNKHRDKPIREQAENIIHLFTLTNLGAPAAFKYFDTTIDRKRY |
| | | TSTKEVLDATLIHQSITGLYETRIDLSQLGGDPKKKRKVSGSETP |
| | | GTSESATPESTGRTLVTFKDVFVDFTREEWKLLDTAQQIVYRNV |
| | | MLENYKNLVSLGYQLTKPDVILRLEKGEEP |
| 24 | Fusion Protein 4 | ATGAACCATGATCAAGAATTCGACCCACCTAAAGTCTACCCA |
| | DNA Sequence | CCTGTGCCCGCCGAAAAAAGGAAACCCATAAGGGTGCTGTC |
| | | ACTCTTTGATGGCATCGCCACTGGTCTCCTGGTTCTTAAGGA |
| | | TCTGGGAATTCAGGTCGATCGGTACATTGCTAGCGAGGTTTG |
| | | TGAGGATAGTATTACAGTGGGTATGGTGCGCCACCAGGGAA |
| | | AGATCATGTATGTTGGTGACGTTAGGAGCGTCACCCAGAAA |
| | | CATATCCAGGAGTGGGGACCCTTTGATTTGGTGATCGGAGGT |
| | | AGTCCCTGCAATGACCTTTCCATCGTGAATCCAGCCAGGAAA |
| | | GGGCTGTATGAAGGGACTGGTAGGCTCTTTTTCGAGTTTTAT |
| | | CGCCTGCTTCACGACGCTAGACCTAAGGAAGGTGACGATAG |
| | | GCCTTTCTTTTGGCTTTTTGAGAACGTCGTGGCAATGGGAGT |
| | | CTCCGACAAAAGGGACATTTCTCGCTTTCTGGAATCTAACCC |
| | | CGTTATGATCGATGCCAAGGAAGTTTCTGCCGCTCACAGGGC |
| | | AAGGTACTTCTGGGGCAATCTGCCCGGAATGAATCGCCCACT |
| | | GGCCAGTACCGTGAATGACAAACTGGAGCTGCAGGAGTGCC |
| | | TGGAGCACGGAAGAATCGCAAAGTTTTCTAAAGTCAGGACC |
| | | ATTACCACTCGCAGTAACTCCATAAAACAGGGTAAGGACCA |
| | | GCATTTTCCCGTCTTCATGAATGAAAAGGAAGATATTCTGTG |
| | | GTGCACTGAAATGGAGAGAGTTTTCGGGTTTCCCGTGCACTA |
| | | TACCGATGTTTCCAACATGTCCCGCCTTGCAAGACAAAGGCT |
| | | TTTGGGCCGCTCTTGGTCTGTGCCAGTGATCCGGCACTTGTTT |
| | | GCTCCCCTCAAAGAGTACTTCGCTTGCGTCAGTTCCGGAAAT |
| | | TCAAACGCTAACTCTCGGGGTCCATCTTTCTCCAGTGGTCTC |
| | | GTGCCACTGTCTCTCCGGGGCTCTCACATGGAAGTCAAGGCT |
| | | AACCAGCGAAATATAGAAGACATCTGCATCTGCTGCGGAAG |
| | | TCTCCAGGTTCACACACAGCACCCTCTGTTTGAGGGAGGGAT |
| | | CTGCGCCCCATGTAAGGACAAGTTCCTGGATGCCCTCTTCCT |
| | | GTACGACGATGACGGGTACCAATCCTACTGCTCCATCTGCTG |
| | | CTCCGGAGAGACGCTGCTCATCTGCGGAAACCCTGATTGCAC |
| | | CCGATGCTACTGCTTCGAGTGTGTGGATAGCCTGGTCGGCCC |
| | | CGGGACCTCGGGGAAGGTGCACGCCATGAGCAACTGGGTGT |
| | | GCTACCTGTGCCTGCCGTCCTCCCGAAGCGGGCTGCTGCAGC |
| | | GTCGGAGGAAGTGGCGCAGCCAGCTCAAGGCCTTCTACGAC |
| | | CGAGAGTCGGAGAATCCCCTGGAGATGTTTGAGACAGTGCC |
| | | AGTCTGGCGGAGGCAGCCCGTTCGCGTTCTCTCTCTGTTCGA |
| | | AGATATTAAAAAGGAACTCACCTCCCTTGGGTTCCTGGAGAG |
| | | CGGGAGCGACCCCGGACAGCTTAAGCACGTGGTCGACGTGA |
| | | CTGACACCGTCCGCAAAGACGTGGAGGAATGGGGCCCCTTC |
| | | GATCTGGTCTATGGGGCAACCCCTCCCCTTGGGCATACATGT |
| | | GATCGGCCTCCATCCTGGTACCTGTTCCAGTTTCACAGACTC |
| | | CTGCAGTATGCCAGGCCAAAGCCAGGGAGCCCAAGGCCCTT |
| | | TTTCTGGATGTTCGTCGACAACCTGGTCCTGAACAAAGAAGA |
| | | TCTCGACGTTGCTAGTCGCTTTCTCGAAATGGAGCCCGTGAC |
| | | CATTCCCGACGTGCATGGCGGTTCCCTCCAGAATGCAGTCAG |
| | | GGTTTGGAGCAATATCCCTGCCATCAGGTCAAGGCACTGGGC |
| | | ACTGGTTTCAGAGGAAGAGCTGTCCCTCCTTGCCCAGAACAA |
| | | GCAGTCATCCAAACTGGCAGCCAAGTGGCCAACTAAGCTGG |
| | | TCAAGAACTGCTTTCTTCCCCTCAGAGAATATTTTAAGTATTT |
| | | CAGTACTGAACTGACTAGCAGTCTGGGAGGGCCGAGCTCTG |
| | | GCGCACCCCCACCAAGTGGAGGGTCTCCTGCCGGGTCCCCA |
| | | ACATCTACTGAAGAAGGCACCAGCGAATCCGCAACGCCCGA |
| | | GTCAGGCCCTGGTACCTCCACAGAACCATCTGAAGGTAGTGC |
| | | GCCTGGTTCCCCAGCTGGAAGCCCTACTTCCACCGAAGAAGG |
| | | CACGTCAACCGAACCAAGTGAAGGATCTGCCCCTGGGACCA |
| | | GCACTGAACCATCTGAGCCAAAAAAGAAGAGAAAGGTAATG |
| | | GACAAGAAGTACAGCATCGGCCTGGCCATCGGCACCAACTC |
| | | TGTGGGCTGGGCCGTGATCACCGACGAGTACAAGGTGCCCA |
| | | GCAAGAAATTCAAGGTGCTGGGCAACACCGACCGGCACAGC |
| | | ATCAAGAAGAACCTGATCGGCGCCCTGCTGTTCGACAGCGG |
| | | AGAAACAGCCGAGGCCACCCGGCTGAAGAGAACCGCCAGA |
| | | AGAAGATACACCAGACGGAAGAACCGGATCTGCTATCTGCA |
| | | AGAGATCTTCAGCAACGAGATGGCCAAGGTGGACGACAGCT |
| | | TCTTCCACAGACTGGAAGAGTCCTTCCTGGTGGAAGAGGATA |
| | | AGAAGCACGAGCGGCACCCCATCTTCGGCAACATCGTGGAC |
| | | GAGGTGGCCTACCACGAGAAGTACCCCACCATCTACCACCT |
| | | GAGAAAGAAACTGGTGGACAGCACCGACAAGGCCGACCTGC |
| | | GGCTGATCTATCTGGCCCTGGCCCACATGATCAAGTTCCGGG |
| | | GCCACTTCCTGATCGAGGGCGACCTGAACCCCGACAACAGC |
| | | GACGTGGACAAGCTGTTCATCCAGCTGGTGCAGACCTACAA |
| | | CCAGCTGTTCGAGGAAAACCCCATCAACGCCAGCGGCGTGG |
| | | ACGCCAAGGCCATCCTGTCTGCCAGACTGAGCAAGAGCAGA |
| | | CGGCTGGAAAATCTGATCGCCCAGCTGCCCGGCGAGAAGAA |
| | | GAATGGCCTGTTCGGCAACCTGATTGCCCTGAGCCTGGGCCT |
| | | GACCCCCAACTTCAAGAGCAACTTCGACCTGGCCGAGGATG |
| | | CCAAACTGCAGCTGAGCAAGGACACCTACGACGACGACCTG |
| | | GACAACCTGCTGGCCCAGATCGGCGACCAGTACGCCGACCT |
| | | GTTTCTGGCCGCCAAGAACCTGTCCGACGCCATCCTGCTGAG |
| | | CGACATCCTGAGAGTGAACACCGAGATCACCAAGGCCCCCC |
| | | TGAGCGCCTCTATGATCAAGAGATACGACGAGCACCACCAG |
| | | GACCTGACCCTGCTGAAAGCTCTCGTGCGGCAGCAGCTGCCT |
| | | GAGAAGTACAAAGAGATTTTCTTCGACCAGAGCAAGAACGG |
| | | CTACGCCGGCTACATCGATGGCGGAGCCAGCCAGGAAGAGT |
| | | TCTACAAGTTCATCAAGCCCATCCTGGAAAAGATGGACGGC |
| | | ACCGAGGAACTGCTCGTGAAGCTGAACAGAGAGGACCTGCT |
| | | GCGGAAGCAGCGGACCTTCGACAACGGCAGCATCCCCCACC |
| | | AGATCCACCTGGGAGAGCTGCACGCCATTCTGCGGCGGCAG |
| | | GAAGATTTTTACCCATTCCTGAAGGACAACCGGGAAAAGAT |
| | | CGAGAAGATCCTGACCTTCCGCATCCCCTACTACGTGGGCCC |
| | | TCTGGCCAGGGGAAACAGCAGATTCGCCTGGATGACCAGAA |
| | | AGAGCGAGGAAACCATCACCCCCTGGAACTTCGAGGAAGTG |
| | | GTGGACAAGGGCGCCAGCGCCCAGAGCTTCATCGAGCGGAT |
| | | GACCAACTTCGATAAGAACCTGCCCAACGAGAAGGTGCTGC |
| | | CCAAGCACAGCCTGCTGTACGAGTACTTCACCGTGTACAACG |
| | | AGCTGACCAAAGTGAAATACGTGACCGAGGGAATGAGAAAG |
| | | CCCGCCTTCCTGAGCGGCGAGCAGAAAAAAGCCATCGTGGA |
| | | CCTGCTGTTCAAGACCAACCGGAAAGTGACCGTGAAGCAGC |
| | | TGAAAGAGGACTACTTCAAGAAAATCGAGTGCTTCGACTCC |
| | | GTGGAAATCTCCGGCGTGGAAGATCGGTTCAACGCCTCCCTG |
| | | GGCACATACCACGATCTGCTGAAAATTATCAAGGACAAGGA |
| | | CTTCCTGGACAATGAGGAAAACGAGGACATTCTGGAAGATA |
| | | TCGTGCTGACCCTGACACTGTTTGAGGACAGAGAGATGATCG |
| | | AGGAACGGCTGAAAACCTATGCCCACCTGTTCGACGACAAA |
| | | GTGATGAAGCAGCTGAAGCGGCGGAGATACACCGGCTGGGG |
| | | CAGGCTGAGCCGGAAGCTGATCAACGGCATCCGGGACAAGC |
| | | AGTCCGGCAAGACAATCCTGGATTTCCTGAAGTCCGACGGCT |
| | | TCGCCAACAGAAACTTCATGCAGCTGATCCACGACGACAGC |
| | | CTGACCTTTAAAGAGGACATCCAGAAAGCCCAGGTGTCCGG |
| | | CCAGGGCGATAGCCTGCACGAGCACATTGCCAATCTGGCCG |
| | | GCAGCCCCGCCATTAAGAAGGGCATCCTGCAGACAGTGAAG |
| | | GTGGTGGACGAGCTCGTGAAAGTGATGGGCCGGCACAAGCC |
| | | CGAGAACATCGTGATCGAAATGGCCAGAGAGAACCAGACCA |
| | | CCCAGAAGGGACAGAAGAACAGCCGCGAGAGAATGAAGCG |
| | | GATCGAAGAGGGCATCAAAGAGCTGGGCAGCCAGATCCTGA |
| | | AAGAACACCCCGTGGAAAACACCCAGCTGCAGAACGAGAAG |
| | | CTGTACCTGTACTACCTGCAGAATGGGCGGGATATGTACGTG |
| | | GACCAGGAACTGGACATCAACCGGCTGTCCGACTACGATGT |
| | | GGACGCTATCGTGCCTCAGAGCTTTCTGAAGGACGACTCCAT |
| | | CGATAACAAAGTGCTGACTCGGAGCGACAAGAACCGGGGCA |
| | | AGAGCGACAACGTGCCCTCCGAAGAGGTCGTGAAGAAGATG |
| | | AAGAACTACTGGCGCCAGCTGCTGAATGCCAAGCTGATTAC |
| | | CCAGAGGAAGTTCGACAATCTGACCAAGGCCGAGAGAGGCG |
| | | GCCTGAGCGAACTGGATAAGGCCGGCTTCATCAAGAGACAG |
| | | CTGGTGGAAACCCGGCAGATCACAAAGCACGTGGCACAGAT |
| | | CCTGGACTCCCGGATGAACACTAAGTACGACGAGAACGACA |
| | | AACTGATCCGGGAAGTGAAAGTGATCACCCTGAAGTCCAAG |
| | | CTGGTGTCCGATTTCCGGAAGGATTTCCAGTTTTACAAAGTG |
| | | CGCGAGATCAACAACTACCACCACGCCCACGACGCCTACCT |
| | | GAACGCCGTCGTGGGAACCGCCCTGATCAAAAAGTACCCTA |
| | | AGCTGGAAAGCGAGTTCGTGTACGGCGACTACAAGGTGTAC |
| | | GACGTGCGGAAGATGATCGCCAAGAGCGAGCAGGAAATCGG |
| | | CAAGGCTACCGCCAAGTACTTCTTCTACAGCAACATCATGAA |
| | | CTTTTTCAAGACCGAGATTACCCTGGCCAACGGCGAGATCCG |
| | | GAAGCGGCCTCTGATCGAGACAAACGGCGAAACAGGCGAGA |
| | | TCGTGTGGGATAAGGGCCGGGACTTTGCCACCGTGCGGAAA |
| | | GTGCTGTCTATGCCCCAAGTGAATATCGTGAAAAAGACCGA |
| | | GGTGCAGACAGGCGGCTTCAGCAAAGAGTCTATCCTGCCCA |
| | | AGAGGAACAGCGACAAGCTGATCGCCAGAAAGAAGGACTG |
| | | GGACCCTAAGAAGTACGGCGGCTTCGACAGCCCCACCGTGG |
| | | CCTATTCTGTGCTGGTGGTGGCCAAAGTGGAAAAGGGCAAG |
| | | TCCAAGAAACTGAAGAGTGTGAAAGAGCTGCTGGGGATCAC |
| | | CATCATGGAAAGAAGCAGCTTCGAGAAGAATCCCATCGACT |
| | | TTCTGGAAGCCAAGGGCTACAAAGAAGTGAAAAAGGACCTG |
| | | ATCATCAAGCTGCCTAAGTACTCCCTGTTCGAGCTGGAAAAC |
| | | GGCCGGAAGAGAATGCTGGCCTCTGCCGGCGAACTGCAGAA |
| | | GGGAAACGAACTGGCCCTGCCCTCCAAATATGTGAACTTCCT |
| | | GTACCTGGCCAGCCACTATGAGAAGCTGAAGGGCTCCCCCG |
| | | AGGATAATGAGCAGAAACAGCTGTTTGTGGAACAGCACAAA |
| | | CACTACCTGGACGAGATCATCGAGCAGATCAGCGAGTTCTCC |
| | | AAGAGAGTGATCCTGGCCGACGCTAATCTGGACAAGGTGCT |
| | | GAGCGCCTACAACAAGCACAGAGACAAGCCTATCAGAGAGC |
| | | AGGCCGAGAATATCATCCACCTGTTTACCCTGACCAATCTGG |
| | | GAGCCCCTGCCGCCTTCAAGTACTTTGACACCACCATCGACC |
| | | GGAAGAGGTACACCAGCACCAAAGAGGTGCTGGACGCCACC |
| | | CTGATCCACCAGAGCATCACCGGCCTGTACGAGACACGGAT |
| | | CGACCTGTCTCAGCTGGGAGGCGACCCAAAAAAGAAGAGAA |
| | | AGGTAAGCGGAAGTGAGACCCCAGGTACATCCGAATCAGCA |
| | | ACGCCTGAAAGCACCGGTCGGACACTGGTGACCTTCAAGGA |
| | | TGTATTTGTGGACTTCACCAGGGAGGAGTGGAAGCTGCTGG |
| | | ACACTGCTCAGCAGATCGTGTACAGAAATGTGATGCTGGAG |
| | | AACTATAAGAACCTGGTTTCCTTGGGTTATCAGCTTACTAAG |
| | | CCAGATGTGATCCTCCGGTTGGAGAAGGGAGAAGAGCCC |
| 25 | 5′ UTR 1 | AGATCAGATCTTTGTCGATCCTACCATCCACTCGACACACCC |
| | | GCCAGCGGCCGCTAATACGACTCACTATAGGGAGACCCAAG |
| | | CTGGCTAGCCGCCACCA |
| 26 | 5′ UTR 2 | TAATACGACTCACTATAGGGGGCGCTCGAGCAGGTTCAGAA |
| | | GGAGATCAAAAACCCCCAAGGATCAAAC |
| 27 | 5′ UTR 3 | CTCTGGCTAACTAGAGAACCCACTGCTTACTGGCTTATCGAA |
| | | ATTAATACGACTCACTATAGGGAGACCCAAGCTGGCTAGCG |
| | | TTTAAACTTAAGCTTGGTACCGAGGTAAGTATCAAGGTTACA |
| | | AGACAGGTTTAAGGAGGCCAATAGAAACTGGGCTTGTCGAG |
| | | ACAGAGAAGATTCTTGCGTTTCTGATAGGCACCTATTGGTCT |
| | | TACTGACATCCACTTTGCCTTTCTCTCCACAGCAACAAGCCG |
| | | CCACC |
| 28 | Fusion Protein 5 | MPKKKRKVPKKKRKVNHDQEFDPPKVYPPVPAEKRKPIRVLSL |
| | Amino Acid | FDGIATGLLVLKDLGIQVDRYIASEVCEDSITVGMVRHQGKIMY |
| | Sequence | VGDVRSVTQKHIQEWGPFDLVIGGSPCNDLSIVNPARKGLYEGT |
| | NLS-3A-3L- | GRLFFEFYRLLHDARPKEGDDRPFFWLFENVVAMGVSDKRDIS |
| | dCas9-KRAB- | RFLESNPVMIDAKEVSAAHRARYFWGNLPGMNRPLASTVNDK |
| | NLS | LELQECLEHGRIAKFSKVRTITTRSNSIKQGKDQHFPVFMNEKE |
| | | DILWCTEMERVFGFPVHYTDVSNMSRLARQRLLGRSWSVPVIR |
| | | HLFAPLKEYFACVSSGNSNANSRGPSFSSGLVPLSLRGSHNPLE |
| | | MFETVPVWRRQPVRVLSLFEDIKKELTSLGFLESGSDPGQLKHV |
| | | VDVTDTVRKDVEEWGPFDLVYGATPPLGHTCDRPPSWYLFQF |
| | | HRLLQYARPKPGSPRPFFWMFVDNLVLNKEDLDVASRFLEMEP |
| | | VTIPDVHGGSLQNAVRVWSNIPAIRSRHWALVSEEELSLLAQNK |
| | | QSSKLAAKWPTKLVKNCFLPLREYFKYFSTELTSSLGGPSSGAP |
| | | PPSGGSPAGSPTSTEEGTSESATPESGPGTSTEPSEGSAPGSPAGS |
| | | PTSTEEGTSTEPSEGSAPGTSTEPSEMDKKYSIGLAIGTNSVGWA |
| | | VITDEYKVPSKKFKVLGNTDRHSIKKNLIGALLFDSGETAEATR |
| | | LKRTARRRYTRRKNRICYLQEIFSNEMAKVDDSFFHRLEESFLV |
| | | EEDKKHERHPIFGNIVDEVAYHEKYPTIYHLRKKLVDSTDKADL |
| | | RLIYLALAHMIKFRGHFLIEGDLNPDNSDVDKLFIQLVQTYNQL |
| | | FEENPINASGVDAKAILSARLSKSRRLENLIAQLPGEKKNGLFGN |
| | | LIALSLGLTPNFKSNFDLAEDAKLQLSKDTYDDDLDNLLAQIGD |
| | | QYADLFLAAKNLSDAILLSDILRVNTEITKAPLSASMIKRYDEH |
| | | HQDLTLLKALVRQQLPEKYKEIFFDQSKNGYAGYIDGGASQEE |
| | | FYKFIKPILEKMDGTEELLVKLNREDLLRKQRTFDNGSIPHQIHL |
| | | GELHAILRRQEDFYPFLKDNREKIEKILTFRIPYYVGPLARGNSR |
| | | FAWMTRKSEETITPWNFEEVVDKGASAQSFIERMTNFDKNLPN |
| | | EKVLPKHSLLYEYFTVYNELTKVKYVTEGMRKPAFLSGEQKKA |
| | | IVDLLFKTNRKVTVKQLKEDYFKKIECFDSVEISGVEDRFNASL |
| | | GTYHDLLKIIKDKDFLDNEENEDILEDIVLTLTLFEDREMIEERL |
| | | KTYAHLFDDKVMKQLKRRRYTGWGRLSRKLINGIRDKQSGKTI |
| | | LDFLKSDGFANRNFMQLIHDDSLTFKEDIQKAQVSGQGDSLHE |
| | | HIANLAGSPAIKKGILQTVKVVDELVKVMGRHKPENIVIEMARE |
| | | NQTTQKGQKNSRERMKRIEEGIKELGSQILKEHPVENTQLQNEK |
| | | LYLYYLQNGRDMYVDQELDINRLSDYDVDAIVPQSFLKDDSID |
| | | NKVLTRSDKNRGKSDNVPSEEVVKKMKNYWRQLLNAKLITQR |
| | | KFDNLTKAERGGLSELDKAGFIKRQLVETRQITKHVAQILDSRM |
| | | NTKYDENDKLIREVKVITLKSKLVSDFRKDFQFYKVREINNYHH |
| | | AHDAYLNAVVGTALIKKYPKLESEFVYGDYKVYDVRKMIAKS |
| | | EQEIGKATAKYFFYSNIMNFFKTEITLANGEIRKRPLIETNGETG |
| | | EIVWDKGRDFATVRKVLSMPQVNIVKKTEVQTGGFSKESILPK |
| | | RNSDKLIARKKDWDPKKYGGFDSPTVAYSVLVVAKVEKGKSK |
| | | KLKSVKELLGITIMERSSFEKNPIDFLEAKGYKEVKKDLIIKLPK |
| | | YSLFELENGRKRMLASAGELQKGNELALPSKYVNFLYLASHYE |
| | | KLKGSPEDNEQKQLFVEQHKHYLDEIIEQISEFSKRVILADANLD |
| | | KVLSAYNKHRDKPIREQAENIIHLFTLTNLGAPAAFKYFDTTIDR |
| | | KRYTSTKEVLDATLIHQSITGLYETRIDLSQLGGDSGSETPGTSE |
| | | SATPESTGRTLVTFKDVFVDFTREEWKLLDTAQQIVYRNVMLE |
| | | NYKNLVSLGYQLTKPDVILRLEKGEEPSADYKDDDDKAPKKKR |
| | | KVPKKKRKV |
| 29 | Fusion Protein 5 | ATGCCAAAAAAGAAGAGAAAGGTACCGAAGAAAAAAAGAA |
| | DNA Sequence | AGGTCAACCACGACCAGGAATTCGACCCTCCAAAGGTTTAC |
| | | CCACCTGTCCCAGCTGAGAAGAGGAAGCCCATCCGGGTGCT |
| | | GTCTCTCTTTGATGGAATCGCTACAGGGCTCCTGGTGCTGAA |
| | | GGACTTGGGCATTCAGGTGGACCGCTACATTGCCTCGGAGGT |
| | | GTGTGAGGACTCCATCACGGTGGGCATGGTGCGGCACCAGG |
| | | GGAAGATCATGTACGTCGGGGACGTCCGCAGCGTCACACAG |
| | | AAGCATATCCAGGAGTGGGGCCCATTCGATCTGGTGATTGG |
| | | GGGCAGTCCCTGCAATGACCTCTCCATCGTCAACCCTGCTCG |
| | | CAAGGGCCTCTACGAGGGCACTGGCCGGCTCTTCTTTGAGTT |
| | | CTACCGCCTCCTGCATGATGCGCGGCCCAAGGAGGGAGATG |
| | | ATCGCCCCTTCTTCTGGCTCTTTGAGAATGTGGTGGCCATGG |
| | | GCGTTAGTGACAAGAGGGACATCTCGCGATTTCTCGAGTCCA |
| | | ACCCTGTGATGATTGATGCCAAAGAAGTGTCAGCTGCACAC |
| | | AGGGCCCGCTACTTCTGGGGTAACCTTCCCGGTATGAACAGG |
| | | CCGTTGGCATCCACTGTGAATGATAAGCTGGAGCTGCAGGA |
| | | GTGTCTGGAGCATGGCAGGATAGCCAAGTTCAGCAAAGTGA |
| | | GGACCATTACTACGAGGTCAAACTCCATAAAGCAGGGCAAA |
| | | GACCAGCATTTTCCTGTCTTCATGAATGAGAAAGAGGACATC |
| | | TTATGGTGCACTGAAATGGAAAGGGTATTTGGTTTCCCAGTC |
| | | CACTATACTGACGTCTCCAACATGAGCCGCTTGGCGAGGCAG |
| | | AGACTGCTGGGCCGGTCATGGAGCGTGCCAGTCATCCGCCA |
| | | CCTCTTCGCTCCGCTGAAGGAGTATTTTGCGTGTGTGTCTAG |
| | | CGGCAATAGTAACGCTAACAGCCGCGGGCCGAGCTTCAGCA |
| | | GCGGCCTGGTGCCGTTAAGCTTGCGCGGCAGCCATAATCCCC |
| | | TTGAGATGTTCGAAACCGTGCCTGTGTGGAGGAGACAGCCA |
| | | GTCCGGGTGCTGTCCCTTTTTGAAGACATCAAGAAAGAGCTG |
| | | ACGAGTTTGGGCTTTTTGGAAAGTGGTTCTGACCCGGGACAA |
| | | CTGAAGCATGTGGTTGATGTCACAGACACAGTGAGGAAGGA |
| | | TGTGGAGGAGTGGGGACCCTTCGATCTTGTGTACGGCGCCAC |
| | | ACCTCCCCTGGGCCACACCTGTGACCGTCCTCCCAGCTGGTA |
| | | CCTGTTCCAGTTCCACCGGCTCCTGCAGTACGCACGGCCCAA |
| | | GCCAGGCAGCCCCAGGCCCTTCTTCTGGATGTTCGTGGACAA |
| | | TCTGGTGCTGAACAAGGAAGACCTGGACGTCGCATCTCGCTT |
| | | CCTGGAGATGGAGCCAGTCACCATCCCAGATGTCCACGGCG |
| | | GATCCTTGCAGAATGCTGTCCGCGTGTGGAGCAACATCCCAG |
| | | CCATAAGGAGCAGGCACTGGGCTCTGGTTTCGGAAGAAGAA |
| | | TTGTCCCTGCTGGCCCAGAACAAGCAGAGCTCGAAGCTCGC |
| | | GGCCAAGTGGCCCACCAAGCTGGTGAAGAACTGCTTTCTCCC |
| | | CCTAAGAGAATATTTCAAGTATTTTTCAACAGAACTCACTTC |
| | | CTCTTTAGGAGGGCCGAGCTCTGGCGCACCCCCACCAAGTGG |
| | | AGGGTCTCCTGCCGGGTCCCCAACATCTACTGAAGAAGGCA |
| | | CCAGCGAATCCGCAACGCCCGAGTCAGGCCCTGGTACCTCC |
| | | ACAGAACCATCTGAAGGTAGTGCGCCTGGTTCCCCAGCTGG |
| | | AAGCCCTACTTCCACCGAAGAAGGCACGTCAACCGAACCAA |
| | | GTGAAGGATCTGCCCCTGGGACCAGCACTGAACCATCTGAG |
| | | ATGGACAAGAAGTACAGCATCGGCCTGGCCATCGGCACCAA |
| | | CTCTGTGGGCTGGGCCGTGATCACCGACGAGTACAAGGTGC |
| | | CCAGCAAGAAATTCAAGGTGCTGGGCAACACCGACCGGCAC |
| | | AGCATCAAGAAGAACCTGATCGGCGCCCTGCTGTTCGACAG |
| | | CGGAGAAACAGCCGAGGCCACCCGGCTGAAGAGAACCGCC |
| | | AGAAGAAGATACACCAGACGGAAGAACCGGATCTGCTATCT |
| | | GCAAGAGATCTTCAGCAACGAGATGGCCAAGGTGGACGACA |
| | | GCTTCTTCCACAGACTGGAAGAGTCCTTCCTGGTGGAAGAGG |
| | | ATAAGAAGCACGAGCGGCACCCCATCTTCGGCAACATCGTG |
| | | GACGAGGTGGCCTACCACGAGAAGTACCCCACCATCTACCA |
| | | CCTGAGAAAGAAACTGGTGGACAGCACCGACAAGGCCGACC |
| | | TGCGGCTGATCTATCTGGCCCTGGCCCACATGATCAAGTTCC |
| | | GGGGCCACTTCCTGATCGAGGGCGACCTGAACCCCGACAAC |
| | | AGCGACGTGGACAAGCTGTTCATCCAGCTGGTGCAGACCTA |
| | | CAACCAGCTGTTCGAGGAAAACCCCATCAACGCCAGCGGCG |
| | | TGGACGCCAAGGCCATCCTGTCTGCCAGACTGAGCAAGAGC |
| | | AGACGGCTGGAAAATCTGATCGCCCAGCTGCCCGGCGAGAA |
| | | GAAGAATGGCCTGTTCGGCAACCTGATTGCCCTGAGCCTGGG |
| | | CCTGACCCCCAACTTCAAGAGCAACTTCGACCTGGCCGAGG |
| | | ATGCCAAACTGCAGCTGAGCAAGGACACCTACGACGACGAC |
| | | CTGGACAACCTGCTGGCCCAGATCGGCGACCAGTACGCCGA |
| | | CCTGTTTCTGGCCGCCAAGAACCTGTCCGACGCCATCCTGCT |
| | | GAGCGACATCCTGAGAGTGAACACCGAGATCACCAAGGCCC |
| | | CCCTGAGCGCCTCTATGATCAAGAGATACGACGAGCACCAC |
| | | CAGGACCTGACCCTGCTGAAAGCTCTCGTGCGGCAGCAGCT |
| | | GCCTGAGAAGTACAAAGAGATTTTCTTCGACCAGAGCAAGA |
| | | ACGGCTACGCCGGCTACATCGATGGCGGAGCCAGCCAGGAA |
| | | GAGTTCTACAAGTTCATCAAGCCCATCCTGGAAAAGATGGA |
| | | CGGCACCGAGGAACTGCTCGTGAAGCTGAACAGAGAGGACC |
| | | TGCTGCGGAAGCAGCGGACCTTCGACAACGGCAGCATCCCC |
| | | CACCAGATCCACCTGGGAGAGCTGCACGCCATTCTGCGGCG |
| | | GCAGGAAGATTTTTACCCATTCCTGAAGGACAACCGGGAAA |
| | | AGATCGAGAAGATCCTGACCTTCCGCATCCCCTACTACGTGG |
| | | GCCCTCTGGCCAGGGGAAACAGCAGATTCGCCTGGATGACC |
| | | AGAAAGAGCGAGGAAACCATCACCCCCTGGAACTTCGAGGA |
| | | AGTGGTGGACAAGGGCGCCAGCGCCCAGAGCTTCATCGAGC |
| | | GGATGACCAACTTCGATAAGAACCTGCCCAACGAGAAGGTG |
| | | CTGCCCAAGCACAGCCTGCTGTACGAGTACTTCACCGTGTAC |
| | | AACGAGCTGACCAAAGTGAAATACGTGACCGAGGGAATGAG |
| | | AAAGCCCGCCTTCCTGAGCGGCGAGCAGAAAAAAGCCATCG |
| | | TGGACCTGCTGTTCAAGACCAACCGGAAAGTGACCGTGAAG |
| | | CAGCTGAAAGAGGACTACTTCAAGAAAATCGAGTGCTTCGA |
| | | CTCCGTGGAAATCTCCGGCGTGGAAGATCGGTTCAACGCCTC |
| | | CCTGGGCACATACCACGATCTGCTGAAAATTATCAAGGACA |
| | | AGGACTTCCTGGACAATGAGGAAAACGAGGACATTCTGGAA |
| | | GATATCGTGCTGACCCTGACACTGTTTGAGGACAGAGAGAT |
| | | GATCGAGGAACGGCTGAAAACCTATGCCCACCTGTTCGACG |
| | | ACAAAGTGATGAAGCAGCTGAAGCGGCGGAGATACACCGGC |
| | | TGGGGCAGGCTGAGCCGGAAGCTGATCAACGGCATCCGGGA |
| | | CAAGCAGTCCGGCAAGACAATCCTGGATTTCCTGAAGTCCG |
| | | ACGGCTTCGCCAACAGAAACTTCATGCAGCTGATCCACGAC |
| | | GACAGCCTGACCTTTAAAGAGGACATCCAGAAAGCCCAGGT |
| | | GTCCGGCCAGGGCGATAGCCTGCACGAGCACATTGCCAATC |
| | | TGGCCGGCAGCCCCGCCATTAAGAAGGGCATCCTGCAGACA |
| | | GTGAAGGTGGTGGACGAGCTCGTGAAAGTGATGGGCCGGCA |
| | | CAAGCCCGAGAACATCGTGATCGAAATGGCCAGAGAGAACC |
| | | AGACCACCCAGAAGGGACAGAAGAACAGCCGCGAGAGAAT |
| | | GAAGCGGATCGAAGAGGGCATCAAAGAGCTGGGCAGCCAG |
| | | ATCCTGAAAGAACACCCCGTGGAAAACACCCAGCTGCAGAA |
| | | CGAGAAGCTGTACCTGTACTACCTGCAGAATGGGCGGGATA |
| | | TGTACGTGGACCAGGAACTGGACATCAACCGGCTGTCCGAC |
| | | TACGATGTGGACGCTATCGTGCCTCAGAGCTTTCTGAAGGAC |
| | | GACTCCATCGATAACAAAGTGCTGACTCGGAGCGACAAGAA |
| | | CCGGGGCAAGAGCGACAACGTGCCCTCCGAAGAGGTCGTGA |
| | | AGAAGATGAAGAACTACTGGCGCCAGCTGCTGAATGCCAAG |
| | | CTGATTACCCAGAGGAAGTTCGACAATCTGACCAAGGCCGA |
| | | GAGAGGCGGCCTGAGCGAACTGGATAAGGCCGGCTTCATCA |
| | | AGAGACAGCTGGTGGAAACCCGGCAGATCACAAAGCACGTG |
| | | GCACAGATCCTGGACTCCCGGATGAACACTAAGTACGACGA |
| | | GAACGACAAACTGATCCGGGAAGTGAAAGTGATCACCCTGA |
| | | AGTCCAAGCTGGTGTCCGATTTCCGGAAGGATTTCCAGTTTT |
| | | ACAAAGTGCGCGAGATCAACAACTACCACCACGCCCACGAC |
| | | GCCTACCTGAACGCCGTCGTGGGAACCGCCCTGATCAAAAA |
| | | GTACCCTAAGCTGGAAAGCGAGTTCGTGTACGGCGACTACA |
| | | AGGTGTACGACGTGCGGAAGATGATCGCCAAGAGCGAGCAG |
| | | GAAATCGGCAAGGCTACCGCCAAGTACTTCTTCTACAGCAAC |
| | | ATCATGAACTTTTTCAAGACCGAGATTACCCTGGCCAACGGC |
| | | GAGATCCGGAAGCGGCCTCTGATCGAGACAAACGGCGAAAC |
| | | AGGCGAGATCGTGTGGGATAAGGGCCGGGACTTTGCCACCG |
| | | TGCGGAAAGTGCTGTCTATGCCCCAAGTGAATATCGTGAAA |
| | | AAGACCGAGGTGCAGACAGGCGGCTTCAGCAAAGAGTCTAT |
| | | CCTGCCCAAGAGGAACAGCGACAAGCTGATCGCCAGAAAGA |
| | | AGGACTGGGACCCTAAGAAGTACGGCGGCTTCGACAGCCCC |
| | | ACCGTGGCCTATTCTGTGCTGGTGGTGGCCAAAGTGGAAAA |
| | | GGGCAAGTCCAAGAAACTGAAGAGTGTGAAAGAGCTGCTGG |
| | | GGATCACCATCATGGAAAGAAGCAGCTTCGAGAAGAATCCC |
| | | ATCGACTTTCTGGAAGCCAAGGGCTACAAAGAAGTGAAAAA |
| | | GGACCTGATCATCAAGCTGCCTAAGTACTCCCTGTTCGAGCT |
| | | GGAAAACGGCCGGAAGAGAATGCTGGCCTCTGCCGGCGAAC |
| | | TGCAGAAGGGAAACGAACTGGCCCTGCCCTCCAAATATGTG |
| | | AACTTCCTGTACCTGGCCAGCCACTATGAGAAGCTGAAGGG |
| | | CTCCCCCGAGGATAATGAGCAGAAACAGCTGTTTGTGGAAC |
| | | AGCACAAACACTACCTGGACGAGATCATCGAGCAGATCAGC |
| | | GAGTTCTCCAAGAGAGTGATCCTGGCCGACGCTAATCTGGAC |
| | | AAGGTGCTGAGCGCCTACAACAAGCACAGAGACAAGCCTAT |
| | | CAGAGAGCAGGCCGAGAATATCATCCACCTGTTTACCCTGAC |
| | | CAATCTGGGAGCCCCTGCCGCCTTCAAGTACTTTGACACCAC |
| | | CATCGACCGGAAGAGGTACACCAGCACCAAAGAGGTGCTGG |
| | | ACGCCACCCTGATCCACCAGAGCATCACCGGCCTGTACGAG |
| | | ACACGGATCGACCTGTCTCAGCTGGGAGGCGACAGCGGAAG |
| | | TGAGACCCCAGGTACATCCGAATCAGCAACGCCTGAAAGCA |
| | | CCGGTCGGACACTGGTGACCTTCAAGGATGTATTTGTGGACT |
| | | TCACCAGGGAGGAGTGGAAGCTGCTGGACACTGCTCAGCAG |
| | | ATCGTGTACAGAAATGTGATGCTGGAGAACTATAAGAACCT |
| | | GGTTTCCTTGGGTTATCAGCTTACTAAGCCAGATGTGATCCT |
| | | CCGGTTGGAGAAGGGAGAAGAGCCCAGCGCTGATTACAAAG |
| | | ATGATGACGATAAAGCCCCAAAAAAGAAGAGAAAGGTACC |
| | | GAAGAAAAAAAGAAAGGTC |
| 30 | Fusion Protein 6 | MPKKKRKVPKKKRKVNHDQEFDPPKVYPPVPAEKRKPIRVLSL |
| | Amino Acid | FDGIATGLLVLKDLGIQVDRYIASEVCEDSITVGMVRHQGKIMY |
| | Sequence | VGDVRSVTQKHIQEWGPFDLVIGGSPCNDLSIVNPARKGLYEGT |
| | NLS-3A-3L- | GRLFFEFYRLLHDARPKEGDDRPFFWLFENVVAMGVSDKRDIS |
| | dCas9-KRAB- | RFLESNPVMIDAKEVSAAHRARYFWGNLPGMNRPLASTVNDK |
| | NLS | LELQECLEHGRIAKFSKVRTITTRSNSIKQGKDQHFPVFMNEKE |
| | | DILWCTEMERVFGFPVHYTDVSNMSRLARQRLLGRSWSVPVIR |
| | | HLFAPLKEYFACVSSGNSNANSRGPSFSSGLVPLSLRGSHMGPM |
| | | EIYKTVSAWKRQPVRVLSLFRNIDKVLKSLGFLESGSGSGGGTL |
| | | KYVEDVTNVVRRDVEKWGPFDLVYGSTQPLGSSCDRCPGWY |
| | | MFQFHRILQYALPRQESQRPFFWIFMDNLLLTEDDQETTTRFLQ |
| | | TEAVTLQDVRGRDYQNAMRVWSNIPGLKSKHAPLTPKEEEYL |
| | | QAQVRSRSKLDAPKVDLLVKNCLLPLREYFKYFSQNSLPLGGP |
| | | SSGAPPPSGGSPAGSPTSTEEGTSESATPESGPGTSTEPSEGSAPG |
| | | SPAGSPTSTEEGTSTEPSEGSAPGTSTEPSEMDKKYSIGLAIGTNS |
| | | VGWAVITDEYKVPSKKFKVLGNTDRHSIKKNLIGALLFDSGET |
| | | AEATRLKRTARRRYTRRKNRICYLQEIFSNEMAKVDDSFFHRLE |
| | | ESFLVEEDKKHERHPIFGNIVDEVAYHEKYPTIYHLRKKLVDST |
| | | DKADLRLIYLALAHMIKFRGHFLIEGDLNPDNSDVDKLFIQLVQ |
| | | TYNQLFEENPINASGVDAKAILSARLSKSRRLENLIAQLPGEKK |
| | | NGLFGNLIALSLGLTPNFKSNFDLAEDAKLQLSKDTYDDDLDN |
| | | LLAQIGDQYADLFLAAKNLSDAILLSDILRVNTEITKAPLSASMI |
| | | KRYDEHHQDLTLLKALVRQQLPEKYKEIFFDQSKNGYAGYIDG |
| | | GASQEEFYKFIKPILEKMDGTEELLVKLNREDLLRKQRTFDNGS |
| | | IPHQIHLGELHAILRRQEDFYPFLKDNREKIEKILTFRIPYYVGPL |
| | | ARGNSRFAWMTRKSEETITPWNFEEVVDKGASAQSFIERMTNF |
| | | DKNLPNEKVLPKHSLLYEYFTVYNELTKVKYVTEGMRKPAFLS |
| | | GEQKKAIVDLLFKTNRKVTVKQLKEDYFKKIECFDSVEISGVED |
| | | RFNASLGTYHDLLKIIKDKDFLDNEENEDILEDIVLTLTLFEDRE |
| | | MIEERLKTYAHLFDDKVMKQLKRRRYTGWGRLSRKLINGIRDK |
| | | QSGKTILDFLKSDGFANRNFMQLIHDDSLTFKEDIQKAQVSGQG |
| | | DSLHEHIANLAGSPAIKKGILQTVKVVDELVKVMGRHKPENIVI |
| | | EMARENQTTQKGQKNSRERMKRIEEGIKELGSQILKEHPVENT |
| | | QLQNEKLYLYYLQNGRDMYVDQELDINRLSDYDVDAIVPQSFL |
| | | KDDSIDNKVLTRSDKNRGKSDNVPSEEVVKKMKNYWRQLLNA |
| | | KLITQRKFDNLTKAERGGLSELDKAGFIKRQLVETRQITKHVAQ |
| | | ILDSRMNTKYDENDKLIREVKVITLKSKLVSDFRKDFQFYKVRE |
| | | INNYHHAHDAYLNAVVGTALIKKYPKLESEFVYGDYKVYDVR |
| | | KMIAKSEQEIGKATAKYFFYSNIMNFFKTEITLANGEIRKRPLIET |
| | | NGETGEIVWDKGRDFATVRKVLSMPQVNIVKKTEVQTGGFSKE |
| | | SILPKRNSDKLIARKKDWDPKKYGGFDSPTVAYSVLVVAKVEK |
| | | GKSKKLKSVKELLGITIMERSSFEKNPIDFLEAKGYKEVKKDLII |
| | | KLPKYSLFELENGRKRMLASAGELQKGNELALPSKYVNFLYLA |
| | | SHYEKLKGSPEDNEQKQLFVEQHKHYLDEIIEQISEFSKRVILAD |
| | | ANLDKVLSAYNKHRDKPIREQAENIIHLFTLTNLGAPAAFKYFD |
| | | TTIDRKRYTSTKEVLDATLIHQSITGLYETRIDLSQLGGDSGSETP |
| | | GTSESATPESTGRTLVTFKDVFVDFTREEWKLLDTAQQIVYRNV |
| | | MLENYKNLVSLGYQLTKPDVILRLEKGEEPSADYKDDDDKAPK |
| | | KKRKVPKKKRKV |
| 31 | Fusion Protein 6 | ATGCCAAAAAAGAAGAGAAAGGTACCGAAGAAAAAAAGAA |
| | DNA Sequence | AGGTATACAACCATGACCAGGAATTCGACCCCCCAAAGGTT |
| | | TACCCACCTGTGCCAGCTGAGAAGAGGAAGCCCATCCGCGT |
| | | GCTGTCTCTCTTTGATGGGATTGCTACAGGGCTCCTGGTGCT |
| | | GAAGGACCTGGGCATCCAAGTGGACCGCTACATTGCCTCCG |
| | | AGGTGTGTGAGGACTCCATCACGGTGGGCATGGTGCGGCAC |
| | | CAGGGAAAGATCATGTACGTCGGGGACGTCCGCAGCGTCAC |
| | | ACAGAAGCATATCCAGGAGTGGGGCCCATTCGACCTGGTGA |
| | | TTGGAGGCAGTCCCTGCAATGACCTCTCCATTGTCAACCCTG |
| | | CCCGCAAGGGACTTTATGAGGGTACTGGCCGCCTCTTCTTTG |
| | | AGTTCTACCGCCTCCTGCATGATGCGCGGCCCAAGGAGGGA |
| | | GATGATCGCCCCTTCTTCTGGCTCTTTGAGAATGTGGTGGCC |
| | | ATGGGCGTTAGTGACAAGAGGGACATCTCGCGATTTCTTGAG |
| | | TCTAACCCCGTGATGATTGACGCCAAAGAAGTGTCTGCTGCA |
| | | CACAGGGCCCGTTACTTCTGGGGTAACCTTCCTGGCATGAAC |
| | | AGGCCTTTGGCATCCACTGTGAATGATAAGCTGGAGCTGCAA |
| | | GAGTGTCTGGAGCACGGCAGAATAGCCAAGTTCAGCAAAGT |
| | | GAGGACCATTACCACCAGGTCAAACTCTATAAAGCAGGGCA |
| | | AAGACCAGCATTTCCCCGTCTTCATGAACGAGAAGGAGGAC |
| | | ATCCTGTGGTGCACTGAAATGGAAAGGGTGTTTGGCTTCCCC |
| | | GTCCACTACACAGACGTCTCCAACATGAGCCGCTTGGCGAG |
| | | GCAGAGACTGCTGGGCCGATCGTGGAGCGTGCCGGTCATCC |
| | | GCCACCTCTTCGCTCCGCTGAAGGAATATTTTGCTTGTGTGTC |
| | | TAGCGGCAATAGTAACGCTAACAGCCGCGGGCCGAGCTTCA |
| | | GCAGCGGCCTGGTGCCGTTAAGCTTGCGCGGCAGCCATATG |
| | | GGCCCTATGGAGATATACAAGACAGTGTCTGCATGGAAGAG |
| | | ACAGCCAGTGCGGGTACTGAGCCTCTTCAGAAACATCGACA |
| | | AGGTACTAAAGAGTTTGGGCTTCTTGGAAAGCGGTTCTGGTT |
| | | CTGGGGGAGGAACGCTGAAGTACGTGGAAGATGTCACAAAT |
| | | GTCGTGAGGAGAGACGTGGAGAAATGGGGCCCCTTTGACCT |
| | | GGTGTACGGCTCGACGCAGCCCCTAGGCAGCTCTTGTGATCG |
| | | CTGTCCCGGCTGGTACATGTTCCAGTTCCACCGGATCCTGCA |
| | | GTATGCGCTGCCTCGCCAGGAGAGTCAGCGGCCCTTCTTCTG |
| | | GATATTCATGGACAATCTGCTGCTGACTGAGGATGACCAAG |
| | | AGACAACTACCCGCTTCCTTCAGACAGAGGCTGTGACCCTCC |
| | | AGGATGTCCGTGGCAGAGACTACCAGAATGCTATGCGGGTG |
| | | TGGAGCAACATTCCAGGGCTGAAGAGCAAGCATGCGCCCCT |
| | | GACCCCAAAGGAAGAAGAGTATCTGCAAGCCCAAGTCAGAA |
| | | GCAGGAGCAAGCTGGACGCCCCGAAAGTTGACCTCCTGGTG |
| | | AAGAACTGCCTTCTCCCGCTGAGAGAGTACTTCAAGTATTTT |
| | | TCTCAAAACTCACTTCCTCTTGGAGGGCCGAGCTCTGGCGCA |
| | | CCCCCACCAAGTGGAGGGTCTCCTGCCGGGTCCCCAACATCT |
| | | ACTGAAGAAGGCACCAGCGAATCCGCAACGCCCGAGTCAGG |
| | | CCCTGGTACCTCCACAGAACCATCTGAAGGTAGTGCGCCTGG |
| | | TTCCCCAGCTGGAAGCCCTACTTCCACCGAAGAAGGCACGTC |
| | | AACCGAACCAAGTGAAGGATCTGCCCCTGGGACCAGCACTG |
| | | AACCATCTGAGATGGACAAGAAGTACAGCATCGGCCTGGCC |
| | | ATCGGCACCAACTCTGTGGGCTGGGCCGTGATCACCGACGA |
| | | GTACAAGGTGCCCAGCAAGAAATTCAAGGTGCTGGGCAACA |
| | | CCGACCGGCACAGCATCAAGAAGAACCTGATCGGCGCCCTG |
| | | CTGTTCGACAGCGGAGAAACAGCCGAGGCCACCCGGCTGAA |
| | | GAGAACCGCCAGAAGAAGATACACCAGACGGAAGAACCGG |
| | | ATCTGCTATCTGCAAGAGATCTTCAGCAACGAGATGGCCAA |
| | | GGTGGACGACAGCTTCTTCCACAGACTGGAAGAGTCCTTCCT |
| | | GGTGGAAGAGGATAAGAAGCACGAGCGGCACCCCATCTTCG |
| | | GCAACATCGTGGACGAGGTGGCCTACCACGAGAAGTACCCC |
| | | ACCATCTACCACCTGAGAAAGAAACTGGTGGACAGCACCGA |
| | | CAAGGCCGACCTGCGGCTGATCTATCTGGCCCTGGCCCACAT |
| | | GATCAAGTTCCGGGGCCACTTCCTGATCGAGGGCGACCTGA |
| | | ACCCCGACAACAGCGACGTGGACAAGCTGTTCATCCAGCTG |
| | | GTGCAGACCTACAACCAGCTGTTCGAGGAAAACCCCATCAA |
| | | CGCCAGCGGCGTGGACGCCAAGGCCATCCTGTCTGCCAGAC |
| | | TGAGCAAGAGCAGACGGCTGGAAAATCTGATCGCCCAGCTG |
| | | CCCGGCGAGAAGAAGAATGGCCTGTTCGGCAACCTGATTGC |
| | | CCTGAGCCTGGGCCTGACCCCCAACTTCAAGAGCAACTTCGA |
| | | CCTGGCCGAGGATGCCAAACTGCAGCTGAGCAAGGACACCT |
| | | ACGACGACGACCTGGACAACCTGCTGGCCCAGATCGGCGAC |
| | | CAGTACGCCGACCTGTTTCTGGCCGCCAAGAACCTGTCCGAC |
| | | GCCATCCTGCTGAGCGACATCCTGAGAGTGAACACCGAGAT |
| | | CACCAAGGCCCCCCTGAGCGCCTCTATGATCAAGAGATACG |
| | | ACGAGCACCACCAGGACCTGACCCTGCTGAAAGCTCTCGTG |
| | | CGGCAGCAGCTGCCTGAGAAGTACAAAGAGATTTTCTTCGA |
| | | CCAGAGCAAGAACGGCTACGCCGGCTACATCGATGGCGGAG |
| | | CCAGCCAGGAAGAGTTCTACAAGTTCATCAAGCCCATCCTGG |
| | | AAAAGATGGACGGCACCGAGGAACTGCTCGTGAAGCTGAAC |
| | | AGAGAGGACCTGCTGCGGAAGCAGCGGACCTTCGACAACGG |
| | | CAGCATCCCCCACCAGATCCACCTGGGAGAGCTGCACGCCA |
| | | TTCTGCGGCGGCAGGAAGATTTTTACCCATTCCTGAAGGACA |
| | | ACCGGGAAAAGATCGAGAAGATCCTGACCTTCCGCATCCCC |
| | | TACTACGTGGGCCCTCTGGCCAGGGGAAACAGCAGATTCGC |
| | | CTGGATGACCAGAAAGAGCGAGGAAACCATCACCCCCTGGA |
| | | ACTTCGAGGAAGTGGTGGACAAGGGCGCCAGCGCCCAGAGC |
| | | TTCATCGAGCGGATGACCAACTTCGATAAGAACCTGCCCAAC |
| | | GAGAAGGTGCTGCCCAAGCACAGCCTGCTGTACGAGTACTT |
| | | CACCGTGTACAACGAGCTGACCAAAGTGAAATACGTGACCG |
| | | AGGGAATGAGAAAGCCCGCCTTCCTGAGCGGCGAGCAGAAA |
| | | AAAGCCATCGTGGACCTGCTGTTCAAGACCAACCGGAAAGT |
| | | GACCGTGAAGCAGCTGAAAGAGGACTACTTCAAGAAAATCG |
| | | AGTGCTTCGACTCCGTGGAAATCTCCGGCGTGGAAGATCGGT |
| | | TCAACGCCTCCCTGGGCACATACCACGATCTGCTGAAAATTA |
| | | TCAAGGACAAGGACTTCCTGGACAATGAGGAAAACGAGGAC |
| | | ATTCTGGAAGATATCGTGCTGACCCTGACACTGTTTGAGGAC |
| | | AGAGAGATGATCGAGGAACGGCTGAAAACCTATGCCCACCT |
| | | GTTCGACGACAAAGTGATGAAGCAGCTGAAGCGGCGGAGAT |
| | | ACACCGGCTGGGGCAGGCTGAGCCGGAAGCTGATCAACGGC |
| | | ATCCGGGACAAGCAGTCCGGCAAGACAATCCTGGATTTCCT |
| | | GAAGTCCGACGGCTTCGCCAACAGAAACTTCATGCAGCTGA |
| | | TCCACGACGACAGCCTGACCTTTAAAGAGGACATCCAGAAA |
| | | GCCCAGGTGTCCGGCCAGGGCGATAGCCTGCACGAGCACAT |
| | | TGCCAATCTGGCCGGCAGCCCCGCCATTAAGAAGGGCATCCT |
| | | GCAGACAGTGAAGGTGGTGGACGAGCTCGTGAAAGTGATGG |
| | | GCCGGCACAAGCCCGAGAACATCGTGATCGAAATGGCCAGA |
| | | GAGAACCAGACCACCCAGAAGGGACAGAAGAACAGCCGCG |
| | | AGAGAATGAAGCGGATCGAAGAGGGCATCAAAGAGCTGGG |
| | | CAGCCAGATCCTGAAAGAACACCCCGTGGAAAACACCCAGC |
| | | TGCAGAACGAGAAGCTGTACCTGTACTACCTGCAGAATGGG |
| | | CGGGATATGTACGTGGACCAGGAACTGGACATCAACCGGCT |
| | | GTCCGACTACGATGTGGACGCTATCGTGCCTCAGAGCTTTCT |
| | | GAAGGACGACTCCATCGATAACAAAGTGCTGACTCGGAGCG |
| | | ACAAGAACCGGGGCAAGAGCGACAACGTGCCCTCCGAAGAG |
| | | GTCGTGAAGAAGATGAAGAACTACTGGCGCCAGCTGCTGAA |
| | | TGCCAAGCTGATTACCCAGAGGAAGTTCGACAATCTGACCA |
| | | AGGCCGAGAGAGGCGGCCTGAGCGAACTGGATAAGGCCGGC |
| | | TTCATCAAGAGACAGCTGGTGGAAACCCGGCAGATCACAAA |
| | | GCACGTGGCACAGATCCTGGACTCCCGGATGAACACTAAGT |
| | | ACGACGAGAACGACAAACTGATCCGGGAAGTGAAAGTGATC |
| | | ACCCTGAAGTCCAAGCTGGTGTCCGATTTCCGGAAGGATTTC |
| | | CAGTTTTACAAAGTGCGCGAGATCAACAACTACCACCACGC |
| | | CCACGACGCCTACCTGAACGCCGTCGTGGGAACCGCCCTGA |
| | | TCAAAAAGTACCCTAAGCTGGAAAGCGAGTTCGTGTACGGC |
| | | GACTACAAGGTGTACGACGTGCGGAAGATGATCGCCAAGAG |
| | | CGAGCAGGAAATCGGCAAGGCTACCGCCAAGTACTTCTTCT |
| | | ACAGCAACATCATGAACTTTTTCAAGACCGAGATTACCCTGG |
| | | CCAACGGCGAGATCCGGAAGCGGCCTCTGATCGAGACAAAC |
| | | GGCGAAACAGGCGAGATCGTGTGGGATAAGGGCCGGGACTT |
| | | TGCCACCGTGCGGAAAGTGCTGTCTATGCCCCAAGTGAATAT |
| | | CGTGAAAAAGACCGAGGTGCAGACAGGCGGCTTCAGCAAAG |
| | | AGTCTATCCTGCCCAAGAGGAACAGCGACAAGCTGATCGCC |
| | | AGAAAGAAGGACTGGGACCCTAAGAAGTACGGCGGCTTCGA |
| | | CAGCCCCACCGTGGCCTATTCTGTGCTGGTGGTGGCCAAAGT |
| | | GGAAAAGGGCAAGTCCAAGAAACTGAAGAGTGTGAAAGAG |
| | | CTGCTGGGGATCACCATCATGGAAAGAAGCAGCTTCGAGAA |
| | | GAATCCCATCGACTTTCTGGAAGCCAAGGGCTACAAAGAAG |
| | | TGAAAAAGGACCTGATCATCAAGCTGCCTAAGTACTCCCTGT |
| | | TCGAGCTGGAAAACGGCCGGAAGAGAATGCTGGCCTCTGCC |
| | | GGCGAACTGCAGAAGGGAAACGAACTGGCCCTGCCCTCCAA |
| | | ATATGTGAACTTCCTGTACCTGGCCAGCCACTATGAGAAGCT |
| | | GAAGGGCTCCCCCGAGGATAATGAGCAGAAACAGCTGTTTG |
| | | TGGAACAGCACAAACACTACCTGGACGAGATCATCGAGCAG |
| | | ATCAGCGAGTTCTCCAAGAGAGTGATCCTGGCCGACGCTAAT |
| | | CTGGACAAGGTGCTGAGCGCCTACAACAAGCACAGAGACAA |
| | | GCCTATCAGAGAGCAGGCCGAGAATATCATCCACCTGTTTAC |
| | | CCTGACCAATCTGGGAGCCCCTGCCGCCTTCAAGTACTTTGA |
| | | CACCACCATCGACCGGAAGAGGTACACCAGCACCAAAGAGG |
| | | TGCTGGACGCCACCCTGATCCACCAGAGCATCACCGGCCTGT |
| | | ACGAGACACGGATCGACCTGTCTCAGCTGGGAGGCGACAGC |
| | | GGAAGTGAGACCCCAGGTACATCCGAATCAGCAACGCCTGA |
| | | AAGCACCGGTCGGACACTGGTGACCTTCAAGGATGTATTTGT |
| | | GGACTTCACCAGGGAGGAGTGGAAGCTGCTGGACACTGCTC |
| | | AGCAGATCGTGTACAGAAATGTGATGCTGGAGAACTATAAG |
| | | AACCTGGTTTCCTTGGGTTATCAGCTTACTAAGCCAGATGTG |
| | | ATCCTCCGGTTGGAGAAGGGAGAAGAGCCCAGCGCTGATTA |
| | | CAAAGATGATGACGATAAAGCCCCAAAAAAGAAGAGAAAG |
| | | GTACCGAAGAAAAAAAGAAAGGTC |
| 32 | Fusion Protein 7 | MPKKKRKVPKKKRKVNHDQEFDPPKVYPPVPAEKRKPIRVLSL |
| | Amino Acid | FDGIATGLLVLKDLGIQVDRYIASEVCEDSITVGMVRHQGKIMY |
| | Sequence | VGDVRSVTQKHIQEWGPFDLVIGGSPCNDLSIVNPARKGLYEGT |
| | NLS-3A-3L- | GRLFFEFYRLLHDARPKEGDDRPFFWLFENVVAMGVSDKRDIS |
| | dCas9-ZIM-NLS | RFLESNPVMIDAKEVSAAHRARYFWGNLPGMNRPLASTVNDK |
| | | LELQECLEHGRIAKFSKVRTITTRSNSIKQGKDQHFPVFMNEKE |
| | | DILWCTEMERVFGFPVHYTDVSNMSRLARQRLLGRSWSVPVIR |
| | | HLFAPLKEYFACVSSGNSNANSRGPSFSSGLVPLSLRGSHMGPM |
| | | EIYKTVSAWKRQPVRVLSLFRNIDKVLKSLGFLESGSGSGGGTL |
| | | KYVEDVTNVVRRDVEKWGPFDLVYGSTQPLGSSCDRCPGWY |
| | | MFQFHRILQYALPRQESQRPFFWIFMDNLLLTEDDQETTTRFLQ |
| | | TEAVTLQDVRGRDYQNAMRVWSNIPGLKSKHAPLTPKEEEYL |
| | | QAQVRSRSKLDAPKVDLLVKNCLLPLREYFKYFSQNSLPLGGP |
| | | SSGAPPPSGGSPAGSPTSTEEGTSESATPESGPGTSTEPSEGSAPG |
| | | SPAGSPTSTEEGTSTEPSEGSAPGTSTEPSEMDKKYSIGLAIGTNS |
| | | VGWAVITDEYKVPSKKFKVLGNTDRHSIKKNLIGALLFDSGET |
| | | AEATRLKRTARRRYTRRKNRICYLQEIFSNEMAKVDDSFFHRLE |
| | | ESFLVEEDKKHERHPIFGNIVDEVAYHEKYPTIYHLRKKLVDST |
| | | DKADLRLIYLALAHMIKFRGHFLIEGDLNPDNSDVDKLFIQLVQ |
| | | TYNQLFEENPINASGVDAKAILSARLSKSRRLENLIAQLPGEKK |
| | | NGLFGNLIALSLGLTPNFKSNFDLAEDAKLQLSKDTYDDDLDN |
| | | LLAQIGDQYADLFLAAKNLSDAILLSDILRVNTEITKAPLSASMI |
| | | KRYDEHHQDLTLLKALVRQQLPEKYKEIFFDQSKNGYAGYIDG |
| | | GASQEEFYKFIKPILEKMDGTEELLVKLNREDLLRKQRTFDNGS |
| | | IPHQIHLGELHAILRRQEDFYPFLKDNREKIEKILTFRIPYYVGPL |
| | | ARGNSRFAWMTRKSEETITPWNFEEVVDKGASAQSFIERMTNF |
| | | DKNLPNEKVLPKHSLLYEYFTVYNELTKVKYVTEGMRKPAFLS |
| | | GEQKKAIVDLLFKTNRKVTVKQLKEDYFKKIECFDSVEISGVED |
| | | RFNASLGTYHDLLKIIKDKDFLDNEENEDILEDIVLTLTLFEDRE |
| | | MIEERLKTYAHLFDDKVMKQLKRRRYTGWGRLSRKLINGIRDK |
| | | QSGKTILDFLKSDGFANRNFMQLIHDDSLTFKEDIQKAQVSGQG |
| | | DSLHEHIANLAGSPAIKKGILQTVKVVDELVKVMGRHKPENIVI |
| | | EMARENQTTQKGQKNSRERMKRIEEGIKELGSQILKEHPVENT |
| | | QLQNEKLYLYYLQNGRDMYVDQELDINRLSDYDVDAIVPQSFL |
| | | KDDSIDNKVLTRSDKNRGKSDNVPSEEVVKKMKNYWRQLLNA |
| | | KLITQRKFDNLTKAERGGLSELDKAGFIKRQLVETRQITKHVAQ |
| | | ILDSRMNTKYDENDKLIREVKVITLKSKLVSDFRKDFQFYKVRE |
| | | INNYHHAHDAYLNAVVGTALIKKYPKLESEFVYGDYKVYDVR |
| | | KMIAKSEQEIGKATAKYFFYSNIMNFFKTEITLANGEIRKRPLIET |
| | | NGETGEIVWDKGRDFATVRKVLSMPQVNIVKKTEVQTGGFSKE |
| | | SILPKRNSDKLIARKKDWDPKKYGGFDSPTVAYSVLVVAKVEK |
| | | GKSKKLKSVKELLGITIMERSSFEKNPIDFLEAKGYKEVKKDLII |
| | | KLPKYSLFELENGRKRMLASAGELQKGNELALPSKYVNFLYLA |
| | | SHYEKLKGSPEDNEQKQLFVEQHKHYLDEIIEQISEFSKRVILAD |
| | | ANLDKVLSAYNKHRDKPIREQAENIIHLFTLTNLGAPAAFKYFD |
| | | TTIDRKRYTSTKEVLDATLIHQSITGLYETRIDLSQLGGDSGSETP |
| | | GTSESATPESTGMNNSQGRVTFEDVTVNFTQGEWQRLNPEQRN |
| | | LYRDVMLENYSNLVSVGQGETTKPDVILRLEQGKEPWLEEEEV |
| | | LGSGRAEKNGDIGGQIWKPKDVKESLSADYKDDDDKAPKKKR |
| | | KVPKKKRKV |
| 33 | Fusion Protein 7 | ATGCCAAAAAAGAAGAGAAAGGTACCGAAGAAAAAAAGAA |
| | DNA Sequence | AGGTATACAACCATGACCAGGAATTCGACCCCCCAAAGGTT |
| | | TACCCACCTGTGCCAGCTGAGAAGAGGAAGCCCATCCGCGT |
| | | GCTGTCTCTCTTTGATGGGATTGCTACAGGGCTCCTGGTGCT |
| | | GAAGGACCTGGGCATCCAAGTGGACCGCTACATTGCCTCCG |
| | | AGGTGTGTGAGGACTCCATCACGGTGGGCATGGTGCGGCAC |
| | | CAGGGAAAGATCATGTACGTCGGGGACGTCCGCAGCGTCAC |
| | | ACAGAAGCATATCCAGGAGTGGGGCCCATTCGACCTGGTGA |
| | | TTGGAGGCAGTCCCTGCAATGACCTCTCCATTGTCAACCCTG |
| | | CCCGCAAGGGACTTTATGAGGGTACTGGCCGCCTCTTCTTTG |
| | | AGTTCTACCGCCTCCTGCATGATGCGCGGCCCAAGGAGGGA |
| | | GATGATCGCCCCTTCTTCTGGCTCTTTGAGAATGTGGTGGCC |
| | | ATGGGCGTTAGTGACAAGAGGGACATCTCGCGATTTCTTGAG |
| | | TCTAACCCCGTGATGATTGACGCCAAAGAAGTGTCTGCTGCA |
| | | CACAGGGCCCGTTACTTCTGGGGTAACCTTCCTGGCATGAAC |
| | | AGGCCTTTGGCATCCACTGTGAATGATAAGCTGGAGCTGCAA |
| | | GAGTGTCTGGAGCACGGCAGAATAGCCAAGTTCAGCAAAGT |
| | | GAGGACCATTACCACCAGGTCAAACTCTATAAAGCAGGGCA |
| | | AAGACCAGCATTTCCCCGTCTTCATGAACGAGAAGGAGGAC |
| | | ATCCTGTGGTGCACTGAAATGGAAAGGGTGTTTGGCTTCCCC |
| | | GTCCACTACACAGACGTCTCCAACATGAGCCGCTTGGCGAG |
| | | GCAGAGACTGCTGGGCCGATCGTGGAGCGTGCCGGTCATCC |
| | | GCCACCTCTTCGCTCCGCTGAAGGAATATTTTGCTTGTGTGTC |
| | | TAGCGGCAATAGTAACGCTAACAGCCGCGGGCCGAGCTTCA |
| | | GCAGCGGCCTGGTGCCGTTAAGCTTGCGCGGCAGCCATATG |
| | | GGCCCTATGGAGATATACAAGACAGTGTCTGCATGGAAGAG |
| | | ACAGCCAGTGCGGGTACTGAGCCTCTTCAGAAACATCGACA |
| | | AGGTACTAAAGAGTTTGGGCTTCTTGGAAAGCGGTTCTGGTT |
| | | CTGGGGGAGGAACGCTGAAGTACGTGGAAGATGTCACAAAT |
| | | GTCGTGAGGAGAGACGTGGAGAAATGGGGCCCCTTTGACCT |
| | | GGTGTACGGCTCGACGCAGCCCCTAGGCAGCTCTTGTGATCG |
| | | CTGTCCCGGCTGGTACATGTTCCAGTTCCACCGGATCCTGCA |
| | | GTATGCGCTGCCTCGCCAGGAGAGTCAGCGGCCCTTCTTCTG |
| | | GATATTCATGGACAATCTGCTGCTGACTGAGGATGACCAAG |
| | | AGACAACTACCCGCTTCCTTCAGACAGAGGCTGTGACCCTCC |
| | | AGGATGTCCGTGGCAGAGACTACCAGAATGCTATGCGGGTG |
| | | TGGAGCAACATTCCAGGGCTGAAGAGCAAGCATGCGCCCCT |
| | | GACCCCAAAGGAAGAAGAGTATCTGCAAGCCCAAGTCAGAA |
| | | GCAGGAGCAAGCTGGACGCCCCGAAAGTTGACCTCCTGGTG |
| | | AAGAACTGCCTTCTCCCGCTGAGAGAGTACTTCAAGTATTTT |
| | | TCTCAAAACTCACTTCCTCTTGGAGGGCCGAGCTCTGGCGCA |
| | | CCCCCACCAAGTGGAGGGTCTCCTGCCGGGTCCCCAACATCT |
| | | ACTGAAGAAGGCACCAGCGAATCCGCAACGCCCGAGTCAGG |
| | | CCCTGGTACCTCCACAGAACCATCTGAAGGTAGTGCGCCTGG |
| | | TTCCCCAGCTGGAAGCCCTACTTCCACCGAAGAAGGCACGTC |
| | | AACCGAACCAAGTGAAGGATCTGCCCCTGGGACCAGCACTG |
| | | AACCATCTGAGATGGACAAGAAGTACAGCATCGGCCTGGCC |
| | | ATCGGCACCAACTCTGTGGGCTGGGCCGTGATCACCGACGA |
| | | GTACAAGGTGCCCAGCAAGAAATTCAAGGTGCTGGGCAACA |
| | | CCGACCGGCACAGCATCAAGAAGAACCTGATCGGCGCCCTG |
| | | CTGTTCGACAGCGGAGAAACAGCCGAGGCCACCCGGCTGAA |
| | | GAGAACCGCCAGAAGAAGATACACCAGACGGAAGAACCGG |
| | | ATCTGCTATCTGCAAGAGATCTTCAGCAACGAGATGGCCAA |
| | | GGTGGACGACAGCTTCTTCCACAGACTGGAAGAGTCCTTCCT |
| | | GGTGGAAGAGGATAAGAAGCACGAGCGGCACCCCATCTTCG |
| | | GCAACATCGTGGACGAGGTGGCCTACCACGAGAAGTACCCC |
| | | ACCATCTACCACCTGAGAAAGAAACTGGTGGACAGCACCGA |
| | | CAAGGCCGACCTGCGGCTGATCTATCTGGCCCTGGCCCACAT |
| | | GATCAAGTTCCGGGGCCACTTCCTGATCGAGGGCGACCTGA |
| | | ACCCCGACAACAGCGACGTGGACAAGCTGTTCATCCAGCTG |
| | | GTGCAGACCTACAACCAGCTGTTCGAGGAAAACCCCATCAA |
| | | CGCCAGCGGCGTGGACGCCAAGGCCATCCTGTCTGCCAGAC |
| | | TGAGCAAGAGCAGACGGCTGGAAAATCTGATCGCCCAGCTG |
| | | CCCGGCGAGAAGAAGAATGGCCTGTTCGGCAACCTGATTGC |
| | | CCTGAGCCTGGGCCTGACCCCCAACTTCAAGAGCAACTTCGA |
| | | CCTGGCCGAGGATGCCAAACTGCAGCTGAGCAAGGACACCT |
| | | ACGACGACGACCTGGACAACCTGCTGGCCCAGATCGGCGAC |
| | | CAGTACGCCGACCTGTTTCTGGCCGCCAAGAACCTGTCCGAC |
| | | GCCATCCTGCTGAGCGACATCCTGAGAGTGAACACCGAGAT |
| | | CACCAAGGCCCCCCTGAGCGCCTCTATGATCAAGAGATACG |
| | | ACGAGCACCACCAGGACCTGACCCTGCTGAAAGCTCTCGTG |
| | | CGGCAGCAGCTGCCTGAGAAGTACAAAGAGATTTTCTTCGA |
| | | CCAGAGCAAGAACGGCTACGCCGGCTACATCGATGGCGGAG |
| | | CCAGCCAGGAAGAGTTCTACAAGTTCATCAAGCCCATCCTGG |
| | | AAAAGATGGACGGCACCGAGGAACTGCTCGTGAAGCTGAAC |
| | | AGAGAGGACCTGCTGCGGAAGCAGCGGACCTTCGACAACGG |
| | | CAGCATCCCCCACCAGATCCACCTGGGAGAGCTGCACGCCA |
| | | TTCTGCGGCGGCAGGAAGATTTTTACCCATTCCTGAAGGACA |
| | | ACCGGGAAAAGATCGAGAAGATCCTGACCTTCCGCATCCCC |
| | | TACTACGTGGGCCCTCTGGCCAGGGGAAACAGCAGATTCGC |
| | | CTGGATGACCAGAAAGAGCGAGGAAACCATCACCCCCTGGA |
| | | ACTTCGAGGAAGTGGTGGACAAGGGCGCCAGCGCCCAGAGC |
| | | TTCATCGAGCGGATGACCAACTTCGATAAGAACCTGCCCAAC |
| | | GAGAAGGTGCTGCCCAAGCACAGCCTGCTGTACGAGTACTT |
| | | CACCGTGTACAACGAGCTGACCAAAGTGAAATACGTGACCG |
| | | AGGGAATGAGAAAGCCCGCCTTCCTGAGCGGCGAGCAGAAA |
| | | AAAGCCATCGTGGACCTGCTGTTCAAGACCAACCGGAAAGT |
| | | GACCGTGAAGCAGCTGAAAGAGGACTACTTCAAGAAAATCG |
| | | AGTGCTTCGACTCCGTGGAAATCTCCGGCGTGGAAGATCGGT |
| | | TCAACGCCTCCCTGGGCACATACCACGATCTGCTGAAAATTA |
| | | TCAAGGACAAGGACTTCCTGGACAATGAGGAAAACGAGGAC |
| | | ATTCTGGAAGATATCGTGCTGACCCTGACACTGTTTGAGGAC |
| | | AGAGAGATGATCGAGGAACGGCTGAAAACCTATGCCCACCT |
| | | GTTCGACGACAAAGTGATGAAGCAGCTGAAGCGGCGGAGAT |
| | | ACACCGGCTGGGGCAGGCTGAGCCGGAAGCTGATCAACGGC |
| | | ATCCGGGACAAGCAGTCCGGCAAGACAATCCTGGATTTCCT |
| | | GAAGTCCGACGGCTTCGCCAACAGAAACTTCATGCAGCTGA |
| | | TCCACGACGACAGCCTGACCTTTAAAGAGGACATCCAGAAA |
| | | GCCCAGGTGTCCGGCCAGGGCGATAGCCTGCACGAGCACAT |
| | | TGCCAATCTGGCCGGCAGCCCCGCCATTAAGAAGGGCATCCT |
| | | GCAGACAGTGAAGGTGGTGGACGAGCTCGTGAAAGTGATGG |
| | | GCCGGCACAAGCCCGAGAACATCGTGATCGAAATGGCCAGA |
| | | GAGAACCAGACCACCCAGAAGGGACAGAAGAACAGCCGCG |
| | | AGAGAATGAAGCGGATCGAAGAGGGCATCAAAGAGCTGGG |
| | | CAGCCAGATCCTGAAAGAACACCCCGTGGAAAACACCCAGC |
| | | TGCAGAACGAGAAGCTGTACCTGTACTACCTGCAGAATGGG |
| | | CGGGATATGTACGTGGACCAGGAACTGGACATCAACCGGCT |
| | | GTCCGACTACGATGTGGACGCTATCGTGCCTCAGAGCTTTCT |
| | | GAAGGACGACTCCATCGATAACAAAGTGCTGACTCGGAGCG |
| | | ACAAGAACCGGGGCAAGAGCGACAACGTGCCCTCCGAAGAG |
| | | GTCGTGAAGAAGATGAAGAACTACTGGCGCCAGCTGCTGAA |
| | | TGCCAAGCTGATTACCCAGAGGAAGTTCGACAATCTGACCA |
| | | AGGCCGAGAGAGGCGGCCTGAGCGAACTGGATAAGGCCGGC |
| | | TTCATCAAGAGACAGCTGGTGGAAACCCGGCAGATCACAAA |
| | | GCACGTGGCACAGATCCTGGACTCCCGGATGAACACTAAGT |
| | | ACGACGAGAACGACAAACTGATCCGGGAAGTGAAAGTGATC |
| | | ACCCTGAAGTCCAAGCTGGTGTCCGATTTCCGGAAGGATTTC |
| | | CAGTTTTACAAAGTGCGCGAGATCAACAACTACCACCACGC |
| | | CCACGACGCCTACCTGAACGCCGTCGTGGGAACCGCCCTGA |
| | | TCAAAAAGTACCCTAAGCTGGAAAGCGAGTTCGTGTACGGC |
| | | GACTACAAGGTGTACGACGTGCGGAAGATGATCGCCAAGAG |
| | | CGAGCAGGAAATCGGCAAGGCTACCGCCAAGTACTTCTTCT |
| | | ACAGCAACATCATGAACTTTTTCAAGACCGAGATTACCCTGG |
| | | CCAACGGCGAGATCCGGAAGCGGCCTCTGATCGAGACAAAC |
| | | GGCGAAACAGGCGAGATCGTGTGGGATAAGGGCCGGGACTT |
| | | TGCCACCGTGCGGAAAGTGCTGTCTATGCCCCAAGTGAATAT |
| | | CGTGAAAAAGACCGAGGTGCAGACAGGCGGCTTCAGCAAAG |
| | | AGTCTATCCTGCCCAAGAGGAACAGCGACAAGCTGATCGCC |
| | | AGAAAGAAGGACTGGGACCCTAAGAAGTACGGCGGCTTCGA |
| | | CAGCCCCACCGTGGCCTATTCTGTGCTGGTGGTGGCCAAAGT |
| | | GGAAAAGGGCAAGTCCAAGAAACTGAAGAGTGTGAAAGAG |
| | | CTGCTGGGGATCACCATCATGGAAAGAAGCAGCTTCGAGAA |
| | | GAATCCCATCGACTTTCTGGAAGCCAAGGGCTACAAAGAAG |
| | | TGAAAAAGGACCTGATCATCAAGCTGCCTAAGTACTCCCTGT |
| | | TCGAGCTGGAAAACGGCCGGAAGAGAATGCTGGCCTCTGCC |
| | | GGCGAACTGCAGAAGGGAAACGAACTGGCCCTGCCCTCCAA |
| | | ATATGTGAACTTCCTGTACCTGGCCAGCCACTATGAGAAGCT |
| | | GAAGGGCTCCCCCGAGGATAATGAGCAGAAACAGCTGTTTG |
| | | TGGAACAGCACAAACACTACCTGGACGAGATCATCGAGCAG |
| | | ATCAGCGAGTTCTCCAAGAGAGTGATCCTGGCCGACGCTAAT |
| | | CTGGACAAGGTGCTGAGCGCCTACAACAAGCACAGAGACAA |
| | | GCCTATCAGAGAGCAGGCCGAGAATATCATCCACCTGTTTAC |
| | | CCTGACCAATCTGGGAGCCCCTGCCGCCTTCAAGTACTTTGA |
| | | CACCACCATCGACCGGAAGAGGTACACCAGCACCAAAGAGG |
| | | TGCTGGACGCCACCCTGATCCACCAGAGCATCACCGGCCTGT |
| | | ACGAGACACGGATCGACCTGTCTCAGCTGGGAGGCGACAGC |
| | | GGAAGTGAGACCCCAGGTACATCCGAATCAGCAACGCCTGA |
| | | AAGCACCGGTATGAACAATTCACAGGGGAGAGTGACATTCG |
| | | AAGACGTGACCGTGAACTTCACCCAGGGAGAATGGCAGCGC |
| | | TTGAACCCAGAACAAAGGAACCTCTATCGGGACGTGATGCT |
| | | GGAAAACTACTCAAATTTGGTGAGCGTTGGGCAGGGTGAGA |
| | | CCACTAAGCCTGACGTGATCCTGAGATTGGAACAGGGCAAG |
| | | GAGCCTTGGCTCGAGGAAGAGGAAGTCCTGGGCTCAGGGAG |
| | | GGCCGAGAAAAACGGTGATATAGGAGGCCAGATATGGAAGC |
| | | CTAAGGACGTCAAGGAGAGCCTGAGCGCTGATTACAAAGAT |
| | | GATGACGATAAAGCCCCAAAAAAGAAGAGAAAGGTACCGA |
| | | AGAAAAAAAGAAAGGTC |
| 34 | Fusion Protein 8 | MPKKKRKVPKKKRKVNHDQEFDPPKVYPPVPAEKRKPIRVLSL |
| | Amino Acid | FDGIATGLLVLKDLGIQVDRYIASEVCEDSITVGMVRHQGKIMY |
| | Sequence | VGDVRSVTQKHIQEWGPFDLVIGGSPCNDLSIVNPARKGLYEGT |
| | NLS-3A-3L- | GRLFFEFYRLLHDARPKEGDDRPFFWLFENVVAMGVSDKRDIS |
| | dCas9-ZFP-NLS | RFLESNPVMIDAKEVSAAHRARYFWGNLPGMNRPLASTVNDK |
| | | LELQECLEHGRIAKFSKVRTITTRSNSIKQGKDQHFPVFMNEKE |
| | | DILWCTEMERVFGFPVHYTDVSNMSRLARQRLLGRSWSVPVIR |
| | | HLFAPLKEYFACVSSGNSNANSRGPSFSSGLVPLSLRGSHMGPM |
| | | EIYKTVSAWKRQPVRVLSLFRNIDKVLKSLGFLESGSGSGGGTL |
| | | KYVEDVTNVVRRDVEKWGPFDLVYGSTQPLGSSCDRCPGWY |
| | | MFQFHRILQYALPRQESQRPFFWIFMDNLLLTEDDQETTTRFLQ |
| | | TEAVTLQDVRGRDYQNAMRVWSNIPGLKSKHAPLTPKEEEYL |
| | | QAQVRSRSKLDAPKVDLLVKNCLLPLREYFKYFSQNSLPLGGP |
| | | SSGAPPPSGGSPAGSPTSTEEGTSESATPESGPGTSTEPSEGSAPG |
| | | SPAGSPTSTEEGTSTEPSEGSAPGTSTEPSEMDKKYSIGLAIGTNS |
| | | VGWAVITDEYKVPSKKFKVLGNTDRHSIKKNLIGALLFDSGET |
| | | AEATRLKRTARRRYTRRKNRICYLQEIFSNEMAKVDDSFFHRLE |
| | | ESFLVEEDKKHERHPIFGNIVDEVAYHEKYPTIYHLRKKLVDST |
| | | DKADLRLIYLALAHMIKFRGHFLIEGDLNPDNSDVDKLFIQLVQ |
| | | TYNQLFEENPINASGVDAKAILSARLSKSRRLENLIAQLPGEKK |
| | | NGLFGNLIALSLGLTPNFKSNFDLAEDAKLQLSKDTYDDDLDN |
| | | LLAQIGDQYADLFLAAKNLSDAILLSDILRVNTEITKAPLSASMI |
| | | KRYDEHHQDLTLLKALVRQQLPEKYKEIFFDQSKNGYAGYIDG |
| | | GASQEEFYKFIKPILEKMDGTEELLVKLNREDLLRKQRTFDNGS |
| | | IPHQIHLGELHAILRRQEDFYPFLKDNREKIEKILTFRIPYYVGPL |
| | | ARGNSRFAWMTRKSEETITPWNFEEVVDKGASAQSFIERMTNF |
| | | DKNLPNEKVLPKHSLLYEYFTVYNELTKVKYVTEGMRKPAFLS |
| | | GEQKKAIVDLLFKTNRKVTVKQLKEDYFKKIECFDSVEISGVED |
| | | RFNASLGTYHDLLKIIKDKDFLDNEENEDILEDIVLTLTLFEDRE |
| | | MIEERLKTYAHLFDDKVMKQLKRRRYTGWGRLSRKLINGIRDK |
| | | QSGKTILDFLKSDGFANRNFMQLIHDDSLTFKEDIQKAQVSGQG |
| | | DSLHEHIANLAGSPAIKKGILQTVKVVDELVKVMGRHKPENIVI |
| | | EMARENQTTQKGQKNSRERMKRIEEGIKELGSQILKEHPVENT |
| | | QLQNEKLYLYYLQNGRDMYVDQELDINRLSDYDVDAIVPQSFL |
| | | KDDSIDNKVLTRSDKNRGKSDNVPSEEVVKKMKNYWRQLLNA |
| | | KLITQRKFDNLTKAERGGLSELDKAGFIKRQLVETRQITKHVAQ |
| | | ILDSRMNTKYDENDKLIREVKVITLKSKLVSDFRKDFQFYKVRE |
| | | INNYHHAHDAYLNAVVGTALIKKYPKLESEFVYGDYKVYDVR |
| | | KMIAKSEQEIGKATAKYFFYSNIMNFFKTEITLANGEIRKRPLIET |
| | | NGETGEIVWDKGRDFATVRKVLSMPQVNIVKKTEVQTGGFSKE |
| | | SILPKRNSDKLIARKKDWDPKKYGGFDSPTVAYSVLVVAKVEK |
| | | GKSKKLKSVKELLGITIMERSSFEKNPIDFLEAKGYKEVKKDLII |
| | | KLPKYSLFELENGRKRMLASAGELQKGNELALPSKYVNFLYLA |
| | | SHYEKLKGSPEDNEQKQLFVEQHKHYLDEIIEQISEFSKRVILAD |
| | | ANLDKVLSAYNKHRDKPIREQAENIIHLFTLTNLGAPAAFKYFD |
| | | TTIDRKRYTSTKEVLDATLIHQSITGLYETRIDLSQLGGDSGSETP |
| | | GTSESATPESTGNKKLEAVGTGIEPKAMSQGLVTFGDVAVDFS |
| | | QEEWEWLNPIQRNLYRKVMLENYRNLASLGLCVSKPDVISSLE |
| | | QGKEPWSADYKDDDDKAPKKKRKVPKKKRKV |
| 35 | Fusion Protein 8 | ATGCCAAAAAAGAAGAGAAAGGTACCGAAGAAAAAAAGAA |
| | DNA Sequence | AGGTATACAACCATGACCAGGAATTCGACCCCCCAAAGGTT |
| | | TACCCACCTGTGCCAGCTGAGAAGAGGAAGCCCATCCGCGT |
| | | GCTGTCTCTCTTTGATGGGATTGCTACAGGGCTCCTGGTGCT |
| | | GAAGGACCTGGGCATCCAAGTGGACCGCTACATTGCCTCCG |
| | | AGGTGTGTGAGGACTCCATCACGGTGGGCATGGTGCGGCAC |
| | | CAGGGAAAGATCATGTACGTCGGGGACGTCCGCAGCGTCAC |
| | | ACAGAAGCATATCCAGGAGTGGGGCCCATTCGACCTGGTGA |
| | | TTGGAGGCAGTCCCTGCAATGACCTCTCCATTGTCAACCCTG |
| | | CCCGCAAGGGACTTTATGAGGGTACTGGCCGCCTCTTCTTTG |
| | | AGTTCTACCGCCTCCTGCATGATGCGCGGCCCAAGGAGGGA |
| | | GATGATCGCCCCTTCTTCTGGCTCTTTGAGAATGTGGTGGCC |
| | | ATGGGCGTTAGTGACAAGAGGGACATCTCGCGATTTCTTGAG |
| | | TCTAACCCCGTGATGATTGACGCCAAAGAAGTGTCTGCTGCA |
| | | CACAGGGCCCGTTACTTCTGGGGTAACCTTCCTGGCATGAAC |
| | | AGGCCTTTGGCATCCACTGTGAATGATAAGCTGGAGCTGCAA |
| | | GAGTGTCTGGAGCACGGCAGAATAGCCAAGTTCAGCAAAGT |
| | | GAGGACCATTACCACCAGGTCAAACTCTATAAAGCAGGGCA |
| | | AAGACCAGCATTTCCCCGTCTTCATGAACGAGAAGGAGGAC |
| | | ATCCTGTGGTGCACTGAAATGGAAAGGGTGTTTGGCTTCCCC |
| | | GTCCACTACACAGACGTCTCCAACATGAGCCGCTTGGCGAG |
| | | GCAGAGACTGCTGGGCCGATCGTGGAGCGTGCCGGTCATCC |
| | | GCCACCTCTTCGCTCCGCTGAAGGAATATTTTGCTTGTGTGTC |
| | | TAGCGGCAATAGTAACGCTAACAGCCGCGGGCCGAGCTTCA |
| | | GCAGCGGCCTGGTGCCGTTAAGCTTGCGCGGCAGCCATATG |
| | | GGCCCTATGGAGATATACAAGACAGTGTCTGCATGGAAGAG |
| | | ACAGCCAGTGCGGGTACTGAGCCTCTTCAGAAACATCGACA |
| | | AGGTACTAAAGAGTTTGGGCTTCTTGGAAAGCGGTTCTGGTT |
| | | CTGGGGGAGGAACGCTGAAGTACGTGGAAGATGTCACAAAT |
| | | GTCGTGAGGAGAGACGTGGAGAAATGGGGCCCCTTTGACCT |
| | | GGTGTACGGCTCGACGCAGCCCCTAGGCAGCTCTTGTGATCG |
| | | CTGTCCCGGCTGGTACATGTTCCAGTTCCACCGGATCCTGCA |
| | | GTATGCGCTGCCTCGCCAGGAGAGTCAGCGGCCCTTCTTCTG |
| | | GATATTCATGGACAATCTGCTGCTGACTGAGGATGACCAAG |
| | | AGACAACTACCCGCTTCCTTCAGACAGAGGCTGTGACCCTCC |
| | | AGGATGTCCGTGGCAGAGACTACCAGAATGCTATGCGGGTG |
| | | TGGAGCAACATTCCAGGGCTGAAGAGCAAGCATGCGCCCCT |
| | | GACCCCAAAGGAAGAAGAGTATCTGCAAGCCCAAGTCAGAA |
| | | GCAGGAGCAAGCTGGACGCCCCGAAAGTTGACCTCCTGGTG |
| | | AAGAACTGCCTTCTCCCGCTGAGAGAGTACTTCAAGTATTTT |
| | | TCTCAAAACTCACTTCCTCTTGGAGGGCCGAGCTCTGGCGCA |
| | | CCCCCACCAAGTGGAGGGTCTCCTGCCGGGTCCCCAACATCT |
| | | ACTGAAGAAGGCACCAGCGAATCCGCAACGCCCGAGTCAGG |
| | | CCCTGGTACCTCCACAGAACCATCTGAAGGTAGTGCGCCTGG |
| | | TTCCCCAGCTGGAAGCCCTACTTCCACCGAAGAAGGCACGTC |
| | | AACCGAACCAAGTGAAGGATCTGCCCCTGGGACCAGCACTG |
| | | AACCATCTGAGATGGACAAGAAGTACAGCATCGGCCTGGCC |
| | | ATCGGCACCAACTCTGTGGGCTGGGCCGTGATCACCGACGA |
| | | GTACAAGGTGCCCAGCAAGAAATTCAAGGTGCTGGGCAACA |
| | | CCGACCGGCACAGCATCAAGAAGAACCTGATCGGCGCCCTG |
| | | CTGTTCGACAGCGGAGAAACAGCCGAGGCCACCCGGCTGAA |
| | | GAGAACCGCCAGAAGAAGATACACCAGACGGAAGAACCGG |
| | | ATCTGCTATCTGCAAGAGATCTTCAGCAACGAGATGGCCAA |
| | | GGTGGACGACAGCTTCTTCCACAGACTGGAAGAGTCCTTCCT |
| | | GGTGGAAGAGGATAAGAAGCACGAGCGGCACCCCATCTTCG |
| | | GCAACATCGTGGACGAGGTGGCCTACCACGAGAAGTACCCC |
| | | ACCATCTACCACCTGAGAAAGAAACTGGTGGACAGCACCGA |
| | | CAAGGCCGACCTGCGGCTGATCTATCTGGCCCTGGCCCACAT |
| | | GATCAAGTTCCGGGGCCACTTCCTGATCGAGGGCGACCTGA |
| | | ACCCCGACAACAGCGACGTGGACAAGCTGTTCATCCAGCTG |
| | | GTGCAGACCTACAACCAGCTGTTCGAGGAAAACCCCATCAA |
| | | CGCCAGCGGCGTGGACGCCAAGGCCATCCTGTCTGCCAGAC |
| | | TGAGCAAGAGCAGACGGCTGGAAAATCTGATCGCCCAGCTG |
| | | CCCGGCGAGAAGAAGAATGGCCTGTTCGGCAACCTGATTGC |
| | | CCTGAGCCTGGGCCTGACCCCCAACTTCAAGAGCAACTTCGA |
| | | CCTGGCCGAGGATGCCAAACTGCAGCTGAGCAAGGACACCT |
| | | ACGACGACGACCTGGACAACCTGCTGGCCCAGATCGGCGAC |
| | | CAGTACGCCGACCTGTTTCTGGCCGCCAAGAACCTGTCCGAC |
| | | GCCATCCTGCTGAGCGACATCCTGAGAGTGAACACCGAGAT |
| | | CACCAAGGCCCCCCTGAGCGCCTCTATGATCAAGAGATACG |
| | | ACGAGCACCACCAGGACCTGACCCTGCTGAAAGCTCTCGTG |
| | | CGGCAGCAGCTGCCTGAGAAGTACAAAGAGATTTTCTTCGA |
| | | CCAGAGCAAGAACGGCTACGCCGGCTACATCGATGGCGGAG |
| | | CCAGCCAGGAAGAGTTCTACAAGTTCATCAAGCCCATCCTGG |
| | | AAAAGATGGACGGCACCGAGGAACTGCTCGTGAAGCTGAAC |
| | | AGAGAGGACCTGCTGCGGAAGCAGCGGACCTTCGACAACGG |
| | | CAGCATCCCCCACCAGATCCACCTGGGAGAGCTGCACGCCA |
| | | TTCTGCGGCGGCAGGAAGATTTTTACCCATTCCTGAAGGACA |
| | | ACCGGGAAAAGATCGAGAAGATCCTGACCTTCCGCATCCCC |
| | | TACTACGTGGGCCCTCTGGCCAGGGGAAACAGCAGATTCGC |
| | | CTGGATGACCAGAAAGAGCGAGGAAACCATCACCCCCTGGA |
| | | ACTTCGAGGAAGTGGTGGACAAGGGCGCCAGCGCCCAGAGC |
| | | TTCATCGAGCGGATGACCAACTTCGATAAGAACCTGCCCAAC |
| | | GAGAAGGTGCTGCCCAAGCACAGCCTGCTGTACGAGTACTT |
| | | CACCGTGTACAACGAGCTGACCAAAGTGAAATACGTGACCG |
| | | AGGGAATGAGAAAGCCCGCCTTCCTGAGCGGCGAGCAGAAA |
| | | AAAGCCATCGTGGACCTGCTGTTCAAGACCAACCGGAAAGT |
| | | GACCGTGAAGCAGCTGAAAGAGGACTACTTCAAGAAAATCG |
| | | AGTGCTTCGACTCCGTGGAAATCTCCGGCGTGGAAGATCGGT |
| | | TCAACGCCTCCCTGGGCACATACCACGATCTGCTGAAAATTA |
| | | TCAAGGACAAGGACTTCCTGGACAATGAGGAAAACGAGGAC |
| | | ATTCTGGAAGATATCGTGCTGACCCTGACACTGTTTGAGGAC |
| | | AGAGAGATGATCGAGGAACGGCTGAAAACCTATGCCCACCT |
| | | GTTCGACGACAAAGTGATGAAGCAGCTGAAGCGGCGGAGAT |
| | | ACACCGGCTGGGGCAGGCTGAGCCGGAAGCTGATCAACGGC |
| | | ATCCGGGACAAGCAGTCCGGCAAGACAATCCTGGATTTCCT |
| | | GAAGTCCGACGGCTTCGCCAACAGAAACTTCATGCAGCTGA |
| | | TCCACGACGACAGCCTGACCTTTAAAGAGGACATCCAGAAA |
| | | GCCCAGGTGTCCGGCCAGGGCGATAGCCTGCACGAGCACAT |
| | | TGCCAATCTGGCCGGCAGCCCCGCCATTAAGAAGGGCATCCT |
| | | GCAGACAGTGAAGGTGGTGGACGAGCTCGTGAAAGTGATGG |
| | | GCCGGCACAAGCCCGAGAACATCGTGATCGAAATGGCCAGA |
| | | GAGAACCAGACCACCCAGAAGGGACAGAAGAACAGCCGCG |
| | | AGAGAATGAAGCGGATCGAAGAGGGCATCAAAGAGCTGGG |
| | | CAGCCAGATCCTGAAAGAACACCCCGTGGAAAACACCCAGC |
| | | TGCAGAACGAGAAGCTGTACCTGTACTACCTGCAGAATGGG |
| | | CGGGATATGTACGTGGACCAGGAACTGGACATCAACCGGCT |
| | | GTCCGACTACGATGTGGACGCTATCGTGCCTCAGAGCTTTCT |
| | | GAAGGACGACTCCATCGATAACAAAGTGCTGACTCGGAGCG |
| | | ACAAGAACCGGGGCAAGAGCGACAACGTGCCCTCCGAAGAG |
| | | GTCGTGAAGAAGATGAAGAACTACTGGCGCCAGCTGCTGAA |
| | | TGCCAAGCTGATTACCCAGAGGAAGTTCGACAATCTGACCA |
| | | AGGCCGAGAGAGGCGGCCTGAGCGAACTGGATAAGGCCGGC |
| | | TTCATCAAGAGACAGCTGGTGGAAACCCGGCAGATCACAAA |
| | | GCACGTGGCACAGATCCTGGACTCCCGGATGAACACTAAGT |
| | | ACGACGAGAACGACAAACTGATCCGGGAAGTGAAAGTGATC |
| | | ACCCTGAAGTCCAAGCTGGTGTCCGATTTCCGGAAGGATTTC |
| | | CAGTTTTACAAAGTGCGCGAGATCAACAACTACCACCACGC |
| | | CCACGACGCCTACCTGAACGCCGTCGTGGGAACCGCCCTGA |
| | | TCAAAAAGTACCCTAAGCTGGAAAGCGAGTTCGTGTACGGC |
| | | GACTACAAGGTGTACGACGTGCGGAAGATGATCGCCAAGAG |
| | | CGAGCAGGAAATCGGCAAGGCTACCGCCAAGTACTTCTTCT |
| | | ACAGCAACATCATGAACTTTTTCAAGACCGAGATTACCCTGG |
| | | CCAACGGCGAGATCCGGAAGCGGCCTCTGATCGAGACAAAC |
| | | GGCGAAACAGGCGAGATCGTGTGGGATAAGGGCCGGGACTT |
| | | TGCCACCGTGCGGAAAGTGCTGTCTATGCCCCAAGTGAATAT |
| | | CGTGAAAAAGACCGAGGTGCAGACAGGCGGCTTCAGCAAAG |
| | | AGTCTATCCTGCCCAAGAGGAACAGCGACAAGCTGATCGCC |
| | | AGAAAGAAGGACTGGGACCCTAAGAAGTACGGCGGCTTCGA |
| | | CAGCCCCACCGTGGCCTATTCTGTGCTGGTGGTGGCCAAAGT |
| | | GGAAAAGGGCAAGTCCAAGAAACTGAAGAGTGTGAAAGAG |
| | | CTGCTGGGGATCACCATCATGGAAAGAAGCAGCTTCGAGAA |
| | | GAATCCCATCGACTTTCTGGAAGCCAAGGGCTACAAAGAAG |
| | | TGAAAAAGGACCTGATCATCAAGCTGCCTAAGTACTCCCTGT |
| | | TCGAGCTGGAAAACGGCCGGAAGAGAATGCTGGCCTCTGCC |
| | | GGCGAACTGCAGAAGGGAAACGAACTGGCCCTGCCCTCCAA |
| | | ATATGTGAACTTCCTGTACCTGGCCAGCCACTATGAGAAGCT |
| | | GAAGGGCTCCCCCGAGGATAATGAGCAGAAACAGCTGTTTG |
| | | TGGAACAGCACAAACACTACCTGGACGAGATCATCGAGCAG |
| | | ATCAGCGAGTTCTCCAAGAGAGTGATCCTGGCCGACGCTAAT |
| | | CTGGACAAGGTGCTGAGCGCCTACAACAAGCACAGAGACAA |
| | | GCCTATCAGAGAGCAGGCCGAGAATATCATCCACCTGTTTAC |
| | | CCTGACCAATCTGGGAGCCCCTGCCGCCTTCAAGTACTTTGA |
| | | CACCACCATCGACCGGAAGAGGTACACCAGCACCAAAGAGG |
| | | TGCTGGACGCCACCCTGATCCACCAGAGCATCACCGGCCTGT |
| | | ACGAGACACGGATCGACCTGTCTCAGCTGGGAGGCGACAGC |
| | | GGAAGTGAGACCCCAGGTACATCCGAATCAGCAACGCCTGA |
| | | AAGCACCGGTAACAAAAAGCTTGAGGCCGTCGGAACCGGAA |
| | | TCGAACCAAAAGCAATGTCCCAGGGTTTGGTGACATTTGGCG |
| | | ACGTGGCTGTCGATTTTTCCCAGGAAGAGTGGGAGTGGCTCA |
| | | ATCCTATCCAGAGGAACTTGTACCGGAAGGTGATGCTGGAG |
| | | AATTATAGAAATTTGGCATCACTGGGGTTGTGCGTTAGCAAA |
| | | CCAGATGTTATATCTTCCCTGGAACAGGGAAAGGAGCCCTG |
| | | GAGCGCTGATTACAAAGATGATGACGATAAAGCCCCAAAAA |
| | | AGAAGAGAAAGGTACCGAAGAAAAAAAGAAAGGTC |
| 36 | M.AluI | MSKANAKYSFVDLFAGIGGFHAALAATGGVCEYAVEIDREAA |
| | methyltransferase | AVYERNWNKPALGDITDDANDEGVTLRGYDGPIDVLTGGFPC |
| | | QPFSKSGAQHGMAETRGTLFWNIARIIEEREPTVLILENVRNLV |
| | | GPRHRHEWLTIIETLRFFGYEVSGAPAIFSPHLLPAWMGGTPQV |
| | | RERVFITATLVPERMRDERSTIRRATGRPLEGFPYWADSWTDFR |
| | | ELSRLVVIRGFQAPEREVVGDRKRYVARTDMPEGFVPASVTRP |
| | | AIDETLPAWKQSHLRRNYDFFERHFAEVVAWAYRWGVYTDLF |
| | | PASRRKLEWQAQDAPRLWDTVMHFRPSGIRAKRPTYLPALVAI |
| | | TQTSIVGPLERRLSPRETARLQGLPEWFDFGEQRAAATYKQMG |
| | | NGVNVGVVRHILREHVRRDRALLKLTPAGQRIINAVLADEPDA |
| | | TVGALGAAE |
| 37 | M.MspI | MKPEILKLIRSKLDLTQKQASEIIEVSDKTWQQWESGKTEMHPA |
| | methyltransferase | YYSFLQEKLKDKINFEELSAQKTLQKKIFDKYNQNQITKNAEEL |
| | | AEITHIEERKDAYSSDFKFIDLFSGIGGIRQSFEVNGGKCVFSSEI |
| | | DPFAKFTYYTNFGVVPFGDITKVEATTIPQHDILCAGFPCQPFSH |
| | | IGKREGFEHPTQGTMFHEIVRIIETKKTPVLFLENVPGLINHDDG |
| | | NTLKVIIETLEDMGYKVHHTVLDASHFGIPQKRKRFYLVAFLNQ |
| | | NIHFEFPKPPMISKDIGEVLESDVTGYSISEHLQKSYLFKKDDGK |
| | | PSLIDKNTTGAVKTLVSTYHKIQRLTGTFVKDGETGIRLLTTNEC |
| | | KAIMGFPKDFVIPVSRTQMYRQMGNSVVVPVVTKIAEQISLALK |
| | | TVNQQSPQENFELELV |
| 38 | M.HaeIII | MNLISLFSGAGGLDLGFQKAGFRIICANEYDKSIWKTYESNHSA |
| | methyltransferase | KLIKGDISKISSDEFPKCDGIIGGPPCQSWSEGGSLRGIDDPRGKL |
| | | FYEYIRILKQKKPIFFLAENVKGMMAQRHNKAVQEFIQEFDNA |
| | | GYDVHIILLNANDYGVAQDRKRVFYIGFRKELNINYLPPIPHLIK |
| | | PTFKDVIWDLKDNPIPALDKNKTNGNKCIYPNHEYFIGSYSTIFM |
| | | SRNRVRQWNEPAFTVQASGRQCQLHPQAPVMLKVSKNLNKFV |
| | | EGKEHLYRRLTVRECARVQGFPDDFIFHYESLNDGYKMIGNAV |
| | | PVNLAYEIAKTIKSALEICKGN |
| 39 | M. HhaI | MIEIKDKQLTGLRFIDLFAGLGGFRLALESCGAECVYSNEWDKY |
| | methyltransferase | AQEVYEMNFGEKPEGDITQVNEKTIPDHDILCAGFPCQAFSISG |
| | | KQKGFEDSRGTLFFDIARIVREKKPKVVFMENVKNFASHDNGN |
| | | TLEVVKNTMNELDYSFHAKVLNALDYGIPQKRERIYMICFRND |
| | | LNIQNFQFPKPFELNTFVKDLLLPDSEVEHLVIDRKDLVMTNQEI |
| | | EQTTPKTVRLGIVGKGGQGERIYSTRGIAITLSAYGGGIFAKTGG |
| | | YLVNGKTRKLHPRECARVMGYPDSYKVHPSTSQAYKQFGNSV |
| | | VINVLQYIAYNIGSSLNFKPY |
| 40 | M. SssI | MSKVENKTKKLRVFEAFAGIGAQRKALEKVRKDEYEIVGLAE |
| | methyltransferase | WYVPAIVMYQAIHNNFHTKLEYKSVSREEMIDYLENKTLSWNS |
| | | KNPVSNGYWKRKKDDELKIIYNAIKLSEKEGNIFDIRDLYKRTL |
| | | KNIDLLTYSFPCQDLSQQGIQKGMKRGSGTRSGLLWEIERALDS |
| | | TEKNDLPKYLLMENVGALLHKKNEEELNQWKQKLESLGYQNS |
| | | IEVLNAADFGSSQARRRVFMISTLNEFVELPKGDKKPKSIKKVL |
| | | NKIVSEKDILNNLLKYNLTEFKKTKSNINKASLIGYSKFNSEGYV |
| | | YDPEFTGPTLTASGANSRIKIKDGSNIRKMNSDETFLYIGFDSQD |
| | | GKRVNEIEFLTENQKIFVCGNSISVEVLEAIIDKIGG |
| 41 | M. bacterium | MAEWYIPAIVSYQAIHNGFTLNKINHKIELQTMIDYLESKTLSM |
| | methyltransferase | NSKEPVKRGFWYKKHLDEIRIVYTAVKMSEQEGNIFDVRTLFE |
| | | RGLSDIDLLTYSFPCQDLSQQGKQKGMGRDSQTRSGLLWEIEK |
| | | ALDTSKKEDLPKYLLMENVVALTHKVNAEELDEWMMKLESL |
| | | GYKNDLRILNAGDFGSSQARRRTFMISTLNEKVELPVGNKKPKS |
| | | MNKILNDEPTRKDFLPALDKFDLTEYKWTKSNINKAKLINYSTF |
| | | NSEAYVYDSNFTGPTLTASGANSRIKFEYNGKIRKIGAEEAYAY |
| | | MGFKKSDYIKVNKLNYLNETKMIYTCGNSISVEVLRSIMTNINN |
| | | NFKENK |
| 42 | M. marinum | MLFLIGTFKYVLIYITKVIRIFEAFAGIGAQRKALRNIKSNYEVSG |
| | methyltransferase | MAEWYIPAIVSYQAIHNGFTLSRVDKKTKLTEMIKYLESKTLSM |
| | | DSKEPVRTGYWFKKHKDMVRIVYSAVKLSEAEGNIFDVRTLHE |
| | | RKLEDIDLLTYSFPCQDLSQQGKQRGMKKDSGTRSGLLWEIEK |
| | | ALEATPKDKLPKYLLMENVVALTHKTNKKDLDNWKRKLRSLG |
| | | YYNDINVLNAGDFGSSQARRRAFMISTLDSKVTLPLGDKKPQAI |
| | | SKILNKETRSQDFMPALDEYEKTDFKRTLSNIKKCKLIDYTSFNS |
| | | EAYVYDPKYTGPTLTASGANSRIKFTHQGKMRKINAEEAYRYM |
| | | GFSTNDYKKVNNLNFLSETKMIYTCGNSISVEVLEEIMLKIIRED |
| | | NNG |
| 43 | S. chinense | MKKIRLFEAFAGIGSQRRALKSVVGNNFEIAGLAEWYVPAIVM |
| | methyltransferase | YQIINNDFSKKNVLDNVPRDEVIDYLNSKCLSWDSKKPVSKNF |
| | | WNRKSQDILNVIYSAVKKSEEEGNIFDVRTLHERTLESIDILTYS |
| | | FPCQDLSQQGIQKGMKKNSGTRSGLLWEIEKAIDNTPKNNLPKI |
| | | LLMENVPALLNKTNELELKEWLIKLENMGYKNSIGILNAADFG |
| | | SPQARRRVFMISSRNKKIELPVGKSKPGKLNDILEKNVEDKFIM |
| | | TNLEKYDFSEFSLTKSNIKKCSLINYTKFNSEAYVYDPDFTGPTL |
| | | TASGANSRIKIYDKGFIRRMSPLESFRYMGFDDEDYKKIDEFEFL |
| | | TDTQKIFVCGNSISIEVLKAIFERIDSNE |
| 44 | ZFP28 | MKKLEAVGTGIEPKAMSQGLVTFGDVAVDFSQEEWEWLNPIQ |
| | | RNLYRKVMLENYRNLASLGLCVSKPDVISSLEQGKEPWTVKRK |
| | | MTRAWCPDLKAVWKIKELPLKKDFCEG |
| 45 | ZN627 | MDSVAFEDVAVNFTLEEWALLDPSQKNLYRDVMRETFRNLAS |
| | | VGKQWEDQNIEDPFKIPRRNISHIPERLCESKEGGQGEETFSQIP |
| | | DGILNKKTPGVKPCESSVCGEVGMGPSSLNRHIRDHTGREPNEY |
| | | QEYGKKSYTRNQCGRALSYHRSFPVRERTHPGGKPYDCKECGE |
| | | TFISLVSIRRHMLTHRGGVPYKCKVCGKAFDYPSLFRIHERSHT |
| | | GEKPYECKQCGKAFSCSSYIRIHERTHTGDKPYECKQCGKAFSC |
| | | SKYIRIHERTHTGEKPYECKQCGKAFRCASSVRSHERTHTGEKL |
| | | FECKECGKALTCLASVRRHMIKHTGNGPYKCKVCGKAFDFPSS |
| | | FRIHERTHTGEKPYDCKQCGKAFSCSSSFRKHERIHTGEKPYKC |
| | | TKCGKAFSRSSYFRIHERTHTGEKPYECKQCGKAFSRSTYFRVH |
| | | EKIHTGEKPYENPNPNASVVPVLS |
| 46 | KAP1 | MAASAAAASAAAASAASGSPGPGEGSAGGEKRSTAPSAAASAS |
| | | ASAAASSPAGGGAEALELLEHCGVCRERLRPEREPRLLPCLHSA |
| | | CSACLGPAAPAAANSSGDGGAAGDGTVVDCPVCKQQCFSKDI |
| | | VENYFMRDSGSKAATDAQDANQCCTSCEDNAPATSYCVECSE |
| | | PLCETCVEAHQRVKYTKDHTVRSTGPAKSRDGERTVYCNVHK |
| | | HEPLVLFCESCDTLTCRDCQLNAHKDHQYQFLEDAVRNQRKLL |
| | | ASLVKRLGDKHATLQKSTKEVRSSIRQVSDVQKRVQVDVKMAI |
| | | LQIMKELNKRGRVLVNDAQKVTEGQQERLERQHWTMTKIQKH |
| | | QEHILRFASWALESDNNTALLLSKKLIYFQLHRALKMIVDPVEP |
| | | HGEMKFQWDLNAWTKSAEAFGKIVAERPGTNSTGPAPMAPPR |
| | | APGPLSKQGSGSSQPMEVQEGYGFGSGDDPYSSAEPHVSGVKR |
| | | SRSGEGEVSGLMRKVPRVSLERLDLDLTADSQPPVFKVFPGSTT |
| | | EDYNLIVIERGAAAAATGQPGTAPAGTPGAPPLAGMAIVKEEET |
| | | EAAIGAPPTATEGPETKPVLMALAEGPGAEGPRLASPSGSTSSG |
| | | LEVVAPEGTSAPGGGPGTLDDSATICRVCQKPGDLVMCNQCEF |
| | | CFHLDCHLPALQDVPGEEWSCSLCHVLPDLKEEDGSLSLDGAD |
| | | STGVVAKLSPANQRKCERVLLALFCHEPCRPLHQLATDSTFSLD |
| | | QPGGTLDLTLIRARLQEKLSPPYSSPQEFAQDVGRMFKQFNKLT |
| | | EDKADVQSIIGLQRFFETRMNEAFGDTKFSAVLVEPPPMSLPGA |
| | | GLSSQELSGGPGDGP |
| 47 | MeCP2 | MVAGMLGLREEKSEDQDLQGLKDKPLKFKKVKKDKKEEKEG |
| | | KHEPVQPSAHHSAEPAEAGKAETSEGSGSAPAVPEASASPKQR |
| | | RSIIRDRGPMYDDPTLPEGWTRKLKQRKSGRSAGKYDVYLINP |
| | | QGKAFRSKVELIAYFEKVGDTSLDPNDFDFTVTGRGSPSRREQK |
| | | PPKKPKSPKAPGTGRGRGRPKGSGTTRPKAATSEGVQVKRVLE |
| | | KSPGKLLVKMPFQTSPGGKAEGGGATTSTQVMVIKRPGRKRK |
| | | AEADPQAIPKKRGRKPGSVVAAAAAEAKKKAVKESSIRSVQET |
| | | VLPIKKRKTRETVSIEVKEVVKPLLVSTLGEKSGKGLKTCKSPG |
| | | RKSKESSPKGRSSSASSPPKKEHHHHHHHSESPKAPVPLLPPLPP |
| | | PPPEPESSEDPTSPPEPQDLSSSVCKEEKMPRGGSLESDGCPKEP |
| | | AKTQPAVATAATAAEKYKHRGEGERKDIVSSSMPRPNREEPVD |
| | | SRTPVTERVS |
| 48 | HP1b | MGKKQNKKKVEEVLEEEEEEYVVEKVLDRRVVKGKVEYLLK |
| | | WKGFSDEDNTWEPEENLDCPDLIAEFLQSQKTAHETDKSEGGK |
| | | RKADSDSEDKGEESKPKKKKEESEKPRGFARGLEPERIIGATDS |
| | | SGELMFLMKWKNSDEADLVPAKEANVKCPQVVISFYEERLTW |
| | | HSYPSEDDDKKDDKN |
| 49 | CBX8 | GSGPPSSGGGLYRDMGAQGGRPSLIARIPVARILGDPEEESWSPS |
| | | LTNLEKVVVTDVTSNFLTVTIKESNTDQGFFKEKR |
| 50 | CDYL2 | ASGDLYEVERIVDKRKNKKGKWEYLIRWKGYGSTEDTWEPEH |
| | | HLLHCEEFIDEFNGLHMSKDKRIKSGKQSSTSKLLRDS |
| 51 | TOX | KDPNEPQKPVSAYALFFRDTQAAIKGQNPNATFGEVSKIVASM |
| | | WDGLGEEQKQVYKKKTEAAKKEYLKQLAAYRASLVSK |
| 52 | TOX3 | KDPNEPQKPVSAYALFFRDTQAAIKGQNPNATFGEVSKIVASM |
| | | WDSLGEEQKQVYKRKTEAAKKEYLKALAAYRASLVSK |
| 53 | TOX4 | KDPNEPQKPVSAYALFFRDTQAAIKGQNPNATFGEVSKIVASM |
| | | WDSLGEEQKQVYKRKTEAAKKEYLKALAAYKDNQECQ |
| 54 | EED | MSEREVSTAPAGTDMPAAKKQKLSSDENSNPDLSGDENDDAV |
| | | SIESGTNTERPDTPTNTPNAPGRKSWGKGKWKSKKCKYSFKCV |
| | | NSLKEDHNQPLFGVQFNWHSKEGDPLVFATVGSNRVTLYECHS |
| | | QGEIRLLQSYVDADADENFYTCAWTYDSNTSHPLLAVAGSRGII |
| | | RIINPITMQCIKHYVGHGNAINELKFHPRDPNLLLSVSKDHALRL |
| | | WNIQTDTLVAIFGGVEGHRDEVLSADYDLLGEKIMSCGMDHSL |
| | | KLWRINSKRMMNAIKESYDYNPNKTNRPFISQKIHFPDFSTRDI |
| | | HRNYVDCVRWLGDLILSKSCENAIVCWKPGKMEDDIDKIKPSE |
| | | SNVTILGRFDYSQCDIWYMRFSMDFWQKMLALGNQVGKLYV |
| | | WDLEVEDPHKAKCTTLTHHKCGAAIRQTSFSRDSSILIAVCDDA |
| | | SIWRWDRLR |
| 55 | RBBP4 | VWDLSKIGEEQSPEDAEDGPPELLFIHGGHTAKISDFSWNPNEP |
| | | WVICSVSEDNIMQVWQMAENIYNDEDPEGSVDPEGQ |
| 56 | RCOR1 | MPAMVEKGPEVSGKRRGRNNAAASASAAAASAAASAACASP |
| | | AATAASGAAASSASAAAASAAAAPNNGQNKSLAAAAPNGNSS |
| | | SNSWEEGSSGSSSDEEHGGGGMRVGPQYQAVVPDFDPAKLAR |
| | | RSQERDNLGMLVWSPNQNLSEAKLDEYIAIAKEKHGYNMEQA |
| | | LGMLFWHKHNIEKSLADLPNFTPFPDEWTVEDKVLFEQAFSFH |
| | | GKTFHRIQQMLPDKSIASLVKFYYSWKKTRTKTSVMDRHARK |
| | | QKREREESEDELEEANGNNPIDIEVDQNKESKKEVPPTETVPQV |
| | | KKEKHSTQAKNRAKRKPPKGMFLSQEDVEAVSANATAATTVL |
| | | RQLDMELVSVKRQIQNIKQTNSALKEKLDGGIEPYRLPEVIQKC |
| | | NARWTTEEQLLAVQAIRKYGRDFQAISDVIGNKSVVQVKNFFV |
| | | NYRRRFNIDEVLQEWEAEHGKEETNGPSNQKPVKSPDNSIKMP |
| | | EEEDEAPVLDVRYASAS |
| 57 | SCML2 | KQGFSKDPSTWSVDEVIQFMKHTDPQISGPLADLFRQHEIDGKA |
| | | LFLLKSDVMMKYMGLKLGPALKLCYYIEKLKEGKYS |
| 58 | KOX1KRAB | RTLVTFKDVFVDFTREEWKLLDTAQQIVYRNVMLENYKNLVS |
| | | LGYQLTKPDVILRLEKGEEP |
| 59 | KOX1KRAB-FL | RTLVTFKDVFVDFTREEWKLLDTAQQIVYRNVMLENYKNLVS |
| | | LGYQLTKPDVILRLEKGEEPWLVEREIHQETHPDSETAFEIKSSV |
| | | SSRSIFKDKQS |
| 60 | ZIM3 | MNNSQGRVTFEDVTVNFTQGEWQRLNPEQRNLYRDVMLENYS |
| | | NLVSVGQGETTKPDVILRLEQGKEPWLEEEEVLGSGRAEKNGD |
| | | IGGQIWKPKDVKESL |
| 61 | KOX1KRAB- | RTLVTFKDVFVDFTREEWKLLDTAQQIVYRNVMLENYKNLVS |
| | ZIM3 chimera 1 | LGYQLTKPDVILRLEKGEEPWLEEEEVLGSGRAEKNGDIGGQI |
| | | WKPKDVKESL |
| 62 | ZIM3- | MNNSQGRVTFEDVTVNFTQGEWQRLNPEQRNLYRDVMLENYS |
| | KOX1KRAB | NLVSVGQGETTKPDVILRLEQGKEPWLVEREIHQETHPDSETAF |
| | chimera 2 | EIKSSVSSRSIFKDKQS |
| 63 | Truncated ZIM3 | MNNSQGRVTFEDVTVNFTQGEWQRLNPEQRNLYRDVMLENYS |
| | | NLVSVGQGETTKPDVILRLEQGKEPWLEEEEVL |
| 64 | KOX1KRAB-FL | RTLVTFKDVAVDFTQEEWQQLDPAQKIVYRDVMLENYSNLVS |
| | Zim3-like | VGYQLTKPDVILRLEQKGEEPWLVEEEIHQETHPDSETAFEIKSS |
| | | VSSRSIFKDKQS |
| 65 | KOX1KRAB-like | MTKSLETVTFKDVAVDLTQEEWQQMKPAQKNLYRDVMLENY |
| | ZIM3 1 | SNLVTVGYQVTKPDVIFKLEQREEPWVIEEEMFG |
| 66 | KOX1KRAB-like | MTKSLETVTFKDVAVDLTQEEWQQMKPAQKNLYRDVMLENY |
| | ZIM3 2 | SNLVTVGYQVTKPDVIFKLEQREEPWVIEEEMFG |
| 67 | KOX1KRAB-like | MNTSLGPLSFKDVAVAFTQEEWQQLGPEEKTTYRDVMLENYS |
| | ZIM3 3 | NLVSVGYHIIKPDVIIKLEQGDEPWVGEE |
| 68 | KOX1KRAB-like | RTLVTFKDVAVDFTQEEWKLLDPAQQDLYRDVMLENYSNLVS |
| | ZIM3 4 | LGYQLTKPDVILRLEQGKEPWLVEREIHQETHPDSETAFEIKSSV |
| | | SSRSIFKDKQS |
| 69 | ZIM-like | VTFRDVAIDFSQEEWKWLQPAQRDLYRCVMLENYGHLVSLAG |
| | KOX1KRAB 1 | LSISKPDLVSLLEQGKEPWLGKGDIRRNLFSVSEPTDEIKEFSPEN |
| | | VIYKGDSS |
| 70 | ZIM-like | VTFRDVAIDFSQEEWKWLQPAQRDLYRCVMLENYGHLVSLGL |
| | KOX1KRAB 2 | SISKPDLVSLLEQGKEPWLGKGDIRRNLFSVSEPTDEIKEFSPEN |
| | | VIYKGDSS |
| 71 | ZIM-like | VTFRDVAIDFSQEEWEWLQPAQRDLYRCVMLENYGHLVSLAG |
| | KOX1KRAB 3 | LSISKPDVVSLLEQGKEPWLRKGDVSRDLFSVSESNCEIKDISSK |
| | | NDIY |
| 72 | A. melanoleuca | MALSPTGTLSVETLDRSDPDPLDEGPWQATCEILLEPDAEHSTD |
| | DNMT3L | VILVGSSELSAPASPGPRRDLLAYEVKVNQRDIEDVCICCGSLR |
| | | VHTQHPLFEGGMCAPCKDKFLDCLFLYDDDGYQSYCSICCAGE |
| | | TLLICENPDCTRPSLMMKLRLFRECACLIFPSEGMLLQTVWFWK |
| | | MTVVWQPGLRHLPQENPLETYKTVPVWKREPVRVLSLFGDIRR |
| | | ELMSLGFLESGSAPGRLKHLDDVTDVVRKDVEGWGPFDLVYG |
| | | STPPIGHACDHPPVWYLLQFHRILQYARPRPGSQQPFFWMFVD |
| | | NLVLSQDDQTAATRFLEADPVTIQDVCGRAVRNTVHVWSNIPA |
| | | VRSRHSALALCEELSLLAQDRQRTKPPAQGPAQLVKNCFLPLR |
| | | EYFKYFSTELTSSL |
| 73 | C. syrichta | MALSCRRTLPLESLHSSNSDLASQLDKEQWRPPCETHGIPVAAA |
| | DNMT3L | PVLDLEAECSLDVILVGSSELSTSSSPRLGRDHIAYEVKVNQRNI |
| | | EDICLCCGSFLVHTQHPLFEGGMCAPCKDKFLDTLFLYDEDGY |
| | | QSYCSICCSGETLLICENPDCTRCYCFECLDTLVSPGTSEKVHA |
| | | MSNWVCFLCLPFTRSGLLQRRRKWRGQLKAFYDRESESSLEM |
| | | YKTVPVWKREPVRVLSLFGDIKKELMSLGFVETGSDPGRLRHL |
| | | DDTTNIVRRNVEEWGPFHLLYGATPPLGHTCDRPPGWYLFQFH |
| | | RLLQYARPQPGSPQPFFWMFVDNVMLTREDRAIASRFLETEPV |
| | | TIPDIHGRALQNAVCVWSNIPAVRSKHSALVSEEELSLLAQDRQ |
| | | RAKLPTQGPTKLVKNCFLPLREYFKYFSTELTSFL |
| 74 | M. unguiculatus | MGSQETPSTRAKTPGTWNLESTDSSSPESLGHLEEQWANSSPDL |
| | DNMT3L | KDEHSKDVEPEDSKELISSASPPSGREIIRYEISVNQRNIEDICLCC |
| | | GTLQVYKQHPLFEGGICAPCKDKFLETFFLYDEDGHQSYCSICC |
| | | SGGTLFICESPDCTRCYCFECVDILVGPGTSERINAMPCWVCFLC |
| | | LPFTRSGLLQRRRKWRHQLKAFFDEGGASPLEMYKTVSAWKR |
| | | KPMRVLSLFKNIDKELKNLGFLESGSGSEEERLKYLEDVINVVR |
| | | RDVEKWGPFDLVYGSTRPRGSSCDHCPAWYMFQFHRILQY AR |
| | | PPSGSEQPFFWVFVDNLLMTEDDQITADRFLQMKAVTLQDVRG |
| | | RVLQNAVRVWSNIPGVKSKHMALTEKEEQSLEAQAGTRTKLS |
| | | AQKVDPLVKNCLLPLREYFKFFSQNSLPLDK |
| 75 | O. princeps | MALPSPETLDSLDRVPASHPDEQHWTVCDNSDPILEVEAEGSM |
| | DNMT3L | DVILVDDSPAPSGRDRIELEVKVNQRSIEDLCLCCGSSQVHRQH |
| | | PLFQGGLCAPCKDKFLEALFLYDEDGYQSYCSICGLGDTLLVCE |
| | | SPDCTRGYCFACVDGLVGAGSSGHMHTVSPWVCFLCVPGSRH |
| | | GLLQRRRRWRTQLKVFHEQEAAQPLEIYETVPACRRKPLRVLS |
| | | LFEHIEKELASLGFLETGSSPGRIRHLDDVTDVVRRDVEQWGPF |
| | | DLVYGSTPPLGHASPRSPGWYLFQFHRMLQYTQPTASTQRPFF |
| | | WMFVDNLLLTRDDLVTATRFLEVEPATLQDVRGRVLQGAMRV |
| | | WSNIPAVNSRHTELAPEAETALLAQSCRRAKASGEGLARLLKS |
| | | CFLPLREYFKYFPQSPLPLRK |
| 76 | N. carolinensis | MGGPRPAAVEESPHEIYKTVPAWKREPMRVLSLFGDIGKELTSL |
| | DNMT3L | GFLETGSEAGRLKHLEDVTDTVRRDVEEWGPFDLVYGSTPALG |
| | | HSCDRSPGWYLFQFHRLLQYARPRLGSPKPFFWMFVDNLLLTK |
| | | DDQAIASRFLEMEPVTLQDVHGRVLQNAVRVWTNVPAVKSRH |
| | | SALASEEELLLVQDGQRGRLPAQGPAALVKHCFLPLREYFKYFS |
| | | QNTLPLYK |
| 77 | B. bison | MARSSPGTLNLEIMDGSDPDPALPPDREQWPPPCEILLDPEPEHS |
| | DNMT3L | LDIILVGSSELSSPPSPGPRRDFIAYEVKVNQRDIEDVCICCGSLQ |
| | | LHTQHPLFEGGMCAPCKDKFLECLFLYDDDGYQSYCSICCAGE |
| | | TLLICENPDCTRCYCFECVDTLVGPGTSGKVHAMSNWVCFLCL |
| | | PFPRSGLLQRRRKWRTWLKAFYDREAESPLVMYKTVPVWKRE |
| | | PIRVLSLFGDIKKELTSLGFLEDGSKPGRLKHLDDVTNIVRRDID |
| | | EWGPFDLTYGSTPTLGHTCDHPPGWYVYQFHRILQYARPLPGS |
| | | PQPFFWMFVDNLVLTEEDLDVATRFLETDPVTIQDVRGRTVQN |
| | | AVHVWSNIPAVKSRHSALVSQEELSLLAQDRQRVKSPVQGPAT |
| | | LVKNCFLPLREYFKYFSTELTSSL |
| 78 | E. przewalskii | MALSSPGTLSLETLDSWDPDVAGQLDEERWQPSSEIVGRPMAA |
| | DNMT3L | APVLDLEEEPSMDIILVDSSELSSPPSPGPSRDMCICCGSFQVHTQ |
| | | HPLFEGGMCAACKDKFLSCLFLYDDDGNQSYCSICCSGETLLIC |
| | | ENPDCTRCYCFECVDTLVSPRTSEKVQAMSNWVCFLCLPFPRS |
| | | GLLQRRRKWRGWLKAFYDQEAVRSRSAWGRRMRSGPHLVGF |
| | | LWLLVAKCPSALESPLEMYKTVPVWKREPVRVLSLFGDIKKEL |
| | | TTLGFLENGSDPGRLKHLDDVTNTVRRDVEEWGPFDLVYGSTP |
| | | PLGHACDHPPGWYLFQFHRVLQYARPRPGSPQAFFWMFVDNL |
| | | VLTEDDRAVATRFLETDPVTIQDVCGRAVRNAVHVWSNIPAVK |
| | | SRHSALFSQEESFLRAQDRQRAKPPARGPAKLVKNCFLPLREYF |
| | | KYFSTEFTSSL |
| 79 | M. caroli | MGSRETPSSFSKTLETLDLETSDSSSPDADSPLEEQWLKSSPALK |
| | DNMT3L | EDNVDMVLEDCKEPLSPSSPPTGREMIRYEVKVNRRSIEDICLC |
| | | CGTLQVYTQHPLFEGGICAPCKDKFLESLFLYDDDGHQSYCTIC |
| | | CSGGTLFICESPDCTRCYCFECVDILVGPGTSERINAMACWVCF |
| | | LCLPFSRSGLLQRRKRWRHQLKAFHDQEGAGPMEIYKTVSTW |
| | | KRQPVRVLSLFGNIDKVLKSLGFLESGSGSGGGTLKYVEDVTN |
| | | VVRRDVEKWGPFDLVYGSTQPLGSSCDRCPGWYMFQFHRILQ |
| | | YALPRQESQRPFFWIFMDNLLMTEDDQETTARFLQTEAVTLQD |
| | | VRGRDYQNVMRVWSNIPGLKSKHVPLTPKEEEYLQAQVRTRS |
| | | KLDAQKVDLLVKNCLLPLREYFKYFS |
| 80 | P. troglodytes | MAAIPALDPEAEPSMDVILVGSSELSSSISPRTGRDLIAYEVKAN |
| | DNMT3L | QRNIEDICICCGSLQVHTQHPLFEGGICAPCKDKSLDALFLYDDD |
| | | GYQSYCSICCSGETLLICGNPDCTRCYCFECVDSLVGPGTSGKV |
| | | HAMSNWVCYLCLPSSRSGLLQRRRKWRSQLKAFYDRESENPL |
| | | EMFETVPVWRRQPVRVLSLFEDIKKELTSLGFLESGSDPGQLKH |
| | | VVDVTDTVRKDVEEWGPFDLVYGATPPLGHTCDRPPSWYLFQ |
| | | FHRLLQYARPKPGSPRPFFWMFVDNLVLNKEDLDVASRFLEME |
| | | PVTIPDVHGGSLQNAVRVWSNIPAIRSSRHWALVSEEELSLLAQ |
| | | NKQSSKLAAKWPTKLVKNCFLPLREYFKYFSTELTSSL |
| 81 | M. penetrans | MNSNKDKIKVIKVFEAFAGIGSQFKALKNIARSKNWEIQHSGM |
| | M MpeI | VEWFVDAIVSYVAIHSKNFNPKIEQLDKDILSISNDSKMPISEYGI |
| | | KKINNTIKASYLNYAKKHFNNLFDIKKVNKDNFPKNIDIFTYSFP |
| | | CQDLSVQGLQKGIDKELNTRSGLLWEIERILEEIKNSFSKEEMPK |
| | | YLLMENVKNLLSHKNKKNYNTWLKQLEKFGYKSKTYLLNSKN |
| | | FDNCQNRERVFCLSIRDDYLEKTGFKFKELEKVKNPPKKIKDIL |
| | | VDSSNYKYLNLNKYETTTFRETKSNIISRSLKNYTTFNSENYVY |
| | | NINGIGPTLTASGANSRIKIETQQGVRYLTPLECFKYMQFDVND |
| | | FKKVQSTNLISENKMIYIAGNSIPVKILEAIFNTLEFVNNEE |
| 82 | S. monobiae | MSKVENKTKKLRVFEAFAGIGAQRKALEKVRKDEYEIVGLAE |
| | M SssI | WYVPAIVMYQAIHNNFHTKLEYKSVSREEMIDYLENKTLSWNS |
| | | KNPVSNGYWKRKKDDELKIIYNAIKLSEKEGNIFDIRDLYKRTL |
| | | KNIDLLTYSFPCQDLSQQGIQKGMKRGSGTRSGLLWEIERALDS |
| | | TEKNDLPKYLLMENVGALLHKKNEEELNQWKQKLESLGYQNS |
| | | IEVLNAADFGSSQARRRVFMISTLNEFVELPKGDKKPKSIKKVL |
| | | NKIVSEKDILNNLLKYNLTEFKKTKSNINKASLIGYSKENSEGYV |
| | | YDPEFTGPTLTASGANSRIKIKDGSNIRKMNSDETFLYIGFDSQD |
| | | GKRVNEIEFLTENQKIFVCGNSISVEVLEAIIDKIGG |
| 83 | H. parainfluenzae | MKDVLDDNLLEEPAAQYSLFEPESNPNLREKFTFIDLFAGIGGF |
| | M HpaII | RIAMQNLGGKCIFSSEWDEQAQKTYEANFGDLPYGDITLEETK |
| | | AFIPEKFDILCAGFPCQAFSIAGKRGGFEDTRGTLFFDVAEIIRRH |
| | | QPKAFFLENVKGLKNHDKGRTLKTILNVLREDLGYFVPEPAIVN |
| | | AKNFGVPQNRERIYIVGFHKSTGVNSFSYPEPLDKIVTFADIREE |
| | | KTVPTKYYLSTQYIDTLRKHKERHESKGNGFGYEIIPDDGIANAI |
| | | VVGGMGRERNLVIDHRITDFTPTTNIKGEVNREGIRKMTPREW |
| | | ARLQGFPDSYVIPVSDASAYKQFGNSVAVPAIQATGKKILEKLG |
| | | NLYD |
| 84 | A. luteus | MSKANAKYSFVDLFAGIGGFHAALAATGGVCEYAVEIDREAA |
| | M AluI | AVYERNWNKPALGDITDDANDEGVTLRGYDGPIDVLTGGFPC |
| | | QPFSKSGAQHGMAETRGTLFWNIARIIEEREPTVLILENVRNLV |
| | | GPRHRHEWLTIIETLRFFGYEVSGAPAIFSPHLLPAWMGGTPQV |
| | | RERVFITATLVPERMRDERIPRTETGEIDAEAIGPKPVATMNDRF |
| | | PIKKGGTELFHPGDRKSGWNLLTSGIIREGDPEPSNVDLRLTETE |
| | | TLWIDAWDDLESTIRRATGRPLEGFPYWADSWTDFRELSRLVVI |
| | | RGFQAPEREVVGDRKRYVARTDMPEGFVPASVTRPAIDETLPA |
| | | WKQSHLRRNYDFFERHFAEVVAWAYRWGVYTDLFPASRRKLE |
| | | WQAQDAPRLWDTVMHFRPSGIRAKRPTYLPALVAITQTSIVGP |
| | | LERRLSPRETARLQGLPEWFDFGEQRAAATYKQMGNGVNVGV |
| | | VRHILREHVRRDRALLKLTPAGQRIINAVLADEPDATVGALGA |
| | | AE |
| 85 | H. aegyptius | MNLISLFSGAGGLDLGFQKAGFRIICANEYDKSIWKTYESNHSA |
| | M HaeIII | KLIKGDISKISSDEFPKCDGIIGGPPCQSWSEGGSLRGIDDPRGKL |
| | | FYEYIRILKQKKPIFFLAENVKGMMAQRHNKAVQEFIQEFDNA |
| | | GYDVHIILLNANDYGVAQDRKRVFYIGFRKELNINYLPPIPHLIK |
| | | PTFKDVIWDLKDNPIPALDKNKTNGNKCIYPNHEYFIGSYSTIFM |
| | | SRNRVRQWNEPAFTVQASGRQCQLHPQAPVMLKVSKNLNKFV |
| | | EGKEHLYRRLTVRECARVQGFPDDFIFHYESLNDGYKMIGNAV |
| | | PVNLAYEIAKTIKSALEICKGN |
| 86 | H. haemolyticus | MIEIKDKQLTGLRFIDLFAGLGGFRLALESCGAECVYSNEWDKY |
| | M HhaI | AQEVYEMNFGEKPEGDITQVNEKTIPDHDILCAGFPCQAFSISG |
| | | KQKGFEDSRGTLFFDIARIVREKKPKVVFMENVKNFASHDNGN |
| | | TLEVVKNTMNELDYSFHAKVLNALDYGIPQKRERIYMICFRND |
| | | LNIQNFQFPKPFELNTFVKDLLLPDSEVEHLVIDRKDLVMTNQEI |
| | | EQTTPKTVRLGIVGKGGQGERIYSTRGIAITLSAYGGGIFAKTGG |
| | | YLVNGKTRKLHPRECARVMGYPDSYKVHPSTSQAYKQFGNSV |
| | | VINVLQYIAYNIGSSLNFKPY |
| 87 | Moraxella | MKPEILKLIRSKLDLTQKQASEIIEVSDKTWQQWESGKTEMHPA |
| | M MspI | YYSFLQEKLKDKINFEELSAQKTLQKKIFDKYNQNQITKNAEEL |
| | | AEITHIEERKDAYSSDFKFIDLFSGIGGIRQSFEVNGGKCVFSSEI |
| | | DPFAKFTYYTNFGVVPFGDITKVEATTIPQHDILCAGFPCQPFSH |
| | | IGKREGFEHPTQGTMFHEIVRIIETKKTPVLFLENVPGLINHDDG |
| | | NTLKVIIETLEDMGYKVHHTVLDASHFGIPQKRKRFYLVAFLNQ |
| | | NIHFEFPKPPMISKDIGEVLESDVTGYSISEHLQKSYLFKKDDGK |
| | | PSLIDKNTTGAVKTLVSTYHKIQRLTGTFVKDGETGIRLLTTNEC |
| | | KAIMGFPKDFVIPVSRTQMYRQMGNSVVVPVVTKIAEQISLALK |
| | | TVNQQSPQENFELELV |
| 88 | E. coli strain 12 | MNNNDLVAKLWKLCDNLRDGGVSYQNYVNELASLLFLKMCK |
| | hsdM | ETGQEAEYLPEGYRWDDLKSRIGQEQLQFYRKMLVHLGEDDK |
| | | KLVQAVFHNVSTTITEPKQITALVSNMDSLDWYNGAHGKSRDD |
| | | FGDMYEGLLQKNANETKSGAGQYFTPRPLIKTIIHLLKPQPREV |
| | | VQDPAAGTAGFLIEADRYVKSQTNDLDDLDGDTQDFQIHRAFI |
| | | GLELVPGTRRLALMNCLLHDIEGNLDHGGAIRLGNTLGSDGEN |
| | | LPKAHIVATNPPFGSAAGTNITRTFVHPTSNKQLCFMQHIIETLH |
| | | PGGRAAVVVPDNVLFEGGKGTDIRRDLMDKCHLHTILRLPTGIF |
| | | YAQGVKTNVLFFTKGTVANPNQDKNCTDDVWVYDLRTNMPS |
| | | FGKRTPFTDEHLQPFERVYGEDPHGLSPRTEGEWSFNAEETEVA |
| | | DSEENKNTDQHLATSRWRKFSREWIRTAKSDSLDISWLKDKDSI |
| | | DADSLPEPDVLAAEAMGELVQALSELDALMRELGASDEADLQ |
| | | RQLLEEAFGGVKE |
| 89 | E. coli strain 12 | MSAGKLPEGWVIAPVSTVTTLIRGVTYKKEQAINYLKDDYLPLI |
| | hsdS | RANNIQNGKFDTTDLVFVPKNLVKESQKISPEDIVIAMSSGSKSV |
| | | VGKSAHQHLPFECSFGAFCGVLRPEKLIFSGFIAHFTKSSLYRNK |
| | | ISSLSAGANINNIKPASFDLINIPIPPLAEQKIIAEKLDTLLAQVDS |
| | | TKARFEQIPQILKRFRQAVLGGAVNGKLTEKWRNFEPQHSVFK |
| | | KLNFESILTELRNGLSSKPNESGVGHPILRISSVRAGHVDQNDIR |
| | | FLECSESELNRHKLQDGDLLFTRYNGSLEFVGVCGLLKKLQHQ |
| | | NLLYPDKLIRARLTKDALPEYIEIFFSSPSARNAMMNCVKTTSG |
| | | QKGISGKDIKSQVVLLPPVKEQAEIVRRVEQLFAYADTIEKQVN |
| | | NALARVNNLTQSILAKAFRGELTAQWRAENPDLISGENSAAAL |
| | | LEKIKAERAASGGKKASRKKS |
| 90 | T. aquaticus | MGLPPLLSLPSNSAPRSLGRVETPPEVVDFMVSLAEAPRGGRVL |
| | M TaqI | EPACAHGPFLRAFREAHGTAYRFVGVEIDPKALDLPPWAEGILA |
| | | DFLLWEPGEAFDLILGNPPYGIVGEASKYPIHVFKAVKDLYKKA |
| | | FSTWKGKYNLYGAFLEKAVRLLKPGGVLVFVVPATWLVLEDF |
| | | ALLREFLAREGKTSVYYLGEVFPQKKVSAVVIRFQKSGKGLSL |
| | | WDTQESESGFTPILWAEYPHWEGEIIRFETEETRKLEISGMPLGD |
| | | LFHIRFAARSPEFKKHPAVRKEPGPGLVPVLTGRNLKPGWVDY |
| | | EKNHSGLWMPKERAKELRDFYATPHLVVAHTKGTRVVAAWD |
| | | ERAYPWREEFHLLPKEGVRLDPSSLVQWLNSEAMQKHVRTLY |
| | | RDFVPHLTLRMLERLPVRREYGFHTSPESARNF |
| 91 | E. coli | MKKNRAFLKWAGGKYPLLDDIKRHLPKGECLVEPFVGAGSVF |
| | M EcoDam | LNTDFSRYILADINSDLISLYNIVKMRTDEYVQAARELFVPETNC |
| | | AEVYYQFREEFNKSQDPFRRAVLFLYLNRYGYNGLCRYNLRGE |
| | | FNVPFGRYKKPYFPEAELYHFAEKAQNAFFYCESYADSMARAD |
| | | DASVVYCDPPYAPLSATANFTAYHTNSFTLEQQAHLAEIAEGL |
| | | VERHIPVLISNHDTMLTREWYQRAKLHVVKVRRSISSNGGTRK |
| | | KVDELLALYKPGVVSPAKK |
| 92 | C. crescentus | MKFGPETIIHGDCIEQMNALPEKSVDLIFADPPYNLQLGGDLLRP |
| | M CcrMI | DNSKVDAVDDHWDQFESFAAYDKFTREWLKAARRVLKDDGA |
| | | IWVIGSYHNIFRVGVAVQDLGFWILNDIVWRKSNPMPNFKGTR |
| | | FANAHETLIWASKSQNAKRYTFNYDALKMANDEVQMRSDWTI |
| | | PLCTGEERIKGADGQKAHPTQKPEALLYRVILSTTKPGDVILDPF |
| | | FGVGTTGAAAKRLGRKFIGIEREAEYLEHAKARIAKVVPIAPED |
| | | LDVMGSKRAEPRVPFGTIVEAGLLSPGDTLYCSKGTHVAKVRP |
| | | DGSITVGDLSGSIHKIGALVQSAPACNGWTYWHFKTDAGLAPI |
| | | DVLRAQVRAGMN |
| 93 | C. difficile | MDDISQDNFLLSKEYENSLDVDTKKASGIYYTPKIIVDYIVKKTL |
| | CamA | KNHDIIKNPYPRILDISCGCGNFLLEVYDILYDLFEENIYELKKK |
| | | YDENYWTVDNIHRHILNYCIYGADIDEKAISILKDSLTNKKVVN |
| | | DLDESDIKINLFCCDSLKKKWRYKFDYIVGNPPYIGHKKLEKKY |
| | | KKFLLEKYSEVYKDKADLYFCFYKKIIDILKQGGIGSVITPRYFL |
| | | ESLSGKDLREYIKSNVNVQEIVDFLGANIFKNIGVSSCILTFDKK |
| | | KTKETYIDVFKIKNEDICINKFETLEELLKSSKFEHFNINQRLLSD |
| | | EWILVNKDDETFYNKIQEKCKYSLEDIAISFQGIITGCDKAFILSK |
| | | DDVKLNLVDDKFLKCWIKSKNINKYIVDKSEYRLIYSNDIDNEN |
| | | TNKRILDEIIGLYKTKLENRRECKSGIRKWYELQWGREKLFFER |
| | | KKIMYPYKSNENRFAIDYDNNFSSADVYSFFIKEEYLDKFSYEY |
| | | LVGILNSSVYDKYFKITAKKMSKNIYDYYPNKVMKIRIFRDNNY |
| | | EEIENLSKQIISILLNKSIDKGKVEKLQIKMDNLIMDSLGI |
| 94 | KOX1 (ZNF10) | MDAKSLTAWSRTLVTFKDVFVDFTREEWKLLDTAQQIVYRNV |
| | | MLENYKNLVSLGYQLTKPDVILRLEKGEEPWLVEREIHQETHP |
| | | DSETAFEIKSSVSSRSIFKDKQSCDIKMEGMARNDLWYLSLEEV |
| | | WKCRDQLDKYQENPERHLRQVAFTQKKVLTQERVSESGKYGG |
| | | NCLLPAQLVLREYFHKRDSHTKSLKHDLVLNGHQDSCASNSNE |
| | | CGQTFCQNIHLIQFARTHTGDKSYKCPDNDNSLTHGSSLGISKGI |
| | | HREKPYECKECGKFFSWRSNLTRHQLIHTGEKPYECKECGKSFS |
| | | RSSHLIGHQKTHTGEEPYECKECGKSFSWFSHLVTHQRTHTGD |
| | | KLYTCNQCGKSFVHSSRLIRHQRTHTGEKPYECPECGKSFRQST |
| | | HLILHQRTHVRVRPYECNECGKSYSQRSHLVVHHRIHTGLKPFE |
| | | CKDCGKCFSRSSHLYSHQRTHTGEKPYECHDCGKSFSQSSALIV |
| | | HQRIHTGEKPYECCQCGKAFIRKNDLIKHQRIHVGEETYKCNQC |
| | | GIIFSQNSPFIVHQIAHTGEQFLTCNQCGTALVNTSNLIGYQTNHI |
| | | RENAY |
| 95 | 2X SV40 NLS | PKKKRKVPKKKRKV |
| 96 | DNMT3A | NHDQEFDPPKVYPPVPAEKRKPIRVLSLFDGIATGLLVLKDLGIQ |
| | catalytic domain | VDRYIASEVCEDSITVGMVRHQGKIMYVGDVRSVTQKHIQEW |
| | | GPFDLVIGGSPCNDLSIVNPARKGLYEGTGRLFFEFYRLLHDAR |
| | | PKEGDDRPFFWLFENVVAMGVSDKRDISRFLESNPVMIDAKEV |
| | | SAAHRARYFWGNLPGMNRPLASTVNDKLELQECLEHGRIAKFS |
| | | KVRTITTRSNSIKQGKDQHFPVFMNEKEDILWCTEMERVFGFPV |
| | | HYTDVSNMSRLARQRLLGRSWSVPVIRHLFAPLKEYFACV |
| 97 | DNMT3L | NPLEMFETVPVWRRQPVRVLSLFEDIKKELTSLGFLESGSDPGQ |
| | catalytic domain | LKHVVDVTDTVRKDVEEWGPFDLVYGATPPLGHTCDRPPSWY |
| | | LFQFHRLLQYARPKPGSPRPFFWMFVDNLVLNKEDLDVASRFL |
| | | EMEPVTIPDVHGGSLQNAVRVWSNIPAIRSRHWALVSEEELSLL |
| | | AQNKQSSKLAAKWPTKLVKNCFLPLREYFKYFSTELTSSL |
| 98 | ADD | EVKVNRRSIEDICLCCGTLQVYTRHPLFEGGLCAPCKDKFLESL |
| | | FLYDDDGHQSYCTICCSGGTLFICESPDCTRCYCFECVDILVGPG |
| | | TSERINAMACWVCFLCLPFSRSGLLQRRKRWRHQLKAFHDQE |
| | | GA |
| 99 | KOX1KRAB | RTLVTFKDVFVDFTREEWKLLDTAQQIVYRNVMLENYKNLVS |
| | | LGYQLTKPDVILRLEKGEEP |
| 100 | ZIM3 | MGRVTFEDVTVNFTQGEWQRLNPEQRNLYRDVMLENYSNLVS |
| | | VGQGETTKPDVILRLEQGKEPWLEEEEVLGSGRAEKNGDIGGQI |
| | | WKPKDVKESL |
| 101 | A. melanoleuca | NPLETYKTVPVWKREPVRVLSLFGDIRRELMSLGFLESGSAPGR |
| | DNMT3L | LKHLDDVTDVVRKDVEGWGPFDLVYGSTPPIGHACDHPPVWY |
| | Catalytic Domain | LLQFHRILQYARPRPGSQQPFFWMFVDNLVLSQDDQTAATRFL |
| | | EADPVTIQDVCGRAVRNTVHVWSNIPAVRSRHSALALCEELSL |
| | | LAQDRQRTKPPAQGPAQLVKNCFLPLREYFKYFSTELTSSL |
| 102 | C. syrichta | SSLEMYKTVPVWKREPVRVLSLFGDIKKELMSLGFVETGSDPG |
| | DNMT3L | RLRHLDDTTNIVRRNVEEWGPFHLLYGATPPLGHTCDRPPGWY |
| | Catalytic Domain | LFQFHRLLQYARPQPGSPQPFFWMFVDNVMLTREDRAIASRFL |
| | | ETEPVTIPDIHGRALQNAVCVWSNIPAVRSKHSALVSEEELSLLA |
| | | QDRQRAKLPTQGPTKLVKNCFLPLREYFKYFSTELTSFL |
| 103 | M. unguiculatus | SPLEMYKTVSAWKRKPMRVLSLFKNIDKELKNLGFLESGSGSE |
| | DNMT3L | EERLKYLEDVINVVRRDVEKWGPFDLVYGSTRPRGSSCDHCPA |
| | Catalytic Domain | WYMFQFHRILQYARPPSGSEQPFFWVFVDNLLMTEDDQITADR |
| | | FLQMKAVTLQDVRGRVLQNAVRVWSNIPGVKSKHMALTEKEE |
| | | QSLEAQAGTRTKLSAQKVDPLVKNCLLPLREYFKFFSQNSLPLD |
| | | K |
| 104 | O. princeps | QPLEIYETVPACRRKPLRVLSLFEHIEKELASLGFLETGSSPGRIR |
| | DNMT3L | HLDDVTDVVRRDVEQWGPFDLVYGSTPPLGHASPRSPGWYLF |
| | Catalytic Domain | QFHRMLQYTQPTASTQRPFFWMFVDNLLLTRDDLVTATRFLEV |
| | | EPATLQDVRGRVLQGAMRVWSNIPAVNSRHTELAPEAETALLA |
| | | QSCRRAKASGEGLARLLKSCFLPLREYFKYFPQSPLPLRK |
| 105 | N. carolinensis | SPHEIYKTVPAWKREPMRVLSLFGDIGKELTSLGFLETGSEAGR |
| | DNMT3L | LKHLEDVTDTVRRDVEEWGPFDLVYGSTPALGHSCDRSPGWY |
| | Catalytic Domain | LFQFHRLLQYARPRLGSPKPFFWMFVDNLLLTKDDQAIASRFLE |
| | | MEPVTLQDVHGRVLQNAVRVWTNVPAVKSRHSALASEEELLL |
| | | VQDGQRGRLPAQGPAALVKHCFLPLREYFKYFSQNTLPLYK |
| 106 | B. bison | SPLVMYKTVPVWKREPIRVLSLFGDIKKELTSLGFLEDGSKPGR |
| | DNMT3L | LKHLDDVTNIVRRDIDEWGPFDLTYGSTPTLGHTCDHPPGWYV |
| | Catalytic Domain | YQFHRILQYARPLPGSPQPFFWMFVDNLVLTEEDLDVATRFLET |
| | | DPVTIQDVRGRTVQNAVHVWSNIPAVKSRHSALVSQEELSLLA |
| | | QDRQRVKSPVQGPATLVKNCFLPLREYFKYFSTELTSSL |
| 107 | E. przewalskii | SPLEMYKTVPVWKREPVRVLSLFGDIKKELTTLGFLENGSDPGR |
| | DNMT3L | LKHLDDVTNTVRRDVEEWGPFDLVYGSTPPLGHACDHPPGWY |
| | Catalytic Domain | LFQFHRVLQYARPRPGSPQAFFWMFVDNLVLTEDDRAVATRFL |
| | | ETDPVTIQDVCGRAVRNAVHVWSNIPAVKSRHSALFSQEESFLR |
| | | AQDRQRAKPPARGPAKLVKNCFLPLREYFKYFSTEFTSSL |
| 108 | M. caroli | GPMEIYKTVSTWKRQPVRVLSLFGNIDKVLKSLGFLESGSGSGG |
| | DNMT3L | GTLKYVEDVTNVVRRDVEKWGPFDLVYGSTQPLGSSCDRCPG |
| | Catalytic Domain | WYMFQFHRILQYALPRQESQRPFFWIFMDNLLMTEDDQETTAR |
| | | FLQTEAVTLQDVRGRDYQNVMRVWSNIPGLKSKHVPLTPKEEE |
| | | YLQAQVRTRSKLDAQKVDLLVKNCLLPLREYFKYFS |
| 109 | P. troglodytes | NPLEMFETVPVWRRQPVRVLSLFEDIKKELTSLGFLESGSDPGQ |
| | DNMT3L | LKHVVDVTDTVRKDVEEWGPFDLVYGATPPLGHTCDRPPSWY |
| | Catalytic Domain | LFQFHRLLQYARPKPGSPRPFFWMFVDNLVLNKEDLDVASRFL |
| | | EMEPVTIPDVHGGSLQNAVRVWSNIPAIRSSRHWALVSEEELSL |
| | | LAQNKQSSKLAAKWPTKLVKNCFLPLREYFKYFSTELTSSL |
| 110 | Fusion Protein 9 | MGTMPKKKRKVPKKKRKVYNHDQEFDPPKVYPPVPAEKRKPI |
| | Amino Acid | RVLSLFDGIATGLLVLKDLGIQVDRYIASEVCEDSITVGMVRHQ |
| | Sequence | GKIMYVGDVRSVTQKHIQEWGPFDLVIGGSPCNDLSIVNPARK |
| | NLS-NLS-3A- | GLYEGTGRLFFEFYRLLHDARPKEGDDRPFFWLFENVVAMGVS |
| | h3L-dCas9- | DKRDISRFLESNPVMIDAKEVSAAHRARYFWGNLPGMNRPLAS |
| | KOX1KRAB- | TVNDKLELQECLEHGRIAKFSKVRTITTRSNSIKQGKDQHFPVF |
| | NLS-NLS | MNEKEDILWCTEMERVFGFPVHYTDVSNMSRLARQRLLGRSW |
| | | SVPVIRHLFAPLKEYFACVSSGNSNANSRGPSFSSGLVPLSLRGS |
| | | HMAAIPALDPEAEPSMDVILVGSSELSSSVSPGTGRDLIAYEVK |
| | | ANQRNIEDICICCGSLQVHTQHPLFEGGICAPCKDKFLDALFLYD |
| | | DDGYQSYCSICCSGETLLICGNPDCTRCYCFECVDSLVGPGTSG |
| | | KVHAMSNWVCYLCLPSSRSGLLQRRRKWRSQLKAFYDRESEN |
| | | PLEMFETVPVWRRQPVRVLSLFEDIKKELTSLGFLESGSDPGQL |
| | | KHVVDVTDTVRKDVEEWGPFDLVYGATPPLGHTCDRPPSWYL |
| | | FQFHRLLQYARPKPGSPRPFFWMFVDNLVLNKEDLDVASRFLE |
| | | MEPVTIPDVHGGSLQNAVRVWSNIPAIRSRHWALVSEEELSLLA |
| | | QNKQSSKLAAKWPTKLVKNCFLPLREYFKYFSTELTSSLGGPSS |
| | | GAPPPSGGSPAGSPTSTEEGTSESATPESGPGTSTEPSEGSAPGSP |
| | | AGSPTSTEEGTSTEPSEGSAPGTSTEPSELEDKKYSIGLAIGTNSV |
| | | GWAVITDEYKVPSKKFKVLGNTDRHSIKKNLIGALLFDSGETAE |
| | | ATRLKRTARRRYTRRKNRICYLQEIFSNEMAKVDDSFFHRLEES |
| | | FLVEEDKKHERHPIFGNIVDEVAYHEKYPTIYHLRKKLVDSTDK |
| | | ADLRLIYLALAHMIKFRGHFLIEGDLNPDNSDVDKLFIQLVQTY |
| | | NQLFEENPINASGVDAKAILSARLSKSRRLENLIAQLPGEKKNG |
| | | LFGNLIALSLGLTPNFKSNFDLAEDAKLQLSKDTYDDDLDNLLA |
| | | QIGDQY ADLFLAAKNLSDAILLSDILRVNTEITKAPLSASMIKRY |
| | | DEHHQDLTLLKALVRQQLPEKYKEIFFDQSKNGYAGYIDGGAS |
| | | QEEFYKFIKPILEKMDGTEELLVKLNREDLLRKQRTFDNGSIPH |
| | | QIHLGELHAILRRQEDFYPFLKDNREKIEKILTFRIPYYVGPLAR |
| | | GNSRFAWMTRKSEETITPWNFEEVVDKGASAQSFIERMTNFDK |
| | | NLPNEKVLPKHSLLYEYFTVYNELTKVKYVTEGMRKPAFLSGE |
| | | QKKAIVDLLFKTNRKVTVKQLKEDYFKKIECFDSVEISGVEDRF |
| | | NASLGTYHDLLKIIKDKDFLDNEENEDILEDIVLTLTLFEDREMI |
| | | EERLKTYAHLFDDKVMKQLKRRRYTGWGRLSRKLINGIRDKQ |
| | | SGKTILDFLKSDGFANRNFMQLIHDDSLTFKEDIQKAQVSGQGD |
| | | SLHEHIANLAGSPAIKKGILQTVKVVDELVKVMGRHKPENIVIE |
| | | MARENQTTQKGQKNSRERMKRIEEGIKELGSQILKEHPVENTQ |
| | | LQNEKLYLYYLQNGRDMYVDQELDINRLSDYDVDAIVPQSFLK |
| | | DDSIDNKVLTRSDKNRGKSDNVPSEEVVKKMKNYWRQLLNAK |
| | | LITQRKFDNLTKAERGGLSELDKAGFIKRQLVETRQITKHVAQI |
| | | LDSRMNTKYDENDKLIREVKVITLKSKLVSDFRKDFQFYKVREI |
| | | NNYHHAHDAYLNAVVGTALIKKYPKLESEFVYGDYKVYDVRK |
| | | MIAKSEQEIGKATAKYFFYSNIMNFFKTEITLANGEIRKRPLIETN |
| | | GETGEIVWDKGRDFATVRKVLSMPQVNIVKKTEVQTGGFSKES |
| | | ILPKRNSDKLIARKKDWDPKKYGGFDSPTVAYSVLVVAKVEKG |
| | | KSKKLKSVKELLGITIMERSSFEKNPIDFLEAKGYKEVKKDLIIK |
| | | LPKYSLFELENGRKRMLASAGELQKGNELALPSKYVNFLYLAS |
| | | HYEKLKGSPEDNEQKQLFVEQHKHYLDEIIEQISEFSKRVILADA |
| | | NLDKVLSAYNKHRDKPIREQAENIIHLFTLTNLGAPAAFKYFDT |
| | | TIDRKRYTSTKEVLDATLIHQSITGLYETRIDLSQLGGDSPKKKR |
| | | KVGVDGSSGSETPGTSESATPESRTLVTFKDVFVDFTREEWKLL |
| | | DTAQQIVYRNVMLENYKNLVSLGYQLTKPDVILRLEKGEEPSA |
| | | DYKDDDDKAPKKKRKVPKKKRKV |
| 111 | Fusion Protein 9 | ATGGGTACCATGCCAAAAAAGAAGAGAAAGGTACCGAAGA |
| | DNA Sequence | AAAAAAGAAAGGTATACAATCACGATCAGGAGTTCGACCCC |
| | | CCTAAGGTGTACCCACCAGTGCCTGCAGAGAAGAGGAAGCC |
| | | AATCCGGGTGCTGAGCCTGTTTGATGGCATCGCCACCGGCCT |
| | | GCTGGTGCTGAAGGATCTGGGCATCCAGGTGGACCGGTACA |
| | | TCGCCTCCGAGGTGTGCGAGGATTCTATCACCGTGGGCATGG |
| | | TGCGCCACCAGGGCAAGATCATGTATGTGGGCGACGTGCGG |
| | | TCCGTGACACAGAAGCACATCCAGGAGTGGGGCCCATTCGA |
| | | TCTGGTGATCGGCGGCAGCCCCTGTAATGACCTGTCCATCGT |
| | | GAACCCTGCAAGGAAGGGACTGTACGAGGGAACCGGCCGGC |
| | | TGTTCTTTGAGTTTTATAGACTGCTGCACGACGCCAGGCCTA |
| | | AGGAGGGCGACGATAGACCATTCTTTTGGCTGTTCGAGAATG |
| | | TGGTGGCTATGGGCGTGAGCGATAAGAGGGACATCTCCAGG |
| | | TTTCTGGAGTCTAACCCCGTGATGATCGATGCAAAGGAGGTG |
| | | TCCGCCGCACACAGAGCCAGGTATTTCTGGGGCAATCTGCCA |
| | | GGAATGAACAGGCCACTGGCAAGCACCGTGAATGACAAGCT |
| | | GGAGCTGCAGGAGTGCCTGGAGCACGGAAGGATCGCCAAGT |
| | | TTTCCAAGGTGCGCACAATCACCACACGGAGCAATTCCATCA |
| | | AGCAGGGCAAGGATCAGCACTTCCCCGTGTTCATGAACGAG |
| | | AAGGAGGACATCCTGTGGTGTACCGAGATGGAGAGAGTGTT |
| | | CGGCTTTCCAGTGCACTACACAGACGTGTCTAACATGAGCAG |
| | | GCTGGCAAGGCAGCGGCTGCTGGGCAGATCTTGGAGCGTGC |
| | | CCGTGATCAGGCACCTGTTCGCCCCTCTGAAGGAGTATTTTG |
| | | CCTGCGTGAGCAGCGGCAACTCCAATGCCAACAGCCGGGGC |
| | | CCCTCTTTCAGCTCCGGATTGGTGCCTCTGAGCCTGAGGGGC |
| | | TCCCACATGGCAGCAATCCCCGCCCTGGACCCCGAGGCCGA |
| | | GCCTAGCATGGACGTGATCCTGGTGGGCTCTAGCGAGCTGTC |
| | | CTCTAGCGTGTCTCCAGGAACCGGAAGGGATCTGATCGCATA |
| | | CGAGGTGAAGGCCAATCAGCGGAACATCGAGGACATCTGTA |
| | | TCTGCTGTGGCAGCCTGCAGGTGCACACACAGCACCCACTGT |
| | | TCGAGGGAGGAATCTGCGCACCCTGTAAGGATAAGTTCCTG |
| | | GACGCCCTGTTTCTGTACGACGATGACGGCTACCAGTCCTAT |
| | | TGCTCTATCTGCTGTTCCGGCGAGACCCTGCTGATCTGCGGC |
| | | AATCCAGATTGTACAAGGTGCTATTGTTTTGAGTGCGTGGAC |
| | | TCTCTGGTGGGACCAGGCACCAGCGGAAAGGTGCACGCCAT |
| | | GTCCAACTGGGTGTGCTACCTGTGCCTGCCATCCTCTCGCAG |
| | | CGGACTGCTGCAGCGGAGAAGGAAGTGGAGATCCCAGCTGA |
| | | AGGCCTTCTATGATAGGGAGTCTGAGAACCCCCTGGAGATGT |
| | | TTGAGACCGTGCCAGTGTGGCGCCGGCAGCCCGTGAGGGTG |
| | | CTGAGCCTGTTCGAGGATATCAAGAAGGAGCTGACATCCCT |
| | | GGGCTTTCTGGAGTCCGGCTCTGACCCCGGACAGCTGAAGCA |
| | | CGTGGTGGATGTGACCGACACAGTGCGGAAGGATGTGGAGG |
| | | AGTGGGGCCCTTTCGACCTGGTGTACGGAGCAACCCCTCCAC |
| | | TGGGACACACATGCGACAGACCCCCTTCTTGGTACCTGTTCC |
| | | AGTTTCACCGCCTGCTGCAGTATGCAAGGCCAAAGCCAGGC |
| | | AGCCCTAGACCATTCTTTTGGATGTTCGTGGATAATCTGGTG |
| | | CTGAACAAGGAGGATCTGGACGTGGCCAGCAGGTTTCTGGA |
| | | GATGGAGCCAGTGACCATCCCAGACGTGCACGGCGGCTCCC |
| | | TGCAGAATGCCGTGCGCGTGTGGTCTAACATCCCTGCCATCA |
| | | GAAGCAGGCACTGGGCACTGGTGAGCGAGGAGGAGCTGTCC |
| | | CTGCTGGCCCAGAATAAGCAGAGCAGCAAGCTGGCCGCCAA |
| | | GTGGCCTACAAAGCTGGTGAAGAACTGCTTCCTGCCACTGCG |
| | | GGAGTACTTCAAGTATTTTTCCACCGAGCTGACATCTAGCCT |
| | | GGGAGGACCCTCCTCTGGCGCCCCACCACCTAGCGGCGGCT |
| | | CCCCTGCCGGCTCTCCAACCAGCACAGAGGAGGGCACCAGC |
| | | GAGTCCGCCACACCAGAGTCTGGACCTGGCACCAGCACAGA |
| | | GCCATCCGAGGGCTCTGCCCCAGGCTCTCCTGCAGGCAGCCC |
| | | TACCTCCACCGAAGAGGGCACCAGCACAGAGCCTTCTGAGG |
| | | GCAGCGCCCCAGGCACCTCTACAGAGCCAAGCGAGCTCGAG |
| | | GACAAGAAGTACAGCATCGGCCTGGCCATCGGCACCAACTC |
| | | TGTGGGCTGGGCCGTGATCACCGACGAGTACAAGGTGCCCA |
| | | GCAAGAAATTCAAGGTGCTGGGCAACACCGACCGGCACAGC |
| | | ATCAAGAAGAACCTGATCGGAGCCCTGCTGTTCGACAGCGG |
| | | CGAAACAGCCGAGGCCACCCGGCTGAAGAGAACCGCCAGA |
| | | AGAAGATACACCAGACGGAAGAACCGGATCTGCTATCTGCA |
| | | AGAGATCTTCAGCAACGAGATGGCCAAGGTGGACGACAGCT |
| | | TCTTCCACAGACTGGAAGAGTCCTTCCTGGTGGAAGAGGATA |
| | | AGAAGCACGAGCGGCACCCCATCTTCGGCAACATCGTGGAC |
| | | GAGGTGGCCTACCACGAGAAGTACCCCACCATCTACCACCT |
| | | GAGAAAGAAACTGGTGGACAGCACCGACAAGGCCGACCTGC |
| | | GGCTGATCTATCTGGCCCTGGCCCACATGATCAAGTTCCGGG |
| | | GCCACTTCCTGATCGAGGGCGACCTGAACCCCGACAACAGC |
| | | GACGTGGACAAGCTGTTCATCCAGCTGGTGCAGACCTACAA |
| | | CCAGCTGTTCGAGGAAAACCCCATCAACGCCAGCGGCGTGG |
| | | ACGCCAAGGCCATCCTGTCTGCCAGACTGAGCAAGAGCAGA |
| | | CGGCTGGAAAATCTGATCGCCCAGCTGCCCGGCGAGAAGAA |
| | | GAATGGCCTGTTCGGCAACCTGATTGCCCTGAGCCTGGGCCT |
| | | GACCCCCAACTTCAAGAGCAACTTCGACCTGGCCGAGGATG |
| | | CCAAACTGCAGCTGAGCAAGGACACCTACGACGACGACCTG |
| | | GACAACCTGCTGGCCCAGATCGGCGACCAGTACGCCGACCT |
| | | GTTTCTGGCCGCCAAGAACCTGTCCGACGCCATCCTGCTGAG |
| | | CGACATCCTGAGAGTGAACACCGAGATCACCAAGGCCCCCC |
| | | TGAGCGCCTCTATGATCAAGAGATACGACGAGCACCACCAG |
| | | GACCTGACCCTGCTGAAAGCTCTCGTGCGGCAGCAGCTGCCT |
| | | GAGAAGTACAAAGAGATTTTCTTCGACCAGAGCAAGAACGG |
| | | CTACGCCGGCTACATTGACGGCGGAGCCAGCCAGGAAGAGT |
| | | TCTACAAGTTCATCAAGCCCATCCTGGAAAAGATGGACGGC |
| | | ACCGAGGAACTGCTCGTGAAGCTGAACAGAGAGGACCTGCT |
| | | GCGGAAGCAGCGGACCTTCGACAACGGCAGCATCCCCCACC |
| | | AGATCCACCTGGGAGAGCTGCACGCCATTCTGCGGCGGCAG |
| | | GAAGATTTTTACCCATTCCTGAAGGACAACCGGGAAAAGAT |
| | | CGAGAAGATCCTGACCTTCCGCATCCCCTACTACGTGGGCCC |
| | | TCTGGCCAGGGGAAACAGCAGATTCGCCTGGATGACCAGAA |
| | | AGAGCGAGGAAACCATCACCCCCTGGAACTTCGAGGAAGTG |
| | | GTGGACAAGGGCGCTTCCGCCCAGAGCTTCATCGAGCGGAT |
| | | GACCAACTTCGATAAGAACCTGCCCAACGAGAAGGTGCTGC |
| | | CCAAGCACAGCCTGCTGTACGAGTACTTCACCGTGTATAACG |
| | | AGCTGACCAAAGTGAAATACGTGACCGAGGGAATGAGAAAG |
| | | CCCGCCTTCCTGAGCGGCGAGCAGAAAAAGGCCATCGTGGA |
| | | CCTGCTGTTCAAGACCAACCGGAAAGTGACCGTGAAGCAGC |
| | | TGAAAGAGGACTACTTCAAGAAAATCGAGTGCTTCGACTCC |
| | | GTGGAAATCTCCGGCGTGGAAGATCGGTTCAACGCCTCCCTG |
| | | GGCACATACCACGATCTGCTGAAAATTATCAAGGACAAGGA |
| | | CTTCCTGGACAATGAGGAAAACGAGGACATTCTGGAAGATA |
| | | TCGTGCTGACCCTGACACTGTTTGAGGACAGAGAGATGATCG |
| | | AGGAACGGCTGAAAACCTATGCCCACCTGTTCGACGACAAA |
| | | GTGATGAAGCAGCTGAAGCGGCGGAGATACACCGGCTGGGG |
| | | CAGGCTGAGCCGGAAGCTGATCAACGGCATCCGGGACAAGC |
| | | AGTCCGGCAAGACAATCCTGGATTTCCTGAAGTCCGACGGCT |
| | | TCGCCAACAGAAACTTCATGCAGCTGATCCACGACGACAGC |
| | | CTGACCTTTAAAGAGGACATCCAGAAAGCCCAGGTGTCCGG |
| | | CCAGGGCGATAGCCTGCACGAGCACATTGCCAATCTGGCCG |
| | | GCAGCCCCGCCATTAAGAAGGGCATCCTGCAGACAGTGAAG |
| | | GTGGTGGACGAGCTCGTGAAAGTGATGGGCCGGCACAAGCC |
| | | CGAGAACATCGTGATCGAAATGGCCAGAGAGAACCAGACCA |
| | | CCCAGAAGGGACAGAAGAACAGCCGCGAGAGAATGAAGCG |
| | | GATCGAAGAGGGCATCAAAGAGCTGGGCAGCCAGATCCTGA |
| | | AAGAACACCCCGTGGAAAACACCCAGCTGCAGAACGAGAAG |
| | | CTGTACCTGTACTACCTGCAGAATGGGGGGATATGTACGTG |
| | | GACCAGGAACTGGACATCAACCGGCTGTCCGACTACGATGT |
| | | GGACGCCATCGTGCCTCAGAGCTTTCTGAAGGACGACTCCAT |
| | | CGACAACAAGGTGCTGACCAGAAGCGACAAGAACCGGGGC |
| | | AAGAGCGACAACGTGCCCTCCGAAGAGGTCGTGAAGAAGAT |
| | | GAAGAACTACTGGCGGCAGCTGCTGAACGCCAAGCTGATTA |
| | | CCCAGAGAAAGTTCGACAATCTGACCAAGGCCGAGAGAGGC |
| | | GGCCTGAGCGAACTGGATAAGGCCGGCTTCATCAAGAGACA |
| | | GCTGGTGGAAACCCGGCAGATCACAAAGCACGTGGCACAGA |
| | | TCCTGGACTCCCGGATGAACACTAAGTACGACGAGAATGAC |
| | | AAGCTGATCCGGGAAGTGAAAGTGATCACCCTGAAGTCCAA |
| | | GCTGGTGTCCGATTTCCGGAAGGATTTCCAGTTTTACAAAGT |
| | | GCGCGAGATCAACAACTACCACCACGCCCACGACGCCTACC |
| | | TGAACGCCGTCGTGGGAACCGCCCTGATCAAAAAGTACCCT |
| | | AAGCTGGAAAGCGAGTTCGTGTACGGCGACTACAAGGTGTA |
| | | CGACGTGCGGAAGATGATCGCCAAGAGCGAGCAGGAAATCG |
| | | GCAAGGCTACCGCCAAGTACTTCTTCTACAGCAACATCATGA |
| | | ACTTTTTCAAGACCGAGATTACCCTGGCCAACGGCGAGATCC |
| | | GGAAGCGGCCTCTGATCGAGACAAACGGCGAAACCGGGGAG |
| | | ATCGTGTGGGATAAGGGCCGGGATTTTGCCACCGTGCGGAA |
| | | AGTGCTGAGCATGCCCCAAGTGAATATCGTGAAAAAGACCG |
| | | AGGTGCAGACAGGCGGCTTCAGCAAAGAGTCTATCCTGCCC |
| | | AAGAGGAACAGCGATAAGCTGATCGCCAGAAAGAAGGACT |
| | | GGGACCCTAAGAAGTACGGCGGCTTCGACAGCCCCACCGTG |
| | | GCCTATTCTGTGCTGGTGGTGGCCAAAGTGGAAAAGGGCAA |
| | | GTCCAAGAAACTGAAGAGTGTGAAAGAGCTGCTGGGGATCA |
| | | CCATCATGGAAAGAAGCAGCTTCGAGAAGAATCCCATCGAC |
| | | TTTCTGGAAGCCAAGGGCTACAAAGAAGTGAAAAAGGACCT |
| | | GATCATCAAGCTGCCTAAGTACTCCCTGTTCGAGCTGGAAAA |
| | | CGGCCGGAAGAGAATGCTGGCCTCTGCCGGCGAACTGCAGA |
| | | AGGGAAACGAACTGGCCCTGCCCTCCAAATATGTGAACTTCC |
| | | TGTACCTGGCCAGCCACTATGAGAAGCTGAAGGGCTCCCCC |
| | | GAGGATAATGAGCAGAAACAGCTGTTTGTGGAACAGCACAA |
| | | GCACTACCTGGACGAGATCATCGAGCAGATCAGCGAGTTCT |
| | | CCAAGAGAGTGATCCTGGCCGACGCTAATCTGGACAAAGTG |
| | | CTGTCCGCCTACAACAAGCACCGGGATAAGCCCATCAGAGA |
| | | GCAGGCCGAGAATATCATCCACCTGTTTACCCTGACCAATCT |
| | | GGGAGCCCCTGCCGCCTTCAAGTACTTTGACACCACCATCGA |
| | | CCGGAAGAGGTACACCAGCACCAAAGAGGTGCTGGACGCCA |
| | | CCCTGATCCACCAGAGCATCACCGGCCTGTACGAGACACGG |
| | | ATCGACCTGTCTCAGCTGGGAGGCGACAGCCCCAAGAAGAA |
| | | GAGAAAGGTGGGAGTCGACGGATCCAGCGGCTCCGAGACCC |
| | | CAGGCACATCTGAGAGCGCCACCCCTGAGTCCCGGACCCTG |
| | | GTGACCTTCAAGGATGTATTTGTGGACTTCACCAGGGAGGAG |
| | | TGGAAGCTGCTGGACACTGCTCAGCAGATCGTGTACAGAAA |
| | | TGTGATGCTGGAGAACTATAAGAACCTGGTTTCCTTGGGTTA |
| | | TCAGCTTACTAAGCCAGATGTGATCCTCCGGTTGGAGAAGGG |
| | | AGAAGAGCCCAGCGCTGATTACAAAGATGATGACGATAAAG |
| | | CCCCAAAAAAGAAGAGAAAGGTACCGAAGAAAAAAAGAAA |
| | | GGTCTGA |
| 112 | Fusion Protein 10 | MGTMPKKKRKVPKKKRKVYNHDQEFDPPKVYPPVPAEKRKPI |
| | Amino Acid | RVLSLFDGIATGLLVLKDLGIQVDRYIASEVCEDSITVGMVRHQ |
| | Sequence | GKIMYVGDVRSVTQKHIQEWGPFDLVIGGSPCNDLSIVNPARK |
| | NLS-NLS-3A- | GLYEGTGRLFFEFYRLLHDARPKEGDDRPFFWLFENVVAMGVS |
| | h3L-dCas9-ZFP- | DKRDISRFLESNPVMIDAKEVSAAHRARYFWGNLPGMNRPLAS |
| | 28-NLS-NLS | TVNDKLELQECLEHGRIAKFSKVRTITTRSNSIKQGKDQHFPVF |
| | | MNEKEDILWCTEMERVFGFPVHYTDVSNMSRLARQRLLGRSW |
| | | SVPVIRHLFAPLKEYFACVSSGNSNANSRGPSFSSGLVPLSLRGS |
| | | HMAAIPALDPEAEPSMDVILVGSSELSSSVSPGTGRDLIAYEVK |
| | | ANQRNIEDICICCGSLQVHTQHPLFEGGICAPCKDKFLDALFLYD |
| | | DDGYQSYCSICCSGETLLICGNPDCTRCYCFECVDSLVGPGTSG |
| | | KVHAMSNWVCYLCLPSSRSGLLQRRRKWRSQLKAFYDRESEN |
| | | PLEMFETVPVWRRQPVRVLSLFEDIKKELTSLGFLESGSDPGQL |
| | | KHVVDVTDTVRKDVEEWGPFDLVYGATPPLGHTCDRPPSWYL |
| | | FQFHRLLQYARPKPGSPRPFFWMFVDNLVLNKEDLDVASRFLE |
| | | MEPVTIPDVHGGSLQNAVRVWSNIPAIRSRHWALVSEEELSLLA |
| | | QNKQSSKLAAKWPTKLVKNCFLPLREYFKYFSTELTSSLGGPSS |
| | | GAPPPSGGSPAGSPTSTEEGTSESATPESGPGTSTEPSEGSAPGSP |
| | | AGSPTSTEEGTSTEPSEGSAPGTSTEPSELEDKKYSIGLAIGTNSV |
| | | GWAVITDEYKVPSKKFKVLGNTDRHSIKKNLIGALLFDSGETAE |
| | | ATRLKRTARRRYTRRKNRICYLQEIFSNEMAKVDDSFFHRLEES |
| | | FLVEEDKKHERHPIFGNIVDEVAYHEKYPTIYHLRKKLVDSTDK |
| | | ADLRLIYLALAHMIKFRGHFLIEGDLNPDNSDVDKLFIQLVQTY |
| | | NQLFEENPINASGVDAKAILSARLSKSRRLENLIAQLPGEKKNG |
| | | LFGNLIALSLGLTPNFKSNFDLAEDAKLQLSKDTYDDDLDNLLA |
| | | QIGDQYADLFLAAKNLSDAILLSDILRVNTEITKAPLSASMIKRY |
| | | DEHHQDLTLLKALVRQQLPEKYKEIFFDQSKNGYAGYIDGGAS |
| | | QEEFYKFIKPILEKMDGTEELLVKLNREDLLRKQRTFDNGSIPH |
| | | QIHLGELHAILRRQEDFYPFLKDNREKIEKILTFRIPYYVGPLAR |
| | | GNSRFAWMTRKSEETITPWNFEEVVDKGASAQSFIERMTNFDK |
| | | NLPNEKVLPKHSLLYEYFTVYNELTKVKYVTEGMRKPAFLSGE |
| | | QKKAIVDLLFKTNRKVTVKQLKEDYFKKIECFDSVEISGVEDRF |
| | | NASLGTYHDLLKIIKDKDFLDNEENEDILEDIVLTLTLFEDREMI |
| | | EERLKTYAHLFDDKVMKQLKRRRYTGWGRLSRKLINGIRDKQ |
| | | SGKTILDFLKSDGFANRNFMQLIHDDSLTFKEDIQKAQVSGQGD |
| | | SLHEHIANLAGSPAIKKGILQTVKVVDELVKVMGRHKPENIVIE |
| | | MARENQTTQKGQKNSRERMKRIEEGIKELGSQILKEHPVENTQ |
| | | LQNEKLYLYYLQNGRDMYVDQELDINRLSDYDVDAIVPQSFLK |
| | | DDSIDNKVLTRSDKNRGKSDNVPSEEVVKKMKNYWRQLLNAK |
| | | LITQRKFDNLTKAERGGLSELDKAGFIKRQLVETRQITKHVAQI |
| | | LDSRMNTKYDENDKLIREVKVITLKSKLVSDFRKDFQFYKVREI |
| | | NNYHHAHDAYLNAVVGTALIKKYPKLESEFVYGDYKVYDVRK |
| | | MIAKSEQEIGKATAKYFFYSNIMNFFKTEITLANGEIRKRPLIETN |
| | | GETGEIVWDKGRDFATVRKVLSMPQVNIVKKTEVQTGGFSKES |
| | | ILPKRNSDKLIARKKDWDPKKYGGFDSPTVAYSVLVVAKVEKG |
| | | KSKKLKSVKELLGITIMERSSFEKNPIDFLEAKGYKEVKKDLIIK |
| | | LPKYSLFELENGRKRMLASAGELQKGNELALPSKYVNFLYLAS |
| | | HYEKLKGSPEDNEQKQLFVEQHKHYLDEIIEQISEFSKRVILADA |
| | | NLDKVLSAYNKHRDKPIREQAENIIHLFTLTNLGAPAAFKYFDT |
| | | TIDRKRYTSTKEVLDATLIHQSITGLYETRIDLSQLGGDSPKKKR |
| | | KVGVDGSSGSETPGTSESATPESTGNKKLEAVGTGIEPKAMSQG |
| | | LVTFGDVAVDFSQEEWEWLNPIQRNLYRKVMLENYRNLASLG |
| | | LCVSKPDVISSLEQGKEPWSADYKDDDDKAPKKKRKVPKKKR |
| | | KV |
| 113 | Fusion Protein 10 | ATGGGTACCATGCCAAAAAAGAAGAGAAAGGTACCGAAGA |
| | DNA Sequence | AAAAAAGAAAGGTATACAATCACGATCAGGAGTTCGACCCC |
| | | CCTAAGGTGTACCCACCAGTGCCTGCAGAGAAGAGGAAGCC |
| | | AATCCGGGTGCTGAGCCTGTTTGATGGCATCGCCACCGGCCT |
| | | GCTGGTGCTGAAGGATCTGGGCATCCAGGTGGACCGGTACA |
| | | TCGCCTCCGAGGTGTGCGAGGATTCTATCACCGTGGGCATGG |
| | | TGCGCCACCAGGGCAAGATCATGTATGTGGGCGACGTGCGG |
| | | TCCGTGACACAGAAGCACATCCAGGAGTGGGGCCCATTCGA |
| | | TCTGGTGATCGGCGGCAGCCCCTGTAATGACCTGTCCATCGT |
| | | GAACCCTGCAAGGAAGGGACTGTACGAGGGAACCGGCCGGC |
| | | TGTTCTTTGAGTTTTATAGACTGCTGCACGACGCCAGGCCTA |
| | | AGGAGGGCGACGATAGACCATTCTTTTGGCTGTTCGAGAATG |
| | | TGGTGGCTATGGGCGTGAGCGATAAGAGGGACATCTCCAGG |
| | | TTTCTGGAGTCTAACCCCGTGATGATCGATGCAAAGGAGGTG |
| | | TCCGCCGCACACAGAGCCAGGTATTTCTGGGGCAATCTGCCA |
| | | GGAATGAACAGGCCACTGGCAAGCACCGTGAATGACAAGCT |
| | | GGAGCTGCAGGAGTGCCTGGAGCACGGAAGGATCGCCAAGT |
| | | TTTCCAAGGTGCGCACAATCACCACACGGAGCAATTCCATCA |
| | | AGCAGGGCAAGGATCAGCACTTCCCCGTGTTCATGAACGAG |
| | | AAGGAGGACATCCTGTGGTGTACCGAGATGGAGAGAGTGTT |
| | | CGGCTTTCCAGTGCACTACACAGACGTGTCTAACATGAGCAG |
| | | GCTGGCAAGGCAGCGGCTGCTGGGCAGATCTTGGAGCGTGC |
| | | CCGTGATCAGGCACCTGTTCGCCCCTCTGAAGGAGTATTTTG |
| | | CCTGCGTGAGCAGCGGCAACTCCAATGCCAACAGCCGGGGC |
| | | CCCTCTTTCAGCTCCGGATTGGTGCCTCTGAGCCTGAGGGGC |
| | | TCCCACATGGCAGCAATCCCCGCCCTGGACCCCGAGGCCGA |
| | | GCCTAGCATGGACGTGATCCTGGTGGGCTCTAGCGAGCTGTC |
| | | CTCTAGCGTGTCTCCAGGAACCGGAAGGGATCTGATCGCATA |
| | | CGAGGTGAAGGCCAATCAGCGGAACATCGAGGACATCTGTA |
| | | TCTGCTGTGGCAGCCTGCAGGTGCACACACAGCACCCACTGT |
| | | TCGAGGGAGGAATCTGCGCACCCTGTAAGGATAAGTTCCTG |
| | | GACGCCCTGTTTCTGTACGACGATGACGGCTACCAGTCCTAT |
| | | TGCTCTATCTGCTGTTCCGGCGAGACCCTGCTGATCTGCGGC |
| | | AATCCAGATTGTACAAGGTGCTATTGTTTTGAGTGCGTGGAC |
| | | TCTCTGGTGGGACCAGGCACCAGCGGAAAGGTGCACGCCAT |
| | | GTCCAACTGGGTGTGCTACCTGTGCCTGCCATCCTCTCGCAG |
| | | CGGACTGCTGCAGCGGAGAAGGAAGTGGAGATCCCAGCTGA |
| | | AGGCCTTCTATGATAGGGAGTCTGAGAACCCCCTGGAGATGT |
| | | TTGAGACCGTGCCAGTGTGGCGCCGGCAGCCCGTGAGGGTG |
| | | CTGAGCCTGTTCGAGGATATCAAGAAGGAGCTGACATCCCT |
| | | GGGCTTTCTGGAGTCCGGCTCTGACCCCGGACAGCTGAAGCA |
| | | CGTGGTGGATGTGACCGACACAGTGCGGAAGGATGTGGAGG |
| | | AGTGGGGCCCTTTCGACCTGGTGTACGGAGCAACCCCTCCAC |
| | | TGGGACACACATGCGACAGACCCCCTTCTTGGTACCTGTTCC |
| | | AGTTTCACCGCCTGCTGCAGTATGCAAGGCCAAAGCCAGGC |
| | | AGCCCTAGACCATTCTTTTGGATGTTCGTGGATAATCTGGTG |
| | | CTGAACAAGGAGGATCTGGACGTGGCCAGCAGGTTTCTGGA |
| | | GATGGAGCCAGTGACCATCCCAGACGTGCACGGCGGCTCCC |
| | | TGCAGAATGCCGTGCGCGTGTGGTCTAACATCCCTGCCATCA |
| | | GAAGCAGGCACTGGGCACTGGTGAGCGAGGAGGAGCTGTCC |
| | | CTGCTGGCCCAGAATAAGCAGAGCAGCAAGCTGGCCGCCAA |
| | | GTGGCCTACAAAGCTGGTGAAGAACTGCTTCCTGCCACTGCG |
| | | GGAGTACTTCAAGTATTTTTCCACCGAGCTGACATCTAGCCT |
| | | GGGAGGACCCTCCTCTGGCGCCCCACCACCTAGCGGCGGCT |
| | | CCCCTGCCGGCTCTCCAACCAGCACAGAGGAGGGCACCAGC |
| | | GAGTCCGCCACACCAGAGTCTGGACCTGGCACCAGCACAGA |
| | | GCCATCCGAGGGCTCTGCCCCGGGCTCTCCTGCAGGCAGCCC |
| | | TACCTCCACCGAAGAGGGCACCAGCACAGAGCCTTCTGAGG |
| | | GCAGCGCCCCAGGCACCTCTACAGAGCCAAGCGAGCTCGAG |
| | | GACAAGAAGTACAGCATCGGCCTGGCCATCGGCACCAACTC |
| | | TGTGGGCTGGGCCGTGATCACCGACGAGTACAAGGTGCCCA |
| | | GCAAGAAATTCAAGGTGCTGGGCAACACCGACCGGCACAGC |
| | | ATCAAGAAGAACCTGATCGGAGCCCTGCTGTTCGACAGCGG |
| | | CGAAACAGCCGAGGCCACCCGGCTGAAGAGAACCGCCAGA |
| | | AGAAGATACACCAGACGGAAGAACCGGATCTGCTATCTGCA |
| | | AGAGATCTTCAGCAACGAGATGGCCAAGGTGGACGACAGCT |
| | | TCTTCCACAGACTGGAAGAGTCCTTCCTGGTGGAAGAGGATA |
| | | AGAAGCACGAGCGGCACCCCATCTTCGGCAACATCGTGGAC |
| | | GAGGTGGCCTACCACGAGAAGTACCCCACCATCTACCACCT |
| | | GAGAAAGAAACTGGTGGACAGCACCGACAAGGCCGACCTGC |
| | | GGCTGATCTATCTGGCCCTGGCCCACATGATCAAGTTCCGGG |
| | | GCCACTTCCTGATCGAGGGCGACCTGAACCCCGACAACAGC |
| | | GACGTGGACAAGCTGTTCATCCAGCTGGTGCAGACCTACAA |
| | | CCAGCTGTTCGAGGAAAACCCCATCAACGCCAGCGGCGTGG |
| | | ACGCCAAGGCCATCCTGTCTGCCAGACTGAGCAAGAGCAGA |
| | | CGGCTGGAAAATCTGATCGCCCAGCTGCCCGGCGAGAAGAA |
| | | GAATGGCCTGTTCGGCAACCTGATTGCCCTGAGCCTGGGCCT |
| | | GACCCCCAACTTCAAGAGCAACTTCGACCTGGCCGAGGATG |
| | | CCAAACTGCAGCTGAGCAAGGACACCTACGACGACGACCTG |
| | | GACAACCTGCTGGCCCAGATCGGCGACCAGTACGCCGACCT |
| | | GTTTCTGGCCGCCAAGAACCTGTCCGACGCCATCCTGCTGAG |
| | | CGACATCCTGAGAGTGAACACCGAGATCACCAAGGCCCCCC |
| | | TGAGCGCCTCTATGATCAAGAGATACGACGAGCACCACCAG |
| | | GACCTGACCCTGCTGAAAGCTCTCGTGCGGCAGCAGCTGCCT |
| | | GAGAAGTACAAAGAGATTTTCTTCGACCAGAGCAAGAACGG |
| | | CTACGCCGGCTACATTGACGGCGGAGCCAGCCAGGAAGAGT |
| | | TCTACAAGTTCATCAAGCCCATCCTGGAAAAGATGGACGGC |
| | | ACCGAGGAACTGCTCGTGAAGCTGAACAGAGAGGACCTGCT |
| | | GCGGAAGCAGCGGACCTTCGACAACGGCAGCATCCCCCACC |
| | | AGATCCACCTGGGAGAGCTGCACGCCATTCTGCGGCGGCAG |
| | | GAAGATTTTTACCCATTCCTGAAGGACAACCGGGAAAAGAT |
| | | CGAGAAGATCCTGACCTTCCGCATCCCCTACTACGTGGGCCC |
| | | TCTGGCCAGGGGAAACAGCAGATTCGCCTGGATGACCAGAA |
| | | AGAGCGAGGAAACCATCACCCCCTGGAACTTCGAGGAAGTG |
| | | GTGGACAAGGGCGCTTCCGCCCAGAGCTTCATCGAGCGGAT |
| | | GACCAACTTCGATAAGAACCTGCCCAACGAGAAGGTGCTGC |
| | | CCAAGCACAGCCTGCTGTACGAGTACTTCACCGTGTATAACG |
| | | AGCTGACCAAAGTGAAATACGTGACCGAGGGAATGAGAAAG |
| | | CCCGCCTTCCTGAGCGGCGAGCAGAAAAAGGCCATCGTGGA |
| | | CCTGCTGTTCAAGACCAACCGGAAAGTGACCGTGAAGCAGC |
| | | TGAAAGAGGACTACTTCAAGAAAATCGAGTGCTTCGACTCC |
| | | GTGGAAATCTCCGGCGTGGAAGATCGGTTCAACGCCTCCCTG |
| | | GGCACATACCACGATCTGCTGAAAATTATCAAGGACAAGGA |
| | | CTTCCTGGACAATGAGGAAAACGAGGACATTCTGGAAGATA |
| | | TCGTGCTGACCCTGACACTGTTTGAGGACAGAGAGATGATCG |
| | | AGGAACGGCTGAAAACCTATGCCCACCTGTTCGACGACAAA |
| | | GTGATGAAGCAGCTGAAGCGGCGGAGATACACCGGCTGGGG |
| | | CAGGCTGAGCCGGAAGCTGATCAACGGCATCCGGGACAAGC |
| | | AGTCCGGCAAGACAATCCTGGATTTCCTGAAGTCCGACGGCT |
| | | TCGCCAACAGAAACTTCATGCAGCTGATCCACGACGACAGC |
| | | CTGACCTTTAAAGAGGACATCCAGAAAGCCCAGGTGTCCGG |
| | | CCAGGGCGATAGCCTGCACGAGCACATTGCCAATCTGGCCG |
| | | GCAGCCCCGCCATTAAGAAGGGCATCCTGCAGACAGTGAAG |
| | | GTGGTGGACGAGCTCGTGAAAGTGATGGGCCGGCACAAGCC |
| | | CGAGAACATCGTGATCGAAATGGCCAGAGAGAACCAGACCA |
| | | CCCAGAAGGGACAGAAGAACAGCCGCGAGAGAATGAAGCG |
| | | GATCGAAGAGGGCATCAAAGAGCTGGGCAGCCAGATCCTGA |
| | | AAGAACACCCCGTGGAAAACACCCAGCTGCAGAACGAGAAG |
| | | CTGTACCTGTACTACCTGCAGAATGGGCGGGATATGTACGTG |
| | | GACCAGGAACTGGACATCAACCGGCTGTCCGACTACGATGT |
| | | GGACGCCATCGTGCCTCAGAGCTTTCTGAAGGACGACTCCAT |
| | | CGACAACAAGGTGCTGACCAGAAGCGACAAGAACCGGGGC |
| | | AAGAGCGACAACGTGCCCTCCGAAGAGGTCGTGAAGAAGAT |
| | | GAAGAACTACTGGCGGCAGCTGCTGAACGCCAAGCTGATTA |
| | | CCCAGAGAAAGTTCGACAATCTGACCAAGGCCGAGAGAGGC |
| | | GGCCTGAGCGAACTGGATAAGGCCGGCTTCATCAAGAGACA |
| | | GCTGGTGGAAACCCGGCAGATCACAAAGCACGTGGCACAGA |
| | | TCCTGGACTCCCGGATGAACACTAAGTACGACGAGAATGAC |
| | | AAGCTGATCCGGGAAGTGAAAGTGATCACCCTGAAGTCCAA |
| | | GCTGGTGTCCGATTTCCGGAAGGATTTCCAGTTTTACAAAGT |
| | | GCGCGAGATCAACAACTACCACCACGCCCACGACGCCTACC |
| | | TGAACGCCGTCGTGGGAACCGCCCTGATCAAAAAGTACCCT |
| | | AAGCTGGAAAGCGAGTTCGTGTACGGCGACTACAAGGTGTA |
| | | CGACGTGCGGAAGATGATCGCCAAGAGCGAGCAGGAAATCG |
| | | GCAAGGCTACCGCCAAGTACTTCTTCTACAGCAACATCATGA |
| | | ACTTTTTCAAGACCGAGATTACCCTGGCCAACGGCGAGATCC |
| | | GGAAGCGGCCTCTGATCGAGACAAACGGCGAAACCGGGGAG |
| | | ATCGTGTGGGATAAGGGCCGGGATTTTGCCACCGTGCGGAA |
| | | AGTGCTGAGCATGCCCCAAGTGAATATCGTGAAAAAGACCG |
| | | AGGTGCAGACAGGCGGCTTCAGCAAAGAGTCTATCCTGCCC |
| | | AAGAGGAACAGCGATAAGCTGATCGCCAGAAAGAAGGACT |
| | | GGGACCCTAAGAAGTACGGCGGCTTCGACAGCCCCACCGTG |
| | | GCCTATTCTGTGCTGGTGGTGGCCAAAGTGGAAAAGGGCAA |
| | | GTCCAAGAAACTGAAGAGTGTGAAAGAGCTGCTGGGGATCA |
| | | CCATCATGGAAAGAAGCAGCTTCGAGAAGAATCCCATCGAC |
| | | TTTCTGGAAGCCAAGGGCTACAAAGAAGTGAAAAAGGACCT |
| | | GATCATCAAGCTGCCTAAGTACTCCCTGTTCGAGCTGGAAAA |
| | | CGGCCGGAAGAGAATGCTGGCCTCTGCCGGCGAACTGCAGA |
| | | AGGGAAACGAACTGGCCCTGCCCTCCAAATATGTGAACTTCC |
| | | TGTACCTGGCCAGCCACTATGAGAAGCTGAAGGGCTCCCCC |
| | | GAGGATAATGAGCAGAAACAGCTGTTTGTGGAACAGCACAA |
| | | GCACTACCTGGACGAGATCATCGAGCAGATCAGCGAGTTCT |
| | | CCAAGAGAGTGATCCTGGCCGACGCTAATCTGGACAAAGTG |
| | | CTGTCCGCCTACAACAAGCACCGGGATAAGCCCATCAGAGA |
| | | GCAGGCCGAGAATATCATCCACCTGTTTACCCTGACCAATCT |
| | | GGGAGCCCCTGCCGCCTTCAAGTACTTTGACACCACCATCGA |
| | | CCGGAAGAGGTACACCAGCACCAAAGAGGTGCTGGACGCCA |
| | | CCCTGATCCACCAGAGCATCACCGGCCTGTACGAGACACGG |
| | | ATCGACCTGTCTCAGCTGGGAGGCGACAGCCCCAAGAAGAA |
| | | GAGAAAGGTGGGAGTCGACGGATCCAGCGGCTCCGAGACCC |
| | | CAGGCACATCTGAGAGCGCCACCCCTGAGTCCACCGGTAAC |
| | | AAAAAGCTTGAGGCCGTCGGAACCGGAATCGAACCAAAAGC |
| | | AATGTCCCAGGGTTTGGTGACATTTGGCGACGTGGCTGTCGA |
| | | TTTTTCCCAGGAAGAGTGGGAGTGGCTCAATCCTATCCAGAG |
| | | GAACTTGTACCGGAAGGTGATGCTGGAGAATTATAGAAATT |
| | | TGGCATCACTGGGGTTGTGCGTTAGCAAACCAGATGTTATAT |
| | | CTTCCCTGGAACAGGGAAAGGAGCCCTGGAGCGCTGATTAC |
| | | AAAGATGATGACGATAAAGCCCCCAAGAAGAAAAGGAAGG |
| | | TCCCAAAGAAAAAAAGAAAGGTGTGA |
| 114 | Fusion Protein 11 | MGTMPKKKRKVPKKKRKVYNHDQEFDPPKVYPPVPAEKRKPI |
| | Amino Acid | RVLSLFDGIATGLLVLKDLGIQVDRYIASEVCEDSITVGMVRHQ |
| | Sequence | GKIMYVGDVRSVTQKHIQEWGPFDLVIGGSPCNDLSIVNPARK |
| | NLS-NLS-3A- | GLYEGTGRLFFEFYRLLHDARPKEGDDRPFFWLFENVVAMGVS |
| | h3L-dCas9- | DKRDISRFLESNPVMIDAKEVSAAHRARYFWGNLPGMNRPLAS |
| | ZIM3-NLS-NLS | TVNDKLELQECLEHGRIAKFSKVRTITTRSNSIKQGKDQHFPVF |
| | | MNEKEDILWCTEMERVFGFPVHYTDVSNMSRLARQRLLGRSW |
| | | SVPVIRHLFAPLKEYFACVSSGNSNANSRGPSFSSGLVPLSLRGS |
| | | HMAAIPALDPEAEPSMDVILVGSSELSSSVSPGTGRDLIAYEVK |
| | | ANQRNIEDICICCGSLQVHTQHPLFEGGICAPCKDKFLDALFLYD |
| | | DDGYQSYCSICCSGETLLICGNPDCTRCYCFECVDSLVGPGTSG |
| | | KVHAMSNWVCYLCLPSSRSGLLQRRRKWRSQLKAFYDRESEN |
| | | PLEMFETVPVWRRQPVRVLSLFEDIKKELTSLGFLESGSDPGQL |
| | | KHVVDVTDTVRKDVEEWGPFDLVYGATPPLGHTCDRPPSWYL |
| | | FQFHRLLQYARPKPGSPRPFFWMFVDNLVLNKEDLDVASRFLE |
| | | MEPVTIPDVHGGSLQNAVRVWSNIPAIRSRHWALVSEEELSLLA |
| | | QNKQSSKLAAKWPTKLVKNCFLPLREYFKYFSTELTSSLGGPSS |
| | | GAPPPSGGSPAGSPTSTEEGTSESATPESGPGTSTEPSEGSAPGSP |
| | | AGSPTSTEEGTSTEPSEGSAPGTSTEPSELEDKKYSIGLAIGTNSV |
| | | GWAVITDEYKVPSKKFKVLGNTDRHSIKKNLIGALLFDSGETAE |
| | | ATRLKRTARRRYTRRKNRICYLQEIFSNEMAKVDDSFFHRLEES |
| | | FLVEEDKKHERHPIFGNIVDEVAYHEKYPTIYHLRKKLVDSTDK |
| | | ADLRLIYLALAHMIKFRGHFLIEGDLNPDNSDVDKLFIQLVQTY |
| | | NQLFEENPINASGVDAKAILSARLSKSRRLENLIAQLPGEKKNG |
| | | LFGNLIALSLGLTPNFKSNFDLAEDAKLQLSKDTYDDDLDNLLA |
| | | QIGDQYADLFLAAKNLSDAILLSDILRVNTEITKAPLSASMIKRY |
| | | DEHHQDLTLLKALVRQQLPEKYKEIFFDQSKNGYAGYIDGGAS |
| | | QEEFYKFIKPILEKMDGTEELLVKLNREDLLRKQRTFDNGSIPH |
| | | QIHLGELHAILRRQEDFYPFLKDNREKIEKILTFRIPYYVGPLAR |
| | | GNSRFAWMTRKSEETITPWNFEEVVDKGASAQSFIERMTNFDK |
| | | NLPNEKVLPKHSLLYEYFTVYNELTKVKYVTEGMRKPAFLSGE |
| | | QKKAIVDLLFKTNRKVTVKQLKEDYFKKIECFDSVEISGVEDRF |
| | | NASLGTYHDLLKIIKDKDFLDNEENEDILEDIVLTLTLFEDREMI |
| | | EERLKTYAHLFDDKVMKQLKRRRYTGWGRLSRKLINGIRDKQ |
| | | SGKTILDFLKSDGFANRNFMQLIHDDSLTFKEDIQKAQVSGQGD |
| | | SLHEHIANLAGSPAIKKGILQTVKVVDELVKVMGRHKPENIVIE |
| | | MARENQTTQKGQKNSRERMKRIEEGIKELGSQILKEHPVENTQ |
| | | LQNEKLYLYYLQNGRDMYVDQELDINRLSDYDVDAIVPQSFLK |
| | | DDSIDNKVLTRSDKNRGKSDNVPSEEVVKKMKNYWRQLLNAK |
| | | LITQRKFDNLTKAERGGLSELDKAGFIKRQLVETRQITKHVAQI |
| | | LDSRMNTKYDENDKLIREVKVITLKSKLVSDFRKDFQFYKVREI |
| | | NNYHHAHDAYLNAVVGTALIKKYPKLESEFVYGDYKVYDVRK |
| | | MIAKSEQEIGKATAKYFFYSNIMNFFKTEITLANGEIRKRPLIETN |
| | | GETGEIVWDKGRDFATVRKVLSMPQVNIVKKTEVQTGGFSKES |
| | | ILPKRNSDKLIARKKDWDPKKYGGFDSPTVAYSVLVVAKVEKG |
| | | KSKKLKSVKELLGITIMERSSFEKNPIDFLEAKGYKEVKKDLIIK |
| | | LPKYSLFELENGRKRMLASAGELQKGNELALPSKYVNFLYLAS |
| | | HYEKLKGSPEDNEQKQLFVEQHKHYLDEIIEQISEFSKRVILADA |
| | | NLDKVLSAYNKHRDKPIREQAENIIHLFTLTNLGAPAAFKYFDT |
| | | TIDRKRYTSTKEVLDATLIHQSITGLYETRIDLSQLGGDSPKKKR |
| | | KVGVDGSSGSETPGTSESATPESTGMNNSQGRVTFEDVTVNFT |
| | | QGEWQRLNPEQRNLYRDVMLENYSNLVSVGQGETTKPDVILR |
| | | LEQGKEPWLEEEEVLGSGRAEKNGDIGGQIWKPKDVKESLSAD |
| | | YKDDDDKAPKKKRKVPKKKRKV |
| 115 | Fusion Protein 11 | ATGGGTACCATGCCAAAAAAGAAGAGAAAGGTACCGAAGA |
| | DNA Sequence | AAAAAAGAAAGGTATACAATCACGATCAGGAGTTCGACCCC |
| | | CCTAAGGTGTACCCACCAGTGCCTGCAGAGAAGAGGAAGCC |
| | | AATCCGGGTGCTGAGCCTGTTTGATGGCATCGCCACCGGCCT |
| | | GCTGGTGCTGAAGGATCTGGGCATCCAGGTGGACCGGTACA |
| | | TCGCCTCCGAGGTGTGCGAGGATTCTATCACCGTGGGCATGG |
| | | TGCGCCACCAGGGCAAGATCATGTATGTGGGCGACGTGCGG |
| | | TCCGTGACACAGAAGCACATCCAGGAGTGGGGCCCATTCGA |
| | | TCTGGTGATCGGCGGCAGCCCCTGTAATGACCTGTCCATCGT |
| | | GAACCCTGCAAGGAAGGGACTGTACGAGGGAACCGGCCGGC |
| | | TGTTCTTTGAGTTTTATAGACTGCTGCACGACGCCAGGCCTA |
| | | AGGAGGGCGACGATAGACCATTCTTTTGGCTGTTCGAGAATG |
| | | TGGTGGCTATGGGCGTGAGCGATAAGAGGGACATCTCCAGG |
| | | TTTCTGGAGTCTAACCCCGTGATGATCGATGCAAAGGAGGTG |
| | | TCCGCCGCACACAGAGCCAGGTATTTCTGGGGCAATCTGCCA |
| | | GGAATGAACAGGCCACTGGCAAGCACCGTGAATGACAAGCT |
| | | GGAGCTGCAGGAGTGCCTGGAGCACGGAAGGATCGCCAAGT |
| | | TTTCCAAGGTGCGCACAATCACCACACGGAGCAATTCCATCA |
| | | AGCAGGGCAAGGATCAGCACTTCCCCGTGTTCATGAACGAG |
| | | AAGGAGGACATCCTGTGGTGTACCGAGATGGAGAGAGTGTT |
| | | CGGCTTTCCAGTGCACTACACAGACGTGTCTAACATGAGCAG |
| | | GCTGGCAAGGCAGCGGCTGCTGGGCAGATCTTGGAGCGTGC |
| | | CCGTGATCAGGCACCTGTTCGCCCCTCTGAAGGAGTATTTTG |
| | | CCTGCGTGAGCAGCGGCAACTCCAATGCCAACAGCCGGGGC |
| | | CCCTCTTTCAGCTCCGGATTGGTGCCTCTGAGCCTGAGGGGC |
| | | TCCCACATGGCAGCAATCCCCGCCCTGGACCCCGAGGCCGA |
| | | GCCTAGCATGGACGTGATCCTGGTGGGCTCTAGCGAGCTGTC |
| | | CTCTAGCGTGTCTCCAGGAACCGGAAGGGATCTGATCGCATA |
| | | CGAGGTGAAGGCCAATCAGCGGAACATCGAGGACATCTGTA |
| | | TCTGCTGTGGCAGCCTGCAGGTGCACACACAGCACCCACTGT |
| | | TCGAGGGAGGAATCTGCGCACCCTGTAAGGATAAGTTCCTG |
| | | GACGCCCTGTTTCTGTACGACGATGACGGCTACCAGTCCTAT |
| | | TGCTCTATCTGCTGTTCCGGCGAGACCCTGCTGATCTGCGGC |
| | | AATCCAGATTGTACAAGGTGCTATTGTTTTGAGTGCGTGGAC |
| | | TCTCTGGTGGGACCAGGCACCAGCGGAAAGGTGCACGCCAT |
| | | GTCCAACTGGGTGTGCTACCTGTGCCTGCCATCCTCTCGCAG |
| | | CGGACTGCTGCAGCGGAGAAGGAAGTGGAGATCCCAGCTGA |
| | | AGGCCTTCTATGATAGGGAGTCTGAGAACCCCCTGGAGATGT |
| | | TTGAGACCGTGCCAGTGTGGCGCCGGCAGCCCGTGAGGGTG |
| | | CTGAGCCTGTTCGAGGATATCAAGAAGGAGCTGACATCCCT |
| | | GGGCTTTCTGGAGTCCGGCTCTGACCCCGGACAGCTGAAGCA |
| | | CGTGGTGGATGTGACCGACACAGTGCGGAAGGATGTGGAGG |
| | | AGTGGGGCCCTTTCGACCTGGTGTACGGAGCAACCCCTCCAC |
| | | TGGGACACACATGCGACAGACCCCCTTCTTGGTACCTGTTCC |
| | | AGTTTCACCGCCTGCTGCAGTATGCAAGGCCAAAGCCAGGC |
| | | AGCCCTAGACCATTCTTTTGGATGTTCGTGGATAATCTGGTG |
| | | CTGAACAAGGAGGATCTGGACGTGGCCAGCAGGTTTCTGGA |
| | | GATGGAGCCAGTGACCATCCCAGACGTGCACGGCGGCTCCC |
| | | TGCAGAATGCCGTGCGCGTGTGGTCTAACATCCCTGCCATCA |
| | | GAAGCAGGCACTGGGCACTGGTGAGCGAGGAGGAGCTGTCC |
| | | CTGCTGGCCCAGAATAAGCAGAGCAGCAAGCTGGCCGCCAA |
| | | GTGGCCTACAAAGCTGGTGAAGAACTGCTTCCTGCCACTGCG |
| | | GGAGTACTTCAAGTATTTTTCCACCGAGCTGACATCTAGCCT |
| | | GGGAGGACCCTCCTCTGGCGCCCCACCACCTAGCGGCGGCT |
| | | CCCCTGCCGGCTCTCCAACCAGCACAGAGGAGGGCACCAGC |
| | | GAGTCCGCCACACCAGAGTCTGGACCTGGCACCAGCACAGA |
| | | GCCATCCGAGGGCTCTGCCCCAGGCTCTCCTGCAGGCAGCCC |
| | | TACCTCCACCGAAGAGGGCACCAGCACAGAGCCTTCTGAGG |
| | | GCAGCGCCCCAGGCACCTCTACAGAGCCAAGCGAGCTCGAG |
| | | GACAAGAAGTACAGCATCGGCCTGGCCATCGGCACCAACTC |
| | | TGTGGGCTGGGCCGTGATCACCGACGAGTACAAGGTGCCCA |
| | | GCAAGAAATTCAAGGTGCTGGGCAACACCGACCGGCACAGC |
| | | ATCAAGAAGAACCTGATCGGAGCCCTGCTGTTCGACAGCGG |
| | | CGAAACAGCCGAGGCCACCCGGCTGAAGAGAACCGCCAGA |
| | | AGAAGATACACCAGACGGAAGAACCGGATCTGCTATCTGCA |
| | | AGAGATCTTCAGCAACGAGATGGCCAAGGTGGACGACAGCT |
| | | TCTTCCACAGACTGGAAGAGTCCTTCCTGGTGGAAGAGGATA |
| | | AGAAGCACGAGCGGCACCCCATCTTCGGCAACATCGTGGAC |
| | | GAGGTGGCCTACCACGAGAAGTACCCCACCATCTACCACCT |
| | | GAGAAAGAAACTGGTGGACAGCACCGACAAGGCCGACCTGC |
| | | GGCTGATCTATCTGGCCCTGGCCCACATGATCAAGTTCCGGG |
| | | GCCACTTCCTGATCGAGGGCGACCTGAACCCCGACAACAGC |
| | | GACGTGGACAAGCTGTTCATCCAGCTGGTGCAGACCTACAA |
| | | CCAGCTGTTCGAGGAAAACCCCATCAACGCCAGCGGCGTGG |
| | | ACGCCAAGGCCATCCTGTCTGCCAGACTGAGCAAGAGCAGA |
| | | CGGCTGGAAAATCTGATCGCCCAGCTGCCCGGCGAGAAGAA |
| | | GAATGGCCTGTTCGGCAACCTGATTGCCCTGAGCCTGGGCCT |
| | | GACCCCCAACTTCAAGAGCAACTTCGACCTGGCCGAGGATG |
| | | CCAAACTGCAGCTGAGCAAGGACACCTACGACGACGACCTG |
| | | GACAACCTGCTGGCCCAGATCGGCGACCAGTACGCCGACCT |
| | | GTTTCTGGCCGCCAAGAACCTGTCCGACGCCATCCTGCTGAG |
| | | CGACATCCTGAGAGTGAACACCGAGATCACCAAGGCCCCCC |
| | | TGAGCGCCTCTATGATCAAGAGATACGACGAGCACCACCAG |
| | | GACCTGACCCTGCTGAAAGCTCTCGTGCGGCAGCAGCTGCCT |
| | | GAGAAGTACAAAGAGATTTTCTTCGACCAGAGCAAGAACGG |
| | | CTACGCCGGCTACATTGACGGCGGAGCCAGCCAGGAAGAGT |
| | | TCTACAAGTTCATCAAGCCCATCCTGGAAAAGATGGACGGC |
| | | ACCGAGGAACTGCTCGTGAAGCTGAACAGAGAGGACCTGCT |
| | | GCGGAAGCAGCGGACCTTCGACAACGGCAGCATCCCCCACC |
| | | AGATCCACCTGGGAGAGCTGCACGCCATTCTGCGGCGGCAG |
| | | GAAGATTTTTACCCATTCCTGAAGGACAACCGGGAAAAGAT |
| | | CGAGAAGATCCTGACCTTCCGCATCCCCTACTACGTGGGCCC |
| | | TCTGGCCAGGGGAAACAGCAGATTCGCCTGGATGACCAGAA |
| | | AGAGCGAGGAAACCATCACCCCCTGGAACTTCGAGGAAGTG |
| | | GTGGACAAGGGCGCTTCCGCCCAGAGCTTCATCGAGCGGAT |
| | | GACCAACTTCGATAAGAACCTGCCCAACGAGAAGGTGCTGC |
| | | CCAAGCACAGCCTGCTGTACGAGTACTTCACCGTGTATAACG |
| | | AGCTGACCAAAGTGAAATACGTGACCGAGGGAATGAGAAAG |
| | | CCCGCCTTCCTGAGCGGCGAGCAGAAAAAGGCCATCGTGGA |
| | | CCTGCTGTTCAAGACCAACCGGAAAGTGACCGTGAAGCAGC |
| | | TGAAAGAGGACTACTTCAAGAAAATCGAGTGCTTCGACTCC |
| | | GTGGAAATCTCCGGCGTGGAAGATCGGTTCAACGCCTCCCTG |
| | | GGCACATACCACGATCTGCTGAAAATTATCAAGGACAAGGA |
| | | CTTCCTGGACAATGAGGAAAACGAGGACATTCTGGAAGATA |
| | | TCGTGCTGACCCTGACACTGTTTGAGGACAGAGAGATGATCG |
| | | AGGAACGGCTGAAAACCTATGCCCACCTGTTCGACGACAAA |
| | | GTGATGAAGCAGCTGAAGCGGCGGAGATACACCGGCTGGGG |
| | | CAGGCTGAGCCGGAAGCTGATCAACGGCATCCGGGACAAGC |
| | | AGTCCGGCAAGACAATCCTGGATTTCCTGAAGTCCGACGGCT |
| | | TCGCCAACAGAAACTTCATGCAGCTGATCCACGACGACAGC |
| | | CTGACCTTTAAAGAGGACATCCAGAAAGCCCAGGTGTCCGG |
| | | CCAGGGCGATAGCCTGCACGAGCACATTGCCAATCTGGCCG |
| | | GCAGCCCCGCCATTAAGAAGGGCATCCTGCAGACAGTGAAG |
| | | GTGGTGGACGAGCTCGTGAAAGTGATGGGCCGGCACAAGCC |
| | | CGAGAACATCGTGATCGAAATGGCCAGAGAGAACCAGACCA |
| | | CCCAGAAGGGACAGAAGAACAGCCGCGAGAGAATGAAGCG |
| | | GATCGAAGAGGGCATCAAAGAGCTGGGCAGCCAGATCCTGA |
| | | AAGAACACCCCGTGGAAAACACCCAGCTGCAGAACGAGAAG |
| | | CTGTACCTGTACTACCTGCAGAATGGGGGGATATGTACGTG |
| | | GACCAGGAACTGGACATCAACCGGCTGTCCGACTACGATGT |
| | | GGACGCCATCGTGCCTCAGAGCTTTCTGAAGGACGACTCCAT |
| | | CGACAACAAGGTGCTGACCAGAAGCGACAAGAACCGGGGC |
| | | AAGAGCGACAACGTGCCCTCCGAAGAGGTCGTGAAGAAGAT |
| | | GAAGAACTACTGGCGGCAGCTGCTGAACGCCAAGCTGATTA |
| | | CCCAGAGAAAGTTCGACAATCTGACCAAGGCCGAGAGAGGC |
| | | GGCCTGAGCGAACTGGATAAGGCCGGCTTCATCAAGAGACA |
| | | GCTGGTGGAAACCCGGCAGATCACAAAGCACGTGGCACAGA |
| | | TCCTGGACTCCCGGATGAACACTAAGTACGACGAGAATGAC |
| | | AAGCTGATCCGGGAAGTGAAAGTGATCACCCTGAAGTCCAA |
| | | GCTGGTGTCCGATTTCCGGAAGGATTTCCAGTTTTACAAAGT |
| | | GCGCGAGATCAACAACTACCACCACGCCCACGACGCCTACC |
| | | TGAACGCCGTCGTGGGAACCGCCCTGATCAAAAAGTACCCT |
| | | AAGCTGGAAAGCGAGTTCGTGTACGGCGACTACAAGGTGTA |
| | | CGACGTGCGGAAGATGATCGCCAAGAGCGAGCAGGAAATCG |
| | | GCAAGGCTACCGCCAAGTACTTCTTCTACAGCAACATCATGA |
| | | ACTTTTTCAAGACCGAGATTACCCTGGCCAACGGCGAGATCC |
| | | GGAAGCGGCCTCTGATCGAGACAAACGGCGAAACCGGGGAG |
| | | ATCGTGTGGGATAAGGGCCGGGATTTTGCCACCGTGCGGAA |
| | | AGTGCTGAGCATGCCCCAAGTGAATATCGTGAAAAAGACCG |
| | | AGGTGCAGACAGGCGGCTTCAGCAAAGAGTCTATCCTGCCC |
| | | AAGAGGAACAGCGATAAGCTGATCGCCAGAAAGAAGGACT |
| | | GGGACCCTAAGAAGTACGGCGGCTTCGACAGCCCCACCGTG |
| | | GCCTATTCTGTGCTGGTGGTGGCCAAAGTGGAAAAGGGCAA |
| | | GTCCAAGAAACTGAAGAGTGTGAAAGAGCTGCTGGGGATCA |
| | | CCATCATGGAAAGAAGCAGCTTCGAGAAGAATCCCATCGAC |
| | | TTTCTGGAAGCCAAGGGCTACAAAGAAGTGAAAAAGGACCT |
| | | GATCATCAAGCTGCCTAAGTACTCCCTGTTCGAGCTGGAAAA |
| | | CGGCCGGAAGAGAATGCTGGCCTCTGCCGGCGAACTGCAGA |
| | | AGGGAAACGAACTGGCCCTGCCCTCCAAATATGTGAACTTCC |
| | | TGTACCTGGCCAGCCACTATGAGAAGCTGAAGGGCTCCCCC |
| | | GAGGATAATGAGCAGAAACAGCTGTTTGTGGAACAGCACAA |
| | | GCACTACCTGGACGAGATCATCGAGCAGATCAGCGAGTTCT |
| | | CCAAGAGAGTGATCCTGGCCGACGCTAATCTGGACAAAGTG |
| | | CTGTCCGCCTACAACAAGCACCGGGATAAGCCCATCAGAGA |
| | | GCAGGCCGAGAATATCATCCACCTGTTTACCCTGACCAATCT |
| | | GGGAGCCCCTGCCGCCTTCAAGTACTTTGACACCACCATCGA |
| | | CCGGAAGAGGTACACCAGCACCAAAGAGGTGCTGGACGCCA |
| | | CCCTGATCCACCAGAGCATCACCGGCCTGTACGAGACACGG |
| | | ATCGACCTGTCTCAGCTGGGAGGCGACAGCCCCAAGAAGAA |
| | | GAGAAAGGTGGGAGTCGACGGATCCAGCGGCTCCGAGACCC |
| | | CAGGCACATCTGAGAGCGCCACCCCTGAGTCCACCGGTATG |
| | | AACAATTCACAGGGGAGAGTGACATTCGAAGACGTGACCGT |
| | | GAACTTCACCCAGGGAGAATGGCAGCGCTTGAACCCAGAAC |
| | | AAAGGAACCTCTATCGGGACGTGATGCTGGAAAACTACTCA |
| | | AATTTGGTGAGCGTTGGGCAGGGTGAGACCACTAAGCCTGA |
| | | CGTGATCCTGAGATTGGAACAGGGCAAGGAGCCTTGGCTCG |
| | | AGGAAGAGGAAGTCCTGGGCTCAGGGAGGGCCGAGAAAAA |
| | | CGGTGATATAGGAGGCCAGATATGGAAGCCTAAGGACGTCA |
| | | AGGAGAGCCTGAGCGCTGATTACAAAGATGATGACGATAAA |
| | | GCCCCCAAGAAGAAAAGGAAGGTCCCAAAGAAAAAAAGAA |
| | | AGGTGTGA |
| 116 | Fusion Protein 12 | MGTMPKKKRKVPKKKRKVYNHDQEFDPPKVYPPVPAEKRKPI |
| | Amino Acid | RVLSLFDGIATGLLVLKDLGIQVDRYIASEVCEDSITVGMVRHQ |
| | Sequence | GKIMYVGDVRSVTQKHIQEWGPFDLVIGGSPCNDLSIVNPARK |
| | NLS-NLS-3A- | GLYEGTGRLFFEFYRLLHDARPKEGDDRPFFWLFENVVAMGVS |
| | h3L-dCas9- | DKRDISRFLESNPVMIDAKEVSAAHRARYFWGNLPGMNRPLAS |
| | ZN627-NLS-NLS | TVNDKLELQECLEHGRIAKFSKVRTITTRSNSIKQGKDQHFPVF |
| | | MNEKEDILWCTEMERVFGFPVHYTDVSNMSRLARQRLLGRSW |
| | | SVPVIRHLFAPLKEYFACVSSGNSNANSRGPSFSSGLVPLSLRGS |
| | | HMAAIPALDPEAEPSMDVILVGSSELSSSVSPGTGRDLIAYEVK |
| | | ANQRNIEDICICCGSLQVHTQHPLFEGGICAPCKDKFLDALFLYD |
| | | DDGYQSYCSICCSGETLLICGNPDCTRCYCFECVDSLVGPGTSG |
| | | KVHAMSNWVCYLCLPSSRSGLLQRRRKWRSQLKAFYDRESEN |
| | | PLEMFETVPVWRRQPVRVLSLFEDIKKELTSLGFLESGSDPGQL |
| | | KHVVDVTDTVRKDVEEWGPFDLVYGATPPLGHTCDRPPSWYL |
| | | FQFHRLLQYARPKPGSPRPFFWMFVDNLVLNKEDLDVASRFLE |
| | | MEPVTIPDVHGGSLQNAVRVWSNIPAIRSRHWALVSEEELSLLA |
| | | QNKQSSKLAAKWPTKLVKNCFLPLREYFKYFSTELTSSLGGPSS |
| | | GAPPPSGGSPAGSPTSTEEGTSESATPESGPGTSTEPSEGSAPGSP |
| | | AGSPTSTEEGTSTEPSEGSAPGTSTEPSELEDKKYSIGLAIGTNSV |
| | | GWAVITDEYKVPSKKFKVLGNTDRHSIKKNLIGALLFDSGETAE |
| | | ATRLKRTARRRYTRRKNRICYLQEIFSNEMAKVDDSFFHRLEES |
| | | FLVEEDKKHERHPIFGNIVDEVAYHEKYPTIYHLRKKLVDSTDK |
| | | ADLRLIYLALAHMIKFRGHFLIEGDLNPDNSDVDKLFIQLVQTY |
| | | NQLFEENPINASGVDAKAILSARLSKSRRLENLIAQLPGEKKNG |
| | | LFGNLIALSLGLTPNFKSNFDLAEDAKLQLSKDTYDDDLDNLLA |
| | | QIGDQYADLFLAAKNLSDAILLSDILRVNTEITKAPLSASMIKRY |
| | | DEHHQDLTLLKALVRQQLPEKYKEIFFDQSKNGYAGYIDGGAS |
| | | QEEFYKFIKPILEKMDGTEELLVKLNREDLLRKQRTFDNGSIPH |
| | | QIHLGELHAILRRQEDFYPFLKDNREKIEKILTFRIPYYVGPLAR |
| | | GNSRFAWMTRKSEETITPWNFEEVVDKGASAQSFIERMTNFDK |
| | | NLPNEKVLPKHSLLYEYFTVYNELTKVKYVTEGMRKPAFLSGE |
| | | QKKAIVDLLFKTNRKVTVKQLKEDYFKKIECFDSVEISGVEDRF |
| | | NASLGTYHDLLKIIKDKDFLDNEENEDILEDIVLTLTLFEDREMI |
| | | EERLKTYAHLFDDKVMKQLKRRRYTGWGRLSRKLINGIRDKQ |
| | | SGKTILDFLKSDGFANRNFMQLIHDDSLTFKEDIQKAQVSGQGD |
| | | SLHEHIANLAGSPAIKKGILQTVKVVDELVKVMGRHKPENIVIE |
| | | MARENQTTQKGQKNSRERMKRIEEGIKELGSQILKEHPVENTQ |
| | | LQNEKLYLYYLQNGRDMYVDQELDINRLSDYDVDAIVPQSFLK |
| | | DDSIDNKVLTRSDKNRGKSDNVPSEEVVKKMKNYWRQLLNAK |
| | | LITQRKFDNLTKAERGGLSELDKAGFIKRQLVETRQITKHVAQI |
| | | LDSRMNTKYDENDKLIREVKVITLKSKLVSDFRKDFQFYKVREI |
| | | NNYHHAHDAYLNAVVGTALIKKYPKLESEFVYGDYKVYDVRK |
| | | MIAKSEQEIGKATAKYFFYSNIMNFFKTEITLANGEIRKRPLIETN |
| | | GETGEIVWDKGRDFATVRKVLSMPQVNIVKKTEVQTGGFSKES |
| | | ILPKRNSDKLIARKKDWDPKKYGGFDSPTVAYSVLVVAKVEKG |
| | | KSKKLKSVKELLGITIMERSSFEKNPIDFLEAKGYKEVKKDLIIK |
| | | LPKYSLFELENGRKRMLASAGELQKGNELALPSKYVNFLYLAS |
| | | HYEKLKGSPEDNEQKQLFVEQHKHYLDEIIEQISEFSKRVILADA |
| | | NLDKVLSAYNKHRDKPIREQAENIIHLFTLTNLGAPAAFKYFDT |
| | | TIDRKRYTSTKEVLDATLIHQSITGLYETRIDLSQLGGDSPKKKR |
| | | KVGVDGSSGSETPGTSESATPESTGDSVAFEDVAVNFTLEEWAL |
| | | LDPSQKNLYRDVMRETFRNLASVGKQWEDQNIEDPFKIPRRNIS |
| | | HIPERLCESKEGGQGEESADYKDDDDKAPKKKRKVPKKKRKV |
| 117 | Fusion Protein 12 | ATGGGTACCATGCCAAAAAAGAAGAGAAAGGTACCGAAGA |
| | DNA Sequence | AAAAAAGAAAGGTATACAATCACGATCAGGAGTTCGACCCC |
| | | CCTAAGGTGTACCCACCAGTGCCTGCAGAGAAGAGGAAGCC |
| | | AATCCGGGTGCTGAGCCTGTTTGATGGCATCGCCACCGGCCT |
| | | GCTGGTGCTGAAGGATCTGGGCATCCAGGTGGACCGGTACA |
| | | TCGCCTCCGAGGTGTGCGAGGATTCTATCACCGTGGGCATGG |
| | | TGCGCCACCAGGGCAAGATCATGTATGTGGGCGACGTGCGG |
| | | TCCGTGACACAGAAGCACATCCAGGAGTGGGGCCCATTCGA |
| | | TCTGGTGATCGGCGGCAGCCCCTGTAATGACCTGTCCATCGT |
| | | GAACCCTGCAAGGAAGGGACTGTACGAGGGAACCGGCCGGC |
| | | TGTTCTTTGAGTTTTATAGACTGCTGCACGACGCCAGGCCTA |
| | | AGGAGGGCGACGATAGACCATTCTTTTGGCTGTTCGAGAATG |
| | | TGGTGGCTATGGGCGTGAGCGATAAGAGGGACATCTCCAGG |
| | | TTTCTGGAGTCTAACCCCGTGATGATCGATGCAAAGGAGGTG |
| | | TCCGCCGCACACAGAGCCAGGTATTTCTGGGGCAATCTGCCA |
| | | GGAATGAACAGGCCACTGGCAAGCACCGTGAATGACAAGCT |
| | | GGAGCTGCAGGAGTGCCTGGAGCACGGAAGGATCGCCAAGT |
| | | TTTCCAAGGTGCGCACAATCACCACACGGAGCAATTCCATCA |
| | | AGCAGGGCAAGGATCAGCACTTCCCCGTGTTCATGAACGAG |
| | | AAGGAGGACATCCTGTGGTGTACCGAGATGGAGAGAGTGTT |
| | | CGGCTTTCCAGTGCACTACACAGACGTGTCTAACATGAGCAG |
| | | GCTGGCAAGGCAGCGGCTGCTGGGCAGATCTTGGAGCGTGC |
| | | CCGTGATCAGGCACCTGTTCGCCCCTCTGAAGGAGTATTTTG |
| | | CCTGCGTGAGCAGCGGCAACTCCAATGCCAACAGCCGGGGC |
| | | CCCTCTTTCAGCTCCGGATTGGTGCCTCTGAGCCTGAGGGGC |
| | | TCCCACATGGCAGCAATCCCCGCCCTGGACCCCGAGGCCGA |
| | | GCCTAGCATGGACGTGATCCTGGTGGGCTCTAGCGAGCTGTC |
| | | CTCTAGCGTGTCTCCAGGAACCGGAAGGGATCTGATCGCATA |
| | | CGAGGTGAAGGCCAATCAGCGGAACATCGAGGACATCTGTA |
| | | TCTGCTGTGGCAGCCTGCAGGTGCACACACAGCACCCACTGT |
| | | TCGAGGGAGGAATCTGCGCACCCTGTAAGGATAAGTTCCTG |
| | | GACGCCCTGTTTCTGTACGACGATGACGGCTACCAGTCCTAT |
| | | TGCTCTATCTGCTGTTCCGGCGAGACCCTGCTGATCTGCGGC |
| | | AATCCAGATTGTACAAGGTGCTATTGTTTTGAGTGCGTGGAC |
| | | TCTCTGGTGGGACCAGGCACCAGCGGAAAGGTGCACGCCAT |
| | | GTCCAACTGGGTGTGCTACCTGTGCCTGCCATCCTCTCGCAG |
| | | CGGACTGCTGCAGCGGAGAAGGAAGTGGAGATCCCAGCTGA |
| | | AGGCCTTCTATGATAGGGAGTCTGAGAACCCCCTGGAGATGT |
| | | TTGAGACCGTGCCAGTGTGGCGCCGGCAGCCCGTGAGGGTG |
| | | CTGAGCCTGTTCGAGGATATCAAGAAGGAGCTGACATCCCT |
| | | GGGCTTTCTGGAGTCCGGCTCTGACCCCGGACAGCTGAAGCA |
| | | CGTGGTGGATGTGACCGACACAGTGCGGAAGGATGTGGAGG |
| | | AGTGGGGCCCTTTCGACCTGGTGTACGGAGCAACCCCTCCAC |
| | | TGGGACACACATGCGACAGACCCCCTTCTTGGTACCTGTTCC |
| | | AGTTTCACCGCCTGCTGCAGTATGCAAGGCCAAAGCCAGGC |
| | | AGCCCTAGACCATTCTTTTGGATGTTCGTGGATAATCTGGTG |
| | | CTGAACAAGGAGGATCTGGACGTGGCCAGCAGGTTTCTGGA |
| | | GATGGAGCCAGTGACCATCCCAGACGTGCACGGCGGCTCCC |
| | | TGCAGAATGCCGTGCGCGTGTGGTCTAACATCCCTGCCATCA |
| | | GAAGCAGGCACTGGGCACTGGTGAGCGAGGAGGAGCTGTCC |
| | | CTGCTGGCCCAGAATAAGCAGAGCAGCAAGCTGGCCGCCAA |
| | | GTGGCCTACAAAGCTGGTGAAGAACTGCTTCCTGCCACTGCG |
| | | GGAGTACTTCAAGTATTTTTCCACCGAGCTGACATCTAGCCT |
| | | GGGAGGACCCTCCTCTGGCGCCCCACCACCTAGCGGCGGCT |
| | | CCCCTGCCGGCTCTCCAACCAGCACAGAGGAGGGCACCAGC |
| | | GAGTCCGCCACACCAGAGTCTGGACCTGGCACCAGCACAGA |
| | | GCCATCCGAGGGCTCTGCCCCAGGCTCTCCTGCAGGCAGCCC |
| | | TACCTCCACCGAAGAGGGCACCAGCACAGAGCCTTCTGAGG |
| | | GCAGCGCCCCAGGCACCTCTACAGAGCCAAGCGAGCTCGAG |
| | | GACAAGAAGTACAGCATCGGCCTGGCCATCGGCACCAACTC |
| | | TGTGGGCTGGGCCGTGATCACCGACGAGTACAAGGTGCCCA |
| | | GCAAGAAATTCAAGGTGCTGGGCAACACCGACCGGCACAGC |
| | | ATCAAGAAGAACCTGATCGGAGCCCTGCTGTTCGACAGCGG |
| | | CGAAACAGCCGAGGCCACCCGGCTGAAGAGAACCGCCAGA |
| | | AGAAGATACACCAGACGGAAGAACCGGATCTGCTATCTGCA |
| | | AGAGATCTTCAGCAACGAGATGGCCAAGGTGGACGACAGCT |
| | | TCTTCCACAGACTGGAAGAGTCCTTCCTGGTGGAAGAGGATA |
| | | AGAAGCACGAGCGGCACCCCATCTTCGGCAACATCGTGGAC |
| | | GAGGTGGCCTACCACGAGAAGTACCCCACCATCTACCACCT |
| | | GAGAAAGAAACTGGTGGACAGCACCGACAAGGCCGACCTGC |
| | | GGCTGATCTATCTGGCCCTGGCCCACATGATCAAGTTCCGGG |
| | | GCCACTTCCTGATCGAGGGCGACCTGAACCCCGACAACAGC |
| | | GACGTGGACAAGCTGTTCATCCAGCTGGTGCAGACCTACAA |
| | | CCAGCTGTTCGAGGAAAACCCCATCAACGCCAGCGGCGTGG |
| | | ACGCCAAGGCCATCCTGTCTGCCAGACTGAGCAAGAGCAGA |
| | | CGGCTGGAAAATCTGATCGCCCAGCTGCCCGGCGAGAAGAA |
| | | GAATGGCCTGTTCGGCAACCTGATTGCCCTGAGCCTGGGCCT |
| | | GACCCCCAACTTCAAGAGCAACTTCGACCTGGCCGAGGATG |
| | | CCAAACTGCAGCTGAGCAAGGACACCTACGACGACGACCTG |
| | | GACAACCTGCTGGCCCAGATCGGCGACCAGTACGCCGACCT |
| | | GTTTCTGGCCGCCAAGAACCTGTCCGACGCCATCCTGCTGAG |
| | | CGACATCCTGAGAGTGAACACCGAGATCACCAAGGCCCCCC |
| | | TGAGCGCCTCTATGATCAAGAGATACGACGAGCACCACCAG |
| | | GACCTGACCCTGCTGAAAGCTCTCGTGCGGCAGCAGCTGCCT |
| | | GAGAAGTACAAAGAGATTTTCTTCGACCAGAGCAAGAACGG |
| | | CTACGCCGGCTACATTGACGGCGGAGCCAGCCAGGAAGAGT |
| | | TCTACAAGTTCATCAAGCCCATCCTGGAAAAGATGGACGGC |
| | | ACCGAGGAACTGCTCGTGAAGCTGAACAGAGAGGACCTGCT |
| | | GCGGAAGCAGCGGACCTTCGACAACGGCAGCATCCCCCACC |
| | | AGATCCACCTGGGAGAGCTGCACGCCATTCTGCGGCGGCAG |
| | | GAAGATTTTTACCCATTCCTGAAGGACAACCGGGAAAAGAT |
| | | CGAGAAGATCCTGACCTTCCGCATCCCCTACTACGTGGGCCC |
| | | TCTGGCCAGGGGAAACAGCAGATTCGCCTGGATGACCAGAA |
| | | AGAGCGAGGAAACCATCACCCCCTGGAACTTCGAGGAAGTG |
| | | GTGGACAAGGGCGCTTCCGCCCAGAGCTTCATCGAGCGGAT |
| | | GACCAACTTCGATAAGAACCTGCCCAACGAGAAGGTGCTGC |
| | | CCAAGCACAGCCTGCTGTACGAGTACTTCACCGTGTATAACG |
| | | AGCTGACCAAAGTGAAATACGTGACCGAGGGAATGAGAAAG |
| | | CCCGCCTTCCTGAGCGGCGAGCAGAAAAAGGCCATCGTGGA |
| | | CCTGCTGTTCAAGACCAACCGGAAAGTGACCGTGAAGCAGC |
| | | TGAAAGAGGACTACTTCAAGAAAATCGAGTGCTTCGACTCC |
| | | GTGGAAATCTCCGGCGTGGAAGATCGGTTCAACGCCTCCCTG |
| | | GGCACATACCACGATCTGCTGAAAATTATCAAGGACAAGGA |
| | | CTTCCTGGACAATGAGGAAAACGAGGACATTCTGGAAGATA |
| | | TCGTGCTGACCCTGACACTGTTTGAGGACAGAGAGATGATCG |
| | | AGGAACGGCTGAAAACCTATGCCCACCTGTTCGACGACAAA |
| | | GTGATGAAGCAGCTGAAGCGGCGGAGATACACCGGCTGGGG |
| | | CAGGCTGAGCCGGAAGCTGATCAACGGCATCCGGGACAAGC |
| | | AGTCCGGCAAGACAATCCTGGATTTCCTGAAGTCCGACGGCT |
| | | TCGCCAACAGAAACTTCATGCAGCTGATCCACGACGACAGC |
| | | CTGACCTTTAAAGAGGACATCCAGAAAGCCCAGGTGTCCGG |
| | | CCAGGGCGATAGCCTGCACGAGCACATTGCCAATCTGGCCG |
| | | GCAGCCCCGCCATTAAGAAGGGCATCCTGCAGACAGTGAAG |
| | | GTGGTGGACGAGCTCGTGAAAGTGATGGGCCGGCACAAGCC |
| | | CGAGAACATCGTGATCGAAATGGCCAGAGAGAACCAGACCA |
| | | CCCAGAAGGGACAGAAGAACAGCCGCGAGAGAATGAAGCG |
| | | GATCGAAGAGGGCATCAAAGAGCTGGGCAGCCAGATCCTGA |
| | | AAGAACACCCCGTGGAAAACACCCAGCTGCAGAACGAGAAG |
| | | CTGTACCTGTACTACCTGCAGAATGGGCGGGATATGTACGTG |
| | | GACCAGGAACTGGACATCAACCGGCTGTCCGACTACGATGT |
| | | GGACGCCATCGTGCCTCAGAGCTTTCTGAAGGACGACTCCAT |
| | | CGACAACAAGGTGCTGACCAGAAGCGACAAGAACCGGGGC |
| | | AAGAGCGACAACGTGCCCTCCGAAGAGGTCGTGAAGAAGAT |
| | | GAAGAACTACTGGCGGCAGCTGCTGAACGCCAAGCTGATTA |
| | | CCCAGAGAAAGTTCGACAATCTGACCAAGGCCGAGAGAGGC |
| | | GGCCTGAGCGAACTGGATAAGGCCGGCTTCATCAAGAGACA |
| | | GCTGGTGGAAACCCGGCAGATCACAAAGCACGTGGCACAGA |
| | | TCCTGGACTCCCGGATGAACACTAAGTACGACGAGAATGAC |
| | | AAGCTGATCCGGGAAGTGAAAGTGATCACCCTGAAGTCCAA |
| | | GCTGGTGTCCGATTTCCGGAAGGATTTCCAGTTTTACAAAGT |
| | | GCGCGAGATCAACAACTACCACCACGCCCACGACGCCTACC |
| | | TGAACGCCGTCGTGGGAACCGCCCTGATCAAAAAGTACCCT |
| | | AAGCTGGAAAGCGAGTTCGTGTACGGCGACTACAAGGTGTA |
| | | CGACGTGCGGAAGATGATCGCCAAGAGCGAGCAGGAAATCG |
| | | GCAAGGCTACCGCCAAGTACTTCTTCTACAGCAACATCATGA |
| | | ACTTTTTCAAGACCGAGATTACCCTGGCCAACGGCGAGATCC |
| | | GGAAGCGGCCTCTGATCGAGACAAACGGCGAAACCGGGGAG |
| | | ATCGTGTGGGATAAGGGCCGGGATTTTGCCACCGTGCGGAA |
| | | AGTGCTGAGCATGCCCCAAGTGAATATCGTGAAAAAGACCG |
| | | AGGTGCAGACAGGCGGCTTCAGCAAAGAGTCTATCCTGCCC |
| | | AAGAGGAACAGCGATAAGCTGATCGCCAGAAAGAAGGACT |
| | | GGGACCCTAAGAAGTACGGCGGCTTCGACAGCCCCACCGTG |
| | | GCCTATTCTGTGCTGGTGGTGGCCAAAGTGGAAAAGGGCAA |
| | | GTCCAAGAAACTGAAGAGTGTGAAAGAGCTGCTGGGGATCA |
| | | CCATCATGGAAAGAAGCAGCTTCGAGAAGAATCCCATCGAC |
| | | TTTCTGGAAGCCAAGGGCTACAAAGAAGTGAAAAAGGACCT |
| | | GATCATCAAGCTGCCTAAGTACTCCCTGTTCGAGCTGGAAAA |
| | | CGGCCGGAAGAGAATGCTGGCCTCTGCCGGCGAACTGCAGA |
| | | AGGGAAACGAACTGGCCCTGCCCTCCAAATATGTGAACTTCC |
| | | TGTACCTGGCCAGCCACTATGAGAAGCTGAAGGGCTCCCCC |
| | | GAGGATAATGAGCAGAAACAGCTGTTTGTGGAACAGCACAA |
| | | GCACTACCTGGACGAGATCATCGAGCAGATCAGCGAGTTCT |
| | | CCAAGAGAGTGATCCTGGCCGACGCTAATCTGGACAAAGTG |
| | | CTGTCCGCCTACAACAAGCACCGGGATAAGCCCATCAGAGA |
| | | GCAGGCCGAGAATATCATCCACCTGTTTACCCTGACCAATCT |
| | | GGGAGCCCCTGCCGCCTTCAAGTACTTTGACACCACCATCGA |
| | | CCGGAAGAGGTACACCAGCACCAAAGAGGTGCTGGACGCCA |
| | | CCCTGATCCACCAGAGCATCACCGGCCTGTACGAGACACGG |
| | | ATCGACCTGTCTCAGCTGGGAGGCGACAGCCCCAAGAAGAA |
| | | GAGAAAGGTGGGAGTCGACGGATCCAGCGGCTCCGAGACCC |
| | | CAGGCACATCTGAGAGCGCCACCCCTGAGTCCACCGGTGAC |
| | | TCCGTTGCTTTCGAGGACGTGGCCGTGAACTTCACACTTGAG |
| | | GAATGGGCCTTGCTCGACCCAAGTCAGAAGAATCTGTACAG |
| | | AGACGTGATGCGGGAGACATTCAGGAATCTCGCCAGTGTCG |
| | | GAAAGCAGTGGGAAGACCAGAACATCGAAGATCCTTTCAAG |
| | | ATACCACGGCGCAATATCTCCCACATTCCTGAGAGGCTGTGT |
| | | GAATCTAAGGAAGGCGGACAAGGTGAGGAAAGCGCTGATTA |
| | | CAAAGATGATGACGATAAAGCCCCCAAGAAGAAAAGGAAG |
| | | GTCCCAAAGAAAAAAAGAAAGGTGTGA |
| 118 | gRNA009 | ACUGCCUGGCUCACUCCUCC |
| 119 | gRNA003 | UGCGGAAACCUUCUAGGGUG |
| 120 | gRNA093 | AACCUGAUCCUCCAGUCCGG |
| 121 | gRNA011 | AUCGUCCGAUGGGGCUCUGG |
| 122 | gRNA007 | GGUGUGGGUGCUUGACGCCU |
| 123 | gRNA077 | CAAAUCCUAACUGGGCUGGA |
| 124 | gRNA113 | GGGCGCCGCCGUUCAGUUCA |
| 125 | gRNA004 | GCGGAAACCUUCUAGGGUGU |
| 126 | gRNA008 | GUGUGGGUGCUUGACGCCUG |
| 127 | gRNA012 | AGGAUCGUCCGAUGGGGCUC |
| 128 | gRNA111 | AGUUCAGGGUCUGAGCCUGG |
| 129 | gRNA005 | UCAAGCACCCACACCCUAGA |
| 130 | gRNA013 | UCAGAUAGGAUCGUCCGAUG |
| 131 | ZFoff-15 Target | GATGGGGCTCTGGTGGCG |
| | Sequence | |
| 132 | ZFoff-15 ZFP | SRPGERPFQCRICMRNFSRTDTLARHLRTHTGEKPFQCRICMRN |
| | Amino Acid | FSRKTALNRHLKTHTGSQKPFQCRICMRNFSRNESLKVHLRTHT |
| | Sequence | GEKPFQCRICMRNFSVKNTLTRHLKTHTGSQKPFQCRICMRNFS |
| | | RREHLVRHLRTHTGEKPFQCRICMRNFSLTHNLRRHLKTHLRG |
| | | S |
| 133 | ZFoff-15 Full | MGTMYNHDQEFDPPKVYPPVPAEKRKPIRVLSLFDGIATGLLVL |
| | Amino Acid | KDLGIQVDRYIASEVCEDSITVGMVRHQGKIMYVGDVRSVTQK |
| | Sequence | HIQEWGPFDLVIGGSPCNDLSIVNPARKGLYEGTGRLFFEFYRLL |
| | | HDARPKEGDDRPFFWLFENVVAMGVSDKRDISRFLESNPVMID |
| | | AKEVSAAHRARYFWGNLPGMNRPLASTVNDKLELQECLEHGR |
| | | IAKFSKVRTITTRSNSIKQGKDQHFPVFMNEKEDILWCTEMERV |
| | | FGFPVHYTDVSNMSRLARQRLLGRSWSVPVIRHLFAPLKEYFA |
| | | CVSSGNSNANSRGPSFSSGLVPLSLRGSHMGPMEIYKTVSAWK |
| | | RQPVRVLSLFRNIDKVLKSLGFLESGSGSGGGTLKYVEDVTNV |
| | | VRRDVEKWGPFDLVYGSTQPLGSSCDRCPGWYMFQFHRILQY |
| | | ALPRQESQRPFFWIFMDNLLLTEDDQETTTRFLQTEAVTLQDVR |
| | | GRDYQNAMRVWSNIPGLKSKHAPLTPKEEEYLQAQVRSRSKL |
| | | DAPKVDLLVKNCLLPLREYFKYFSQNSLPLGGPSSGAPPPSGGS |
| | | PAGSPTSTEEGTSESATPESGPGTSTEPSEGSAPGSPAGSPTSTEE |
| | | GTSTEPSEGSAPGTSTEPSEPKKKRKVSRPGERPFQCRICMRNFS |
| | | RTDTLARHLRTHTGEKPFQCRICMRNFSRKTALNRHLKTHTGS |
| | | QKPFQCRICMRNFSRNESLKVHLRTHTGEKPFQCRICMRNFSVK |
| | | NTLTRHLKTHTGSQKPFQCRICMRNFSRREHLVRHLRTHTGEKP |
| | | FQCRICMRNFSLTHNLRRHLKTHLRGSPKKKRKVSGSETPGTSE |
| | | SATPESTGRTLVTFKDVFVDFTREEWKLLDTAQQIVYRNVMLE |
| | | NYKNLVSLGYQLTKPDVILRLEKGEEPSADYKDDDDKA |
| 134 | ZFoff-15 with | MGTMPKKKRKVPKKKRKVYNHDQEFDPPKVYPPVPAEKRKPI |
| | 2xNLS | RVLSLFDGIATGLLVLKDLGIQVDRYIASEVCEDSITVGMVRHQ |
| | | GKIMYVGDVRSVTQKHIQEWGPFDLVIGGSPCNDLSIVNPARK |
| | | GLYEGTGRLFFEFYRLLHDARPKEGDDRPFFWLFENVVAMGVS |
| | | DKRDISRFLESNPVMIDAKEVSAAHRARYFWGNLPGMNRPLAS |
| | | TVNDKLELQECLEHGRIAKFSKVRTITTRSNSIKQGKDQHFPVF |
| | | MNEKEDILWCTEMERVFGFPVHYTDVSNMSRLARQRLLGRSW |
| | | SVPVIRHLFAPLKEYFACVSSGNSNANSRGPSFSSGLVPLSLRGS |
| | | HMGPMEIYKTVSAWKRQPVRVLSLFRNIDKVLKSLGFLESGSG |
| | | SGGGTLKYVEDVTNVVRRDVEKWGPFDLVYGSTQPLGSSCDR |
| | | CPGWYMFQFHRILQYALPRQESQRPFFWIFMDNLLLTEDDQET |
| | | TTRFLQTEAVTLQDVRGRDYQNAMRVWSNIPGLKSKHAPLTPK |
| | | EEEYLQAQVRSRSKLDAPKVDLLVKNCLLPLREYFKYFSQNSLP |
| | | LGGPSSGAPPPSGGSPAGSPTSTEEGTSESATPESGPGTSTEPSEG |
| | | SAPGSPAGSPTSTEEGTSTEPSEGSAPGTSTEPSESRPGERPFQCR |
| | | ICMRNFSRTDTLARHLRTHTGEKPFQCRICMRNFSRKTALNRHL |
| | | KTHTGSQKPFQCRICMRNFSRNESLKVHLRTHTGEKPFQCRICM |
| | | RNFSVKNTLTRHLKTHTGSQKPFQCRICMRNFSRREHLVRHLR |
| | | THTGEKPFQCRICMRNFSLTHNLRRHLKTHLRGSSGSETPGTSE |
| | | SATPESTGRTLVTFKDVFVDFTREEWKLLDTAQQIVYRNVMLE |
| | | NYKNLVSLGYQLTKPDVILRLEKGEEPSADYKDDDDKAPKKKR |
| | | KVPKKKRKV |
| 135 | ZFoff-60 Target | GCAGAGGCCGGAGGGGGTC |
| | Sequence | |
| 136 | ZFoff-60 ZFP | SRPGERPFQCRICMRNFSDPSVLKRHLRTHTGEKPFQCRICMRN |
| | Amino Acid | FSRTEHLARHLKTHTGGGGSQKPFQCRICMRNFSQSPHLKRHL |
| | Sequence | RTHTGEKPFQCRICMRNFSDQTTLRRHLKTHTGSQKPFQCRICM |
| | | RNFSKHSNLTRHTRTHTGEKPFQCRICMRNFSQMETLKRHLRT |
| | | HLRGS |
| 137 | ZFoff-60 Full | MGTMYNHDQEFDPPKVYPPVPAEKRKPIRVLSLFDGIATGLLVL |
| | Amino Acid | KDLGIQVDRYIASEVCEDSITVGMVRHQGKIMYVGDVRSVTQK |
| | Sequence | HIQEWGPFDLVIGGSPCNDLSIVNPARKGLYEGTGRLFFEFYRLL |
| | | HDARPKEGDDRPFFWLFENVVAMGVSDKRDISRFLESNPVMID |
| | | AKEVSAAHRARYFWGNLPGMNRPLASTVNDKLELQECLEHGR |
| | | IAKFSKVRTITTRSNSIKQGKDQHFPVFMNEKEDILWCTEMERV |
| | | FGFPVHYTDVSNMSRLARQRLLGRSWSVPVIRHLFAPLKEYFA |
| | | CVSSGNSNANSRGPSFSSGLVPLSLRGSHMGPMEIYKTVSAWK |
| | | RQPVRVLSLFRNIDKVLKSLGFLESGSGSGGGTLKYVEDVTNV |
| | | VRRDVEKWGPFDLVYGSTQPLGSSCDRCPGWYMFQFHRILQY |
| | | ALPRQESQRPFFWIFMDNLLLTEDDQETTTRFLQTEAVTLQDVR |
| | | GRDYQNAMRVWSNIPGLKSKHAPLTPKEEEYLQAQVRSRSKL |
| | | DAPKVDLLVKNCLLPLREYFKYFSQNSLPLGGPSSGAPPPSGGS |
| | | PAGSPTSTEEGTSESATPESGPGTSTEPSEGSAPGSPAGSPTSTEE |
| | | GTSTEPSEGSAPGTSTEPSEPKKKRKVSRPGERPFQCRICMRNFS |
| | | DPSVLKRHLRTHTGEKPFQCRICMRNFSRTEHLARHLKTHTGG |
| | | GGSQKPFQCRICMRNFSQSPHLKRHLRTHTGEKPFQCRICMRNF |
| | | SDQTTLRRHLKTHTGSQKPFQCRICMRNFSKHSNLTRHTRTHT |
| | | GEKPFQCRICMRNFSQMETLKRHLRTHLRGSPKKKRKVSGSET |
| | | PGTSESATPESTGRTLVTFKDVFVDFTREEWKLLDTAQQIVYRN |
| | | VMLENYKNLVSLGYQLTKPDVILRLEKGEEPSADYKDDDDKA |
| 138 | ZFoff-60 with | MGTMPKKKRKVPKKKRKVYNHDQEFDPPKVYPPVPAEKRKPI |
| | 2xNLS | RVLSLFDGIATGLLVLKDLGIQVDRYIASEVCEDSITVGMVRHQ |
| | | GKIMYVGDVRSVTQKHIQEWGPFDLVIGGSPCNDLSIVNPARK |
| | | GLYEGTGRLFFEFYRLLHDARPKEGDDRPFFWLFENVVAMGVS |
| | | DKRDISRFLESNPVMIDAKEVSAAHRARYFWGNLPGMNRPLAS |
| | | TVNDKLELQECLEHGRIAKFSKVRTITTRSNSIKQGKDQHFPVF |
| | | MNEKEDILWCTEMERVFGFPVHYTDVSNMSRLARQRLLGRSW |
| | | SVPVIRHLFAPLKEYFACVSSGNSNANSRGPSFSSGLVPLSLRGS |
| | | HMGPMEIYKTVSAWKRQPVRVLSLFRNIDKVLKSLGFLESGSG |
| | | SGGGTLKYVEDVTNVVRRDVEKWGPFDLVYGSTQPLGSSCDR |
| | | CPGWYMFQFHRILQY ALPRQESQRPFFWIFMDNLLLTEDDQET |
| | | TTRFLQTEAVTLQDVRGRDYQNAMRVWSNIPGLKSKHAPLTPK |
| | | EEEYLQAQVRSRSKLDAPKVDLLVKNCLLPLREYFKYFSQNSLP |
| | | LGGPSSGAPPPSGGSPAGSPTSTEEGTSESATPESGPGTSTEPSEG |
| | | SAPGSPAGSPTSTEEGTSTEPSEGSAPGTSTEPSESRPGERPFQCR |
| | | ICMRNFSDPSVLKRHLRTHTGEKPFQCRICMRNFSRTEHLARHL |
| | | KTHTGGGGSQKPFQCRICMRNFSQSPHLKRHLRTHTGEKPFQC |
| | | RICMRNFSDQTTLRRHLKTHTGSQKPFQCRICMRNFSKHSNLTR |
| | | HTRTHTGEKPFQCRICMRNFSQMETLKRHLRTHLRGSSGSETPG |
| | | TSESATPESTGRTLVTFKDVFVDFTREEWKLLDTAQQIVYRNV |
| | | MLENYKNLVSLGYQLTKPDVILRLEKGEEPSADYKDDDDKAPK |
| | | KKRKVPKKKRKV |
| 139 | ZFoff-137 Target | GGTGGGGAGGACTGTGCA |
| | Sequence | |
| 140 | ZFoff-137 ZFP | SRPGERPFQCRICMRNFSQSTTLKRHLRTHTGEKPFQCRICMRN |
| | Amino Acid | FSGAHGLAGHLKTHTGSQKPFQCRICMRNFSDQTNLRRHLRTH |
| | Sequence | TGEKPFQCRICMRNFSRQDNLQRHLKTHTGSQKPFQCRICMRN |
| | | FSKGDHLRRHTRTHTGEKPFQCRICMRNFSEAHHLSRHLRTHL |
| | | RGS |
| 141 | ZFoff-137 Full | MGTMYNHDQEFDPPKVYPPVPAEKRKPIRVLSLFDGIATGLLVL |
| | Amino Acid | KDLGIQVDRYIASEVCEDSITVGMVRHQGKIMYVGDVRSVTQK |
| | Sequence | HIQEWGPFDLVIGGSPCNDLSIVNPARKGLYEGTGRLFFEFYRLL |
| | | HDARPKEGDDRPFFWLFENVVAMGVSDKRDISRFLESNPVMID |
| | | AKEVSAAHRARYFWGNLPGMNRPLASTVNDKLELQECLEHGR |
| | | IAKFSKVRTITTRSNSIKQGKDQHFPVFMNEKEDILWCTEMERV |
| | | FGFPVHYTDVSNMSRLARQRLLGRSWSVPVIRHLFAPLKEYFA |
| | | CVSSGNSNANSRGPSFSSGLVPLSLRGSHMGPMEIYKTVSAWK |
| | | RQPVRVLSLFRNIDKVLKSLGFLESGSGSGGGTLKYVEDVTNV |
| | | VRRDVEKWGPFDLVYGSTQPLGSSCDRCPGWYMFQFHRILQY |
| | | ALPRQESQRPFFWIFMDNLLLTEDDQETTTRFLQTEAVTLQDVR |
| | | GRDYQNAMRVWSNIPGLKSKHAPLTPKEEEYLQAQVRSRSKL |
| | | DAPKVDLLVKNCLLPLREYFKYFSQNSLPLGGPSSGAPPPSGGS |
| | | PAGSPTSTEEGTSESATPESGPGTSTEPSEGSAPGSPAGSPTSTEE |
| | | GTSTEPSEGSAPGTSTEPSEPKKKRKVSRPGERPFQCRICMRNFS |
| | | QSTTLKRHLRTHTGEKPFQCRICMRNFSGAHGLAGHLKTHTGS |
| | | QKPFQCRICMRNFSDQTNLRRHLRTHTGEKPFQCRICMRNFSRQ |
| | | DNLQRHLKTHTGSQKPFQCRICMRNFSKGDHLRRHTRTHTGEK |
| | | PFQCRICMRNFSEAHHLSRHLRTHLRGSPKKKRKVSGSETPGTS |
| | | ESATPESTGRTLVTFKDVFVDFTREEWKLLDTAQQIVYRNVML |
| | | ENYKNLVSLGYQLTKPDVILRLEKGEEPSADYKDDDDKA |
| 142 | ZFoff-137 with | MGTMPKKKRKVPKKKRKVYNHDQEFDPPKVYPPVPAEKRKPI |
| | 2xNLS | RVLSLFDGIATGLLVLKDLGIQVDRYIASEVCEDSITVGMVRHQ |
| | | GKIMYVGDVRSVTQKHIQEWGPFDLVIGGSPCNDLSIVNPARK |
| | | GLYEGTGRLFFEFYRLLHDARPKEGDDRPFFWLFENVVAMGVS |
| | | DKRDISRFLESNPVMIDAKEVSAAHRARYFWGNLPGMNRPLAS |
| | | TVNDKLELQECLEHGRIAKFSKVRTITTRSNSIKQGKDQHFPVF |
| | | MNEKEDILWCTEMERVFGFPVHYTDVSNMSRLARQRLLGRSW |
| | | SVPVIRHLFAPLKEYFACVSSGNSNANSRGPSFSSGLVPLSLRGS |
| | | HMGPMEIYKTVSAWKRQPVRVLSLFRNIDKVLKSLGFLESGSG |
| | | SGGGTLKYVEDVTNVVRRDVEKWGPFDLVYGSTQPLGSSCDR |
| | | CPGWYMFQFHRILQY ALPRQESQRPFFWIFMDNLLLTEDDQET |
| | | TTRFLQTEAVTLQDVRGRDYQNAMRVWSNIPGLKSKHAPLTPK |
| | | EEEYLQAQVRSRSKLDAPKVDLLVKNCLLPLREYFKYFSQNSLP |
| | | LGGPSSGAPPPSGGSPAGSPTSTEEGTSESATPESGPGTSTEPSEG |
| | | SAPGSPAGSPTSTEEGTSTEPSEGSAPGTSTEPSESRPGERPFQCR |
| | | ICMRNFSQSTTLKRHLRTHTGEKPFQCRICMRNFSGAHGLAGH |
| | | LKTHTGSQKPFQCRICMRNFSDQTNLRRHLRTHTGEKPFQCRIC |
| | | MRNFSRQDNLQRHLKTHTGSQKPFQCRICMRNFSKGDHLRRHT |
| | | RTHTGEKPFQCRICMRNFSEAHHLSRHLRTHLRGSSGSETPGTS |
| | | ESATPESTGRTLVTFKDVFVDFTREEWKLLDTAQQIVYRNVML |
| | | ENYKNLVSLGYQLTKPDVILRLEKGEEPSADYKDDDDKAPKKK |
| | | RKVPKKKRKV |
| 143 | ZFoff-152 Target | GCAGGAGGACGAGGACGGC |
| | Sequence | |
| 144 | ZFoff-152 ZFP | SRPGERPFQCRICMRNFSTPSKLDRHTRTHTGEKPFQCRICMRN |
| | Amino Acid | FSLAENLRRHLRTHTGSQKPFQCRICMRNFSRQDNLGRHLRTH |
| | Sequence | TGEKPFQCRICMRNFSDGGNLGRHLKTHTGGGGSQKPFQCRIC |
| | | MRNFSQSPHLKRHLRTHTGEKPFQCRICMRNFSQSTSLQRHLKT |
| | | HLRGS |
| 145 | ZFoff-152 Full | MGTMYNHDQEFDPPKVYPPVPAEKRKPIRVLSLFDGIATGLLVL |
| | Amino Acid | KDLGIQVDRYIASEVCEDSITVGMVRHQGKIMYVGDVRSVTQK |
| | Sequence | HIQEWGPFDLVIGGSPCNDLSIVNPARKGLYEGTGRLFFEFYRLL |
| | | HDARPKEGDDRPFFWLFENVVAMGVSDKRDISRFLESNPVMID |
| | | AKEVSAAHRARYFWGNLPGMNRPLASTVNDKLELQECLEHGR |
| | | IAKFSKVRTITTRSNSIKQGKDQHFPVFMNEKEDILWCTEMERV |
| | | FGFPVHYTDVSNMSRLARQRLLGRSWSVPVIRHLFAPLKEYFA |
| | | CVSSGNSNANSRGPSFSSGLVPLSLRGSHMGPMEIYKTVSAWK |
| | | RQPVRVLSLFRNIDKVLKSLGFLESGSGSGGGTLKYVEDVTNV |
| | | VRRDVEKWGPFDLVYGSTQPLGSSCDRCPGWYMFQFHRILQY |
| | | ALPRQESQRPFFWIFMDNLLLTEDDQETTTRFLQTEAVTLQDVR |
| | | GRDYQNAMRVWSNIPGLKSKHAPLTPKEEEYLQAQVRSRSKL |
| | | DAPKVDLLVKNCLLPLREYFKYFSQNSLPLGGPSSGAPPPSGGS |
| | | PAGSPTSTEEGTSESATPESGPGTSTEPSEGSAPGSPAGSPTSTEE |
| | | GTSTEPSEGSAPGTSTEPSEPKKKRKVSRPGERPFQCRICMRNFS |
| | | TPSKLDRHTRTHTGEKPFQCRICMRNFSLAENLRRHLRTHTGSQ |
| | | KPFQCRICMRNFSRQDNLGRHLRTHTGEKPFQCRICMRNFSDG |
| | | GNLGRHLKTHTGGGGSQKPFQCRICMRNFSQSPHLKRHLRTHT |
| | | GEKPFQCRICMRNFSQSTSLQRHLKTHLRGSPKKKRKVSGSETP |
| | | GTSESATPESTGRTLVTFKDVFVDFTREEWKLLDTAQQIVYRNV |
| | | MLENYKNLVSLGYQLTKPDVILRLEKGEEPSADYKDDDDKA |
| 146 | ZFoff-152 with | MGTMPKKKRKVPKKKRKVYNHDQEFDPPKVYPPVPAEKRKPI |
| | 2xNLS | RVLSLFDGIATGLLVLKDLGIQVDRYIASEVCEDSITVGMVRHQ |
| | | GKIMYVGDVRSVTQKHIQEWGPFDLVIGGSPCNDLSIVNPARK |
| | | GLYEGTGRLFFEFYRLLHDARPKEGDDRPFFWLFENVVAMGVS |
| | | DKRDISRFLESNPVMIDAKEVSAAHRARYFWGNLPGMNRPLAS |
| | | TVNDKLELQECLEHGRIAKFSKVRTITTRSNSIKQGKDQHFPVF |
| | | MNEKEDILWCTEMERVFGFPVHYTDVSNMSRLARQRLLGRSW |
| | | SVPVIRHLFAPLKEYFACVSSGNSNANSRGPSFSSGLVPLSLRGS |
| | | HMGPMEIYKTVSAWKRQPVRVLSLFRNIDKVLKSLGFLESGSG |
| | | SGGGTLKYVEDVTNVVRRDVEKWGPFDLVYGSTQPLGSSCDR |
| | | CPGWYMFQFHRILQYALPRQESQRPFFWIFMDNLLLTEDDQET |
| | | TTRFLQTEAVTLQDVRGRDYQNAMRVWSNIPGLKSKHAPLTPK |
| | | EEEYLQAQVRSRSKLDAPKVDLLVKNCLLPLREYFKYFSQNSLP |
| | | LGGPSSGAPPPSGGSPAGSPTSTEEGTSESATPESGPGTSTEPSEG |
| | | SAPGSPAGSPTSTEEGTSTEPSEGSAPGTSTEPSESRPGERPFQCR |
| | | ICMRNFSTPSKLDRHTRTHTGEKPFQCRICMRNFSLAENLRRHL |
| | | RTHTGSQKPFQCRICMRNFSRQDNLGRHLRTHTGEKPFQCRIC |
| | | MRNFSDGGNLGRHLKTHTGGGGSQKPFQCRICMRNFSQSPHLK |
| | | RHLRTHTGEKPFQCRICMRNFSQSTSLQRHLKTHLRGSSGSETP |
| | | GTSESATPESTGRTLVTFKDVFVDFTREEWKLLDTAQQIVYRNV |
| | | MLENYKNLVSLGYQLTKPDVILRLEKGEEPSADYKDDDDKAPK |
| | | KKRKVPKKKRKV |
| 147 | ZFoff-153 Target | GCAGGAGGACGAGGACGGC |
| | Sequence | |
| 148 | ZFoff-153 ZFP | SRPGERPFQCRICMRNFSERAKLIRHLRTHTGEKPFQCRICMRNF |
| | Amino Acid | SDPSNLRRHLKTHTGSQKPFQCRICMRNFSRQDNLGRHLRTHT |
| | Sequence | GEKPFQCRICMRNFSDQGNLGRHLKTHTGGGGSQKPFQCRICM |
| | | RNFSQSAHLKRHLRTHTGEKPFQCRICMRNFSQDVSLVRHLKT |
| | | HLRGS |
| 149 | ZFoff-153 Full | MGTMYNHDQEFDPPKVYPPVPAEKRKPIRVLSLFDGIATGLLVL |
| | Amino Acid | KDLGIQVDRYIASEVCEDSITVGMVRHQGKIMYVGDVRSVTQK |
| | Sequence | HIQEWGPFDLVIGGSPCNDLSIVNPARKGLYEGTGRLFFEFYRLL |
| | | HDARPKEGDDRPFFWLFENVVAMGVSDKRDISRFLESNPVMID |
| | | AKEVSAAHRARYFWGNLPGMNRPLASTVNDKLELQECLEHGR |
| | | IAKFSKVRTITTRSNSIKQGKDQHFPVFMNEKEDILWCTEMERV |
| | | FGFPVHYTDVSNMSRLARQRLLGRSWSVPVIRHLFAPLKEYFA |
| | | CVSSGNSNANSRGPSFSSGLVPLSLRGSHMGPMEIYKTVSAWK |
| | | RQPVRVLSLFRNIDKVLKSLGFLESGSGSGGGTLKYVEDVTNV |
| | | VRRDVEKWGPFDLVYGSTQPLGSSCDRCPGWYMFQFHRILQY |
| | | ALPRQESQRPFFWIFMDNLLLTEDDQETTTRFLQTEAVTLQDVR |
| | | GRDYQNAMRVWSNIPGLKSKHAPLTPKEEEYLQAQVRSRSKL |
| | | DAPKVDLLVKNCLLPLREYFKYFSQNSLPLGGPSSGAPPPSGGS |
| | | PAGSPTSTEEGTSESATPESGPGTSTEPSEGSAPGSPAGSPTSTEE |
| | | GTSTEPSEGSAPGTSTEPSEPKKKRKVSRPGERPFQCRICMRNFS |
| | | ERAKLIRHLRTHTGEKPFQCRICMRNFSDPSNLRRHLKTHTGSQ |
| | | KPFQCRICMRNFSRQDNLGRHLRTHTGEKPFQCRICMRNFSDQ |
| | | GNLGRHLKTHTGGGGSQKPFQCRICMRNFSQSAHLKRHLRTHT |
| | | GEKPFQCRICMRNFSQDVSLVRHLKTHLRGSPKKKRKVSGSET |
| | | PGTSESATPESTGRTLVTFKDVFVDFTREEWKLLDTAQQIVYRN |
| | | VMLENYKNLVSLGYQLTKPDVILRLEKGEEPSADYKDDDDKA |
| 150 | ZFoff-153 with | MGTMPKKKRKVPKKKRKVYNHDQEFDPPKVYPPVPAEKRKPI |
| | 2xNLS | RVLSLFDGIATGLLVLKDLGIQVDRYIASEVCEDSITVGMVRHQ |
| | | GKIMYVGDVRSVTQKHIQEWGPFDLVIGGSPCNDLSIVNPARK |
| | | GLYEGTGRLFFEFYRLLHDARPKEGDDRPFFWLFENVVAMGVS |
| | | DKRDISRFLESNPVMIDAKEVSAAHRARYFWGNLPGMNRPLAS |
| | | TVNDKLELQECLEHGRIAKFSKVRTITTRSNSIKQGKDQHFPVF |
| | | MNEKEDILWCTEMERVFGFPVHYTDVSNMSRLARQRLLGRSW |
| | | SVPVIRHLFAPLKEYFACVSSGNSNANSRGPSFSSGLVPLSLRGS |
| | | HMGPMEIYKTVSAWKRQPVRVLSLFRNIDKVLKSLGFLESGSG |
| | | SGGGTLKYVEDVTNVVRRDVEKWGPFDLVYGSTQPLGSSCDR |
| | | CPGWYMFQFHRILQYALPRQESQRPFFWIFMDNLLLTEDDQET |
| | | TTRFLQTEAVTLQDVRGRDYQNAMRVWSNIPGLKSKHAPLTPK |
| | | EEEYLQAQVRSRSKLDAPKVDLLVKNCLLPLREYFKYFSQNSLP |
| | | LGGPSSGAPPPSGGSPAGSPTSTEEGTSESATPESGPGTSTEPSEG |
| | | SAPGSPAGSPTSTEEGTSTEPSEGSAPGTSTEPSESRPGERPFQCR |
| | | ICMRNFSERAKLIRHLRTHTGEKPFQCRICMRNFSDPSNLRRHL |
| | | KTHTGSQKPFQCRICMRNFSRQDNLGRHLRTHTGEKPFQCRIC |
| | | MRNFSDQGNLGRHLKTHTGGGGSQKPFQCRICMRNFSQSAHL |
| | | KRHLRTHTGEKPFQCRICMRNFSQDVSLVRHLKTHLRGSSGSET |
| | | PGTSESATPESTGRTLVTFKDVFVDFTREEWKLLDTAQQIVYRN |
| | | VMLENYKNLVSLGYQLTKPDVILRLEKGEEPSADYKDDDDKAP |
| | | KKKRKVPKKKRKV |
| 151 | TAR087 Target | GGCGGAAGTGCTGCCTGATA |
| | DNA Sequence | |
| 152 | TAR089 Target | GCAACACCGCCTAGACCGAC |
| | DNA Sequence | |
| 153 | TAR090 Target | GGAGGTTCAGAAAGCCTAAG |
| | DNA Sequence | |
| 154 | TAR091 Target | GCAGCACTAGAGTCCCCTCA |
| | DNA Sequence | |
| 155 | TAR092 Target | GAACAACTCGTGACTGGGGT |
| | DNA Sequence | |
| 156 | TAR093 Target | GGGAGAGGAGGACAAGCGCC |
| | DNA Sequence | |
| 157 | TAR094 Target | GGACCCACCGACACCACGCC |
| | DNA Sequence | |
| 158 | TAR114 Target | CGCGGGAAAATGCAAGACGA |
| | DNA Sequence | |
| 159 | 5′ UTR 4 | TAATACGACTCACTATAAGGGGCGCTCGAGCAGGTTCAGAA |
| | | GGAGATCAAAAACCCCCAAGGATCAAAC |
| 160 | Fusion Protein 13 | MPKKKRKVPKKKRKVYNHDQEFDPPKVYPPVPAEKRKPIRVLS |
| | Amino Acid | LFDGIATGLLVLKDLGIQVDRYIASEVCEDSITVGMVRHQGKIM |
| | Sequence | YVGDVRSVTQKHIQEWGPFDLVIGGSPCNDLSIVNPARKGLYE |
| | NLS-NLS-3A- | GTGRLFFEFYRLLHDARPKEGDDRPFFWLFENVVAMGVSDKR |
| | h3L-dCas9-NLS- | DISRFLESNPVMIDAKEVSAAHRARYFWGNLPGMNRPLASTVN |
| | ZIM3-NLS-NLS | DKLELQECLEHGRIAKFSKVRTITTRSNSIKQGKDQHFPVFMNE |
| | | KEDILWCTEMERVFGFPVHYTDVSNMSRLARQRLLGRSWSVP |
| | | VIRHLFAPLKEYFACVSSGNSNANSRGPSFSSGLVPLSLRGSHM |
| | | AAIPALDPEAEPSMDVILVGSSELSSSVSPGTGRDLIAYEVKANQ |
| | | RNIEDICICCGSLQVHTQHPLFEGGICAPCKDKFLDALFLYDDDG |
| | | YQSYCSICCSGETLLICGNPDCTRCYCFECVDSLVGPGTSGKVH |
| | | AMSNWVCYLCLPSSRSGLLQRRRKWRSQLKAFYDRESENPLE |
| | | MFETVPVWRRQPVRVLSLFEDIKKELTSLGFLESGSDPGQLKHV |
| | | VDVTDTVRKDVEEWGPFDLVYGATPPLGHTCDRPPSWYLFQF |
| | | HRLLQYARPKPGSPRPFFWMFVDNLVLNKEDLDVASRFLEMEP |
| | | VTIPDVHGGSLQNAVRVWSNIPAIRSRHWALVSEEELSLLAQNK |
| | | QSSKLAAKWPTKLVKNCFLPLREYFKYFSTELTSSLGGPSSGAP |
| | | PPSGGSPAGSPTSTEEGTSESATPESGPGTSTEPSEGSAPGSPAGS |
| | | PTSTEEGTSTEPSEGSAPGTSTEPSELEDKKYSIGLAIGTNSVGW |
| | | AVITDEYKVPSKKFKVLGNTDRHSIKKNLIGALLFDSGETAEAT |
| | | RLKRTARRRYTRRKNRICYLQEIFSNEMAKVDDSFFHRLEESFL |
| | | VEEDKKHERHPIFGNIVDEVAYHEKYPTIYHLRKKLVDSTDKA |
| | | DLRLIYLALAHMIKFRGHFLIEGDLNPDNSDVDKLFIQLVQTYN |
| | | QLFEENPINASGVDAKAILSARLSKSRRLENLIAQLPGEKKNGLF |
| | | GNLIALSLGLTPNFKSNFDLAEDAKLQLSKDTYDDDLDNLLAQI |
| | | GDQYADLFLAAKNLSDAILLSDILRVNTEITKAPLSASMIKRYD |
| | | EHHQDLTLLKALVRQQLPEKYKEIFFDQSKNGYAGYIDGGASQ |
| | | EEFYKFIKPILEKMDGTEELLVKLNREDLLRKQRTFDNGSIPHQI |
| | | HLGELHAILRRQEDFYPFLKDNREKIEKILTFRIPYYVGPLARGN |
| | | SRFAWMTRKSEETITPWNFEEVVDKGASAQSFIERMTNFDKNL |
| | | PNEKVLPKHSLLYEYFTVYNELTKVKYVTEGMRKPAFLSGEQK |
| | | KAIVDLLFKTNRKVTVKQLKEDYFKKIECFDSVEISGVEDRFNA |
| | | SLGTYHDLLKIIKDKDFLDNEENEDILEDIVLTLTLFEDREMIEER |
| | | LKTYAHLFDDKVMKQLKRRRYTGWGRLSRKLINGIRDKQSGK |
| | | TILDFLKSDGFANRNFMQLIHDDSLTFKEDIQKAQVSGQGDSLH |
| | | EHIANLAGSPAIKKGILQTVKVVDELVKVMGRHKPENIVIEMAR |
| | | ENQTTQKGQKNSRERMKRIEEGIKELGSQILKEHPVENTQLQNE |
| | | KLYLYYLQNGRDMYVDQELDINRLSDYDVDAIVPQSFLKDDSI |
| | | DNKVLTRSDKNRGKSDNVPSEEVVKKMKNYWRQLLNAKLITQ |
| | | RKFDNLTKAERGGLSELDKAGFIKRQLVETRQITKHVAQILDSR |
| | | MNTKYDENDKLIREVKVITLKSKLVSDFRKDFQFYKVREINNY |
| | | HHAHDAYLNAVVGTALIKKYPKLESEFVYGDYKVYDVRKMIA |
| | | KSEQEIGKATAKYFFYSNIMNFFKTEITLANGEIRKRPLIETNGET |
| | | GEIVWDKGRDFATVRKVLSMPQVNIVKKTEVQTGGFSKESILP |
| | | KRNSDKLIARKKDWDPKKYGGFDSPTVAYSVLVVAKVEKGKS |
| | | KKLKSVKELLGITIMERSSFEKNPIDFLEAKGYKEVKKDLIIKLP |
| | | KYSLFELENGRKRMLASAGELQKGNELALPSKYVNFLYLASHY |
| | | EKLKGSPEDNEQKQLFVEQHKHYLDEIIEQISEFSKRVILADANL |
| | | DKVLSAYNKHRDKPIREQAENIIHLFTLTNLGAPAAFKYFDTTID |
| | | RKRYTSTKEVLDATLIHQSITGLYETRIDLSQLGGDSPKKKRKV |
| | | GVDGSSGSETPGTSESATPESTGMNNSQGRVTFEDVTVNFTQG |
| | | EWQRLNPEQRNLYRDVMLENYSNLVSVGQGETTKPDVILRLEQ |
| | | GKEPWLEEEEVLGSGRAEKNGDIGGQIWKPKDVKESLSAPKKK |
| | | RKVPKKKRKV |
| 161 | Fusion Protein 13 | ATGCCAAAAAAGAAGAGAAAGGTACCGAAGAAAAAAAGAA |
| | DNA Sequence | AGGTATACAATCACGATCAGGAGTTCGACCCCCCTAAGGTGT |
| | | ACCCACCAGTGCCTGCAGAGAAGAGGAAGCCAATCCGGGTG |
| | | CTGAGCCTGTTTGATGGCATCGCCACCGGCCTGCTGGTGCTG |
| | | AAGGATCTGGGCATCCAGGTGGACCGGTACATCGCCTCCGA |
| | | GGTGTGCGAGGATTCTATCACCGTGGGCATGGTGCGCCACCA |
| | | GGGCAAGATCATGTATGTGGGCGACGTGCGGTCCGTGACAC |
| | | AGAAGCACATCCAGGAGTGGGGCCCATTCGATCTGGTGATC |
| | | GGCGGCAGCCCCTGTAATGACCTGTCCATCGTGAACCCTGCA |
| | | AGGAAGGGACTGTACGAGGGAACCGGCCGGCTGTTCTTTGA |
| | | GTTTTATAGACTGCTGCACGACGCCAGGCCTAAGGAGGGCG |
| | | ACGATAGACCATTCTTTTGGCTGTTCGAGAATGTGGTGGCTA |
| | | TGGGCGTGAGCGATAAGAGGGACATCTCCAGGTTTCTGGAG |
| | | TCTAACCCCGTGATGATCGATGCAAAGGAGGTGTCCGCCGC |
| | | ACACAGAGCCAGGTATTTCTGGGGCAATCTGCCAGGAATGA |
| | | ACAGGCCACTGGCAAGCACCGTGAATGACAAGCTGGAGCTG |
| | | CAGGAGTGCCTGGAGCACGGAAGGATCGCCAAGTTTTCCAA |
| | | GGTGCGCACAATCACCACACGGAGCAATTCCATCAAGCAGG |
| | | GCAAGGATCAGCACTTCCCCGTGTTCATGAACGAGAAGGAG |
| | | GACATCCTGTGGTGTACCGAGATGGAGAGAGTGTTCGGCTTT |
| | | CCAGTGCACTACACAGACGTGTCTAACATGAGCAGGCTGGC |
| | | AAGGCAGCGGCTGCTGGGCAGATCTTGGAGCGTGCCCGTGA |
| | | TCAGGCACCTGTTCGCCCCTCTGAAGGAGTATTTTGCCTGCG |
| | | TGAGCAGCGGCAACTCCAATGCCAACAGCCGGGGCCCCTCT |
| | | TTCAGCTCCGGATTGGTGCCTCTGAGCCTGAGGGGCTCCCAC |
| | | ATGGCAGCAATCCCCGCCCTGGACCCCGAGGCCGAGCCTAG |
| | | CATGGACGTGATCCTGGTGGGCTCTAGCGAGCTGTCCTCTAG |
| | | CGTGTCTCCAGGAACCGGAAGGGATCTGATCGCATACGAGG |
| | | TGAAGGCCAATCAGCGGAACATCGAGGACATCTGTATCTGC |
| | | TGTGGCAGCCTGCAGGTGCACACACAGCACCCACTGTTCGA |
| | | GGGAGGAATCTGCGCACCCTGTAAGGATAAGTTCCTGGACG |
| | | CCCTGTTTCTGTACGACGATGACGGCTACCAGTCCTATTGCT |
| | | CTATCTGCTGTTCCGGCGAGACCCTGCTGATCTGCGGCAATC |
| | | CAGATTGTACAAGGTGCTATTGTTTTGAGTGCGTGGACTCTC |
| | | TGGTGGGACCAGGCACCAGCGGAAAGGTGCACGCCATGTCC |
| | | AACTGGGTGTGCTACCTGTGCCTGCCATCCTCTCGCAGCGGA |
| | | CTGCTGCAGCGGAGAAGGAAGTGGAGATCCCAGCTGAAGGC |
| | | CTTCTATGATAGGGAGTCTGAGAACCCCCTGGAGATGTTTGA |
| | | GACCGTGCCAGTGTGGCGCCGGCAGCCCGTGAGGGTGCTGA |
| | | GCCTGTTCGAGGATATCAAGAAGGAGCTGACATCCCTGGGC |
| | | TTTCTGGAGTCCGGCTCTGACCCCGGACAGCTGAAGCACGTG |
| | | GTGGATGTGACCGACACAGTGCGGAAGGATGTGGAGGAGTG |
| | | GGGCCCTTTCGACCTGGTGTACGGAGCAACCCCTCCACTGGG |
| | | ACACACATGCGACAGACCCCCTTCTTGGTACCTGTTCCAGTT |
| | | TCACCGCCTGCTGCAGTATGCAAGGCCAAAGCCAGGCAGCC |
| | | CTAGACCATTCTTTTGGATGTTCGTGGATAATCTGGTGCTGA |
| | | ACAAGGAGGATCTGGACGTGGCCAGCAGGTTTCTGGAGATG |
| | | GAGCCAGTGACCATCCCAGACGTGCACGGCGGCTCCCTGCA |
| | | GAATGCCGTGCGCGTGTGGTCTAACATCCCTGCCATCAGAAG |
| | | CAGGCACTGGGCACTGGTGAGCGAGGAGGAGCTGTCCCTGC |
| | | TGGCCCAGAATAAGCAGAGCAGCAAGCTGGCCGCCAAGTGG |
| | | CCTACAAAGCTGGTGAAGAACTGCTTCCTGCCACTGCGGGA |
| | | GTACTTCAAGTATTTTTCCACCGAGCTGACATCTAGCCTGGG |
| | | AGGACCCTCCTCTGGCGCCCCACCACCTAGCGGCGGCTCCCC |
| | | TGCCGGCTCTCCAACCAGCACAGAGGAGGGCACCAGCGAGT |
| | | CCGCCACACCAGAGTCTGGACCTGGCACCAGCACAGAGCCA |
| | | TCCGAGGGCTCTGCCCCAGGCTCTCCTGCAGGCAGCCCTACC |
| | | TCCACCGAAGAGGGCACCAGCACAGAGCCTTCTGAGGGCAG |
| | | CGCCCCAGGCACCTCTACAGAGCCAAGCGAGCTCGAGGACA |
| | | AGAAGTACAGCATCGGCCTGGCCATCGGCACCAACTCTGTG |
| | | GGCTGGGCCGTGATCACCGACGAGTACAAGGTGCCCAGCAA |
| | | GAAATTCAAGGTGCTGGGCAACACCGACCGGCACAGCATCA |
| | | AGAAGAACCTGATCGGAGCCCTGCTGTTCGACAGCGGCGAA |
| | | ACAGCCGAGGCCACCCGGCTGAAGAGAACCGCCAGAAGAA |
| | | GATACACCAGACGGAAGAACCGGATCTGCTATCTGCAAGAG |
| | | ATCTTCAGCAACGAGATGGCCAAGGTGGACGACAGCTTCTTC |
| | | CACAGACTGGAAGAGTCCTTCCTGGTGGAAGAGGATAAGAA |
| | | GCACGAGCGGCACCCCATCTTCGGCAACATCGTGGACGAGG |
| | | TGGCCTACCACGAGAAGTACCCCACCATCTACCACCTGAGA |
| | | AAGAAACTGGTGGACAGCACCGACAAGGCCGACCTGCGGCT |
| | | GATCTATCTGGCCCTGGCCCACATGATCAAGTTCCGGGGCCA |
| | | CTTCCTGATCGAGGGCGACCTGAACCCCGACAACAGCGACG |
| | | TGGACAAGCTGTTCATCCAGCTGGTGCAGACCTACAACCAGC |
| | | TGTTCGAGGAAAACCCCATCAACGCCAGCGGCGTGGACGCC |
| | | AAGGCCATCCTGTCTGCCAGACTGAGCAAGAGCAGACGGCT |
| | | GGAAAATCTGATCGCCCAGCTGCCCGGCGAGAAGAAGAATG |
| | | GCCTGTTCGGCAACCTGATTGCCCTGAGCCTGGGCCTGACCC |
| | | CCAACTTCAAGAGCAACTTCGACCTGGCCGAGGATGCCAAA |
| | | CTGCAGCTGAGCAAGGACACCTACGACGACGACCTGGACAA |
| | | CCTGCTGGCCCAGATCGGCGACCAGTACGCCGACCTGTTTCT |
| | | GGCCGCCAAGAACCTGTCCGACGCCATCCTGCTGAGCGACA |
| | | TCCTGAGAGTGAACACCGAGATCACCAAGGCCCCCCTGAGC |
| | | GCCTCTATGATCAAGAGATACGACGAGCACCACCAGGACCT |
| | | GACCCTGCTGAAAGCTCTCGTGCGGCAGCAGCTGCCTGAGA |
| | | AGTACAAAGAGATTTTCTTCGACCAGAGCAAGAACGGCTAC |
| | | GCCGGCTACATTGACGGCGGAGCCAGCCAGGAAGAGTTCTA |
| | | CAAGTTCATCAAGCCCATCCTGGAAAAGATGGACGGCACCG |
| | | AGGAACTGCTCGTGAAGCTGAACAGAGAGGACCTGCTGCGG |
| | | AAGCAGCGGACCTTCGACAACGGCAGCATCCCCCACCAGAT |
| | | CCACCTGGGAGAGCTGCACGCCATTCTGCGGCGGCAGGAAG |
| | | ATTTTTACCCATTCCTGAAGGACAACCGGGAAAAGATCGAG |
| | | AAGATCCTGACCTTCCGCATCCCCTACTACGTGGGCCCTCTG |
| | | GCCAGGGGAAACAGCAGATTCGCCTGGATGACCAGAAAGAG |
| | | CGAGGAAACCATCACCCCCTGGAACTTCGAGGAAGTGGTGG |
| | | ACAAGGGCGCTTCCGCCCAGAGCTTCATCGAGCGGATGACC |
| | | AACTTCGATAAGAACCTGCCCAACGAGAAGGTGCTGCCCAA |
| | | GCACAGCCTGCTGTACGAGTACTTCACCGTGTATAACGAGCT |
| | | GACCAAAGTGAAATACGTGACCGAGGGAATGAGAAAGCCCG |
| | | CCTTCCTGAGCGGCGAGCAGAAAAAGGCCATCGTGGACCTG |
| | | CTGTTCAAGACCAACCGGAAAGTGACCGTGAAGCAGCTGAA |
| | | AGAGGACTACTTCAAGAAAATCGAGTGCTTCGACTCCGTGG |
| | | AAATCTCCGGCGTGGAAGATCGGTTCAACGCCTCCCTGGGCA |
| | | CATACCACGATCTGCTGAAAATTATCAAGGACAAGGACTTCC |
| | | TGGACAATGAGGAAAACGAGGACATTCTGGAAGATATCGTG |
| | | CTGACCCTGACACTGTTTGAGGACAGAGAGATGATCGAGGA |
| | | ACGGCTGAAAACCTATGCCCACCTGTTCGACGACAAAGTGA |
| | | TGAAGCAGCTGAAGCGGCGGAGATACACCGGCTGGGGCAGG |
| | | CTGAGCCGGAAGCTGATCAACGGCATCCGGGACAAGCAGTC |
| | | CGGCAAGACAATCCTGGATTTCCTGAAGTCCGACGGCTTCGC |
| | | CAACAGAAACTTCATGCAGCTGATCCACGACGACAGCCTGA |
| | | CCTTTAAAGAGGACATCCAGAAAGCCCAGGTGTCCGGCCAG |
| | | GGCGATAGCCTGCACGAGCACATTGCCAATCTGGCCGGCAG |
| | | CCCCGCCATTAAGAAGGGCATCCTGCAGACAGTGAAGGTGG |
| | | TGGACGAGCTCGTGAAAGTGATGGGCCGGCACAAGCCCGAG |
| | | AACATCGTGATCGAAATGGCCAGAGAGAACCAGACCACCCA |
| | | GAAGGGACAGAAGAACAGCCGCGAGAGAATGAAGCGGATC |
| | | GAAGAGGGCATCAAAGAGCTGGGCAGCCAGATCCTGAAAGA |
| | | ACACCCCGTGGAAAACACCCAGCTGCAGAACGAGAAGCTGT |
| | | ACCTGTACTACCTGCAGAATGGGGGGATATGTACGTGGAC |
| | | CAGGAACTGGACATCAACCGGCTGTCCGACTACGATGTGGA |
| | | CGCCATCGTGCCTCAGAGCTTTCTGAAGGACGACTCCATCGA |
| | | CAACAAGGTGCTGACCAGAAGCGACAAGAACCGGGGCAAG |
| | | AGCGACAACGTGCCCTCCGAAGAGGTCGTGAAGAAGATGAA |
| | | GAACTACTGGCGGCAGCTGCTGAACGCCAAGCTGATTACCC |
| | | AGAGAAAGTTCGACAATCTGACCAAGGCCGAGAGAGGCGGC |
| | | CTGAGCGAACTGGATAAGGCCGGCTTCATCAAGAGACAGCT |
| | | GGTGGAAACCCGGCAGATCACAAAGCACGTGGCACAGATCC |
| | | TGGACTCCCGGATGAACACTAAGTACGACGAGAATGACAAG |
| | | CTGATCCGGGAAGTGAAAGTGATCACCCTGAAGTCCAAGCT |
| | | GGTGTCCGATTTCCGGAAGGATTTCCAGTTTTACAAAGTGCG |
| | | CGAGATCAACAACTACCACCACGCCCACGACGCCTACCTGA |
| | | ACGCCGTCGTGGGAACCGCCCTGATCAAAAAGTACCCTAAG |
| | | CTGGAAAGCGAGTTCGTGTACGGCGACTACAAGGTGTACGA |
| | | CGTGCGGAAGATGATCGCCAAGAGCGAGCAGGAAATCGGCA |
| | | AGGCTACCGCCAAGTACTTCTTCTACAGCAACATCATGAACT |
| | | TTTTCAAGACCGAGATTACCCTGGCCAACGGCGAGATCCGG |
| | | AAGCGGCCTCTGATCGAGACAAACGGCGAAACCGGGGAGAT |
| | | CGTGTGGGATAAGGGCCGGGATTTTGCCACCGTGCGGAAAG |
| | | TGCTGAGCATGCCCCAAGTGAATATCGTGAAAAAGACCGAG |
| | | GTGCAGACAGGCGGCTTCAGCAAAGAGTCTATCCTGCCCAA |
| | | GAGGAACAGCGATAAGCTGATCGCCAGAAAGAAGGACTGG |
| | | GACCCTAAGAAGTACGGCGGCTTCGACAGCCCCACCGTGGC |
| | | CTATTCTGTGCTGGTGGTGGCCAAAGTGGAAAAGGGCAAGT |
| | | CCAAGAAACTGAAGAGTGTGAAAGAGCTGCTGGGGATCACC |
| | | ATCATGGAAAGAAGCAGCTTCGAGAAGAATCCCATCGACTT |
| | | TCTGGAAGCCAAGGGCTACAAAGAAGTGAAAAAGGACCTGA |
| | | TCATCAAGCTGCCTAAGTACTCCCTGTTCGAGCTGGAAAACG |
| | | GCCGGAAGAGAATGCTGGCCTCTGCCGGCGAACTGCAGAAG |
| | | GGAAACGAACTGGCCCTGCCCTCCAAATATGTGAACTTCCTG |
| | | TACCTGGCCAGCCACTATGAGAAGCTGAAGGGCTCCCCCGA |
| | | GGATAATGAGCAGAAACAGCTGTTTGTGGAACAGCACAAGC |
| | | ACTACCTGGACGAGATCATCGAGCAGATCAGCGAGTTCTCC |
| | | AAGAGAGTGATCCTGGCCGACGCTAATCTGGACAAAGTGCT |
| | | GTCCGCCTACAACAAGCACCGGGATAAGCCCATCAGAGAGC |
| | | AGGCCGAGAATATCATCCACCTGTTTACCCTGACCAATCTGG |
| | | GAGCCCCTGCCGCCTTCAAGTACTTTGACACCACCATCGACC |
| | | GGAAGAGGTACACCAGCACCAAAGAGGTGCTGGACGCCACC |
| | | CTGATCCACCAGAGCATCACCGGCCTGTACGAGACACGGAT |
| | | CGACCTGTCTCAGCTGGGAGGCGACAGCCCCAAGAAGAAGA |
| | | GAAAGGTGGGAGTCGACGGATCCAGCGGCTCCGAGACCCCA |
| | | GGCACATCTGAGAGCGCCACCCCTGAGTCCACCGGTATGAA |
| | | CAATTCACAGGGGAGAGTGACATTCGAAGACGTGACCGTGA |
| | | ACTTCACCCAGGGAGAATGGCAGCGCTTGAACCCAGAACAA |
| | | AGGAACCTCTATCGGGACGTGATGCTGGAAAACTACTCAAA |
| | | TTTGGTGAGCGTTGGGCAGGGTGAGACCACTAAGCCTGACG |
| | | TGATCCTGAGATTGGAACAGGGCAAGGAGCCTTGGCTCGAG |
| | | GAAGAGGAAGTCCTGGGCTCAGGGAGGGCCGAGAAAAACG |
| | | GTGATATAGGAGGCCAGATATGGAAGCCTAAGGACGTCAAG |
| | | GAGAGCCTGAGCGCTCCCAAGAAGAAAAGGAAGGTCCCAAA |
| | | GAAAAAAAGAAAGGTGTGA |
| 162 | Fusion Protein 14 | MPKKKRKVPKKKRKVYNHDQEFDPPKVYPPVPAEKRKPIRVLS |
| | Amino Acid | LFDGIATGLLVLKDLGIQVDRYIASEVCEDSITVGMVRHQGKIM |
| | Sequence | YVGDVRSVTQKHIQEWGPFDLVIGGSPCNDLSIVNPARKGLYE |
| | NLS-NLS-3A- | GTGRLFFEFYRLLHDARPKEGDDRPFFWLFENVVAMGVSDKR |
| | h3L-dCas9-NLS- | DISRFLESNPVMIDAKEVSAAHRARYFWGNLPGMNRPLASTVN |
| | ZN627-NLS-NLS | DKLELQECLEHGRIAKFSKVRTITTRSNSIKQGKDQHFPVFMNE |
| | | KEDILWCTEMERVFGFPVHYTDVSNMSRLARQRLLGRSWSVP |
| | | VIRHLFAPLKEYFACVSSGNSNANSRGPSFSSGLVPLSLRGSHM |
| | | AAIPALDPEAEPSMDVILVGSSELSSSVSPGTGRDLIAYEVKANQ |
| | | RNIEDICICCGSLQVHTQHPLFEGGICAPCKDKFLDALFLYDDDG |
| | | YQSYCSICCSGETLLICGNPDCTRCYCFECVDSLVGPGTSGKVH |
| | | AMSNWVCYLCLPSSRSGLLQRRRKWRSQLKAFYDRESENPLE |
| | | MFETVPVWRRQPVRVLSLFEDIKKELTSLGFLESGSDPGQLKHV |
| | | VDVTDTVRKDVEEWGPFDLVYGATPPLGHTCDRPPSWYLFQF |
| | | HRLLQYARPKPGSPRPFFWMFVDNLVLNKEDLDVASRFLEMEP |
| | | VTIPDVHGGSLQNAVRVWSNIPAIRSRHWALVSEEELSLLAQNK |
| | | QSSKLAAKWPTKLVKNCFLPLREYFKYFSTELTSSLGGPSSGAP |
| | | PPSGGSPAGSPTSTEEGTSESATPESGPGTSTEPSEGSAPGSPAGS |
| | | PTSTEEGTSTEPSEGSAPGTSTEPSELEDKKYSIGLAIGTNSVGW |
| | | AVITDEYKVPSKKFKVLGNTDRHSIKKNLIGALLFDSGETAEAT |
| | | RLKRTARRRYTRRKNRICYLQEIFSNEMAKVDDSFFHRLEESFL |
| | | VEEDKKHERHPIFGNIVDEVAYHEKYPTIYHLRKKLVDSTDKA |
| | | DLRLIYLALAHMIKFRGHFLIEGDLNPDNSDVDKLFIQLVQTYN |
| | | QLFEENPINASGVDAKAILSARLSKSRRLENLIAQLPGEKKNGLF |
| | | GNLIALSLGLTPNFKSNFDLAEDAKLQLSKDTYDDDLDNLLAQI |
| | | GDQYADLFLAAKNLSDAILLSDILRVNTEITKAPLSASMIKRYD |
| | | EHHQDLTLLKALVRQQLPEKYKEIFFDQSKNGYAGYIDGGASQ |
| | | EEFYKFIKPILEKMDGTEELLVKLNREDLLRKQRTFDNGSIPHQI |
| | | HLGELHAILRRQEDFYPFLKDNREKIEKILTFRIPYYVGPLARGN |
| | | SRFAWMTRKSEETITPWNFEEVVDKGASAQSFIERMTNFDKNL |
| | | PNEKVLPKHSLLYEYFTVYNELTKVKYVTEGMRKPAFLSGEQK |
| | | KAIVDLLFKTNRKVTVKQLKEDYFKKIECFDSVEISGVEDRFNA |
| | | SLGTYHDLLKIIKDKDFLDNEENEDILEDIVLTLTLFEDREMIEER |
| | | LKTYAHLFDDKVMKQLKRRRYTGWGRLSRKLINGIRDKQSGK |
| | | TILDFLKSDGFANRNFMQLIHDDSLTFKEDIQKAQVSGQGDSLH |
| | | EHIANLAGSPAIKKGILQTVKVVDELVKVMGRHKPENIVIEMAR |
| | | ENQTTQKGQKNSRERMKRIEEGIKELGSQILKEHPVENTQLQNE |
| | | KLYLYYLQNGRDMYVDQELDINRLSDYDVDAIVPQSFLKDDSI |
| | | DNKVLTRSDKNRGKSDNVPSEEVVKKMKNYWRQLLNAKLITQ |
| | | RKFDNLTKAERGGLSELDKAGFIKRQLVETRQITKHVAQILDSR |
| | | MNTKYDENDKLIREVKVITLKSKLVSDFRKDFQFYKVREINNY |
| | | HHAHDAYLNAVVGTALIKKYPKLESEFVYGDYKVYDVRKMIA |
| | | KSEQEIGKATAKYFFYSNIMNFFKTEITLANGEIRKRPLIETNGET |
| | | GEIVWDKGRDFATVRKVLSMPQVNIVKKTEVQTGGFSKESILP |
| | | KRNSDKLIARKKDWDPKKYGGFDSPTVAYSVLVVAKVEKGKS |
| | | KKLKSVKELLGITIMERSSFEKNPIDFLEAKGYKEVKKDLIIKLP |
| | | KYSLFELENGRKRMLASAGELQKGNELALPSKYVNFLYLASHY |
| | | EKLKGSPEDNEQKQLFVEQHKHYLDEIIEQISEFSKRVILADANL |
| | | DKVLSAYNKHRDKPIREQAENIIHLFTLTNLGAPAAFKYFDTTID |
| | | RKRYTSTKEVLDATLIHQSITGLYETRIDLSQLGGDSPKKKRKV |
| | | GVDGSSGSETPGTSESATPESTGDSVAFEDVAVNFTLEEWALLD |
| | | PSQKNLYRDVMRETFRNLASVGKQWEDQNIEDPFKIPRRNISHI |
| | | PERLCESKEGGQGEESAPKKKRKVPKKKRKV |
| 163 | Fusion Protein 14 | ATGCCAAAAAAGAAGAGAAAGGTACCGAAGAAAAAAAGAA |
| | DNA Sequence | AGGTATACAATCACGATCAGGAGTTCGACCCCCCTAAGGTGT |
| | | ACCCACCAGTGCCTGCAGAGAAGAGGAAGCCAATCCGGGTG |
| | | CTGAGCCTGTTTGATGGCATCGCCACCGGCCTGCTGGTGCTG |
| | | AAGGATCTGGGCATCCAGGTGGACCGGTACATCGCCTCCGA |
| | | GGTGTGCGAGGATTCTATCACCGTGGGCATGGTGCGCCACCA |
| | | GGGCAAGATCATGTATGTGGGCGACGTGCGGTCCGTGACAC |
| | | AGAAGCACATCCAGGAGTGGGGCCCATTCGATCTGGTGATC |
| | | GGCGGCAGCCCCTGTAATGACCTGTCCATCGTGAACCCTGCA |
| | | AGGAAGGGACTGTACGAGGGAACCGGCCGGCTGTTCTTTGA |
| | | GTTTTATAGACTGCTGCACGACGCCAGGCCTAAGGAGGGCG |
| | | ACGATAGACCATTCTTTTGGCTGTTCGAGAATGTGGTGGCTA |
| | | TGGGCGTGAGCGATAAGAGGGACATCTCCAGGTTTCTGGAG |
| | | TCTAACCCCGTGATGATCGATGCAAAGGAGGTGTCCGCCGC |
| | | ACACAGAGCCAGGTATTTCTGGGGCAATCTGCCAGGAATGA |
| | | ACAGGCCACTGGCAAGCACCGTGAATGACAAGCTGGAGCTG |
| | | CAGGAGTGCCTGGAGCACGGAAGGATCGCCAAGTTTTCCAA |
| | | GGTGCGCACAATCACCACACGGAGCAATTCCATCAAGCAGG |
| | | GCAAGGATCAGCACTTCCCCGTGTTCATGAACGAGAAGGAG |
| | | GACATCCTGTGGTGTACCGAGATGGAGAGAGTGTTCGGCTTT |
| | | CCAGTGCACTACACAGACGTGTCTAACATGAGCAGGCTGGC |
| | | AAGGCAGCGGCTGCTGGGCAGATCTTGGAGCGTGCCCGTGA |
| | | TCAGGCACCTGTTCGCCCCTCTGAAGGAGTATTTTGCCTGCG |
| | | TGAGCAGCGGCAACTCCAATGCCAACAGCCGGGGCCCCTCT |
| | | TTCAGCTCCGGATTGGTGCCTCTGAGCCTGAGGGGCTCCCAC |
| | | ATGGCAGCAATCCCCGCCCTGGACCCCGAGGCCGAGCCTAG |
| | | CATGGACGTGATCCTGGTGGGCTCTAGCGAGCTGTCCTCTAG |
| | | CGTGTCTCCAGGAACCGGAAGGGATCTGATCGCATACGAGG |
| | | TGAAGGCCAATCAGCGGAACATCGAGGACATCTGTATCTGC |
| | | TGTGGCAGCCTGCAGGTGCACACACAGCACCCACTGTTCGA |
| | | GGGAGGAATCTGCGCACCCTGTAAGGATAAGTTCCTGGACG |
| | | CCCTGTTTCTGTACGACGATGACGGCTACCAGTCCTATTGCT |
| | | CTATCTGCTGTTCCGGCGAGACCCTGCTGATCTGCGGCAATC |
| | | CAGATTGTACAAGGTGCTATTGTTTTGAGTGCGTGGACTCTC |
| | | TGGTGGGACCAGGCACCAGCGGAAAGGTGCACGCCATGTCC |
| | | AACTGGGTGTGCTACCTGTGCCTGCCATCCTCTCGCAGCGGA |
| | | CTGCTGCAGCGGAGAAGGAAGTGGAGATCCCAGCTGAAGGC |
| | | CTTCTATGATAGGGAGTCTGAGAACCCCCTGGAGATGTTTGA |
| | | GACCGTGCCAGTGTGGCGCCGGCAGCCCGTGAGGGTGCTGA |
| | | GCCTGTTCGAGGATATCAAGAAGGAGCTGACATCCCTGGGC |
| | | TTTCTGGAGTCCGGCTCTGACCCCGGACAGCTGAAGCACGTG |
| | | GTGGATGTGACCGACACAGTGCGGAAGGATGTGGAGGAGTG |
| | | GGGCCCTTTCGACCTGGTGTACGGAGCAACCCCTCCACTGGG |
| | | ACACACATGCGACAGACCCCCTTCTTGGTACCTGTTCCAGTT |
| | | TCACCGCCTGCTGCAGTATGCAAGGCCAAAGCCAGGCAGCC |
| | | CTAGACCATTCTTTTGGATGTTCGTGGATAATCTGGTGCTGA |
| | | ACAAGGAGGATCTGGACGTGGCCAGCAGGTTTCTGGAGATG |
| | | GAGCCAGTGACCATCCCAGACGTGCACGGCGGCTCCCTGCA |
| | | GAATGCCGTGCGCGTGTGGTCTAACATCCCTGCCATCAGAAG |
| | | CAGGCACTGGGCACTGGTGAGCGAGGAGGAGCTGTCCCTGC |
| | | TGGCCCAGAATAAGCAGAGCAGCAAGCTGGCCGCCAAGTGG |
| | | CCTACAAAGCTGGTGAAGAACTGCTTCCTGCCACTGCGGGA |
| | | GTACTTCAAGTATTTTTCCACCGAGCTGACATCTAGCCTGGG |
| | | AGGACCCTCCTCTGGCGCCCCACCACCTAGCGGCGGCTCCCC |
| | | TGCCGGCTCTCCAACCAGCACAGAGGAGGGCACCAGCGAGT |
| | | CCGCCACACCAGAGTCTGGACCTGGCACCAGCACAGAGCCA |
| | | TCCGAGGGCTCTGCCCCAGGCTCTCCTGCAGGCAGCCCTACC |
| | | TCCACCGAAGAGGGCACCAGCACAGAGCCTTCTGAGGGCAG |
| | | CGCCCCAGGCACCTCTACAGAGCCAAGCGAGCTCGAGGACA |
| | | AGAAGTACAGCATCGGCCTGGCCATCGGCACCAACTCTGTG |
| | | GGCTGGGCCGTGATCACCGACGAGTACAAGGTGCCCAGCAA |
| | | GAAATTCAAGGTGCTGGGCAACACCGACCGGCACAGCATCA |
| | | AGAAGAACCTGATCGGAGCCCTGCTGTTCGACAGCGGCGAA |
| | | ACAGCCGAGGCCACCCGGCTGAAGAGAACCGCCAGAAGAA |
| | | GATACACCAGACGGAAGAACCGGATCTGCTATCTGCAAGAG |
| | | ATCTTCAGCAACGAGATGGCCAAGGTGGACGACAGCTTCTTC |
| | | CACAGACTGGAAGAGTCCTTCCTGGTGGAAGAGGATAAGAA |
| | | GCACGAGCGGCACCCCATCTTCGGCAACATCGTGGACGAGG |
| | | TGGCCTACCACGAGAAGTACCCCACCATCTACCACCTGAGA |
| | | AAGAAACTGGTGGACAGCACCGACAAGGCCGACCTGCGGCT |
| | | GATCTATCTGGCCCTGGCCCACATGATCAAGTTCCGGGGCCA |
| | | CTTCCTGATCGAGGGCGACCTGAACCCCGACAACAGCGACG |
| | | TGGACAAGCTGTTCATCCAGCTGGTGCAGACCTACAACCAGC |
| | | TGTTCGAGGAAAACCCCATCAACGCCAGCGGCGTGGACGCC |
| | | AAGGCCATCCTGTCTGCCAGACTGAGCAAGAGCAGACGGCT |
| | | GGAAAATCTGATCGCCCAGCTGCCCGGCGAGAAGAAGAATG |
| | | GCCTGTTCGGCAACCTGATTGCCCTGAGCCTGGGCCTGACCC |
| | | CCAACTTCAAGAGCAACTTCGACCTGGCCGAGGATGCCAAA |
| | | CTGCAGCTGAGCAAGGACACCTACGACGACGACCTGGACAA |
| | | CCTGCTGGCCCAGATCGGCGACCAGTACGCCGACCTGTTTCT |
| | | GGCCGCCAAGAACCTGTCCGACGCCATCCTGCTGAGCGACA |
| | | TCCTGAGAGTGAACACCGAGATCACCAAGGCCCCCCTGAGC |
| | | GCCTCTATGATCAAGAGATACGACGAGCACCACCAGGACCT |
| | | GACCCTGCTGAAAGCTCTCGTGCGGCAGCAGCTGCCTGAGA |
| | | AGTACAAAGAGATTTTCTTCGACCAGAGCAAGAACGGCTAC |
| | | GCCGGCTACATTGACGGCGGAGCCAGCCAGGAAGAGTTCTA |
| | | CAAGTTCATCAAGCCCATCCTGGAAAAGATGGACGGCACCG |
| | | AGGAACTGCTCGTGAAGCTGAACAGAGAGGACCTGCTGCGG |
| | | AAGCAGCGGACCTTCGACAACGGCAGCATCCCCCACCAGAT |
| | | CCACCTGGGAGAGCTGCACGCCATTCTGCGGCGGCAGGAAG |
| | | ATTTTTACCCATTCCTGAAGGACAACCGGGAAAAGATCGAG |
| | | AAGATCCTGACCTTCCGCATCCCCTACTACGTGGGCCCTCTG |
| | | GCCAGGGGAAACAGCAGATTCGCCTGGATGACCAGAAAGAG |
| | | CGAGGAAACCATCACCCCCTGGAACTTCGAGGAAGTGGTGG |
| | | ACAAGGGCGCTTCCGCCCAGAGCTTCATCGAGCGGATGACC |
| | | AACTTCGATAAGAACCTGCCCAACGAGAAGGTGCTGCCCAA |
| | | GCACAGCCTGCTGTACGAGTACTTCACCGTGTATAACGAGCT |
| | | GACCAAAGTGAAATACGTGACCGAGGGAATGAGAAAGCCCG |
| | | CCTTCCTGAGCGGCGAGCAGAAAAAGGCCATCGTGGACCTG |
| | | CTGTTCAAGACCAACCGGAAAGTGACCGTGAAGCAGCTGAA |
| | | AGAGGACTACTTCAAGAAAATCGAGTGCTTCGACTCCGTGG |
| | | AAATCTCCGGCGTGGAAGATCGGTTCAACGCCTCCCTGGGCA |
| | | CATACCACGATCTGCTGAAAATTATCAAGGACAAGGACTTCC |
| | | TGGACAATGAGGAAAACGAGGACATTCTGGAAGATATCGTG |
| | | CTGACCCTGACACTGTTTGAGGACAGAGAGATGATCGAGGA |
| | | ACGGCTGAAAACCTATGCCCACCTGTTCGACGACAAAGTGA |
| | | TGAAGCAGCTGAAGCGGCGGAGATACACCGGCTGGGGCAGG |
| | | CTGAGCCGGAAGCTGATCAACGGCATCCGGGACAAGCAGTC |
| | | CGGCAAGACAATCCTGGATTTCCTGAAGTCCGACGGCTTCGC |
| | | CAACAGAAACTTCATGCAGCTGATCCACGACGACAGCCTGA |
| | | CCTTTAAAGAGGACATCCAGAAAGCCCAGGTGTCCGGCCAG |
| | | GGCGATAGCCTGCACGAGCACATTGCCAATCTGGCCGGCAG |
| | | CCCCGCCATTAAGAAGGGCATCCTGCAGACAGTGAAGGTGG |
| | | TGGACGAGCTCGTGAAAGTGATGGGCCGGCACAAGCCCGAG |
| | | AACATCGTGATCGAAATGGCCAGAGAGAACCAGACCACCCA |
| | | GAAGGGACAGAAGAACAGCCGCGAGAGAATGAAGCGGATC |
| | | GAAGAGGGCATCAAAGAGCTGGGCAGCCAGATCCTGAAAGA |
| | | ACACCCCGTGGAAAACACCCAGCTGCAGAACGAGAAGCTGT |
| | | ACCTGTACTACCTGCAGAATGGGCGGGATATGTACGTGGAC |
| | | CAGGAACTGGACATCAACCGGCTGTCCGACTACGATGTGGA |
| | | CGCCATCGTGCCTCAGAGCTTTCTGAAGGACGACTCCATCGA |
| | | CAACAAGGTGCTGACCAGAAGCGACAAGAACCGGGGCAAG |
| | | AGCGACAACGTGCCCTCCGAAGAGGTCGTGAAGAAGATGAA |
| | | GAACTACTGGCGGCAGCTGCTGAACGCCAAGCTGATTACCC |
| | | AGAGAAAGTTCGACAATCTGACCAAGGCCGAGAGAGGCGGC |
| | | CTGAGCGAACTGGATAAGGCCGGCTTCATCAAGAGACAGCT |
| | | GGTGGAAACCCGGCAGATCACAAAGCACGTGGCACAGATCC |
| | | TGGACTCCCGGATGAACACTAAGTACGACGAGAATGACAAG |
| | | CTGATCCGGGAAGTGAAAGTGATCACCCTGAAGTCCAAGCT |
| | | GGTGTCCGATTTCCGGAAGGATTTCCAGTTTTACAAAGTGCG |
| | | CGAGATCAACAACTACCACCACGCCCACGACGCCTACCTGA |
| | | ACGCCGTCGTGGGAACCGCCCTGATCAAAAAGTACCCTAAG |
| | | CTGGAAAGCGAGTTCGTGTACGGCGACTACAAGGTGTACGA |
| | | CGTGCGGAAGATGATCGCCAAGAGCGAGCAGGAAATCGGCA |
| | | AGGCTACCGCCAAGTACTTCTTCTACAGCAACATCATGAACT |
| | | TTTTCAAGACCGAGATTACCCTGGCCAACGGCGAGATCCGG |
| | | AAGCGGCCTCTGATCGAGACAAACGGCGAAACCGGGGAGAT |
| | | CGTGTGGGATAAGGGCCGGGATTTTGCCACCGTGCGGAAAG |
| | | TGCTGAGCATGCCCCAAGTGAATATCGTGAAAAAGACCGAG |
| | | GTGCAGACAGGCGGCTTCAGCAAAGAGTCTATCCTGCCCAA |
| | | GAGGAACAGCGATAAGCTGATCGCCAGAAAGAAGGACTGG |
| | | GACCCTAAGAAGTACGGCGGCTTCGACAGCCCCACCGTGGC |
| | | CTATTCTGTGCTGGTGGTGGCCAAAGTGGAAAAGGGCAAGT |
| | | CCAAGAAACTGAAGAGTGTGAAAGAGCTGCTGGGGATCACC |
| | | ATCATGGAAAGAAGCAGCTTCGAGAAGAATCCCATCGACTT |
| | | TCTGGAAGCCAAGGGCTACAAAGAAGTGAAAAAGGACCTGA |
| | | TCATCAAGCTGCCTAAGTACTCCCTGTTCGAGCTGGAAAACG |
| | | GCCGGAAGAGAATGCTGGCCTCTGCCGGCGAACTGCAGAAG |
| | | GGAAACGAACTGGCCCTGCCCTCCAAATATGTGAACTTCCTG |
| | | TACCTGGCCAGCCACTATGAGAAGCTGAAGGGCTCCCCCGA |
| | | GGATAATGAGCAGAAACAGCTGTTTGTGGAACAGCACAAGC |
| | | ACTACCTGGACGAGATCATCGAGCAGATCAGCGAGTTCTCC |
| | | AAGAGAGTGATCCTGGCCGACGCTAATCTGGACAAAGTGCT |
| | | GTCCGCCTACAACAAGCACCGGGATAAGCCCATCAGAGAGC |
| | | AGGCCGAGAATATCATCCACCTGTTTACCCTGACCAATCTGG |
| | | GAGCCCCTGCCGCCTTCAAGTACTTTGACACCACCATCGACC |
| | | GGAAGAGGTACACCAGCACCAAAGAGGTGCTGGACGCCACC |
| | | CTGATCCACCAGAGCATCACCGGCCTGTACGAGACACGGAT |
| | | CGACCTGTCTCAGCTGGGAGGCGACAGCCCCAAGAAGAAGA |
| | | GAAAGGTGGGAGTCGACGGATCCAGCGGCTCCGAGACCCCA |
| | | GGCACATCTGAGAGCGCCACCCCTGAGTCCACCGGTGACTC |
| | | CGTTGCTTTCGAGGACGTGGCCGTGAACTTCACACTTGAGGA |
| | | ATGGGCCTTGCTCGACCCAAGTCAGAAGAATCTGTACAGAG |
| | | ACGTGATGCGGGAGACATTCAGGAATCTCGCCAGTGTCGGA |
| | | AAGCAGTGGGAAGACCAGAACATCGAAGATCCTTTCAAGAT |
| | | ACCACGGCGCAATATCTCCCACATTCCTGAGAGGCTGTGTGA |
| | | ATCTAAGGAAGGCGGACAAGGTGAGGAAAGCGCTCCCAAGA |
| | | AGAAAAGGAAGGTCCCAAAGAAAAAAAGAAAGGTGTGA |
| 164 | Fusion Protein 15 | MPKKKRKVPKKKRKVYNHDQEFDPPKVYPPVPAEKRKPIRVLS |
| | Amino Acid | LFDGIATGLLVLKDLGIQVDRYIASEVCEDSITVGMVRHQGKIM |
| | Sequence | YVGDVRSVTQKHIQEWGPFDLVIGGSPCNDLSIVNPARKGLYE |
| | NLS-NLS-3A- | GTGRLFFEFYRLLHDARPKEGDDRPFFWLFENVVAMGVSDKR |
| | h3L-dCas9-NLS | DISRFLESNPVMIDAKEVSAAHRARYFWGNLPGMNRPLASTVN |
| | KOX1-NLS-NLS | DKLELQECLEHGRIAKFSKVRTITTRSNSIKQGKDQHFPVFMNE |
| | | KEDILWCTEMERVFGFPVHYTDVSNMSRLARQRLLGRSWSVP |
| | | VIRHLFAPLKEYFACVSSGNSNANSRGPSFSSGLVPLSLRGSHM |
| | | AAIPALDPEAEPSMDVILVGSSELSSSVSPGTGRDLIAYEVKANQ |
| | | RNIEDICICCGSLQVHTQHPLFEGGICAPCKDKFLDALFLYDDDG |
| | | YQSYCSICCSGETLLICGNPDCTRCYCFECVDSLVGPGTSGKVH |
| | | AMSNWVCYLCLPSSRSGLLQRRRKWRSQLKAFYDRESENPLE |
| | | MFETVPVWRRQPVRVLSLFEDIKKELTSLGFLESGSDPGQLKHV |
| | | VDVTDTVRKDVEEWGPFDLVYGATPPLGHTCDRPPSWYLFQF |
| | | HRLLQYARPKPGSPRPFFWMFVDNLVLNKEDLDVASRFLEMEP |
| | | VTIPDVHGGSLQNAVRVWSNIPAIRSRHWALVSEEELSLLAQNK |
| | | QSSKLAAKWPTKLVKNCFLPLREYFKYFSTELTSSLGGPSSGAP |
| | | PPSGGSPAGSPTSTEEGTSESATPESGPGTSTEPSEGSAPGSPAGS |
| | | PTSTEEGTSTEPSEGSAPGTSTEPSELEDKKYSIGLAIGTNSVGW |
| | | AVITDEYKVPSKKFKVLGNTDRHSIKKNLIGALLFDSGETAEAT |
| | | RLKRTARRRYTRRKNRICYLQEIFSNEMAKVDDSFFHRLEESFL |
| | | VEEDKKHERHPIFGNIVDEVAYHEKYPTIYHLRKKLVDSTDKA |
| | | DLRLIYLALAHMIKFRGHFLIEGDLNPDNSDVDKLFIQLVQTYN |
| | | QLFEENPINASGVDAKAILSARLSKSRRLENLIAQLPGEKKNGLF |
| | | GNLIALSLGLTPNFKSNFDLAEDAKLQLSKDTYDDDLDNLLAQI |
| | | GDQYADLFLAAKNLSDAILLSDILRVNTEITKAPLSASMIKRYD |
| | | EHHQDLTLLKALVRQQLPEKYKEIFFDQSKNGYAGYIDGGASQ |
| | | EEFYKFIKPILEKMDGTEELLVKLNREDLLRKQRTFDNGSIPHQI |
| | | HLGELHAILRRQEDFYPFLKDNREKIEKILTFRIPYYVGPLARGN |
| | | SRFAWMTRKSEETITPWNFEEVVDKGASAQSFIERMTNFDKNL |
| | | PNEKVLPKHSLLYEYFTVYNELTKVKYVTEGMRKPAFLSGEQK |
| | | KAIVDLLFKTNRKVTVKQLKEDYFKKIECFDSVEISGVEDRFNA |
| | | SLGTYHDLLKIIKDKDFLDNEENEDILEDIVLTLTLFEDREMIEER |
| | | LKTYAHLFDDKVMKQLKRRRYTGWGRLSRKLINGIRDKQSGK |
| | | TILDFLKSDGFANRNFMQLIHDDSLTFKEDIQKAQVSGQGDSLH |
| | | EHIANLAGSPAIKKGILQTVKVVDELVKVMGRHKPENIVIEMAR |
| | | ENQTTQKGQKNSRERMKRIEEGIKELGSQILKEHPVENTQLQNE |
| | | KLYLYYLQNGRDMYVDQELDINRLSDYDVDAIVPQSFLKDDSI |
| | | DNKVLTRSDKNRGKSDNVPSEEVVKKMKNYWRQLLNAKLITQ |
| | | RKFDNLTKAERGGLSELDKAGFIKRQLVETRQITKHVAQILDSR |
| | | MNTKYDENDKLIREVKVITLKSKLVSDFRKDFQFYKVREINNY |
| | | HHAHDAYLNAVVGTALIKKYPKLESEFVYGDYKVYDVRKMIA |
| | | KSEQEIGKATAKYFFYSNIMNFFKTEITLANGEIRKRPLIETNGET |
| | | GEIVWDKGRDFATVRKVLSMPQVNIVKKTEVQTGGFSKESILP |
| | | KRNSDKLIARKKDWDPKKYGGFDSPTVAYSVLVVAKVEKGKS |
| | | KKLKSVKELLGITIMERSSFEKNPIDFLEAKGYKEVKKDLIIKLP |
| | | KYSLFELENGRKRMLASAGELQKGNELALPSKYVNFLYLASHY |
| | | EKLKGSPEDNEQKQLFVEQHKHYLDEIIEQISEFSKRVILADANL |
| | | DKVLSAYNKHRDKPIREQAENIIHLFTLTNLGAPAAFKYFDTTID |
| | | RKRYTSTKEVLDATLIHQSITGLYETRIDLSQLGGDSPKKKRKV |
| | | GVDGSSGSETPGTSESATPESRTLVTFKDVFVDFTREEWKLLDT |
| | | AQQIVYRNVMLENYKNLVSLGYQLTKPDVILRLEKGEEPSAPK |
| | | KKRKVPKKKRKV |
| 165 | Fusion Protein 15 | ATGCCAAAAAAGAAGAGAAAGGTACCGAAGAAAAAAAGAA |
| | DNA Sequence | AGGTATACAATCACGATCAGGAGTTCGACCCCCCTAAGGTGT |
| | | ACCCACCAGTGCCTGCAGAGAAGAGGAAGCCAATCCGGGTG |
| | | CTGAGCCTGTTTGATGGCATCGCCACCGGCCTGCTGGTGCTG |
| | | AAGGATCTGGGCATCCAGGTGGACCGGTACATCGCCTCCGA |
| | | GGTGTGCGAGGATTCTATCACCGTGGGCATGGTGCGCCACCA |
| | | GGGCAAGATCATGTATGTGGGCGACGTGCGGTCCGTGACAC |
| | | AGAAGCACATCCAGGAGTGGGGCCCATTCGATCTGGTGATC |
| | | GGCGGCAGCCCCTGTAATGACCTGTCCATCGTGAACCCTGCA |
| | | AGGAAGGGACTGTACGAGGGAACCGGCCGGCTGTTCTTTGA |
| | | GTTTTATAGACTGCTGCACGACGCCAGGCCTAAGGAGGGCG |
| | | ACGATAGACCATTCTTTTGGCTGTTCGAGAATGTGGTGGCTA |
| | | TGGGCGTGAGCGATAAGAGGGACATCTCCAGGTTTCTGGAG |
| | | TCTAACCCCGTGATGATCGATGCAAAGGAGGTGTCCGCCGC |
| | | ACACAGAGCCAGGTATTTCTGGGGCAATCTGCCAGGAATGA |
| | | ACAGGCCACTGGCAAGCACCGTGAATGACAAGCTGGAGCTG |
| | | CAGGAGTGCCTGGAGCACGGAAGGATCGCCAAGTTTTCCAA |
| | | GGTGCGCACAATCACCACACGGAGCAATTCCATCAAGCAGG |
| | | GCAAGGATCAGCACTTCCCCGTGTTCATGAACGAGAAGGAG |
| | | GACATCCTGTGGTGTACCGAGATGGAGAGAGTGTTCGGCTTT |
| | | CCAGTGCACTACACAGACGTGTCTAACATGAGCAGGCTGGC |
| | | AAGGCAGCGGCTGCTGGGCAGATCTTGGAGCGTGCCCGTGA |
| | | TCAGGCACCTGTTCGCCCCTCTGAAGGAGTATTTTGCCTGCG |
| | | TGAGCAGCGGCAACTCCAATGCCAACAGCCGGGGCCCCTCT |
| | | TTCAGCTCCGGATTGGTGCCTCTGAGCCTGAGGGGCTCCCAC |
| | | ATGGCAGCAATCCCCGCCCTGGACCCCGAGGCCGAGCCTAG |
| | | CATGGACGTGATCCTGGTGGGCTCTAGCGAGCTGTCCTCTAG |
| | | CGTGTCTCCAGGAACCGGAAGGGATCTGATCGCATACGAGG |
| | | TGAAGGCCAATCAGCGGAACATCGAGGACATCTGTATCTGC |
| | | TGTGGCAGCCTGCAGGTGCACACACAGCACCCACTGTTCGA |
| | | GGGAGGAATCTGCGCACCCTGTAAGGATAAGTTCCTGGACG |
| | | CCCTGTTTCTGTACGACGATGACGGCTACCAGTCCTATTGCT |
| | | CTATCTGCTGTTCCGGCGAGACCCTGCTGATCTGCGGCAATC |
| | | CAGATTGTACAAGGTGCTATTGTTTTGAGTGCGTGGACTCTC |
| | | TGGTGGGACCAGGCACCAGCGGAAAGGTGCACGCCATGTCC |
| | | AACTGGGTGTGCTACCTGTGCCTGCCATCCTCTCGCAGCGGA |
| | | CTGCTGCAGCGGAGAAGGAAGTGGAGATCCCAGCTGAAGGC |
| | | CTTCTATGATAGGGAGTCTGAGAACCCCCTGGAGATGTTTGA |
| | | GACCGTGCCAGTGTGGCGCCGGCAGCCCGTGAGGGTGCTGA |
| | | GCCTGTTCGAGGATATCAAGAAGGAGCTGACATCCCTGGGC |
| | | TTTCTGGAGTCCGGCTCTGACCCCGGACAGCTGAAGCACGTG |
| | | GTGGATGTGACCGACACAGTGCGGAAGGATGTGGAGGAGTG |
| | | GGGCCCTTTCGACCTGGTGTACGGAGCAACCCCTCCACTGGG |
| | | ACACACATGCGACAGACCCCCTTCTTGGTACCTGTTCCAGTT |
| | | TCACCGCCTGCTGCAGTATGCAAGGCCAAAGCCAGGCAGCC |
| | | CTAGACCATTCTTTTGGATGTTCGTGGATAATCTGGTGCTGA |
| | | ACAAGGAGGATCTGGACGTGGCCAGCAGGTTTCTGGAGATG |
| | | GAGCCAGTGACCATCCCAGACGTGCACGGCGGCTCCCTGCA |
| | | GAATGCCGTGCGCGTGTGGTCTAACATCCCTGCCATCAGAAG |
| | | CAGGCACTGGGCACTGGTGAGCGAGGAGGAGCTGTCCCTGC |
| | | TGGCCCAGAATAAGCAGAGCAGCAAGCTGGCCGCCAAGTGG |
| | | CCTACAAAGCTGGTGAAGAACTGCTTCCTGCCACTGCGGGA |
| | | GTACTTCAAGTATTTTTCCACCGAGCTGACATCTAGCCTGGG |
| | | AGGACCCTCCTCTGGCGCCCCACCACCTAGCGGCGGCTCCCC |
| | | TGCCGGCTCTCCAACCAGCACAGAGGAGGGCACCAGCGAGT |
| | | CCGCCACACCAGAGTCTGGACCTGGCACCAGCACAGAGCCA |
| | | TCCGAGGGCTCTGCCCCAGGCTCTCCTGCAGGCAGCCCTACC |
| | | TCCACCGAAGAGGGCACCAGCACAGAGCCTTCTGAGGGCAG |
| | | CGCCCCAGGCACCTCTACAGAGCCAAGCGAGCTCGAGGACA |
| | | AGAAGTACAGCATCGGCCTGGCCATCGGCACCAACTCTGTG |
| | | GGCTGGGCCGTGATCACCGACGAGTACAAGGTGCCCAGCAA |
| | | GAAATTCAAGGTGCTGGGCAACACCGACCGGCACAGCATCA |
| | | AGAAGAACCTGATCGGAGCCCTGCTGTTCGACAGCGGCGAA |
| | | ACAGCCGAGGCCACCCGGCTGAAGAGAACCGCCAGAAGAA |
| | | GATACACCAGACGGAAGAACCGGATCTGCTATCTGCAAGAG |
| | | ATCTTCAGCAACGAGATGGCCAAGGTGGACGACAGCTTCTTC |
| | | CACAGACTGGAAGAGTCCTTCCTGGTGGAAGAGGATAAGAA |
| | | GCACGAGCGGCACCCCATCTTCGGCAACATCGTGGACGAGG |
| | | TGGCCTACCACGAGAAGTACCCCACCATCTACCACCTGAGA |
| | | AAGAAACTGGTGGACAGCACCGACAAGGCCGACCTGCGGCT |
| | | GATCTATCTGGCCCTGGCCCACATGATCAAGTTCCGGGGCCA |
| | | CTTCCTGATCGAGGGCGACCTGAACCCCGACAACAGCGACG |
| | | TGGACAAGCTGTTCATCCAGCTGGTGCAGACCTACAACCAGC |
| | | TGTTCGAGGAAAACCCCATCAACGCCAGCGGCGTGGACGCC |
| | | AAGGCCATCCTGTCTGCCAGACTGAGCAAGAGCAGACGGCT |
| | | GGAAAATCTGATCGCCCAGCTGCCCGGCGAGAAGAAGAATG |
| | | GCCTGTTCGGCAACCTGATTGCCCTGAGCCTGGGCCTGACCC |
| | | CCAACTTCAAGAGCAACTTCGACCTGGCCGAGGATGCCAAA |
| | | CTGCAGCTGAGCAAGGACACCTACGACGACGACCTGGACAA |
| | | CCTGCTGGCCCAGATCGGCGACCAGTACGCCGACCTGTTTCT |
| | | GGCCGCCAAGAACCTGTCCGACGCCATCCTGCTGAGCGACA |
| | | TCCTGAGAGTGAACACCGAGATCACCAAGGCCCCCCTGAGC |
| | | GCCTCTATGATCAAGAGATACGACGAGCACCACCAGGACCT |
| | | GACCCTGCTGAAAGCTCTCGTGCGGCAGCAGCTGCCTGAGA |
| | | AGTACAAAGAGATTTTCTTCGACCAGAGCAAGAACGGCTAC |
| | | GCCGGCTACATTGACGGCGGAGCCAGCCAGGAAGAGTTCTA |
| | | CAAGTTCATCAAGCCCATCCTGGAAAAGATGGACGGCACCG |
| | | AGGAACTGCTCGTGAAGCTGAACAGAGAGGACCTGCTGCGG |
| | | AAGCAGCGGACCTTCGACAACGGCAGCATCCCCCACCAGAT |
| | | CCACCTGGGAGAGCTGCACGCCATTCTGCGGCGGCAGGAAG |
| | | ATTTTTACCCATTCCTGAAGGACAACCGGGAAAAGATCGAG |
| | | AAGATCCTGACCTTCCGCATCCCCTACTACGTGGGCCCTCTG |
| | | GCCAGGGGAAACAGCAGATTCGCCTGGATGACCAGAAAGAG |
| | | CGAGGAAACCATCACCCCCTGGAACTTCGAGGAAGTGGTGG |
| | | ACAAGGGCGCTTCCGCCCAGAGCTTCATCGAGCGGATGACC |
| | | AACTTCGATAAGAACCTGCCCAACGAGAAGGTGCTGCCCAA |
| | | GCACAGCCTGCTGTACGAGTACTTCACCGTGTATAACGAGCT |
| | | GACCAAAGTGAAATACGTGACCGAGGGAATGAGAAAGCCCG |
| | | CCTTCCTGAGCGGCGAGCAGAAAAAGGCCATCGTGGACCTG |
| | | CTGTTCAAGACCAACCGGAAAGTGACCGTGAAGCAGCTGAA |
| | | AGAGGACTACTTCAAGAAAATCGAGTGCTTCGACTCCGTGG |
| | | AAATCTCCGGCGTGGAAGATCGGTTCAACGCCTCCCTGGGCA |
| | | CATACCACGATCTGCTGAAAATTATCAAGGACAAGGACTTCC |
| | | TGGACAATGAGGAAAACGAGGACATTCTGGAAGATATCGTG |
| | | CTGACCCTGACACTGTTTGAGGACAGAGAGATGATCGAGGA |
| | | ACGGCTGAAAACCTATGCCCACCTGTTCGACGACAAAGTGA |
| | | TGAAGCAGCTGAAGCGGCGGAGATACACCGGCTGGGGCAGG |
| | | CTGAGCCGGAAGCTGATCAACGGCATCCGGGACAAGCAGTC |
| | | CGGCAAGACAATCCTGGATTTCCTGAAGTCCGACGGCTTCGC |
| | | CAACAGAAACTTCATGCAGCTGATCCACGACGACAGCCTGA |
| | | CCTTTAAAGAGGACATCCAGAAAGCCCAGGTGTCCGGCCAG |
| | | GGCGATAGCCTGCACGAGCACATTGCCAATCTGGCCGGCAG |
| | | CCCCGCCATTAAGAAGGGCATCCTGCAGACAGTGAAGGTGG |
| | | TGGACGAGCTCGTGAAAGTGATGGGCCGGCACAAGCCCGAG |
| | | AACATCGTGATCGAAATGGCCAGAGAGAACCAGACCACCCA |
| | | GAAGGGACAGAAGAACAGCCGCGAGAGAATGAAGCGGATC |
| | | GAAGAGGGCATCAAAGAGCTGGGCAGCCAGATCCTGAAAGA |
| | | ACACCCCGTGGAAAACACCCAGCTGCAGAACGAGAAGCTGT |
| | | ACCTGTACTACCTGCAGAATGGGCGGGATATGTACGTGGAC |
| | | CAGGAACTGGACATCAACCGGCTGTCCGACTACGATGTGGA |
| | | CGCCATCGTGCCTCAGAGCTTTCTGAAGGACGACTCCATCGA |
| | | CAACAAGGTGCTGACCAGAAGCGACAAGAACCGGGGCAAG |
| | | AGCGACAACGTGCCCTCCGAAGAGGTCGTGAAGAAGATGAA |
| | | GAACTACTGGCGGCAGCTGCTGAACGCCAAGCTGATTACCC |
| | | AGAGAAAGTTCGACAATCTGACCAAGGCCGAGAGAGGCGGC |
| | | CTGAGCGAACTGGATAAGGCCGGCTTCATCAAGAGACAGCT |
| | | GGTGGAAACCCGGCAGATCACAAAGCACGTGGCACAGATCC |
| | | TGGACTCCCGGATGAACACTAAGTACGACGAGAATGACAAG |
| | | CTGATCCGGGAAGTGAAAGTGATCACCCTGAAGTCCAAGCT |
| | | GGTGTCCGATTTCCGGAAGGATTTCCAGTTTTACAAAGTGCG |
| | | CGAGATCAACAACTACCACCACGCCCACGACGCCTACCTGA |
| | | ACGCCGTCGTGGGAACCGCCCTGATCAAAAAGTACCCTAAG |
| | | CTGGAAAGCGAGTTCGTGTACGGCGACTACAAGGTGTACGA |
| | | CGTGCGGAAGATGATCGCCAAGAGCGAGCAGGAAATCGGCA |
| | | AGGCTACCGCCAAGTACTTCTTCTACAGCAACATCATGAACT |
| | | TTTTCAAGACCGAGATTACCCTGGCCAACGGCGAGATCCGG |
| | | AAGCGGCCTCTGATCGAGACAAACGGCGAAACCGGGGAGAT |
| | | CGTGTGGGATAAGGGCCGGGATTTTGCCACCGTGCGGAAAG |
| | | TGCTGAGCATGCCCCAAGTGAATATCGTGAAAAAGACCGAG |
| | | GTGCAGACAGGCGGCTTCAGCAAAGAGTCTATCCTGCCCAA |
| | | GAGGAACAGCGATAAGCTGATCGCCAGAAAGAAGGACTGG |
| | | GACCCTAAGAAGTACGGCGGCTTCGACAGCCCCACCGTGGC |
| | | CTATTCTGTGCTGGTGGTGGCCAAAGTGGAAAAGGGCAAGT |
| | | CCAAGAAACTGAAGAGTGTGAAAGAGCTGCTGGGGATCACC |
| | | ATCATGGAAAGAAGCAGCTTCGAGAAGAATCCCATCGACTT |
| | | TCTGGAAGCCAAGGGCTACAAAGAAGTGAAAAAGGACCTGA |
| | | TCATCAAGCTGCCTAAGTACTCCCTGTTCGAGCTGGAAAACG |
| | | GCCGGAAGAGAATGCTGGCCTCTGCCGGCGAACTGCAGAAG |
| | | GGAAACGAACTGGCCCTGCCCTCCAAATATGTGAACTTCCTG |
| | | TACCTGGCCAGCCACTATGAGAAGCTGAAGGGCTCCCCCGA |
| | | GGATAATGAGCAGAAACAGCTGTTTGTGGAACAGCACAAGC |
| | | ACTACCTGGACGAGATCATCGAGCAGATCAGCGAGTTCTCC |
| | | AAGAGAGTGATCCTGGCCGACGCTAATCTGGACAAAGTGCT |
| | | GTCCGCCTACAACAAGCACCGGGATAAGCCCATCAGAGAGC |
| | | AGGCCGAGAATATCATCCACCTGTTTACCCTGACCAATCTGG |
| | | GAGCCCCTGCCGCCTTCAAGTACTTTGACACCACCATCGACC |
| | | GGAAGAGGTACACCAGCACCAAAGAGGTGCTGGACGCCACC |
| | | CTGATCCACCAGAGCATCACCGGCCTGTACGAGACACGGAT |
| | | CGACCTGTCTCAGCTGGGAGGCGACAGCCCCAAGAAGAAGA |
| | | GAAAGGTGGGAGTCGACGGATCCAGCGGCTCCGAGACCCCA |
| | | GGCACATCTGAGAGCGCCACCCCTGAGTCCCGGACCCTGGT |
| | | GACCTTCAAGGATGTATTTGTGGACTTCACCAGGGAGGAGTG |
| | | GAAGCTGCTGGACACTGCTCAGCAGATCGTGTACAGAAATG |
| | | TGATGCTGGAGAACTATAAGAACCTGGTTTCCTTGGGTTATC |
| | | AGCTTACTAAGCCAGATGTGATCCTCCGGTTGGAGAAGGGA |
| | | GAAGAGCCCAGCGCTCCAAAAAAGAAGAGAAAGGTACCGA |
| | | AGAAAAAAAGAAAGGTCTGA |
| 166 | Fusion Protein 16 | MSPKKKRKVNHDQEFDPPKVYPPVPAEKRKPIRVLSLFDGIATG |
| | Amino Acid | LLVLKDLGIQVDRYIASEVCEDSITVGMVRHQGKIMYVGDVRS |
| | Sequence | VTQKHIQEWGPFDLVIGGSPCNDLSIVNPARKGLYEGTGRLFFE |
| | NLS-3A-h3L- | FYRLLHDARPKEGDDRPFFWLFENVVAMGVSDKRDISRFLESN |
| | dCas9-NLS | PVMIDAKEVSAAHRARYFWGNLPGMNRPLASTVNDKLELQEC |
| | KOX1 | LEHGRIAKFSKVRTITTRSNSIKQGKDQHFPVFMNEKEDILWCT |
| | | EMERVFGFPVHYTDVSNMSRLARQRLLGRSWSVPVIRHLFAPL |
| | | KEYFACVSSGNSNANSRGPSFSSGLVPLSLRGSHMAAIPALDPE |
| | | AEPSMDVILVGSSELSSSVSPGTGRDLIAYEVKANQRNIEDICIC |
| | | CGSLQVHTQHPLFEGGICAPCKDKFLDALFLYDDDGYQSYCSIC |
| | | CSGETLLICGNPDCTRCYCFECVDSLVGPGTSGKVHAMSNWVC |
| | | YLCLPSSRSGLLQRRRKWRSQLKAFYDRESENPLEMFETVPVW |
| | | RRQPVRVLSLFEDIKKELTSLGFLESGSDPGQLKHVVDVTDTVR |
| | | KDVEEWGPFDLVYGATPPLGHTCDRPPSWYLFQFHRLLQYARP |
| | | KPGSPRPFFWMFVDNLVLNKEDLDVASRFLEMEPVTIPDVHGG |
| | | SLQNAVRVWSNIPAIRSRHWALVSEEELSLLAQNKQSSKLAAK |
| | | WPTKLVKNCFLPLREYFKYFSTELTSSLGGPSSGAPPPSGGSPAG |
| | | SPTSTEEGTSESATPESGPGTSTEPSEGSAPGSPAGSPTSTEEGTS |
| | | TEPSEGSAPGTSTEPSELEDKKYSIGLAIGTNSVGWAVITDEYKV |
| | | PSKKFKVLGNTDRHSIKKNLIGALLFDSGETAEATRLKRTARRR |
| | | YTRRKNRICYLQEIFSNEMAKVDDSFFHRLEESFLVEEDKKHER |
| | | HPIFGNIVDEVAYHEKYPTIYHLRKKLVDSTDKADLRLIYLALA |
| | | HMIKFRGHFLIEGDLNPDNSDVDKLFIQLVQTYNQLFEENPINAS |
| | | GVDAKAILSARLSKSRRLENLIAQLPGEKKNGLFGNLIALSLGLT |
| | | PNFKSNFDLAEDAKLQLSKDTYDDDLDNLLAQIGDQYADLFLA |
| | | AKNLSDAILLSDILRVNTEITKAPLSASMIKRYDEHHQDLTLLKA |
| | | LVRQQLPEKYKEIFFDQSKNGYAGYIDGGASQEEFYKFIKPILEK |
| | | MDGTEELLVKLNREDLLRKQRTFDNGSIPHQIHLGELHAILRRQ |
| | | EDFYPFLKDNREKIEKILTFRIPYYVGPLARGNSRFAWMTRKSE |
| | | ETITPWNFEEVVDKGASAQSFIERMTNFDKNLPNEKVLPKHSLL |
| | | YEYFTVYNELTKVKYVTEGMRKPAFLSGEQKKAIVDLLFKTNR |
| | | KVTVKQLKEDYFKKIECFDSVEISGVEDRFNASLGTYHDLLKIIK |
| | | DKDFLDNEENEDILEDIVLTLTLFEDREMIEERLKTYAHLFDDK |
| | | VMKQLKRRRYTGWGRLSRKLINGIRDKQSGKTILDFLKSDGFA |
| | | NRNFMQLIHDDSLTFKEDIQKAQVSGQGDSLHEHIANLAGSPAI |
| | | KKGILQTVKVVDELVKVMGRHKPENIVIEMARENQTTQKGQK |
| | | NSRERMKRIEEGIKELGSQILKEHPVENTQLQNEKLYLYYLQNG |
| | | RDMYVDQELDINRLSDYDVDAIVPQSFLKDDSIDNKVLTRSDK |
| | | NRGKSDNVPSEEVVKKMKNYWRQLLNAKLITQRKFDNLTKAE |
| | | RGGLSELDKAGFIKRQLVETRQITKHVAQILDSRMNTKYDEND |
| | | KLIREVKVITLKSKLVSDFRKDFQFYKVREINNYHHAHDAYLN |
| | | AVVGTALIKKYPKLESEFVYGDYKVYDVRKMIAKSEQEIGKAT |
| | | AKYFFYSNIMNFFKTEITLANGEIRKRPLIETNGETGEIVWDKGR |
| | | DFATVRKVLSMPQVNIVKKTEVQTGGFSKESILPKRNSDKLIAR |
| | | KKDWDPKKYGGFDSPTVAYSVLVVAKVEKGKSKKLKSVKELL |
| | | GITIMERSSFEKNPIDFLEAKGYKEVKKDLIIKLPKYSLFELENGR |
| | | KRMLASAGELQKGNELALPSKYVNFLYLASHYEKLKGSPEDNE |
| | | QKQLFVEQHKHYLDEIIEQISEFSKRVILADANLDKVLSAYNKH |
| | | RDKPIREQAENIIHLFTLTNLGAPAAFKYFDTTIDRKRYTSTKEV |
| | | LDATLIHQSITGLYETRIDLSQLGGDSPKKKRKVGVDGSSGSETP |
| | | GTSESATPESRTLVTFKDVFVDFTREEWKLLDTAQQIVYRNVM |
| | | LENYKNLVSLGYQLTKPDVILRLEKGEEPWLV |
| 167 | Fusion Protein 16 | ATGTCGCCGAAGAAAAAGCGCAAGGTCAATCACGATCAGGA |
| | DNA Sequence | GTTCGACCCCCCTAAGGTGTACCCACCAGTGCCTGCAGAGAA |
| | | GAGGAAGCCAATCCGGGTGCTGAGCCTGTTTGATGGCATCG |
| | | CCACCGGCCTGCTGGTGCTGAAGGATCTGGGCATCCAGGTG |
| | | GACCGGTACATCGCCTCCGAGGTGTGCGAGGATTCTATCACC |
| | | GTGGGCATGGTGCGCCACCAGGGCAAGATCATGTATGTGGG |
| | | CGACGTGCGGTCCGTGACACAGAAGCACATCCAGGAGTGGG |
| | | GCCCATTCGATCTGGTGATCGGCGGCAGCCCCTGTAATGACC |
| | | TGTCCATCGTGAACCCTGCAAGGAAGGGACTGTACGAGGGA |
| | | ACCGGCCGGCTGTTCTTTGAGTTTTATAGACTGCTGCACGAC |
| | | GCCAGGCCTAAGGAGGGCGACGATAGACCATTCTTTTGGCT |
| | | GTTCGAGAATGTGGTGGCTATGGGCGTGAGCGATAAGAGGG |
| | | ACATCTCCAGGTTTCTGGAGTCTAACCCCGTGATGATCGATG |
| | | CAAAGGAGGTGTCCGCCGCACACAGAGCCAGGTATTTCTGG |
| | | GGCAATCTGCCAGGAATGAACAGGCCACTGGCAAGCACCGT |
| | | GAATGACAAGCTGGAGCTGCAGGAGTGCCTGGAGCACGGAA |
| | | GGATCGCCAAGTTTTCCAAGGTGCGCACAATCACCACACGG |
| | | AGCAATTCCATCAAGCAGGGCAAGGATCAGCACTTCCCCGT |
| | | GTTCATGAACGAGAAGGAGGACATCCTGTGGTGTACCGAGA |
| | | TGGAGAGAGTGTTCGGCTTTCCAGTGCACTACACAGACGTGT |
| | | CTAACATGAGCAGGCTGGCAAGGCAGCGGCTGCTGGGCAGA |
| | | TCTTGGAGCGTGCCCGTGATCAGGCACCTGTTCGCCCCTCTG |
| | | AAGGAGTATTTTGCCTGCGTGAGCAGCGGCAACTCCAATGCC |
| | | AACAGCCGGGGCCCCTCTTTCAGCTCCGGATTGGTGCCTCTG |
| | | AGCCTGAGGGGCTCCCACATGGCAGCAATCCCCGCCCTGGA |
| | | CCCCGAGGCCGAGCCTAGCATGGACGTGATCCTGGTGGGCT |
| | | CTAGCGAGCTGTCCTCTAGCGTGTCTCCAGGAACCGGAAGG |
| | | GATCTGATCGCATACGAGGTGAAGGCCAATCAGCGGAACAT |
| | | CGAGGACATCTGTATCTGCTGTGGCAGCCTGCAGGTGCACAC |
| | | ACAGCACCCACTGTTCGAGGGAGGAATCTGCGCACCCTGTA |
| | | AGGATAAGTTCCTGGACGCCCTGTTTCTGTACGACGATGACG |
| | | GCTACCAGTCCTATTGCTCTATCTGCTGTTCCGGCGAGACCC |
| | | TGCTGATCTGCGGCAATCCAGATTGTACAAGGTGCTATTGTT |
| | | TTGAGTGCGTGGACTCTCTGGTGGGACCAGGCACCAGCGGA |
| | | AAGGTGCACGCCATGTCCAACTGGGTGTGCTACCTGTGCCTG |
| | | CCATCCTCTCGCAGCGGACTGCTGCAGCGGAGAAGGAAGTG |
| | | GAGATCCCAGCTGAAGGCCTTCTATGATAGGGAGTCTGAGA |
| | | ACCCCCTGGAGATGTTTGAGACCGTGCCAGTGTGGCGCCGGC |
| | | AGCCCGTGAGGGTGCTGAGCCTGTTCGAGGATATCAAGAAG |
| | | GAGCTGACATCCCTGGGCTTTCTGGAGTCCGGCTCTGACCCC |
| | | GGACAGCTGAAGCACGTGGTGGATGTGACCGACACAGTGCG |
| | | GAAGGATGTGGAGGAGTGGGGCCCTTTCGACCTGGTGTACG |
| | | GAGCAACCCCTCCACTGGGACACACATGCGACAGACCCCCT |
| | | TCTTGGTACCTGTTCCAGTTTCACCGCCTGCTGCAGTATGCA |
| | | AGGCCAAAGCCAGGCAGCCCTAGACCATTCTTTTGGATGTTC |
| | | GTGGATAATCTGGTGCTGAACAAGGAGGATCTGGACGTGGC |
| | | CAGCAGGTTTCTGGAGATGGAGCCAGTGACCATCCCAGACG |
| | | TGCACGGCGGCTCCCTGCAGAATGCCGTGCGCGTGTGGTCTA |
| | | ACATCCCTGCCATCAGAAGCAGGCACTGGGCACTGGTGAGC |
| | | GAGGAGGAGCTGTCCCTGCTGGCCCAGAATAAGCAGAGCAG |
| | | CAAGCTGGCCGCCAAGTGGCCTACAAAGCTGGTGAAGAACT |
| | | GCTTCCTGCCACTGCGGGAGTACTTCAAGTATTTTTCCACCG |
| | | AGCTGACATCTAGCCTGGGAGGACCCTCCTCTGGCGCCCCAC |
| | | CACCTAGCGGCGGCTCCCCTGCCGGCTCTCCAACCAGCACAG |
| | | AGGAGGGCACCAGCGAGTCCGCCACACCAGAGTCTGGACCT |
| | | GGCACCAGCACAGAGCCATCCGAGGGCTCTGCCCCAGGCTC |
| | | TCCTGCAGGCAGCCCTACCTCCACCGAAGAGGGCACCAGCA |
| | | CAGAGCCTTCTGAGGGCAGCGCCCCAGGCACCTCTACAGAG |
| | | CCAAGCGAGCTCGAGGACAAGAAGTACAGCATCGGCCTGGC |
| | | CATCGGCACCAACTCTGTGGGCTGGGCCGTGATCACCGACG |
| | | AGTACAAGGTGCCCAGCAAGAAATTCAAGGTGCTGGGCAAC |
| | | ACCGACCGGCACAGCATCAAGAAGAACCTGATCGGAGCCCT |
| | | GCTGTTCGACAGCGGCGAAACAGCCGAGGCCACCCGGCTGA |
| | | AGAGAACCGCCAGAAGAAGATACACCAGACGGAAGAACCG |
| | | GATCTGCTATCTGCAAGAGATCTTCAGCAACGAGATGGCCA |
| | | AGGTGGACGACAGCTTCTTCCACAGACTGGAAGAGTCCTTCC |
| | | TGGTGGAAGAGGATAAGAAGCACGAGCGGCACCCCATCTTC |
| | | GGCAACATCGTGGACGAGGTGGCCTACCACGAGAAGTACCC |
| | | CACCATCTACCACCTGAGAAAGAAACTGGTGGACAGCACCG |
| | | ACAAGGCCGACCTGCGGCTGATCTATCTGGCCCTGGCCCACA |
| | | TGATCAAGTTCCGGGGCCACTTCCTGATCGAGGGCGACCTGA |
| | | ACCCCGACAACAGCGACGTGGACAAGCTGTTCATCCAGCTG |
| | | GTGCAGACCTACAACCAGCTGTTCGAGGAAAACCCCATCAA |
| | | CGCCAGCGGCGTGGACGCCAAGGCCATCCTGTCTGCCAGAC |
| | | TGAGCAAGAGCAGACGGCTGGAAAATCTGATCGCCCAGCTG |
| | | CCCGGCGAGAAGAAGAATGGCCTGTTCGGCAACCTGATTGC |
| | | CCTGAGCCTGGGCCTGACCCCCAACTTCAAGAGCAACTTCGA |
| | | CCTGGCCGAGGATGCCAAACTGCAGCTGAGCAAGGACACCT |
| | | ACGACGACGACCTGGACAACCTGCTGGCCCAGATCGGCGAC |
| | | CAGTACGCCGACCTGTTTCTGGCCGCCAAGAACCTGTCCGAC |
| | | GCCATCCTGCTGAGCGACATCCTGAGAGTGAACACCGAGAT |
| | | CACCAAGGCCCCCCTGAGCGCCTCTATGATCAAGAGATACG |
| | | ACGAGCACCACCAGGACCTGACCCTGCTGAAAGCTCTCGTG |
| | | CGGCAGCAGCTGCCTGAGAAGTACAAAGAGATTTTCTTCGA |
| | | CCAGAGCAAGAACGGCTACGCCGGCTACATTGACGGCGGAG |
| | | CCAGCCAGGAAGAGTTCTACAAGTTCATCAAGCCCATCCTGG |
| | | AAAAGATGGACGGCACCGAGGAACTGCTCGTGAAGCTGAAC |
| | | AGAGAGGACCTGCTGCGGAAGCAGCGGACCTTCGACAACGG |
| | | CAGCATCCCCCACCAGATCCACCTGGGAGAGCTGCACGCCA |
| | | TTCTGCGGCGGCAGGAAGATTTTTACCCATTCCTGAAGGACA |
| | | ACCGGGAAAAGATCGAGAAGATCCTGACCTTCCGCATCCCC |
| | | TACTACGTGGGCCCTCTGGCCAGGGGAAACAGCAGATTCGC |
| | | CTGGATGACCAGAAAGAGCGAGGAAACCATCACCCCCTGGA |
| | | ACTTCGAGGAAGTGGTGGACAAGGGCGCTTCCGCCCAGAGC |
| | | TTCATCGAGCGGATGACCAACTTCGATAAGAACCTGCCCAAC |
| | | GAGAAGGTGCTGCCCAAGCACAGCCTGCTGTACGAGTACTT |
| | | CACCGTGTATAACGAGCTGACCAAAGTGAAATACGTGACCG |
| | | AGGGAATGAGAAAGCCCGCCTTCCTGAGCGGCGAGCAGAAA |
| | | AAGGCCATCGTGGACCTGCTGTTCAAGACCAACCGGAAAGT |
| | | GACCGTGAAGCAGCTGAAAGAGGACTACTTCAAGAAAATCG |
| | | AGTGCTTCGACTCCGTGGAAATCTCCGGCGTGGAAGATCGGT |
| | | TCAACGCCTCCCTGGGCACATACCACGATCTGCTGAAAATTA |
| | | TCAAGGACAAGGACTTCCTGGACAATGAGGAAAACGAGGAC |
| | | ATTCTGGAAGATATCGTGCTGACCCTGACACTGTTTGAGGAC |
| | | AGAGAGATGATCGAGGAACGGCTGAAAACCTATGCCCACCT |
| | | GTTCGACGACAAAGTGATGAAGCAGCTGAAGCGGCGGAGAT |
| | | ACACCGGCTGGGGCAGGCTGAGCCGGAAGCTGATCAACGGC |
| | | ATCCGGGACAAGCAGTCCGGCAAGACAATCCTGGATTTCCT |
| | | GAAGTCCGACGGCTTCGCCAACAGAAACTTCATGCAGCTGA |
| | | TCCACGACGACAGCCTGACCTTTAAAGAGGACATCCAGAAA |
| | | GCCCAGGTGTCCGGCCAGGGCGATAGCCTGCACGAGCACAT |
| | | TGCCAATCTGGCCGGCAGCCCCGCCATTAAGAAGGGCATCCT |
| | | GCAGACAGTGAAGGTGGTGGACGAGCTCGTGAAAGTGATGG |
| | | GCCGGCACAAGCCCGAGAACATCGTGATCGAAATGGCCAGA |
| | | GAGAACCAGACCACCCAGAAGGGACAGAAGAACAGCCGCG |
| | | AGAGAATGAAGCGGATCGAAGAGGGCATCAAAGAGCTGGG |
| | | CAGCCAGATCCTGAAAGAACACCCCGTGGAAAACACCCAGC |
| | | TGCAGAACGAGAAGCTGTACCTGTACTACCTGCAGAATGGG |
| | | CGGGATATGTACGTGGACCAGGAACTGGACATCAACCGGCT |
| | | GTCCGACTACGATGTGGACGCCATCGTGCCTCAGAGCTTTCT |
| | | GAAGGACGACTCCATCGACAACAAGGTGCTGACCAGAAGCG |
| | | ACAAGAACCGGGGCAAGAGCGACAACGTGCCCTCCGAAGAG |
| | | GTCGTGAAGAAGATGAAGAACTACTGGCGGCAGCTGCTGAA |
| | | CGCCAAGCTGATTACCCAGAGAAAGTTCGACAATCTGACCA |
| | | AGGCCGAGAGAGGCGGCCTGAGCGAACTGGATAAGGCCGGC |
| | | TTCATCAAGAGACAGCTGGTGGAAACCCGGCAGATCACAAA |
| | | GCACGTGGCACAGATCCTGGACTCCCGGATGAACACTAAGT |
| | | ACGACGAGAATGACAAGCTGATCCGGGAAGTGAAAGTGATC |
| | | ACCCTGAAGTCCAAGCTGGTGTCCGATTTCCGGAAGGATTTC |
| | | CAGTTTTACAAAGTGCGCGAGATCAACAACTACCACCACGC |
| | | CCACGACGCCTACCTGAACGCCGTCGTGGGAACCGCCCTGA |
| | | TCAAAAAGTACCCTAAGCTGGAAAGCGAGTTCGTGTACGGC |
| | | GACTACAAGGTGTACGACGTGCGGAAGATGATCGCCAAGAG |
| | | CGAGCAGGAAATCGGCAAGGCTACCGCCAAGTACTTCTTCT |
| | | ACAGCAACATCATGAACTTTTTCAAGACCGAGATTACCCTGG |
| | | CCAACGGCGAGATCCGGAAGCGGCCTCTGATCGAGACAAAC |
| | | GGCGAAACCGGGGAGATCGTGTGGGATAAGGGCCGGGATTT |
| | | TGCCACCGTGCGGAAAGTGCTGAGCATGCCCCAAGTGAATA |
| | | TCGTGAAAAAGACCGAGGTGCAGACAGGCGGCTTCAGCAAA |
| | | GAGTCTATCCTGCCCAAGAGGAACAGCGATAAGCTGATCGC |
| | | CAGAAAGAAGGACTGGGACCCTAAGAAGTACGGCGGCTTCG |
| | | ACAGCCCCACCGTGGCCTATTCTGTGCTGGTGGTGGCCAAAG |
| | | TGGAAAAGGGCAAGTCCAAGAAACTGAAGAGTGTGAAAGA |
| | | GCTGCTGGGGATCACCATCATGGAAAGAAGCAGCTTCGAGA |
| | | AGAATCCCATCGACTTTCTGGAAGCCAAGGGCTACAAAGAA |
| | | GTGAAAAAGGACCTGATCATCAAGCTGCCTAAGTACTCCCTG |
| | | TTCGAGCTGGAAAACGGCCGGAAGAGAATGCTGGCCTCTGC |
| | | CGGCGAACTGCAGAAGGGAAACGAACTGGCCCTGCCCTCCA |
| | | AATATGTGAACTTCCTGTACCTGGCCAGCCACTATGAGAAGC |
| | | TGAAGGGCTCCCCCGAGGATAATGAGCAGAAACAGCTGTTT |
| | | GTGGAACAGCACAAGCACTACCTGGACGAGATCATCGAGCA |
| | | GATCAGCGAGTTCTCCAAGAGAGTGATCCTGGCCGACGCTA |
| | | ATCTGGACAAAGTGCTGTCCGCCTACAACAAGCACCGGGAT |
| | | AAGCCCATCAGAGAGCAGGCCGAGAATATCATCCACCTGTT |
| | | TACCCTGACCAATCTGGGAGCCCCTGCCGCCTTCAAGTACTT |
| | | TGACACCACCATCGACCGGAAGAGGTACACCAGCACCAAAG |
| | | AGGTGCTGGACGCCACCCTGATCCACCAGAGCATCACCGGC |
| | | CTGTACGAGACACGGATCGACCTGTCTCAGCTGGGAGGCGA |
| | | CAGCCCCAAGAAGAAGAGAAAGGTGGGAGTCGACGGATCC |
| | | AGCGGCTCCGAGACCCCAGGCACATCTGAGAGCGCCACCCC |
| | | TGAGTCCCGGACCCTGGTGACATTCAAGGACGTGTTCGTGGA |
| | | CTTCACCCGGGAGGAGTGGAAGCTGCTGGACACAGCCCAGC |
| | | AGATCGTGTACAGGAACGTGATGCTGGAGAACTATAAGAAT |
| | | CTGGTGTCTCTGGGCTACCAGCTGACAAAGCCAGATGTGATC |
| | | CTGCGGCTGGAGAAGGGAGAGGAGCCCTGGCTGGTGTAG |
| 168 | gRNA041 | ACUGCCUGGCUCACUCCUCCGUUUAAGAGCUAAGCUGGAA |
| | | ACAGCAUAGCAAGUUUAAAUAAGGCUAGUCCGUUAUCAAC |
| | | UUGAAAAAGUGGCACCGAGUCGGUGCUUUUUU |
| 169 | gRNA041 target | ACTGCCTGGCTCACTCCTCC |
| | domain sequence | |
| 170 | gRNA049 | AUCGUCCGAUGGGGCUCUGGGUUUAAGAGCUAAGCUGGAA |
| | | ACAGCAUAGCAAGUUUAAAUAAGGCUAGUCCGUUAUCAAC |
| | | UUGAAAAAGUGGCACCGAGUCGGUGCUUUUUU |
| 171 | gRNA049 target | ATCGTCCGATGGGGCTCTGG |
| | domain sequence | |

1.-40. (canceled)

41. An epigenetic editing system comprising:
(a) a fusion protein comprising:
a DNA-binding domain, and
a DNMT3L domain, wherein the DNMT3L domain is from a species selected from the group consisting of Ailuropoda melanoleuca, Carlito syrichta, Meriones unguiculatus, Ochotona princeps, Neosciurus carolinensis, Bison bison, Equus przewalskii, Mus caroli and Pan troglodytes; or
(b) a nucleic acid molecule encoding the fusion protein of (a).

42. The epigenetic editing system of claim 41, wherein the DNMT3L domain comprises an amino acid sequence that is at least 90% homologous to any one of SEQ ID NOs: 72-80 and 101-109.

43. The epigenetic editing system of claim 42, wherein the DNMT3L domain comprises an amino acid sequence of any one of SEQ ID NOs: 72-80 and 101-109.

44. The epigenetic editing system of claim 42, wherein the DNMT3L domain comprises an amino acid sequence that is at least 90% homologous to SEQ ID NO: 107.

45. The epigenetic editing system of claim 44, comprising an amino acid sequence of SEQ ID NO: 107.

46. The epigenetic editing system of claim 41, wherein the fusion protein further comprises a repressor domain.

47. The epigenetic editing system of claim 46, wherein the repressor domain comprises a KRAB domain.

48. The epigenetic editing system of claim 47, wherein the KRAB domain is derived from KOX1, ZIM3, ZFP28, or ZN627.

49. The epigenetic editing system of claim 48, wherein the KRAB domain is derived from the ZIM3.

50. The epigenetic editing system of claim 49, wherein the ZIM3 comprises an amino acid sequence that is at least 90% homologous to SEQ ID NO: 60 or 100.

51. The epigenetic editing system of claim 41, wherein the fusion protein further comprises a DNMT3A domain.

52. The epigenetic editing system of claim 51, wherein the DNMT3A domain comprises an amino acid sequence that is at least 90% homologous to SEQ ID NO: 96.

53. The epigenetic editing system of claim 52, wherein the DNMT3A domain comprises an amino acid sequence of SEQ ID NO: 96.

54. The epigenetic editing system of claim 41, wherein the fusion protein comprises two nuclear localization sequences (NLSs).

55. The epigenetic editing system of claim 54, wherein a first NLS of the two NLSs is positioned at the amino (N) terminus and a second NLS of the two NLSs is positioned at the carboxy (C) terminus of the fusion protein.

56. The epigenetic editing system of claim 41, wherein the fusion protein comprises one or more peptide linkers.

57. The epigenetic editing system of claim 56, wherein the one or more peptide linkers comprise XTEN80 or XTEN16.

58. The epigenetic editing system of claim 41, wherein the DNA-binding domain is a nuclease inactive Cas9 (dCas9) domain.

59. The epigenetic editing system of claim 58, wherein the dCas9 domain comprises an amino acid sequence that is at least 90% homologous to SEQ ID NO: 9.

60. The epigenetic editing system of claim 41, further comprising one or more guide RNAs (gRNAs) or nucleic acid molecule(s) coding the gRNAs.


SEQUENCE LISTING (180 sequences)

SEQ ID NO: 1 - protein, 5 aa, synthetic construct
GSGGG

SEQ ID NO: 2 - protein, 16 aa, synthetic construct
SGSETPGTSESATPES

SEQ ID NO: 3 - protein, 80 aa, synthetic construct
GGPSSGAPPPSGGSPAGSPTSTEEGTSESATPESGPGTSTEPSEGSAPGSPAGSPTSTEEGTSTEPSEGSAPGTSTEPSE

SEQ ID NO: 4 - protein, 150 aa, synthetic construct
  VARIANT 6..150: note = GGGGS repeats may be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 5 - protein, 27 aa, synthetic construct
SSGNSNANSRGPSFSSGLVPLSLRGSH

SEQ ID NO: 6 - protein, 9 aa, synthetic construct
YPYDVPDYA

SEQ ID NO: 7 - protein, 7 aa, synthetic construct
PKKKRKV

SEQ ID NO: 8 - protein, 1369 aa, synthetic construct [sequence omitted]

SEQ ID NO: 9 - protein, 77 aa, synthetic construct
TGRTLVTFKDVFVDFTREEWKLLDTAQQIVYRNVMLENYKNLVSLGYQLTKPDVILRLEKGEEPLEINLWITKFVKD

SEQ ID NO: 10 - protein, 120 aa, synthetic construct
MYPYDVPDYASPKKKRKVGGGASMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 11 - protein, 117 aa, synthetic construct
ALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 12 - protein, 322 aa, Homo sapiens
TYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 13 - protein, 386 aa, Homo sapiens
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 14 - protein, 214 aa, Homo sapiens
ENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 15 - protein, 421 aa, Mus sp.
MGSRETPSSCSKTLETLDLETSDSSSPDADSPLEEQWLKSSPALKEDSVDVVLEDCKEPLSPSSPPTGREMIRYEVKVNRRSIEDICLCCGTLQVYTRHPLFEGGLCAPCKDKFLESLFLYDDDGHQSYCTICCSGGTLFICESPDCTRCYCFECVDILVGPGTSERINAMACWVCFLCLPFSRSGLLQRRKRWRHQLKAFHDQEGAGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 16 - protein, 215 aa, Mus sp.
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 17 - protein, 2098 aa, synthetic construct
MPKKKRKVPKKKRKVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGSETPGTSESATPESTGRTLVTFKDVFVDFTREEWKLLDTAQQIVYRNVMLENYKNLVSLGYQLTKPDVILRLEKGEEPPKKKRKVPKKKRKV

SEQ ID NO: 18 - DNA, 6294 bp, synthetic construct
  source 1..6294: mol_type = other DNA
atgccaaaaaagaagagaaaggtaccgaagaaaaaaagaaaggtcaaccatgatcaagaattcgacccacctaaagtctacccacctgtgcccgccgaaaaaaggaaacccataagggtgctgtcactctttgatggcatcgccactggtctcctggttcttaaggatctgggaattcaggtcgatcggtacattgctagcgaggtttgtgaggatagtattacagtgggtatggtgcgccaccagggaaagatcatgtatgttggtgacgttaggagcgtcacccagaaacatatccaggagtggggaccctttgatttggtgatcggaggtagtccctgcaatgacctttccatcgtgaatccagccaggaaagggctgtatgaagggactggtaggctctttttcgagttttatcgcctgcttcacgacgctagacctaaggaaggtgacgataggcctttcttttggctttttgagaacgtcgtggcaatgggagtctccgacaaaagggacatttctcgctttctggaatctaaccccgttatgatcgatgccaaggaagtttctgccgctcacagggcaaggtacttctggggcaatctgcccggaatgaatcgcccactggccagtaccgtgaatgacaaactggagctgcaggagtgcctggagcacggaagaatcgcaaagttttctaaagtcaggaccattaccactcgcagtaactccataaaacagggtaaggaccagcattttcccgtcttcatgaatgaaaaggaagatattctgtggtgcactgaaatggagagagttttcgggtttcccgtgcactataccgatgtttccaacatgtcccgccttgcaagacaaaggcttttgggccgctcttggtctgtgccagtgatccggcacttgtttgctcccctcaaagagtacttcgcttgcgtcagttccggaaattcaaacgctaactctcggggtccatctttctccagtggtctcgtgccactgtctctccggggctctcacaatcccctggagatgtttgagacagtgccagtctggcggaggcagcccgttcgcgttctctctctgttcgaagatattaaaaaggaactcacctcccttgggttcctggagagcgggagcgaccccggacagcttaagcacgtggtcgacgtgactgacaccgtccgcaaagacgtggaggaatggggccccttcgatctggtctatggggcaacccctccccttgggcatacatgtgatcggcctccatcctggtacctgttccagtttcacagactcctgcagtatgccaggccaaagccagggagcccaaggccctttttctggatgttcgtcgacaacctggtcctgaacaaagaagatctcgacgttgctagtcgctttctcgaaatggagcccgtgaccattcccgacgtgcatggcggttccctccagaatgcagtcagggtttggagcaatatccctgccatcaggtcaaggcactgggcactggtttcagaggaagagctgtccctccttgcccagaacaagcagtcatccaaactggcagccaagtggccaactaagctggtcaagaactgctttcttcccctcagagaatattttaagtatttcagtactgaactgactagcagtctgggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcggaagtgagaccccaggtacatccgaatcagcaacgcctgaaagcaccggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccccaaaaaagaagagaaaggtaccgaagaaaaaaagaaaggtc

SEQ ID NO: 19 - protein, 2084 aa, synthetic construct
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVSGSETPGTSESATPESTGRTLVTFKDVFVDFTREEWKLLDTAQQIVYRNVMLENYKNLVSLGYQLTKPDVILRLEKGEEP

SEQ ID NO: 20 - DNA, 6252 bp, synthetic construct
  source 1..6252: mol_type = other DNA
atgaaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtctagcggcaatagtaacgctaacagccgcgggccgagcttcagcagcggcctggtgccgttaagcttgcgcggcagccataatccccttgagatgttcgaaaccgtgcctgtgtggaggagacagccagtccgggtgctgtccctttttgaagacatcaagaaagagctgacgagtttgggctttttggaaagtggttctgacccgggacaactgaagcatgtggttgatgtcacagacacagtgaggaaggatgtggaggagtggggacccttcgatcttgtgtacggcgccacacctcccctgggccacacctgtgaccgtcctcccagctggtacctgttccagttccaccggctcctgcagtacgcacggcccaagccaggcagccccaggcccttcttctggatgttcgtggacaatctggtgctgaacaaggaagacctggacgtcgcatctcgcttcctggagatggagccagtcaccatcccagatgtccacggcggatccttgcagaatgctgtccgcgtgtggagcaacatcccagccataaggagcaggcactgggctctggtttcggaagaagaattgtccctgctggcccagaacaagcagagctcgaagctcgcggccaagtggcccaccaagctggtgaagaactgctttctccccctaagagaatatttcaagtatttttcaacagaactcacttcctctttaggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacccaaaaaagaagagaaaggtaagcggaagtgagaccccaggtacatccgaatcagcaacgcctgaaagcaccggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 21 - protein, 2219 aa, synthetic construct
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMEVKVNRRSIEDICLCCGTLQVYTRHPLFEGGLCAPCKDKFLESLFLYDDDGHQSYCTICCSGGTLFICESPDCTRCYCFECVDILVGPGTSERINAMACWVCFLCLPFSRSGLLQRRKRWRHQLKAFHDQEGAGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVSGSETPGTSESATPESTGRTLVTFKDVFVDFTREEWKLLDTAQQIVYRNVMLENYKNLVSLGYQLTKPDVILRLEKGEEP

SEQ ID NO: 22 - DNA, 6657 bp, synthetic construct
  source 1..6657: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatggaagtcaaagtgaaccgacggagcattgaagacatctgcctctgctgtggaactctccaggtgtacactcggcaccccttgtttgagggagggttatgtgccccatgtaaggataagttcctggagtccctcttcctgtatgatgatgatggacaccagagttactgcaccatctgctgttccgggggtaccctgttcatctgtgagagccccgactgtaccagatgctactgtttcgagtgtgtggacatcctggtgggccccgggacctcagagaggatcaatgccatggcctgctgggtttgcttcctgtgcctgcccttctcacggagtggactgctgcagaggcgcaagaggtggcggcaccagctgaaggccttccatgatcaagagggagcgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacccaaaaaagaagagaaaggtaagcggaagtgagaccccaggtacatccgaatcagcaacgcctgaaagcaccggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 23 - protein, 2218 aa, synthetic construct
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVSGSETPGTSESATPESTGRTLVTFKDVFVDFTREEWKLLDTAQQIVYRNVMLENYKNLVSLGYQLTKPDVILRLEKGEEP

SEQ ID NO: 24 - DNA, 6654 bp, synthetic construct
  source 1..6654: mol_type = other DNA
atgaaccatgatcaagaattcgacccacctaaagtctacccacctgtgcccgccgaaaaaaggaaacccataagggtgctgtcactctttgatggcatcgccactggtctcctggttcttaaggatctgggaattcaggtcgatcggtacattgctagcgaggtttgtgaggatagtattacagtgggtatggtgcgccaccagggaaagatcatgtatgttggtgacgttaggagcgtcacccagaaacatatccaggagtggggaccctttgatttggtgatcggaggtagtccctgcaatgacctttccatcgtgaatccagccaggaaagggctgtatgaagggactggtaggctctttttcgagttttatcgcctgcttcacgacgctagacctaaggaaggtgacgataggcctttcttttggctttttgagaacgtcgtggcaatgggagtctccgacaaaagggacatttctcgctttctggaatctaaccccgttatgatcgatgccaaggaagtttctgccgctcacagggcaaggtacttctggggcaatctgcccggaatgaatcgcccactggccagtaccgtgaatgacaaactggagctgcaggagtgcctggagcacggaagaatcgcaaagttttctaaagtcaggaccattaccactcgcagtaactccataaaacagggtaaggaccagcattttcccgtcttcatgaatgaaaaggaagatattctgtggtgcactgaaatggagagagttttcgggtttcccgtgcactataccgatgtttccaacatgtcccgccttgcaagacaaaggcttttgggccgctcttggtctgtgccagtgatccggcacttgtttgctcccctcaaagagtacttcgcttgcgtcagttccggaaattcaaacgctaactctcggggtccatctttctccagtggtctcgtgccactgtctctccggggctctcacatggaagtcaaggctaaccagcgaaatatagaagacatctgcatctgctgcggaagtctccaggttcacacacagcaccctctgtttgagggagggatctgcgccccatgtaaggacaagttcctggatgccctcttcctgtacgacgatgacgggtaccaatcctactgctccatctgctgctccggagagacgctgctcatctgcggaaaccctgattgcacccgatgctactgcttcgagtgtgtggatagcctggtcggccccgggacctcggggaaggtgcacgccatgagcaactgggtgtgctacctgtgcctgccgtcctcccgaagcgggctgctgcagcgtcggaggaagtggcgcagccagctcaaggccttctacgaccgagagtcggagaatcccctggagatgtttgagacagtgccagtctggcggaggcagcccgttcgcgttctctctctgttcgaagatattaaaaaggaactcacctcccttgggttcctggagagcgggagcgaccccggacagcttaagcacgtggtcgacgtgactgacaccgtccgcaaagacgtggaggaatggggccccttcgatctggtctatggggcaacccctccccttgggcatacatgtgatcggcctccatcctggtacctgttccagtttcacagactcctgcagtatgccaggccaaagccagggagcccaaggccctttttctggatgttcgtcgacaacctggtcctgaacaaagaagatctcgacgttgctagtcgctttctcgaaatggagcccgtgaccattcccgacgtgcatggcggttccctccagaatgcagtcagggtttggagcaatatccctgccatcaggtcaaggcactgggcactggtttcagaggaagagctgtccctccttgcccagaacaagcagtcatccaaactggcagccaagtggccaactaagctggtcaagaactgctttcttcccctcagagaatattttaagtatttcagtactgaactgactagcagtctgggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagccaaaaaagaagagaaaggtaatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacccaaaaaagaagagaaaggtaagcggaagtgagaccccaggtacatccgaatcagcaacgcctgaaagcaccggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 25 - RNA, 100 nt, synthetic construct
  source 1..100: mol_type = other RNA
agatcagatctttgtcgatcctaccatccactcgacacacccgccagcggccgctaatacgactcactatagggagacccaagctggctagccgccacca

SEQ ID NO: 26 - RNA, 69 nt, synthetic construct
  source 1..69: mol_type = other RNA
taatacgactcactatagggggcgctcgagcaggttcagaaggagatcaaaaacccccaaggatcaaac

SEQ ID NO: 27 - RNA, 255 nt, synthetic construct
  source 1..255: mol_type = other RNA
ctctggctaactagagaacccactgcttactggcttatcgaaattaatacgactcactatagggagacccaagctggctagcgtttaaacttaagcttggtaccgaggtaagtatcaaggttacaagacaggtttaaggaggccaatagaaactgggcttgtcgagacagagaagattcttgcgtttctgataggcacctattggtcttactgacatccactttgcctttctctccacagcaacaagccgccacc

SEQ ID NO: 28 - protein, 2109 aa, synthetic construct
MPKKKRKVPKKKRKVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGSETPGTSESATPESTGRTLVTFKDVFVDFTREEWKLLDTAQQIVYRNVMLENYKNLVSLGYQLTKPDVILRLEKGEEPSADYKDDDDKAPKKKRKVPKKKRKV

SEQ ID NO: 29 - DNA, 6327 bp, synthetic construct
  source 1..6327: mol_type = other DNA
atgccaaaaaagaagagaaaggtaccgaagaaaaaaagaaaggtcaaccacgaccaggaattcgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtctagcggcaatagtaacgctaacagccgcgggccgagcttcagcagcggcctggtgccgttaagcttgcgcggcagccataatccccttgagatgttcgaaaccgtgcctgtgtggaggagacagccagtccgggtgctgtccctttttgaagacatcaagaaagagctgacgagtttgggctttttggaaagtggttctgacccgggacaactgaagcatgtggttgatgtcacagacacagtgaggaaggatgtggaggagtggggacccttcgatcttgtgtacggcgccacacctcccctgggccacacctgtgaccgtcctcccagctggtacctgttccagttccaccggctcctgcagtacgcacggcccaagccaggcagccccaggcccttcttctggatgttcgtggacaatctggtgctgaacaaggaagacctggacgtcgcatctcgcttcctggagatggagccagtcaccatcccagatgtccacggcggatccttgcagaatgctgtccgcgtgtggagcaacatcccagccataaggagcaggcactgggctctggtttcggaagaagaattgtccctgctggcccagaacaagcagagctcgaagctcgcggccaagtggcccaccaagctggtgaagaactgctttctccccctaagagaatatttcaagtatttttcaacagaactcacttcctctttaggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcggaagtgagaccccaggtacatccgaatcagcaacgcctgaaagcaccggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcgctgattacaaagatgatgacgataaagccccaaaaaagaagagaaaggtaccgaagaaaaaaagaaaggtc

SEQ ID NO: 30 - protein, 2111 aa, synthetic construct
MPKKKRKVPKKKRKVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGSETPGTSESATPESTGRTLVTFKDVFVDFTREEWKLLDTAQQIVYRNVMLENYKNLVSLGYQLTKPDVILRLEKGEEPSADYKDDDDKAPKKKRKVPKKKRKV

SEQ ID NO: 31 - DNA, 6336 bp, synthetic construct
  source 1..6336: mol_type = other DNA
atgccaaaaaagaagagaaaggtaccgaagaaaaaaagaaaggtatacaaccatgaccaggaattcgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcggaagtgagaccccaggtacatccgaatcagcaacgcctgaaagcaccggtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcgctgattacaaagatgatgacgataaagccccaaaaaagaagagaaaggtaccgaagaaaaaaagaaaggtc

SEQ ID NO: 32 - protein, 2149 aa, synthetic construct
MPKKKRKVPKKKRKVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGSETPGTSESATPESTGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLSADYKDDDDKAPKKKRKVPKKKRKV

SEQ ID NO: 33 - DNA, 6450 bp, synthetic construct
  source 1..6450: mol_type = other DNA
atgccaaaaaagaagagaaaggtaccgaagaaaaaaagaaaggtatacaaccatgaccaggaattcgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcggaagtgagaccccaggtacatccgaatcagcaacgcctgaaagcaccggtatgaacaattcacaggggagagtgacattcgaagacgtgaccgtgaacttcacccagggagaatggcagcgcttgaacccagaacaaaggaacctctatcgggacgtgatgctggaaaactactcaaatttggtgagcgttgggcagggtgagaccactaagcctgacgtgatcctgagattggaacagggcaaggagccttggctcgaggaagaggaagtcctgggctcagggagggccgagaaaaacggtgatataggaggccagatatggaagcctaaggacgtcaaggagagcctgagcgctgattacaaagatgatgacgataaagccccaaaaaagaagagaaaggtaccgaagaaaaaaagaaaggtc

SEQ ID NO: 34 - protein, 2129 aa, synthetic construct
MPKKKRKVPKKKRKVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGSETPGTSESATPESTGNKKLEAVGTGIEPKAMSQGLVTFGDVAVDFSQEEWEWLNPIQRNLYRKVMLENYRNLASLGLCVSKPDVISSLEQGKEPWSADYKDDDDKAPKKKRKVPKKKRKV

SEQ ID NO: 35 - DNA, 6390 bp, synthetic construct
  source 1..6390: mol_type = other DNA
atgccaaaaaagaagagaaaggtaccgaagaaaaaaagaaaggtatacaaccatgaccaggaattcgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgagatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcggaagtgagaccccaggtacatccgaatcagcaacgcctgaaagcaccggtaacaaaaagcttgaggccgtcggaaccggaatcgaaccaaaagcaatgtcccagggtttggtgacatttggcgacgtggctgtcgatttttcccaggaagagtgggagtggctcaatcctatccagaggaacttgtaccggaaggtgatgctggagaattatagaaatttggcatcactggggttgtgcgttagcaaaccagatgttatatcttccctggaacagggaaaggagccctggagcgctgattacaaagatgatgacgataaagccccaaaaaagaagagaaaggtaccgaagaaaaaaagaaaggtc

SEQ ID NO: 36 - protein, 439 aa, Arthrobacter luteus
MSKANAKYSFVDLFAGIGGFHAALAATGGVCEYAVEIDREAAAVYERNWNKPALGDITDDANDEGVTLRGYDGPIDVLTGGFPCQPFSKSGAQHGMAETRGTLFWNIARIIEEREPTVLILENVRNLVGPRHRHEWLTIIETLRFFGYEVSGAPAIFSPHLLPAWMGGTPQVRERVFITATLVPERMRDERSTIRRATGRPLEGFPYWADSWTDFRELSRLVVIRGFQAPEREVVGDRKRYVARTDMPEGFVPASVTRPAIDETLPAWKQSHLRRNYDFFERHFAEVVAWAYRWGVYTDLFPASRRKLEWQAQDAPRLWDTVMHFRPSGIRAKRPTYLPALVAITQTSIVGPLERRLSPRETARLQGLPEWFDFGEQRAAATYKQMGNGVNVGVVRHILREHVRRDRALLKLTPAGQRIINAVLADEPDATVGALGAAE

SEQ ID NO: 37 - protein, 418 aa, Moraxella sp.
MKPEILKLIRSKLDLTQKQASEIIEVSDKTWQQWESGKTEMHPAYYSFLQEKLKDKINFEELSAQKTLQKKIFDKYNQNQITKNAEELAEITHIEERKDAYSSDFKFIDLFSGIGGIRQSFEVNGGKCVFSSEIDPFAKFTYYTNFGVVPFGDITKVEATTIPQHDILCAGFPCQPFSHIGKREGFEHPTQGTMFHEIVRIIETKKTPVLFLENVPGLINHDDGNTLKVIIETLEDMGYKVHHTVLDASHFGIPQKRKRFYLVAFLNQNIHFEFPKPPMISKDIGEVLESDVTGYSISEHLQKSYLFKKDDGKPSLIDKNTTGAVKTLVSTYHKIQRLTGTFVKDGETGIRLLTTNECKAIMGFPKDFVIPVSRTQMYRQMGNSVVVPVVTKIAEQISLALKTVNQQSPQENFELELV

SEQ ID NO: 38 - protein, 330 aa, Haemophilus influenzae
MNLISLFSGAGGLDLGFQKAGFRIICANEYDKSIWKTYESNHSAKLIKGDISKISSDEFPKCDGIIGGPPCQSWSEGGSLRGIDDPRGKLFYEYIRILKQKKPIFFLAENVKGMMAQRHNKAVQEFIQEFDNAGYDVHIILLNANDYGVAQDRKRVFYIGFRKELNINYLPPIPHLIKPTFKDVIWDLKDNPIPALDKNKTNGNKCIYPNHEYFIGSYSTIFMSRNRVRQWNEPAFTVQASGRQCQLHPQAPVMLKVSKNLNKFVEGKEHLYRRLTVRECARVQGFPDDFIFHYESLNDGYKMIGNAVPVNLAYEIAKTIKSALEICKGN

SEQ ID NO: 39 - protein, 327 aa, Haemophilus haemolyticus
MIEIKDKQLTGLRFIDLFAGLGGFRLALESCGAECVYSNEWDKYAQEVYEMNFGEKPEGDITQVNEKTIPDHDILCAGFPCQAFSISGKQKGFEDSRGTLFFDIARIVREKKPKVVFMENVKNFASHDNGNTLEVVKNTMNELDYSFHAKVLNALDYGIPQKRERIYMICFRNDLNIQNFQFPKPFELNTFVKDLLLPDSEVEHLVIDRKDLVMTNQEIEQTTPKTVRLGIVGKGGQGERIYSTRGIAITLSAYGGGIFAKTGGYLVNGKTRKLHPRECARVMGYPDSYKVHPSTSQAYKQFGNSVVINVLQYIAYNIGSSLNFKPY

SEQ ID NO: 40 - protein, 386 aa, Spiroplasma monobiae
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 41 - protein, 353 aa, Mycoplasmatales bacterium
MAEWYIPAIVSYQAIHNGFTLNKINHKIELQTMIDYLESKTLSMNSKEPVKRGFWYKKHLDEIRIVYTAVKMSEQEGNIFDVRTLFERGLSDIDLLTYSFPCQDLSQQGKQKGMGRDSQTRSGLLWEIEKALDTSKKEDLPKYLLMENVVALTHKVNAEELDEWMMKLESLGYKNDLRILNAGDFGSSQARRRTFMISTLNEKVELPVGNKKPKSMNKILNDEPTRKDFLPALDKFDLTEYKWTKSNINKAKLINYSTFNSEAYVYDSNFTGPTLTASGANSRIKFEYNGKIRKIGAEEAYAYMGFKKSDYIKVNKLNYLNETKMIYTCGNSISVEVLRSIMTNINNNFKENK

SEQ ID NO: 42 - protein, 398 aa, Mycoplasma marinum
MLFLIGTFKYVLIYITKVIRIFEAFAGIGAQRKALRNIKSNYEVSGMAEWYIPAIVSYQAIHNGFTLSRVDKKTKLTEMIKYLESKTLSMDSKEPVRTGYWFKKHKDMVRIVYSAVKLSEAEGNIFDVRTLHERKLEDIDLLTYSFPCQDLSQQGKQRGMKKDSGTRSGLLWEIEKALEATPKDKLPKYLLMENVVALTHKTNKKDLDNWKRKLRSLGYYNDINVLNAGDFGSSQARRRAFMISTLDSKVTLPLGDKKPQAISKILNKETRSQDFMPALDEYEKTDFKRTLSNIKKCKLIDYTSFNSEAYVYDPKYTGPTLTASGANSRIKFTHQGKMRKINAEEAYRYMGFSTNDYKKVNNLNFLSETKMIYTCGNSISVEVLEEIMLKIIREDNNG

SEQ ID NO: 43 - protein, 381 aa, Spiroplasma chinense
MKKIRLFEAFAGIGSQRRALKSVVGNNFEIAGLAEWYVPAIVMYQIINNDFSKKNVLDNVPRDEVIDYLNSKCLSWDSKKPVSKNFWNRKSQDILNVIYSAVKKSEEEGNIFDVRTLHERTLESIDILTYSFPCQDLSQQGIQKGMKKNSGTRSGLLWEIEKAIDNTPKNNLPKILLMENVPALLNKTNELELKEWLIKLENMGYKNSIGILNAADFGSPQARRRVFMISSRNKKIELPVGKSKPGKLNDILEKNVEDKFIMTNLEKYDFSEFSLTKSNIKKCSLINYTKFNSEAYVYDPDFTGPTLTASGANSRIKIYDKGFIRRMSPLESFRYMGFDDEDYKKIDEFEFLTDTQKIFVCGNSISIEVLKAIFERIDSNE

SEQ ID NO: 44 - protein, 112 aa, synthetic construct
MKKLEAVGTGIEPKAMSQGLVTFGDVAVDFSQEEWEWLNPIQRNLYRKVMLENYRNLASLGLCVSKPDVISSLEQGKEPWTVKRKMTRAWCPDLKAVWKIKELPLKKDFCEG

SEQ ID NO: 45 - protein, 461 aa, synthetic construct
MDSVAFEDVAVNFTLEEWALLDPSQKNLYRDVMRETFRNLASVGKQWEDQNIEDPFKIPRRNISHIPERLCESKEGGQGEETFSQIPDGILNKKTPGVKPCESSVCGEVGMGPSSLNRHIRDHTGREPNEYQEYGKKSYTRNQCGRALSYHRSFPVRERTHPGGKPYDCKECGETFISLVSIRRHMLTHRGGVPYKCKVCGKAFDYPSLFRIHERSHTGEKPYECKQCGKAFSCSSYIRIHERTHTGDKPYECKQCGKAFSCSKYIRIHERTHTGEKPYECKQCGKAFRCASSVRSHERTHTGEKLFECKECGKALTCLASVRRHMIKHTGNGPYKCKVCGKAFDFPSSFRIHERTHTGEKPYDCKQCGKAFSCSSSFRKHERIHTGEKPYKCTKCGKAFSRSSYFRIHERTHTGEKPYECKQCGKAFSRSTYFRVHEKIHTGEKPYENPNPNASVVPVLS

SEQ ID NO: 46 - protein, 835 aa, synthetic construct
MAASAAAASAAAASAASGSPGPGEGSAGGEKRSTAPSAAASASASAAASSPAGGGAEALELLEHCGVCRERLRPEREPRLLPCLHSACSACLGPAAPAAANSSGDGGAAGDGTVVDCPVCKQQCFSKDIVENYFMRDSGSKAATDAQDANQCCTSCEDNAPATSYCVECSEPLCETCVEAHQRVKYTKDHTVRSTGPAKSRDGERTVYCNVHKHEPLVLFCESCDTLTCRDCQLNAHKDHQYQFLEDAVRNQRKLLASLVKRLGDKHATLQKSTKEVRSSIRQVSDVQKRVQVDVKMAILQIMKELNKRGRVLVNDAQKVTEGQQERLERQHWTMTKIQKHQEHILRFASWALESDNNTALLLSKKLIYFQLHRALKMIVDPVEPHGEMKFQWDLNAWTKSAEAFGKIVAERPGTNSTGPAPMAPPRAPGPLSKQGSGSSQPMEVQEGYGFGSGDDPYSSAEPHVSGVKRSRSGEGEVSGLMRKVPRVSLERLDLDLTADSQPPVFKVFPGSTTEDYNLIVIERGAAAAATGQPGTAPAGTPGAPPLAGMAIVKEEETEAAIGAPPTATEGPETKPVLMALAEGPGAEGPRLASPSGSTSSGLEVVAPEGTSAPGGGPGTLDDSATICRVCQKPGDLVMCNQCEFCFHLDCHLPALQDVPGEEWSCSLCHVLPDLKEEDGSLSLDGADSTGVVAKLSPANQRKCERVLLALFCHEPCRPLHQLATDSTFSLDQPGGTLDLTLIRARLQEKLSPPYSSPQEFAQDVGRMFKQFNKLTEDKADVQSIIGLQRFFETRMNEAFGDTKFSAVLVEPPPMSLPGAGLSSQELSGGPGDGP

SEQ ID NO: 47 - protein, 486 aa, synthetic construct
MVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 48 - protein, 185 aa, synthetic construct
MGKKQNKKKVEEVLEEEEEEYVVEKVLDRRVVKGKVEYLLKWKGFSDEDNTWEPEENLDCPDLIAEFLQSQKTAHETDKSEGGKRKADSDSEDKGEESKPKKKKEESEKPRGFARGLEPERIIGATDSSGELMFLMKWKNSDEADLVPAKEANVKCPQVVISFYEERLTWHSYPSEDDDKKDDKN

SEQ ID NO: 49 - protein, 80 aa, synthetic construct
GSGPPSSGGGLYRDMGAQGGRPSLIARIPVARILGDPEEESWSPSLTNLEKVVVTDVTSNFLTVTIKESNTDQGFFKEKR

SEQ ID NO: 50 - protein, 80 aa, synthetic construct
ASGDLYEVERIVDKRKNKKGKWEYLIRWKGYGSTEDTWEPEHHLLHCEEFIDEFNGLHMSKDKRIKSGKQSSTSKLLRDS

SEQ ID NO: 51 - protein, 80 aa, synthetic construct
KDPNEPQKPVSAYALFFRDTQAAIKGQNPNATFGEVSKIVASMWDGLGEEQKQVYKKKTEAAKKEYLKQLAAYRASLVSK

SEQ ID NO: 52 - protein, 80 aa, synthetic construct
KDPNEPQKPVSAYALFFRDTQAAIKGQNPNATFGEVSKIVASMWDSLGEEQKQVYKRKTEAAKKEYLKALAAYRASLVSK

SEQ ID NO: 53 - protein, 80 aa, synthetic construct
KDPNEPQKPVSAYALFFRDTQAAIKGQNPNATFGEVSKIVASMWDSLGEEQKQVYKRKTEAAKKEYLKALAAYKDNQECQ

SEQ ID NO: 54 - protein, 441 aa, synthetic construct
MSEREVSTAPAGTDMPAAKKQKLSSDENSNPDLSGDENDDAVSIESGTNTERPDTPTNTPNAPGRKSWGKGKWKSKKCKYSFKCVNSLKEDHNQPLFGVQFNWHSKEGDPLVFATVGSNRVTLYECHSQGEIRLLQSYVDADADENFYTCAWTYDSNTSHPLLAVAGSRGIIRIINPITMQCIKHYVGHGNAINELKFHPRDPNLLLSVSKDHALRLWNIQTDTLVAIFGGVEGHRDEVLSADYDLLGEKIMSCGMDHSLKLWRINSKRMMNAIKESYDYNPNKTNRPFISQKIHFPDFSTRDIHRNYVDCVRWLGDLILSKSCENAIVCWKPGKMEDDIDKIKPSESNVTILGRFDYSQCDIWYMRFSMDFWQKMLALGNQVGKLYVWDLEVEDPHKAKCTTLTHHKCGAAIRQTSFSRDSSILIAVCDDASIWRWDRLR

SEQ ID NO: 55 - protein, 80 aa, synthetic construct
VWDLSKIGEEQSPEDAEDGPPELLFIHGGHTAKISDFSWNPNEPWVICSVSEDNIMQVWQMAENIYNDEDPEGSVDPEGQ

SEQ ID NO: 56 - protein, 485 aa, synthetic construct
MPAMVEKGPEVSGKRRGRNNAAASASAAAASAAASAACASPAATAASGAAASSASAAAASAAAAPNNGQNKSLAAAAPNGNSSSNSWEEGSSGSSSDEEHGGGGMRVGPQYQAVVPDFDPAKLARRSQERDNLGMLVWSPNQNLSEAKLDEYIAIAKEKHGYNMEQALGMLFWHKHNIEKSLADLPNFTPFPDEWTVEDKVLFEQAFSFHGKTFHRIQQMLPDKSIASLVKFYYSWKKTRTKTSVMDRHARKQKREREESEDELEEANGNNPIDIEVDQNKESKKEVPPTETVPQVKKEKHSTQAKNRAKRKPPKGMFLSQEDVEAVSANATAATTVLRQLDMELVSVKRQIQNIKQTNSALKEKLDGGIEPYRLPEVIQKCNARWTTEEQLLAVQAIRKYGRDFQAISDVIGNKSVVQVKNFFVNYRRRFNIDEVLQEWEAEHGKEETNGPSNQKPVKSPDNSIKMPEEEDEAPVLDVRYASAS

SEQ ID NO: 57 - protein, 80 aa, synthetic construct
KQGFSKDPSTWSVDEVIQFMKHTDPQISGPLADLFRQHEIDGKALFLLKSDVMMKYMGLKLGPALKLCYYIEKLKEGKYS

SEQ ID NO: 58 - protein, 62 aa, synthetic construct
RTLVTFKDVFVDFTREEWKLLDTAQQIVYRNVMLENYKNLVSLGYQLTKPDVILRLEKGEEP

SEQ ID NO: 59 - protein, 98 aa, synthetic construct
RTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQS

SEQ ID NO: 60 - protein, 100 aa, synthetic construct
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 61 - protein, 95 aa, synthetic construct
RTLVTFKDVFVDFTREEWKLLDTAQQIVYRNVMLENYKNLVSLGYQLTKPDVILRLEKGEEPWLEEEEVLGSGRAEKNGDIGGQIWKPKDVKESL

SEQ ID NO: 62 - protein, 103 aa, synthetic construct
MNNSQGRVTFEDVTVNFTQGEWQRLNPEQRNLYRDVMLENYSNLVSVGQGETTKPDVILRLEQGKEPWLVEREIHQETHPDSETAFEIKSSVSSRSIFKDKQS

SEQ ID NO: 63 - protein, 75 aa, synthetic construct
MNNSQGRVTFEDVTVNFTQGEWQRLNPEQRNLYRDVMLENYSNLVSVGQGETTKPDVILRLEQGKEPWLEEEEVL

SEQ ID NO: 64 - protein, 99 aa, synthetic construct
RTLVTFKDVAVDFTQEEWQQLDPAQKIVYRDVMLENYSNLVSVGYQLTKPDVILRLEQKGEEPWLVEEEIHQETHPDSETAFEIKSSVSSRSIFKDKQS

SEQ ID NO: 65 - protein, 75 aa, synthetic construct
MTKSLETVTFKDVAVDLTQEEWQQMKPAQKNLYRDVMLENYSNLVTVGYQVTKPDVIFKLEQREEPWVIEEEMFG

SEQ ID NO: 66 - protein, 75 aa, synthetic construct; identical to SEQ ID NO: 65

SEQ ID NO: 67 - protein, 71 aa, synthetic construct
MNTSLGPLSFKDVAVAFTQEEWQQLGPEEKTTYRDVMLENYSNLVSVGYHIIKPDVIIKLEQGDEPWVGEE

SEQ ID NO: 68 - protein, 98 aa, synthetic construct
RTLVTFKDVAVDFTQEEWKLLDPAQQDLYRDVMLENYSNLVSLGYQLTKPDVILRLEQGKEPWLVEREIHQETHPDSETAFEIKSSVSSRSIFKDKQS

SEQ ID NO: 69 - protein, 96 aa, synthetic construct
VTFRDVAIDFSQEEWKWLQPAQRDLYRCVMLENYGHLVSLAGLSISKPDLVSLLEQGKEPWLGKGDIRRNLFSVSEPTDEIKEFSPENVIYKGDSS

SEQ ID NO: 70 - protein, 95 aa, synthetic construct
VTFRDVAIDFSQEEWKWLQPAQRDLYRCVMLENYGHLVSLGLSISKPDLVSLLEQGKEPWLGKGDIRRNLFSVSEPTDEIKEFSPENVIYKGDSS

SEQ ID NO: 71 - protein, 91 aa, synthetic construct
VTFRDVAIDFSQEEWEWLQPAQRDLYRCVMLENYGHLVSLAGLSISKPDVVSLLEQGKEPWLRKGDVSRDLFSVSESNCEIKDISSKNDIY

SEQ ID NO: 72 - protein, 403 aa, Ailuropoda melanoleuca
MALSPTGTLSVETLDRSDPDPLDEGPWQATCEILLEPDAEHSTDVILVGSSELSAPASPGPRRDLLAYEVKVNQRDIEDVCICCGSLRVHTQHPLFEGGMCAPCKDKFLDCLFLYDDDGYQSYCSICCAGETLLICENPDCTRPSLMMKLRLFRECACLIFPSEGMLLQTVWFWKMTVVWQPGLRHLPQENPLETYKTVPVWKREPVRVLSLFGDIRRELMSLGFLESGSAPGRLKHLDDVTDVVRKDVEGWGPFDLVYGSTPPIGHACDHPPVWYLLQFHRILQYARPRPGSQQPFFWMFVDNLVLSQDDQTAATRFLEADPVTIQDVCGRAVRNTVHVWSNIPAVRSRHSALALCEELSLLAQDRQRTKPPAQGPAQLVKNCFLPLREYFKYFSTELTSSL

SEQ ID NO: 73 - protein, 426 aa, Carlito syrichta
MALSCRRTLPLESLHSSNSDLASQLDKEQWRPPCETHGIPVAAAPVLDLEAECSLDVILVGSSELSTSSSPRLGRDHIAYEVKVNQRNIEDICLCCGSFLVHTQHPLFEGGMCAPCKDKFLDTLFLYDEDGYQSYCSICCSGETLLICENPDCTRCYCFECLDTLVSPGTSEKVHAMSNWVCFLCLPFTRSGLLQRRRKWRGQLKAFYDRESESSLEMYKTVPVWKREPVRVLSLFGDIKKELMSLGFVETGSDPGRLRHLDDTTNIVRRNVEEWGPFHLLYGATPPLGHTCDRPPGWYLFQFHRLLQYARPQPGSPQPFFWMFVDNVMLTREDRAIASRFLETEPVTIPDIHGRALQNAVCVWSNIPAVRSKHSALVSEEELSLLAQDRQRAKLPTQGPTKLVKNCFLPLREYFKYFSTELTSFL

SEQ ID NO: 74 - protein, 424 aa, Meriones unguiculatus
MGSQETPSTRAKTPGTWNLESTDSSSPESLGHLEEQWANSSPDLKDEHSKDVEPEDSKELISSASPPSGREIIRYEISVNQRNIEDICLCCGTLQVYKQHPLFEGGICAPCKDKFLETFFLYDEDGHQSYCSICCSGGTLFICESPDCTRCYCFECVDILVGPGTSERINAMPCWVCFLCLPFTRSGLLQRRRKWRHQLKAFFDEGGASPLEMYKTVSAWKRKPMRVLSLFKNIDKELKNLGFLESGSGSEEERLKYLEDVTNVVRRDVEKWGPFDLVYGSTRPRGSSCDHCPAWYMFQFHRILQYARPPSGSEQPFFWVFVDNLLMTEDDQITADRFLQMKAVTLQDVRGRVLQNAVRVWSNIPGVKSKHMALTEKEEQSLEAQAGTRTKLSAQKVDPLVKNCLLPLREYFKFFSQNSLPLDK

SEQ ID NO: 75 - protein, 409 aa, Ochotona princeps
MALPSPETLDSLDRVPASHPDEQHWTVCDNSDPILEVEAEGSMDVILVDDSPAPSGRDRIELEVKVNQRSIEDLCLCCGSSQVHRQHPLFQGGLCAPCKDKFLEALFLYDEDGYQSYCSICGLGDTLLVCESPDCTRGYCFACVDGLVGAGSSGHMHTVSPWVCFLCVPGSRHGLLQRRRRWRTQLKVFHEQEAAQPLEIYETVPACRRKPLRVLSLFEHIEKELASLGFLETGSSPGRIRHLDDVTDVVRRDVEQWGPFDLVYGSTPPLGHASPRSPGWYLFQFHRMLQYTQPTASTQRPFFWMFVDNLLLTRDDLVTATRFLEVEPATLQDVRGRVLQGAMRVWSNIPAVNSRHTELAPEAETALLAQSCRRAKASGEGLARLLKSCFLPLREYFKYFPQSPLPLRK

SEQ ID NO: 76 - protein, 224 aa, Neosciurus carolinensis
MGGPRPAAVEESPHEIYKTVPAWKREPMRVLSLFGDIGKELTSLGFLETGSEAGRLKHLEDVTDTVRRDVEEWGPFDLVYGSTPALGHSCDRSPGWYLFQFHRLLQYARPRLGSPKPFFWMFVDNLLLTKDDQAIASRFLEMEPVTLQDVHGRVLQNAVRVWTNVPAVKSRHSALASEEELLLVQDGQRGRLPAQGPAALVKHCFLPLREYFKYFSQNTLPLYK

SEQ ID NO: 77 - protein, 417 aa, Bison bison
MARSSPGTLNLEIMDGSDPDPALPPDREQWPPPCEILLDPEPEHSLDIILVGSSELSSPPSPGPRRDFIAYEVKVNQRDIEDVCICCGSLQLHTQHPLFEGGMCAPCKDKFLECLFLYDDDGYQSYCSICCAGETLLICENPDCTRCYCFECVDTLVGPGTSGKVHAMSNWVCFLCLPFPRSGLLQRRRKWRTWLKAFYDREAESPLVMYKTVPVWKREPIRVLSLFGDIKKELTSLGFLEDGSKPGRLKHLDDVTNIVRRDIDEWGPFDLTYGSTPTLGHTCDHPPGWYVYQFHRILQYARPLPGSPQPFFWMFVDNLVLTEEDLDVATRFLETDPVTIQDVRGRTVQNAVHVWSNIPAVKSRHSALVSQEELSLLAQDRQRVKSPVQGPATLVKNCFLPLREYFKYFSTELTSSL

SEQ ID NO: 78 - protein, 443 aa, Equus przewalskii
MALSSPGTLSLETLDSWDPDVAGQLDEERWQPSSEIVGRPMAAAPVLDLEEEPSMDIILVDSSELSSPPSPGPSRDMCICCGSFQVHTQHPLFEGGMCAACKDKFLSCLFLYDDDGNQSYCSICCSGETLLICENPDCTRCYCFECVDTLVSPRTSEKVQAMSNWVCFLCLPFPRSGLLQRRRKWRGWLKAFYDQEAVRSRSAWGRRMRSGPHLVGFLWLLVAKCPSALESPLEMYKTVPVWKREPVRVLSLFGDIKKELTTLGFLENGSDPGRLKHLDDVTNTVRRDVEEWGPFDLVYGSTPPLGHACDHPPGWYLFQFHRVLQYARPRPGSPQAFFWMFVDNLVLTEDDRAVATRFLETDPVTIQDVCGRAVRNAVHVWSNIPAVKSRHSALFSQEESFLRAQDRQRAKPPARGPAKLVKNCFLPLREYFKYFSTEFTSSL

SEQ ID NO: 79 - protein, 415 aa, Mus caroli
MGSRETPSSFSKTLETLDLETSDSSSPDADSPLEEQWLKSSPALKEDNVDMVLEDCKEPLSPSSPPTGREMIRYEVKVNRRSIEDICLCCGTLQVYTQHPLFEGGICAPCKDKFLESLFLYDDDGHQSYCTICCSGGTLFICESPDCTRCYCFECVDILVGPGTSERINAMACWVCFLCLPFSRSGLLQRRKRWRHQLKAFHDQEGAGPMEIYKTVSTWKRQPVRVLSLFGNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLMTEDDQETTARFLQTEAVTLQDVRGRDYQNVMRVWSNIPGLKSKHVPLTPKEEEYLQAQVRTRSKLDAQKVDLLVKNCLLPLREYFKYFS

SEQ ID NO: 80 - protein, 387 aa, Pan troglodytes
MAAIPALDPEAEPSMDVILVGSSELSSSISPRTGRDLIAYEVKANQRNIEDICICCGSLQVHTQHPLFEGGICAPCKDKSLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSSRHWALVSEEELSLLAQNKQSSKLAAKWPTKLVKNCFLPLREYFKYFSTELTSSL

SEQ ID NO: 81 - protein, 395 aa, Mycoplasma penetrans
MNSNKDKIKVIKVFEAFAGIGSQFKALKNIARSKNWEIQHSGMVEWFVDAIVSYVAIHSKNFNPKIEQLDKDILSISNDSKMPISEYGIKKINNTIKASYLNYAKKHFNNLFDIKKVNKDNFPKNIDIFTYSFPCQDLSVQGLQKGIDKELNTRSGLLWEIERILEEIKNSFSKEEMPKYLLMENVKNLLSHKNKKNYNTWLKQLEKFGYKSKTYLLNSKNFDNCQNRERVFCLSIRDDYLEKTGFKFKELEKVKNPPKKIKDILVDSSNYKYLNLNKYETTTFRETKSNIISRSLKNYTTFNSENYVYNINGIGPTLTASGANSRIKIETQQGVRYLTPLECFKYMQFDVNDFKKVQSTNLISENKMIYIAGNSIPVKILEAIFNTLEFVNNEE

SEQ ID NO: 82 - protein, 386 aa, Spiroplasma monobiae; identical to SEQ ID NO: 40

SEQ ID NO: 83 - protein, 358 aa, Haemophilus parainfluenzae
MKDVLDDNLLEEPAAQYSLFEPESNPNLREKFTFIDLFAGIGGFRIAMQNLGGKCIFSSEWDEQAQKTYEANFGDLPYGDITLEETKAFIPEKFDILCAGFPCQAFSIAGKRGGFEDTRGTLFFDVAEIIRRHQPKAFFLENVKGLKNHDKGRTLKTILNVLREDLGYFVPEPAIVNAKNFGVPQNRERIYIVGFHKSTGVNSFSYPEPLDKIVTFADIREEKTVPTKYYLSTQYIDTLRKHKERHESKGNGFGYEIIPDDGIANAIVVGGMGRERNLVIDHRITDFTPTTNIKGEVNREGIRKMTPREWARLQGFPDSYVIPVSDASAYKQFGNSVAVPAIQATGKKILEKLGNLYD

SEQ ID NO: 84 - protein, 521 aa, Arthrobacter luteus
MSKANAKYSFVDLFAGIGGFHAALAATGGVCEYAVEIDREAAAVYERNWNKPALGDITDDANDEGVTLRGYDGPIDVLTGGFPCQPFSKSGAQHGMAETRGTLFWNIARIIEEREPTVLILENVRNLVGPRHRHEWLTIIETLRFFGYEVSGAPAIFSPHLLPAWMGGTPQVRERVFITATLVPERMRDERIPRTETGEIDAEAIGPKPVATMNDRFPIKKGGTELFHPGDRKSGWNLLTSGIIREGDPEPSNVDLRLTETETLWIDAWDDLESTIRRATGRPLEGFPYWADSWTDFRELSRLVVIRGFQAPEREVVGDRKRYVARTDMPEGFVPASVTRPAIDETLPAWKQSHLRRNYDFFERHFAEVVAWAYRWGVYTDLFPASRRKLEWQAQDAPRLWDTVMHFRPSGIRAKRPTYLPALVAITQTSIVGPLERRLSPRETARLQGLPEWFDFGEQRAAATYKQMGNGVNVGVVRHILREHVRRDRALLKLTPAGQRIINAVLADEPDATVGALGAAE

SEQ ID NO: 85 - protein, 330 aa, Haemophilus aegyptius; identical to SEQ ID NO: 38

SEQ ID NO: 86 - protein, 327 aa, Haemophilus haemolyticus; identical to SEQ ID NO: 39

SEQ ID NO: 87 - protein, 418 aa, Moraxella sp.; identical to SEQ ID NO: 37

SEQ ID NO: 88 - protein, 529 aa, Escherichia coli
MNNNDLVAKLWKLCDNLRDGGVSYQNYVNELASLLFLKMCKETGQEAEYLPEGYRWDDLKSRIGQEQLQFYRKMLVHLGEDDKKLVQAVFHNVSTTITEPKQITALVSNMDSLDWYNGAHGKSRDDFGDMYEGLLQKNANETKSGAGQYFTPRPLIKTIIHLLKPQPREVVQDPAAGTAGFLIEADRYVKSQTNDLDDLDGDTQDFQIHRAFIGLELVPGTRRLALMNCLLHDIEGNLDHGGAIRLGNTLGSDGENLPKAHIVATNPPFGSAAGTNITRTFVHPTSNKQLCFMQHIIETLHPGGRAAVVVPDNVLFEGGKGTDIRRDLMDKCHLHTILRLPTGIFYAQGVKTNVLFFTKGTVANPNQDKNCTDDVWVYDLRTNMPSFGKRTPFTDEHLQPFERVYGEDPHGLSPRTEGEWSFNAEETEVADSEENKNTDQHLATSRWRKFSREWIRTAKSDSLDISWLKDKDSIDADSLPEPDVLAAEAMGELVQALSELDALMRELGASDEADLQRQLLEEAFGGVKE

SEQ ID NO: 89 - protein, 464 aa, Escherichia coli
MSAGKLPEGWVIAPVSTVTTLIRGVTYKKEQAINYLKDDYLPLIRANNIQNGKFDTTDLVFVPKNLVKESQKISPEDIVIAMSSGSKSVVGKSAHQHLPFECSFGAFCGVLRPEKLIFSGFIAHFTKSSLYRNKISSLSAGANINNIKPASFDLINIPIPPLAEQKIIAEKLDTLLAQVDSTKARFEQIPQILKRFRQAVLGGAVNGKLTEKWRNFEPQHSVFKKLNFESILTELRNGLSSKPNESGVGHPILRISSVRAGHVDQNDIRFLECSESELNRHKLQDGDLLFTRYNGSLEFVGVCGLLKKLQHQNLLYPDKLIRARLTKDALPEYIEIFFSSPSARNAMMNCVKTTSGQKGISGKDIKSQVVLLPPVKEQAEIVRRVEQLFAYADTIEKQVNNALARVNNLTQSILAKAFRGELTAQWRAENPDLISGENSAAALLEKIKAERAASGGKKASRKKS

SEQ ID NO: 90 - protein, 421 aa, Thermus aquaticus
MGLPPLLSLPSNSAPRSLGRVETPPEVVDFMVSLAEAPRGGRVLEPACAHGPFLRAFREAHGTAYRFVGVEIDPKALDLPPWAEGILADFLLWEPGEAFDLILGNPPYGIVGEASKYPIHVFKAVKDLYKKAFSTWKGKYNLYGAFLEKAVRLLKPGGVLVFVVPATWLVLEDFALLREFLAREGKTSVYYLGEVFPQKKVSAVVIRFQKSGKGLSLWDTQESESGFTPILWAEYPHWEGEIIRFETEETRKLEISGMPLGDLFHIRFAARSPEFKKHPAVRKEPGPGLVPVLTGRNLKPGWVDYEKNHSGLWMPKERAKELRDFYATPHLVVAHTKGTRVVAAWDERAYPWREEFHLLPKEGVRLDPSSLVQWLNSEAMQKHVRTLYRDFVPHLTLRMLERLPVRREYGFHTSPESARNF

SEQ ID NO: 91 - protein, 278 aa, Escherichia coli
MKKNRAFLKWAGGKYPLLDDIKRHLPKGECLVEPFVGAGSVFLNTDFSRYILADINSDLISLYNIVKMRTDEYVQAARELFVPETNCAEVYYQFREEFNKSQDPFRRAVLFLYLNRYGYNGLCRYNLRGEFNVPFGRYKKPYFPEAELYHFAEKAQNAFFYCESYADSMARADDASVVYCDPPYAPLSATANFTAYHTNSFTLEQQAHLAEIAEGLVERHIPVLISNHDTMLTREWYQRAKLHVVKVRRSISSNGGTRKKVDELLALYKPGVVSPAKK

SEQ ID NO: 92 - protein, 358 aa, Caulobacter crescentus
MKFGPETIIHGDCIEQMNALPEKSVDLIFADPPYNLQLGGDLLRPDNSKVDAVDDHWDQFESFAAYDKFTREWLKAARRVLKDDGAIWVIGSYHNIFRVGVAVQDLGFWILNDIVWRKSNPMPNFKGTRFANAHETLIWASKSQNAKRYTFNYDALKMANDEVQMRSDWTIPLCTGEERIKGADGQKAHPTQKPEALLYRVILSTTKPGDVILDPFFGVGTTGAAAKRLGRKFIGIEREAEYLEHAKARIAKVVPIAPEDLDVMGSKRAEPRVPFGTIVEAGLLSPGDTLYCSKGTHVAKVRPDGSITVGDLSGSIHKIGALVQSAPACNGWTYWHFKTDAGLAPIDVLRAQVRAGMN

SEQ ID NO: 93 - protein, 577 aa, Clostridioides difficile
MDDISQDNFLLSKEYENSLDVDTKKASGIYYTPKIIVDYIVKKTLKNHDIIKNPYPRILDISCGCGNFLLEVYDILYDLFEENIYELKKKYDENYWTVDNIHRHILNYCIYGADIDEKAISILKDSLTNKKVVNDLDESDIKINLFCCDSLKKKWRYKFDYIVGNPPYIGHKKLEKKYKKFLLEKYSEVYKDKADLYFCFYKKIIDILKQGGIGSVITPRYFLESLSGKDLREYIKSNVNVQEIVDFLGANIFKNIGVSSCILTFDKKKTKETYIDVFKIKNEDICINKFETLEELLKSSKFEHFNINQRLLSDEWILVNKDDETFYNKIQEKCKYSLEDIAISFQGIITGCDKAFILSKDDVKLNLVDDKFLKCWIKSKNINKYIVDKSEYRLIYSNDIDNENTNKRILDEIIGLYKTKLENRRECKSGIRKWYELQWGREKLFFERKKIMYPYKSNENRFAIDYDNNFSSADVYSFFIKEEYLDKFSYEYLVGILNSSVYDKYFKITAKKMSKNIYDYYPNKVMKIRIFRDNNYEEIENLSKQIISILLNKSIDKGKVEKLQIKMDNLIMDSLGI

SEQ ID NO: 94 - protein, 573 aa, synthetic construct
MDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQSCDIKMEGMARNDLWYLSLEEVWKCRDQLDKYQENPERHLRQVAFTQKKVLTQERVSESGKYGGNCLLPAQLVLREYFHKRDSHTKSLKHDLVLNGHQDSCASNSNECGQTFCQNIHLIQFARTHTGDKSYKCPDNDNSLTHGSSLGISKGIHREKPYECKECGKFFSWRSNLTRHQLIHTGEKPYECKECGKSFSRSSHLIGHQKTHTGEEPYECKECGKSFSWFSHLVTHQRTHTGDKLYTCNQCGKSFVHSSRLIRHQRTHTGEKPYECPECGKSFRQSTHLILHQRTHVRVRPYECNECGKSYSQRSHLVVHHRIHTGLKPFECKDCGKCFSRSSHLYSHQRTHTGEKPYECHDCGKSFSQSSALIVHQRIHTGEKPYECCQCGKAFIRKNDLIKHQRIHVGEETYKCNQCGIIFSQNSPFIVHQIAHTGEQFLTCNQCGTALVNTSNLIGYQTNHIRENAY

SEQ ID NO: 95 - protein, 14 aa, synthetic construct
PKKKRKVPKKKRKV

SEQ ID NO: 96 - protein, 301 aa, synthetic construct
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 97 - protein, 213 aa, synthetic construct
NPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 98 - protein, 133 aa, synthetic construct
EVKVNRRSIEDICLCCGTLQVYTRHPLFEGGLCAPCKDKFLESLFLYDDDGHQSYCTICCSGGTLFICESPDCTRCYCFECVDILVGPGTSERINAMACWVCFLCLPFSRSGLLQRRKRWRHQLKAFHDQEGA

SEQ ID NO: 99 - protein, 62 aa, synthetic construct; identical to SEQ ID NO: 58

SEQ ID NO: 100 - protein, 96 aa, synthetic construct
MGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 101 - protein, 213 aa, Ailuropoda melanoleuca
NPLETYKTVPVWKREPVRVLSLFGDIRRELMSLGFLESGSAPGRLKHLDDVTDVVRKDVEGWGPFDLVYGSTPPIGHACDHPPVWYLLQFHRILQYARPRPGSQQPFFWMFVDNLVLSQDDQTAATRFLEADPVTIQDVCGRAVRNTVHVWSNIPAVRSRHSALALCEELSLLAQDRQRTKPPAQGPAQLVKNCFLPLREYFKYFSTELTSSL

SEQ ID NO: 102 - protein, 213 aa, Carlito syrichta
SSLEMYKTVPVWKREPVRVLSLFGDIKKELMSLGFVETGSDPGRLRHLDDTTNIVRRNVEEWGPFHLLYGATPPLGHTCDRPPGWYLFQFHRLLQYARPQPGSPQPFFWMFVDNVMLTREDRAIASRFLETEPVTIPDIHGRALQNAVCVWSNIPAVRSKHSALVSEEELSLLAQDRQRAKLPTQGPTKLVKNCFLPLREYFKYFSTELTSFL

SEQ ID NO: 103 - protein, 216 aa, Meriones unguiculatus
SPLEMYKTVSAWKRKPMRVLSLFKNIDKELKNLGFLESGSGSEEERLKYLEDVTNVVRRDVEKWGPFDLVYGSTRPRGSSCDHCPAWYMFQFHRILQYARPPSGSEQPFFWVFVDNLLMTEDDQITADRFLQMKAVTLQDVRGRVLQNAVRVWSNIPGVKSKHMALTEKEEQSLEAQAGTRTKLSAQKVDPLVKNCLLPLREYFKFFSQNSLPLDK

SEQ ID NO: 104 - protein, 214 aa, Ochotona princeps
QPLEIYETVPACRRKPLRVLSLFEHIEKELASLGFLETGSSPGRIRHLDDVTDVVRRDVEQWGPFDLVYGSTPPLGHASPRSPGWYLFQFHRMLQYTQPTASTQRPFFWMFVDNLLLTRDDLVTATRFLEVEPATLQDVRGRVLQGAMRVWSNIPAVNSRHTELAPEAETALLAQSCRRAKASGEGLARLLKSCFLPLREYFKYFPQSPLPLRK

SEQ ID NO: 105 - protein, 213 aa, Neosciurus carolinensis
SPHEIYKTVPAWKREPMRVLSLFGDIGKELTSLGFLETGSEAGRLKHLEDVTDTVRRDVEEWGPFDLVYGSTPALGHSCDRSPGWYLFQFHRLLQYARPRLGSPKPFFWMFVDNLLLTKDDQAIASRFLEMEPVTLQDVHGRVLQNAVRVWTNVPAVKSRHSALASEEELLLVQDGQRGRLPAQGPAALVKHCFLPLREYFKYFSQNTLPLYK

SEQ ID NO: 106 - protein, 213 aa, Bison bison
SPLVMYKTVPVWKREPIRVLSLFGDIKKELTSLGFLEDGSKPGRLKHLDDVTNIVRRDIDEWGPFDLTYGSTPTLGHTCDHPPGWYVYQFHRILQYARPLPGSPQPFFWMFVDNLVLTEEDLDVATRFLETDPVTIQDVRGRTVQNAVHVWSNIPAVKSRHSALVSQEELSLLAQDRQRVKSPVQGPATLVKNCFLPLREYFKYFSTELTSSL

SEQ ID NO: 107 - protein, 213 aa, Equus przewalskii
SPLEMYKTVPVWKREPVRVLSLFGDIKKELTTLGFLENGSDPGRLKHLDDVTNTVRRDVEEWGPFDLVYGSTPPLGHACDHPPGWYLFQFHRVLQYARPRPGSPQAFFWMFVDNLVLTEDDRAVATRFLETDPVTIQDVCGRAVRNAVHVWSNIPAVKSRHSALFSQEESFLRAQDRQRAKPPARGPAKLVKNCFLPLREYFKYFSTEFTSSL

SEQ ID NO: 108 - protein, 208 aa, Mus caroli
GPMEIYKTVSTWKRQPVRVLSLFGNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLMTEDDQETTARFLQTEAVTLQDVRGRDYQNVMRVWSNIPGLKSKHVPLTPKEEEYLQAQVRTRSKLDAQKVDLLVKNCLLPLREYFKYFS

SEQ ID NO: 109 - protein, 214 aa, Pan troglodytes
NPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSSRHWALVSEEELSLLAQNKQSSKLAAKWPTKLVKNCFLPLREYFKYFSTELTSSL

SEQ ID NO: 110 - protein, 2298 aa, synthetic construct
MGTMPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSELEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVGVDGSSGSETPGTSESATPESRTLVTFKDVFVDFTREEWKLLDTAQQIVYRNVMLENYKNLVSLGYQLTKPDVILRLEKGEEPSADYKDDDDKAPKKKRKVPKKKRKV

SEQ ID NO: 111 - DNA, 6897 bp, synthetic construct
  source 1..6897: mol_type = other DNA
atgggtaccatgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtcccggaccctggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcgctgattacaaagatgatgacgataaagccccaaaaaagaagagaaaggtaccgaagaaaaaaagaaaggtctga

SEQ ID NO: 112 - protein, 2318 aa, synthetic construct
MGTMPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSELEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVGVDGSSGSETPGTSESATPESTGNKKLEAVGTGIEPKAMSQGLVTFGDVAVDFSQEEWEWLNPIQRNLYRKVMLENYRNLASLGLCVSKPDVISSLEQGKEPWSADYKDDDDKAPKKKRKVPKKKRKV

SEQ ID NO: 113 - DNA, 6957 bp, synthetic construct
  source 1..6957: mol_type = other DNA
atgggtaccatgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccgggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtccaccggtaacaaaaagcttgaggccgtcggaaccggaatcgaaccaaaagcaatgtcccagggtttggtgacatttggcgacgtggctgtcgatttttcccaggaagagtgggagtggctcaatcctatccagaggaacttgtaccggaaggtgatgctggagaattatagaaatttggcatcactggggttgtgcgttagcaaaccagatgttatatcttccctggaacagggaaaggagccctggagcgctgattacaaagatgatgacgataaagcccccaagaagaaaaggaaggtcccaaagaaaaaaagaaaggtgtga

SEQ ID NO: 114 - protein, 2338 aa, synthetic construct
MGTMPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSELEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVGVDGSSGSETPGTSESATPESTGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLSADYKDDDDKAPKKKRKVPKKKRKV

SEQ ID NO: 115 - DNA, 7017 bp, synthetic construct
  source 1..7017: mol_type = other DNA
atgggtaccatgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtccaccggtatgaacaattcacaggggagagtgacattcgaagacgtgaccgtgaacttcacccagggagaatggcagcgcttgaacccagaacaaaggaacctctatcgggacgtgatgctggaaaactactcaaatttggtgagcgttgggcagggtgagaccactaagcctgacgtgatcctgagattggaacagggcaaggagccttggctcgaggaagaggaagtcctgggctcagggagggccgagaaaaacggtgatataggaggccagatatggaagcctaaggacgtcaaggagagcctgagcgctgattacaaagatgatgacgataaagcccccaagaagaaaaggaaggtcccaaagaaaaaaagaaaggtgtga

SEQ ID NO: 116 - protein, 2318 aa, synthetic construct
MGTMPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSELEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESADYKDDDDKAPKKKRKVPKKKRKV

SEQ ID NO: 117 - DNA, 6957 bp, synthetic construct
  source 1..6957: mol_type = other DNA
atgggtaccatgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctgattacaaagatgatgacgataaagcccccaagaagaaaaggaaggtcccaaagaaaaaaagaaaggtgtga

SEQ ID NO: 118 - RNA, 20 nt, synthetic construct
  source 1..20: mol_type = other RNA
actgcctggctcactcctcc

SEQ ID NO: 119 - RNA, 20 nt, synthetic construct
  source 1..20: mol_type = other RNA
tgcggaaaccttctagggtg

SEQ ID NO: 120 - RNA, 20 nt, synthetic construct
  source 1..20: mol_type = other RNA
aacctgatcctccagtccgg

SEQ ID NO: 121 - RNA, 20 nt, synthetic construct
  source 1..20: mol_type = other RNA
atcgtccgatggggctctgg

SEQ ID NO: 122 - RNA, 20 nt, synthetic construct
  source 1..20: mol_type = other RNA
ggtgtgggtgcttgacgcct

SEQ ID NO: 123 - RNA, 20 nt, synthetic construct
  source 1..20: mol_type = other RNA
caaatcctaactgggctgga

SEQ ID NO: 124 - RNA, 20 nt, synthetic construct
  source 1..20: mol_type = other RNA
gggcgccgccgttcagttca

SEQ ID NO: 125 - RNA, 20 nt, synthetic construct
  source 1..20: mol_type = other RNA
gcggaaaccttctagggtgt

SEQ ID NO: 126 - RNA, 20 nt, synthetic construct
  source 1..20: mol_type = other RNA
gtgtgggtgcttgacgcctg

SEQ ID NO: 127 - RNA, 20 nt, synthetic construct
  source 1..20: mol_type = other RNA
aggatcgtccgatggggctc

SEQ ID NO: 128 - RNA, 20 nt, synthetic construct
  source 1..20: mol_type = other RNA
agttcagggtctgagcctgg

SEQ ID NO: 129 - RNA, 20 nt, synthetic construct
  source 1..20: mol_type = other RNA
tcaagcacccacaccctaga

SEQ ID NO: 130 - RNA, 20 nt, synthetic construct
  source 1..20: mol_type = other RNA
tcagataggatcgtccgatg

SEQ ID NO: 131 - DNA, 18 bp, synthetic construct
  source 1..18: mol_type = other DNA
gatggggctctggtggcg

SEQ ID NO: 132 - protein, 176 aa, synthetic construct
SRPGERPFQCRICMRNFSRTDTLARHLRTHTGEKPFQCRICMRNFSRKTALNRHLKTHTGSQKPFQCRICMRNFSRNESLKVHLRTHTGEKPFQCRICMRNFSVKNTLTRHLKTHTGSQKPFQCRICMRNFSRREHLVRHLRTHTGEKPFQCRICMRNFSLTHNLRRHLKTHLRGS

SEQ ID NO: 133 - protein, 909 aa, synthetic construct
MGTMYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVSRPGERPFQCRICMRNFSRTDTLARHLRTHTGEKPFQCRICMRNFSRKTALNRHLKTHTGSQKPFQCRICMRNFSRNESLKVHLRTHTGEKPFQCRICMRNFSVKNTLTRHLKTHTGSQKPFQCRICMRNFSRREHLVRHLRTHTGEKPFQCRICMRNFSLTHNLRRHLKTHLRGSPKKKRKVSGSETPGTSESATPESTGRTLVTFKDVFVDFTREEWKLLDTAQQIVYRNVMLENYKNLVSLGYQLTKPDVILRLEKGEEPSADYKDDDDKA

SEQ ID NO: 134 - protein, 923 aa, synthetic construct
MGTMPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSRTDTLARHLRTHTGEKPFQCRICMRNFSRKTALNRHLKTHTGSQKPFQCRICMRNFSRNESLKVHLRTHTGEKPFQCRICMRNFSVKNTLTRHLKTHTGSQKPFQCRICMRNFSRREHLVRHLRTHTGEKPFQCRICMRNFSLTHNLRRHLKTHLRGSSGSETPGTSESATPESTGRTLVTFKDVFVDFTREEWKLLDTAQQIVYRNVMLENYKNLVSLGYQLTKPDVILRLEKGEEPSADYKDDDDKAPKKKRKVPKKKRKV

SEQ ID NO: 135 - DNA, 19 bp, unidentified
  source 1..19: mol_type = unassigned DNA; note = mammalian target sequence
gcagaggccggagggggtc

SEQ ID NO: 136 - protein, 179 aa, synthetic construct
SRPGERPFQCRICMRNFSDPSVLKRHLRTHTGEKPFQCRICMRNFSRTEHLARHLKTHTGGGGSQKPFQCRICMRNFSQSPHLKRHLRTHTGEKPFQCRICMRNFSDQTTLRRHLKTHTGSQKPFQCRICMRNFSKHSNLTRHTRTHTGEKPFQCRICMRNFSQMETLKRHLRTHLRGS

SEQ ID NO: 137 - protein, 912 aa, synthetic construct
MGTMYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVSRPGERPFQCRICMRNFSDPSVLKRHLRTHTGEKPFQCRICMRNFSRTEHLARHLKTHTGGGGSQKPFQCRICMRNFSQSPHLKRHLRTHTGEKPFQCRICMRNFSDQTTLRRHLKTHTGSQKPFQCRICMRNFSKHSNLTRHTRTHTGEKPFQCRICMRNFSQMETLKRHLRTHLRGSPKKKRKVSGSETPGTSESATPESTGRTLVTFKDVFVDFTREEWKLLDTAQQIVYRNVMLENYKNLVSLGYQLTKPDVILRLEKGEEPSADYKDDDDKA

SEQ ID NO: 138 - protein, 926 aa, synthetic construct
MGTMPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSDPSVLKRHLRTHTGEKPFQCRICMRNFSRTEHLARHLKTHTGGGGSQKPFQCRICMRNFSQSPHLKRHLRTHTGEKPFQCRICMRNFSDQTTLRRHLKTHTGSQKPFQCRICMRNFSKHSNLTRHTRTHTGEKPFQCRICMRNFSQMETLKRHLRTHLRGSSGSETPGTSESATPESTGRTLVTFKDVFVDFTREEWKLLDTAQQIVYRNVMLENYKNLVSLGYQLTKPDVILRLEKGEEPSADYKDDDDKAPKKKRKVPKKKRKV

SEQ ID NO: 139 - DNA, 18 bp, unidentified
  source 1..18: mol_type = unassigned DNA; note = mammalian target sequence
ggtggggaggactgtgca

SEQ ID NO: 140 - protein, 176 aa, synthetic construct
SRPGERPFQCRICMRNFSQSTTLKRHLRTHTGEKPFQCRICMRNFSGAHGLAGHLKTHTGSQKPFQCRICMRNFSDQTNLRRHLRTHTGEKPFQCRICMRNFSRQDNLQRHLKTHTGSQKPFQCRICMRNFSKGDHLRRHTRTHTGEKPFQCRICMRNFSEAHHLSRHLRTHLRGS

SEQ ID NO: 141 - protein, 909 aa, synthetic construct
MGTMYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVSRPGERPFQCRICMRNFSQSTTLKRHLRTHTGEKPFQCRICMRNFSGAHGLAGHLKTHTGSQKPFQCRICMRNFSDQTNLRRHLRTHTGEKPFQCRICMRNFSRQDNLQRHLKTHTGSQKPFQCRICMRNFSKGDHLRRHTRTHTGEKPFQCRICMRNFSEAHHLSRHLRTHLRGSPKKKRKVSGSETPGTSESATPESTGRTLVTFKDVFVDFTREEWKLLDTAQQIVYRNVMLENYKNLVSLGYQLTKPDVILRLEKGEEPSADYKDDDDKA

SEQ ID NO: 142 - protein, 923 aa, synthetic construct
MGTMPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSQSTTLKRHLRTHTGEKPFQCRICMRNFSGAHGLAGHLKTHTGSQKPFQCRICMRNFSDQTNLRRHLRTHTGEKPFQCRICMRNFSRQDNLQRHLKTHTGSQKPFQCRICMRNFSKGDHLRRHTRTHTGEKPFQCRICMRNFSEAHHLSRHLRTHLRGSSGSETPGTSESATPESTGRTLVTFKDVFVDFTREEWKLLDTAQQIVYRNVMLENYKNLVSLGYQLTKPDVILRLEKGEEPSADYKDDDDKAPKKKRKVPKKKRKV

SEQ ID NO: 143 - DNA, 19 bp, unidentified
  source 1..19: mol_type = unassigned DNA; note = mammalian target sequence
gcaggaggacgaggacggc

SEQ ID NO: 144 - protein, 179 aa, synthetic construct
SRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGS

SEQ ID NO: 145 - protein, 912 aa, synthetic construct
MGTMYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVSRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGSPKKKRKVSGSETPGTSESATPESTGRTLVTFKDVFVDFTREEWKLLDTAQQIVYRNVMLENYKNLVSLGYQLTKPDVILRLEKGEEPSADYKDDDDKA

SEQ ID NO: 146 - protein, 926 aa, synthetic construct
MGTMPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSTPSKLDRHTRTHTGEKPFQCRICMRNFSLAENLRRHLRTHTGSQKPFQCRICMRNFSRQDNLGRHLRTHTGEKPFQCRICMRNFSDGGNLGRHLKTHTGGGGSQKPFQCRICMRNFSQSPHLKRHLRTHTGEKPFQCRICMRNFSQSTSLQRHLKTHLRGSSGSETPGTSESATPESTGRTLVTFKDVFVDFTREEWKLLDTAQQIVYRNVMLENYKNLVSLGYQLTKPDVILRLEKGEEPSADYKDDDDKAPKKKRKVPKKKRKV

SEQ ID NO: 147 - DNA, 19 bp, unidentified
  source 1..19: mol_type = unassigned DNA; note = mammalian target sequence
gcaggaggacgaggacggc

SEQ ID NO: 148 - protein, 179 aa, synthetic construct
SRPGERPFQCRICMRNFSERAKLIRHLRTHTGEKPFQCRICMRNFSDPSNLRRHLKTHTGSQKPFQCRICMRNFSRQDNLGRHLRTHTGEKPFQCRICMRNFSDQGNLGRHLKTHTGGGGSQKPFQCRICMRNFSQSAHLKRHLRTHTGEKPFQCRICMRNFSQDVSLVRHLKTHLRGS

SEQ ID NO: 149 - protein, 912 aa, synthetic construct
MGTMYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVSRPGERPFQCRICMRNFSERAKLIRHLRTHTGEKPFQCRICMRNFSDPSNLRRHLKTHTGSQKPFQCRICMRNFSRQDNLGRHLRTHTGEKPFQCRICMRNFSDQGNLGRHLKTHTGGGGSQKPFQCRICMRNFSQSAHLKRHLRTHTGEKPFQCRICMRNFSQDVSLVRHLKTHLRGSPKKKRKVSGSETPGTSESATPESTGRTLVTFKDVFVDFTREEWKLLDTAQQIVYRNVMLENYKNLVSLGYQLTKPDVILRLEKGEEPSADYKDDDDKA

SEQ ID NO: 150 - protein, 926 aa, synthetic construct
MGTMPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSESRPGERPFQCRICMRNFSERAKLIRHLRTHTGEKPFQCRICMRNFSDPSNLRRHLKTHTGSQKPFQCRICMRNFSRQDNLGRHLRTHTGEKPFQCRICMRNFSDQGNLGRHLKTHTGGGGSQKPFQCRICMRNFSQSAHLKRHLRTHTGEKPFQCRICMRNFSQDVSLVRHLKTHLRGSSGSETPGTSESATPESTGRTLVTFKDVFVDFTREEWKLLDTAQQIVYRNVMLENYKNLVSLGYQLTKPDVILRLEKGEEPSADYKDDDDKAPKKKRKVPKKKRKV

SEQ ID NO: 151 - DNA, 20 bp, unidentified
  source 1..20: mol_type = unassigned DNA; note = mammalian target sequence
ggcggaagtgctgcctgata

SEQ ID NO: 152 - DNA, 20 bp, unidentified
  source 1..20: mol_type = unassigned DNA; note = mammalian target sequence
gcaacaccgcctagaccgac

SEQ ID NO: 153 - DNA, 20 bp, unidentified
  source 1..20: mol_type = unassigned DNA; note = mammalian target sequence
ggaggttcagaaagcctaag

SEQ ID NO: 154 - DNA, 20 bp, unidentified
  source 1..20: mol_type = unassigned DNA; note = mammalian target sequence
gcagcactagagtcccctca

SEQ ID NO: 155 - DNA, 20 bp, unidentified
  source 1..20: mol_type = unassigned DNA; note = mammalian target sequence
gaacaactcgtgactggggt

SEQ ID NO: 156 - DNA, 20 bp, unidentified
  source 1..20: mol_type = unassigned DNA; note = mammalian target sequence
gggagaggaggacaagcgcc

SEQ ID NO: 157 - DNA, 20 bp, unidentified
  source 1..20: mol_type = unassigned DNA; note = mammalian target sequence
ggacccaccgacaccacgcc

SEQ ID NO: 158 - DNA, 20 bp, unidentified
  source 1..20: mol_type = unassigned DNA; note = mammalian target sequence
cgcgggaaaatgcaagacga

SEQ ID NO: 159 - RNA, 69 nt, synthetic construct
  source 1..69: mol_type = other RNA
taatacgactcactataaggggcgctcgagcaggttcagaaggagatcaaaaacccccaaggatcaaac

SEQ ID NO: 160 - protein, 2326 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSELEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVGVDGSSGSETPGTSESATPESTGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLSAPKKKRKVPKKKRKV

SEQ ID NO: 161 - DNA, 6981 bp, synthetic construct
  source 1..6981: mol_type = other DNA
atgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtccaccggtatgaacaattcacaggggagagtgacattcgaagacgtgaccgtgaacttcacccagggagaatggcagcgcttgaacccagaacaaaggaacctctatcgggacgtgatgctggaaaactactcaaatttggtgagcgttgggcagggtgagaccactaagcctgacgtgatcctgagattggaacagggcaaggagccttggctcgaggaagaggaagtcctgggctcagggagggccgagaaaaacggtgatataggaggccagatatggaagcctaaggacgtcaaggagagcctgagcgctcccaagaagaaaaggaaggtcccaaagaaaaaaagaaaggtgtga

SEQ ID NO: 162 - protein, 2306 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSELEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVGVDGSSGSETPGTSESATPESTGDSVAFEDVAVNFTLEEWALLDPSQKNLYRDVMRETFRNLASVGKQWEDQNIEDPFKIPRRNISHIPERLCESKEGGQGEESAPKKKRKVPKKKRKV

SEQ ID NO: 163 - DNA, 6921 bp, synthetic construct
  source 1..6921: mol_type = other DNA
atgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtccaccggtgactccgttgctttcgaggacgtggccgtgaacttcacacttgaggaatgggccttgctcgacccaagtcagaagaatctgtacagagacgtgatgcgggagacattcaggaatctcgccagtgtcggaaagcagtgggaagaccagaacatcgaagatcctttcaagataccacggcgcaatatctcccacattcctgagaggctgtgtgaatctaaggaaggcggacaaggtgaggaaagcgctcccaagaagaaaaggaaggtcccaaagaaaaaaagaaaggtgtga

SEQ ID NO: 164 - protein, 2286 aa, synthetic construct
MPKKKRKVPKKKRKVYNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSELEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVGVDGSSGSETPGTSESATPESRTLVTFKDVFVDFTREEWKLLDTAQQIVYRNVMLENYKNLVSLGYQLTKPDVILRLEKGEEPSAPKKKRKVPKKKRKV

SEQ ID NO: 165 - DNA, 6861 bp, synthetic construct
  source 1..6861: mol_type = other DNA
atgccaaaaaagaagagaaaggtaccgaagaaaaaaagaaaggtatacaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtcccggaccctggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccagcgctccaaaaaagaagagaaaggtaccgaagaaaaaaagaaaggtctga

SEQ ID NO: 166 - protein, 2266 aa, synthetic construct
MSPKKKRKVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSLGGPSSGAPPPSGGSPAGSPTSTEEGTSESATPESGPGTSTEPSEGSAPGSPAGSPTSTEEGTSTEPSEGSAPGTSTEPSELEDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVGVDGSSGSETPGTSESATPESRTLVTFKDVFVDFTREEWKLLDTAQQIVYRNVMLENYKNLVSLGYQLTKPDVILRLEKGEEPWLV

SEQ ID NO: 167 - DNA, 6801 bp, synthetic construct
  source 1..6801: mol_type = other DNA
atgtcgccgaagaaaaagcgcaaggtcaatcacgatcaggagttcgacccccctaaggtgtacccaccagtgcctgcagagaagaggaagccaatccgggtgctgagcctgtttgatggcatcgccaccggcctgctggtgctgaaggatctgggcatccaggtggaccggtacatcgcctccgaggtgtgcgaggattctatcaccgtgggcatggtgcgccaccagggcaagatcatgtatgtgggcgacgtgcggtccgtgacacagaagcacatccaggagtggggcccattcgatctggtgatcggcggcagcccctgtaatgacctgtccatcgtgaaccctgcaaggaagggactgtacgagggaaccggccggctgttctttgagttttatagactgctgcacgacgccaggcctaaggagggcgacgatagaccattcttttggctgttcgagaatgtggtggctatgggcgtgagcgataagagggacatctccaggtttctggagtctaaccccgtgatgatcgatgcaaaggaggtgtccgccgcacacagagccaggtatttctggggcaatctgccaggaatgaacaggccactggcaagcaccgtgaatgacaagctggagctgcaggagtgcctggagcacggaaggatcgccaagttttccaaggtgcgcacaatcaccacacggagcaattccatcaagcagggcaaggatcagcacttccccgtgttcatgaacgagaaggaggacatcctgtggtgtaccgagatggagagagtgttcggctttccagtgcactacacagacgtgtctaacatgagcaggctggcaaggcagcggctgctgggcagatcttggagcgtgcccgtgatcaggcacctgttcgcccctctgaaggagtattttgcctgcgtgagcagcggcaactccaatgccaacagccggggcccctctttcagctccggattggtgcctctgagcctgaggggctcccacatggcagcaatccccgccctggaccccgaggccgagcctagcatggacgtgatcctggtgggctctagcgagctgtcctctagcgtgtctccaggaaccggaagggatctgatcgcatacgaggtgaaggccaatcagcggaacatcgaggacatctgtatctgctgtggcagcctgcaggtgcacacacagcacccactgttcgagggaggaatctgcgcaccctgtaaggataagttcctggacgccctgtttctgtacgacgatgacggctaccagtcctattgctctatctgctgttccggcgagaccctgctgatctgcggcaatccagattgtacaaggtgctattgttttgagtgcgtggactctctggtgggaccaggcaccagcggaaaggtgcacgccatgtccaactgggtgtgctacctgtgcctgccatcctctcgcagcggactgctgcagcggagaaggaagtggagatcccagctgaaggccttctatgatagggagtctgagaaccccctggagatgtttgagaccgtgccagtgtggcgccggcagcccgtgagggtgctgagcctgttcgaggatatcaagaaggagctgacatccctgggctttctggagtccggctctgaccccggacagctgaagcacgtggtggatgtgaccgacacagtgcggaaggatgtggaggagtggggccctttcgacctggtgtacggagcaacccctccactgggacacacatgcgacagacccccttcttggtacctgttccagtttcaccgcctgctgcagtatgcaaggccaaagccaggcagccctagaccattcttttggatgttcgtggataatctggtgctgaacaaggaggatctggacgtggccagcaggtttctggagatggagccagtgaccatcccagacgtgcacggcggctccctgcagaatgccgtgcgcgtgtggtctaacatccctgccatcagaagcaggcactgggcactggtgagcgaggaggagctgtccctgctggcccagaataagcagagcagcaagctggccgccaagtggcctacaaagctggtgaagaactgcttcctgccactgcgggagtacttcaagtatttttccaccgagctgacatctagcctgggaggaccctcctctggcgccccaccacctagcggcggctcccctgccggctctccaaccagcacagaggagggcaccagcgagtccgccacaccagagtctggacctggcaccagcacagagccatccgagggctctgccccaggctctcctgcaggcagccctacctccaccgaagagggcaccagcacagagccttctgagggcagcgccccaggcacctctacagagccaagcgagctcgaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtgggagtcgacggatccagcggctccgagaccccaggcacatctgagagcgccacccctgagtcccggaccctggtgacattcaaggacgtgttcgtggacttcacccgggaggagtggaagctgctggacacagcccagcagatcgtgtacaggaacgtgatgctggagaactataagaatctggtgtctctgggctaccagctgacaaagccagatgtgatcctgcggctggagaagggagaggagccctggctggtgtag

SEQ ID NO: 168 - RNA, 112 nt, synthetic construct
  source 1..112: mol_type = other RNA
actgcctggctcactcctccgtttaagagctaagctggaaacagcatagcaagtttaaataaggctagtccgttatcaacttgaaaaagtggcaccgagtcggtgctttttt

SEQ ID NO: 169 - DNA, 20 bp, Mus sp.
  source 1..20: mol_type = genomic DNA
actgcctggctcactcctcc

SEQ ID NO: 170 - RNA, 112 nt, synthetic construct
  source 1..112: mol_type = other RNA
atcgtccgatggggctctgggtttaagagctaagctggaaacagcatagcaagtttaaataaggctagtccgttatcaacttgaaaaagtggcaccgagtcggtgctttttt

SEQ ID NO: 171 - DNA, 20 bp, Mus sp.
  source 1..20: mol_type = genomic DNA
atcgtccgatggggctctgg

SEQ ID NO: 172 - protein, 150 aa, synthetic construct
  VARIANT 6..150: note = EAAAK repeats may be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 173 - protein, 120 aa, synthetic construct
  VARIANT 5..120: note = SGGS repeats may be deleted
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 174 - protein, 60 aa, synthetic construct
  VARIANT 3..60: note = XP repeats may be deleted and wherein X can be any amino acid
XPXPXPXPXPXPXPXPXPXPXPXPXPXPXPXPXPXPXPXPXPXPXPXPXPXPXPXPXPXP

SEQ ID NO: 175 - protein, 21 aa, synthetic construct
  VARIANT 4..21: note = 4 or 6 GGS repeats may be deleted
GGSGGSGGSGGSGGSGGSGGS

SEQ ID NO: 176 - protein, 25 aa, synthetic construct
  VARIANT 2..5: note = Any amino acid
  VARIANT 4..5: note = Residue may be deleted
  VARIANT 7..18: note = Any amino acid
  VARIANT 20..24: note = Any amino acid
  VARIANT 23..24: note = Residue may be deleted
CXXXXCXXXXXXXXXXXXHXXXXXH

SEQ ID NO: 177 - protein, 20 aa, synthetic construct
GGGGSGGGGSGGGGSGGGGS

SEQ ID NO: 178 - RNA, 10 nt, synthetic construct
  source 1..10: mol_type = other RNA
aaaaaaaaaa

SEQ ID NO: 179 - protein, 30 aa, synthetic construct
  VARIANT 2..30: note = Residue may be deleted
GGGGGGGGGGGGGGGGGGGGGGGGGGGGGG

SEQ ID NO: 180 - protein, 4 aa, unidentified
  source 1..4: note = WRPW motif of the hairy-related basic helix-loop-helix (bHLH) repressor proteins
WRPW